SEQUENCE LISTING (22 sequences)

SEQ ID NO: 1 - protein, 84 aa, Pisum sativum
MASMISSSAVTTVSRASRGQSAAMAPFGGLKSMTGFPVRKVNTDITSITSNGGRVKCMQVWPPIGKKKFETLSYLPPLTRDSRA

SEQ ID NO: 2 - protein, 57 aa, Pisum sativum
MASMISSSAVTTVSRASRGQSAAMAPFGGLKSMTGFPVRKVNTDITSITSNGGRVKC

SEQ ID NO: 3 - protein, 85 aa, Arabidopsis thaliana
MASSMLSSATMVASPAQATMVAPFNGLKSSAAFPATRKANNDITSITSNGGRVNCMQVWPPIEKKKFETLSYLPDLTDSGGRVNC

SEQ ID NO: 4 - protein, 76 aa, Arabidopsis thaliana
MAQVSRICNGVQNPSLISNLSKSSQRKSPLSVSLKTQQHPRAYPISSSWGLKKSGMTLIGSELRPLKVMSSVSTAC

SEQ ID NO: 5 - protein, 76 aa, Arabidopsis thaliana; identical to SEQ ID NO: 4

SEQ ID NO: 6 - protein, 72 aa, Petunia x hybrida
MAQINNMAQGIQTLNPNSNFHKPQVPKSSSFLVFGSKKLKNSANSMLVLKKDSIFMQKFCSFRISASVATAC

SEQ ID NO: 7 - protein, 69 aa, Triticum aestivum
MAALVTSQLATSGTVLSVTDRFRRPGFQGLRPRNPADAALGMRTVGASAAPKQSRKPHRFDRRCLSMVV

SEQ ID NO: 8 - protein, 77 aa, Oryza sativa
MAALTTSQLATSATGFGIADRSAPSSLLRHGFQGLKPRSPAGGDATSLSVTTSARATPKQQRSVQRGSRRFPSVVVC

SEQ ID NO: 9 - protein, 57 aa, Nicotiana tabacum
MASSVLSSAAVATRSNVAQANMVAPFTGLKSAASFPVSRKQNLDITSIASNGGRVQC

SEQ ID NO: 10 - protein, 65 aa, Zea mays
MESLAATSVFAPSRVAVPAARALVRAGTVVPTRRTSSRSGTSGVKCSAAVTPQASPVISRSAAAA

SEQ ID NO: 11 - protein, 72 aa, Ruta graveolens
MGAAATSMQSLKFSNRLVPPSRRLSPVPNNVTCNNLPKSAAPVRTVKCCASSWNSTINGAAATTNGASAASS

SEQ ID NO: 12 - DNA, 252 bp, Pisum sativum
atggcttctatgatatcctcttccgctgtgacaacagtcagccgtgcctctagggggcaatccgccgcaatggctccattcggcggcctcaaatccatgactggattcccagtgaggaaggtcaacactgacattacttccattacaagcaatggtggaagagtaaagtgcatgcaggtgtggcctccaattggaaagaagaagtttgagactctttcctatttgccaccattgacgagagattcccgggcc

SEQ ID NO: 13 - DNA, 171 bp, Pisum sativum
atggcttctatgatatcctcttccgctgtgacaacagtcagccgtgcctctagggggcaatccgccgcaatggctccattcggcggcctcaaatccatgactggattcccagtgaggaaggtcaacactgacattacttccattacaagcaatggtggaagagtaaagtgc

SEQ ID NO: 14 - DNA, 255 bp, Arabidopsis thaliana
atggcttcctctatgctctcttccgctactatggttgcctctccggctcaggccactatggtcgctcctttcaacggacttaagtcctccgctgccttcccagccacccgcaaggctaacaacgacattacttccatcacaagcaacggcggaagagttaactgtatgcaggtgtggcctccgattgaaaagaagaagtttgagactctctcttaccttcctgaccttaccgattccggtggtcgcgtcaactgc

SEQ ID NO: 15 - DNA, 228 bp, Arabidopsis thaliana
atggcgcaagttagcagaatctgcaatggtgtgcagaacccatctcttatctccaatctctcgaaatccagtcaacgcaaatctcccttatcggtttctctgaagacgcagcagcatccacgagcttatccgatttcgtcgtcgtggggattgaagaagagtgggatgacgttaattggctctgagcttcgtcctcttaaggtcatgtcttctgtttccacggcgtgc

SEQ ID NO: 16 - DNA, 228 bp, Artificial Sequence; identical to SEQ ID NO: 15
  Synthetic codon optimized nucleotide sequence with CTP2

SEQ ID NO: 17 - DNA, 216 bp, Artificial Sequence
  Synthetic codon optimized nucleotide sequence with CTP4
atggcccagatcaacaacatggcccagggcatccagaccctgaaccctaactctaacttccacaagccgcaagtgcccaagtctagctccttcctcgtgttcggctccaagaagctcaagaatagcgccaattccatgctggtcctgaagaaagactcgatcttcatgcagaagttctgctcctttcgcatcagtgcttcggttgcgactgcctgc

SEQ ID NO: 18 - DNA, 207 bp, Artificial Sequence
  Synthetic codon optimized nucleotide sequence with TaWaxy CTP
atggcggcactggtgacctcccagctcgcgacaagcggcaccgtcctgtcggtgacggaccgcttccggcgtcccggcttccagggactgaggccacggaacccagccgatgccgctctcgggatgaggacggtgggcgcgtccgcggctcccaagcagagcaggaagccacaccgtttcgaccgccggtgcttgagcatggtcgtc

SEQ ID NO: 19 - DNA, 231 bp, Oryza sativa
atggcggctctcaccacgtcccagctcgccacctcggccaccggcttcggcatcgccgacaggtcggcgccgtcgtcgctgctccgccacgggttccagggcctcaagccccgcagccccgccggcggcgacgcgacgtcgctcagcgtgacgaccagcgcgcgcgcgacgcccaagcagcagcggtcggtgcagcgtggcagccggaggttcccctccgtcgtcgtgtgc

SEQ ID NO: 20 - DNA, 171 bp, Nicotiana tabacum
atggcttcctcagttctttcctctgcagcagttgccacccgcagcaatgttgctcaagctaacatggttgcacctttcactggccttaagtcagctgcctcattccctgtttcaaggaagcaaaaccttgacatcacttccattgccagcaacggcggaagagtgcaatgc

SEQ ID NO: 21 - DNA, 195 bp, Zea mays
atggaatccctagccgccacctccgtgttcgcgccctcccgcgtcgccgtcccggcggcgcgggccctggttagggcggggacggtggtaccaaccaggcggacgagcagccggagcggaaccagcggggtgaaatgctctgctgccgtgacgccgcaggcgagcccagtgattagcaggagcgctgcggcggcc

SEQ ID NO: 22 - DNA, 216 bp, Ruta graveolens
atgggtgcagcggcaacgtcgatgcaatcccttaaattctccaaccgtctggtcccacccagtcgccgtctgtctccggttccgaacaatgtcacctgcaataacctccccaagtctgcagctcccgtccggacagtcaaatgctgcgcttcttcctggaacagtaccatcaacggcgcggccgccacgaccaacggtgcgtccgccgccagtagc

(19) United States
(12) Patent Application Publication
(10) Pub. No.: US 2019/0150383 A1
(43) Pub. Date: May 23, 2019

(54) METHODS FOR HYBRID CORN SEED PRODUCTION AND COMPOSITIONS PRODUCED THEREFROM

(71) Applicant: Monsanto Technology LLC, St. Louis, MO (US)

(72) Inventors: Rajendra D. Ketkar, Wildwood, MO (US); Jay Cartmell Pershing, St. Louis, MO (US); Michael A. Stephens, Chesterfield, MO (US)

(21) Appl. No.: 16/266,827

(22) Filed: Feb. 4, 2019

Related U.S. Application Data
(63) Continuation of application No. 12/440,160, filed on Jun. 15, 2010, now abandoned.
(60) Provisional application No. 60/922,013, filed on Apr. 5, 2007.
(60) Provisional application No. 60/848,952, filed on Oct. 3, 2006.

(51) Int. Cl. A01H 1/00; A01H 1/04; A01H 5/10

(52) U.S. Cl. CPC A01H 1/00; A01H 5/10; A01H 1/04

(57) ABSTRACT

A method for plant breeding by identifying, balancing, and combining multiple traits is provided. The method enhances the profitability of a hybrid corn seed production system by providing traits having high impact on decreasing cost of production on female parent and traits having low impact on decreasing cost of production and end users traits on the female and/or the male parent. The invention also provides a hybrid corn seed produced by such method.

### CROSS REFERENCE TO RELATED APPLICATIONS

This application claims the priority of U.S. provisional application Ser. Nos. 60/848,952 (filed Oct. 3, 2006) and 60/922,013 (filed Apr. 5, 2007), the entire disclosure of which is incorporated herein by reference.

### BACKGROUND

### Field of the invention

The invention relates to the field of manufacturing methods and manufactured compositions. More specifically, it relates to methods for hybrid corn seed production and hybrid corn compositions produced through such methods.

### Related art

Commercial hybrid corn seed production involves planting male and female inbred lines in separate rows or blocks in an isolated field where possibility of foreign pollen contamination is rare. The female inbred is normally detasselled before pollen shed to ensure cross-pollination by the male inbred. Male inbreds are destroyed following pollination to prevent seed mixing during harvest. Ears from the cross-pollinated female inbred are harvested, processed, and sold to farmers for planting as hybrid seed. Production of hybrid corn seed is an expensive process due to manual or mechanical detasseling and input costs of herbicides, insecticides, fungicides, nutrients, and irrigation. The yield of hybrid seed tends to be lower resulting in lower revenues. Lower revenues and increasing cost of production result in lower profitability for manufacturers. Manufacturers of hybrid corn seed, thus, are always interested in enhancing profitability of a hybrid corn seed production system.

It is also known in the art that the cost of producing homozygous inbreds with multiple traits for creating hybrids is quite high and becomes higher with the introduction of each additional trait as breeders need to ensure a right combination of traits, right position of these traits in the genome, and integration of these traits in the right germplasm. The probability of obtaining the right homozygous output is 1:4 in case of producing an inbred with one trait and is 1:65,536 in case of producing an inbred with eight traits. These probabilities exponentially increase the difficulty in identifying a homozygous inbred with multiple traits and consequently raising the breeding costs substantially. It is important then to identify and combine suitable traits that will result in enhanced profitability of a corn seed production system.

The cost of breeding an uneven number of traits into a hybrid is similar to the breeding cost of even number of traits. Also, in certain situations, a trait may have adverse effect on a parent. There is also a need to balance the number of traits on the male and the female inbreds to reduce the cost of breeding these traits into a hybrid per se and to provide the trait causing agronomic adverse effect in one parent to another parent.

Further, due to evolving market and environmental conditions, there is increasing demand for well-adapted germplasm by end users, such as farmers and processors, comprising traits that enhance yield, promote stress resistance, and enhance grain quality.

Although, hybrid corn seeds with up to three traits are known in the market place and different methods of introducing the multiple traits are known, the current state of the art lacks a systematic method for delivering at least four transgenic traits in hybrid corn seed, such as ones described herein. That is, the art lacks a systematic means for identification and selection of cost decreasing traits, deciding which trait or traits should be provided on which parent, and then selecting a combination of traits from each parent to enhance the profitability of a hybrid corn seed production system.

### SUMMARY OF THE INVENTION

A method for hybrid corn seed production system comprising: identifying at least one transgenic trait having a high impact on decreasing cost of production, wherein the trait is introduced into germplasm of a female parent; identifying at least three transgenic traits having a low impact on decreasing cost of production, wherein the traits are introduced into germplasm of a male parent and/or the female parent; planting and crossing the male and the female parent; and harvesting a hybrid seed, wherein the production of the hybrid seed results in enhanced profitability over a hybrid seed not produced by providing the transgenic traits on the female and the male parent.

The method further comprising balancing the transgenic traits on the male parent and the female parent, wherein the production of the hybrid seed results in an enhanced profitability as compared with the profitability of a hybrid seed produced by not balancing the transgenic traits.

In one embodiment, the method comprises identifying at least four transgenic traits having a low impact on decreasing cost of production, wherein the traits are introduced into germplasm of the male parent and/or the female parent.

In another embodiment, the method identifying at least five transgenic traits having a low impact on decreasing cost of production, wherein the traits are introduced into germplasm of the male parent and/or the female parent.

In another embodiment, the trait providing the high impact on decreasing cost of production is selected from the group consisting of herbicide tolerance, male sterility system, enhanced yield, and nutrient use efficiency, and a combination thereof. Examples of these and other traits having the high impact on decreasing cost of production are also given in one or more of Tables 2A-9.

In another embodiment, the trait providing the low impact on decreasing cost of production is selected from the group consisting of cold tolerance, drought tolerance, diseases resistance, insect resistance, and a combination thereof. Examples of these and other traits having the low impact on decreasing cost of production are also given in one or more of Tables 2A-9.

In another embodiment, the enhanced profitability is achieved by increasing yield of the hybrid corn seed.

In another embodiment, the method of present invention further comprises providing an end user transgenic trait on the female and/or the male parent. The end user trait is selected from the group consisting of enhanced amino acid content, enhanced protein content, modified or enhanced fatty acid composition, enhanced oil content, enhanced carbohydrate content, and a combination thereof. Examples of these and other end users traits are also given in Tables 10A and 10B.

The present invention also discloses a hybrid corn seed produced by the method of present invention.

In one embodiment the traits in the hybrid seed consist of one or more of the herbicide tolerance traits and three or more of the insect resistance traits.

In another embodiment the traits in the hybrid seed consists of one or more of the herbicide tolerance traits, two or more of the insect resistance traits, and the drought tolerance trait.

In another embodiment the traits in the hybrid seed consist of one or more of the herbicide tolerance traits, two or more of the insect resistance traits, the drought tolerance trait, and the male sterility system trait.

In another embodiment the traits in the hybrid seed consist of one or more of the herbicide tolerance traits, two or more of the insect resistance traits, the drought tolerance trait, the male sterility system trait, and the intrinsic yield trait.

In another embodiment the traits in the hybrid seed consist of one or more of the herbicide tolerance traits, two or more of the insect resistance traits, one or more of the drought tolerance traits, the male sterility system trait, the intrinsic yield trait, and the nutrient use efficiency trait.

In another embodiment the traits in the hybrid seed consist of one or more of the herbicide tolerance trait, two or more of the insect resistance traits, one or more of drought tolerance traits, the male sterility system trait, the intrinsic yield trait, the nutrient use efficiency trait, and the cold tolerance trait.

In another embodiment the traits in the hybrid seed consist of one or more of the herbicide tolerance traits, three or more of the insect resistance traits, and the enhanced amino acid content.

In another embodiment the traits in the hybrid seed consist of one or more of the herbicide tolerance traits, two or more of the insect resistance traits, the enhanced amino acid content trait, and the drought tolerance trait.

In another embodiment the traits in the hybrid seed consist of one or more of the herbicide tolerance traits, two or more of the insect resistance traits, the enhanced amino acid content trait, the drought tolerance trait, and the male sterility system trait.

In another embodiment the traits in the hybrid seed consist of one or more of the herbicide tolerance traits, one two or more of the insect resistance traits, the enhanced amino acid content trait, the drought tolerance trait, the male sterility system trait, and the intrinsic yield trait.

In another embodiment the traits in the hybrid seed consist of one or more of the herbicide tolerance traits, two or more of the insect resistance traits, the enhanced amino acid content trait, the drought tolerance trait, the male sterility system trait, the intrinsic yield trait, and the enhanced oil content trait.

In another embodiment the traits in the hybrid seed consist of one or more of the herbicide tolerance traits, one two or more of the insect resistance traits, one or more of the enhanced amino acid content trait, one or more of the drought tolerance trait, the male sterility system trait, the intrinsic yield trait, nutrient use efficiency trait, the enhanced oil content trait, the enhanced protein content trait, and the cold tolerance trait.

In another embodiment the traits in the hybrid seed consist of one or more of the herbicide tolerance traits, two or more of the insect resistance traits, one or more of the enhanced amino acid content trait, one or more of the drought tolerance trait, the male sterility system trait, the intrinsic yield trait, the nutrient use efficiency trait, the enhanced oil content trait, the enhanced protein content trait, and the cold tolerance trait.

### BRIEF DESCRIPTION OF THE FIGURES

FIG. 1 illustrates arrangement of various cost decreasing traits on a scale, right to left, ranging from having high impact on decreasing cost of production to low impact on decreasing cost of production. HT: herbicide tolerance, MSS: male sterility system, IY: intrinsic yield, NUE: nutrient use efficiency, CT: cold tolerance, DR: disease resistance, DT: drought tolerance, and IR: insect resistance. In general, traits having high impact on decreasing cost of production are provided on the female parent whereas traits having low impact on decreasing cost of production are provided on the male and/or the female parent.

### DETAILED DESCRIPTION OF THE INVENTION

The following is a detailed description of the invention provided to aid those skilled in the art in practicing the present invention. Those of ordinary skill in the art may make modifications and variations in the embodiments described herein without departing from the spirit or scope of the present invention.

Unless otherwise defined, all technical and scientific terms used herein have the same meaning as commonly understood by one of ordinary skill in the art to which this invention belongs. The terminology used in the description of the invention herein is for describing particular embodiments only and is not intended to be limiting of the invention. As used in the description of the invention and the appended claims, the singular forms “a,” “an,” and “the” are intended to include the plural forms as well, unless the context clearly indicates otherwise. All publications, patent applications, patents, and other references mentioned herein are expressly incorporated by reference in their entirety.

The word “comprising” means including but not limited to.

The present invention provides a method for enhancing profitability of a hybrid corn seed production system. This is achieved by identifying and providing traits having high impact on decreasing cost of production or low impact on decreasing cost of production. Some of the cost decreasing traits may also influence yield directly or indirectly thereby enhancing revenues and profitability. For example, an intrinsic yield trait can decrease costs associated with production land by enhancing yield i.e., more units of hybrid corn seed can be produced on less land. Although, hybrid corn seeds with up to three traits are known in the market place, there does not appear to be a systematic method such as the one described herein available for identifying, selecting which cost decreasing traits should be provided on which parent and then selecting a combination of traits on each parent to enhance profitability of the hybrid corn seed production system.

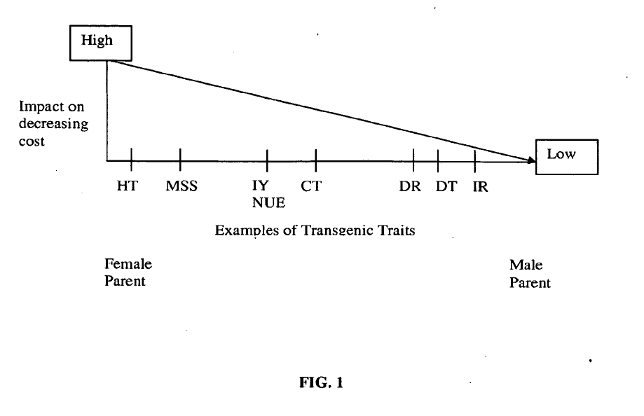

As shown in FIG. 1, exemplary cost decreasing traits are ranked on a scale, left to right, ranging from having a high impact on decreasing cost of production to having a low impact on decreasing cost of production, in the following order: herbicide tolerance, male sterility system, intrinsic yield, nutrient use efficiency, cold tolerance, disease resistance, drought tolerance, and insect resistance. Any other trait may also be ranked on this gradient based on their high or low impact on decreasing cost of production. In general, traits having high impact on decreasing cost of production are provided on the female parent whereas traits having low impact on decreasing cost of production are provided on the male or the female parent.

Traits having high impact on decreasing cost of production may be balanced by inclusion of traits having a low impact on decreasing cost of production, by selecting certain traits having high impact and certain traits having low impact from a gradient of traits such that the profitability of the hybrid corn seed production system is enhanced. For example, if the goal is to combine 7 traits in a hybrid, one may select, for example from Table 1, herbicide resistance, male sterility system, an intrinsic yield trait, and nitrogen use efficiency as high impact traits and introduce them into the female parent. One may also select cold tolerance, disease resistance, and drought resistance traits as low impact traits, and introduce them into the female or the male parent. In some embodiments, the traits may be balanced as±one or two or three traits on the female or the male parent to enhance profitability. In other embodiments, the traits may be balanced as±one or two or three traits on the female or the male parent to enhance profitability.

In some instances, provision of a trait on female parent may cause a yield penalty, e.g., due to its insertion into an important endogenous gene. In such situations, the trait may be provided on the male parent.

Thus, one aspect of the present invention provides a method for hybrid corn seed production comprising: identifying at least one transgenic trait having a high impact on decreasing cost of production, wherein the trait is introduced into germplasm of a female parent; identifying at least three transgenic traits having a low impact on decreasing cost of production, wherein the traits are introduced into germplasm of a male parent and/or the female parent; planting and crossing the male and the female parent; and harvesting a hybrid seed, wherein the production of the hybrid seed results in enhanced profitability over a hybrid seed not produced by providing the transgenic traits on the female and the male parent.

The method facilitates crop breeding decisions, for instance by allowing for balancing of transgenic traits on the male parent and the female parent, wherein production of the resulting hybrid seed results in an enhanced profitability over a hybrid seed produced by not balancing the transgenic traits.

The method may comprise identifying at least four transgenic traits having a low impact on decreasing cost of production, wherein the traits are introduced into germplasm of the male parent and/or the female parent. Alternatively, the method allows for identifying at least five transgenic traits having a low impact on decreasing cost of production, wherein the traits are introduced into germplasm of the male parent and/or the female parent.

In another embodiment of the method, a trait or traits providing a high impact on decreasing cost of production may be selected from the group consisting of herbicide tolerance, male sterility system, enhanced yield, and nutrient use efficiency, and a combination thereof. Examples of traits having the high impact on decreasing cost of production are also given in one or more of Tables 2A-9.

In another embodiment, a trait providing a low impact on decreasing cost of production may be selected from the group consisting of cold tolerance, drought tolerance, disease resistance, insect resistance, and a combination thereof. Examples of traits having the low impact on decreasing cost of production are also given in one or more of Tables 2A-9.

Enhanced profitability may also be achieved by increasing the yield of a hybrid corn seed production system.

The present invention also discloses hybrid corn seed produced by the method of the present invention. In one embodiment, traits in the hybrid seed may consist of one or more herbicide tolerance traits and three or more insect resistance traits. In another embodiment traits in the hybrid seed may consist of one or more herbicide tolerance traits, two or more insect resistance traits, and a drought tolerance trait. In yet another embodiment, traits in the hybrid seed may consist of one or more herbicide tolerance traits, two or more insect resistance traits, a drought tolerance trait, and a male sterility system trait. In another embodiment the traits in the hybrid seed may consist of one or more herbicide tolerance traits, two or more insect resistance traits, a drought tolerance trait, a male sterility system trait, and an intrinsic yield trait. In a further embodiment, traits in the hybrid seed may consist of one or more herbicide tolerance traits, two or more insect resistance traits, one or more drought tolerance traits, a male sterility system trait, an intrinsic yield trait, and a nutrient use efficiency trait. In yet another embodiment the traits in the hybrid seed may consist of one or more herbicide tolerance trait, two or more insect resistance traits, one or more drought tolerance traits, a male sterility system trait, an intrinsic yield trait, a nutrient use efficiency trait, and a cold tolerance trait. Examples of such traits may be found in one or more of Tables 2A-9.

A trait specifying a phenotype (e.g. an agronomic trait such as herbicide tolerance) may be encoded by one gene or by more than one gene specifying the same or a different mode of action or mechanism.

Nucleic acids encoding for a trait or traits specifying abiotic stress resistance, such as drought, cold, salt, or nutrient stress resistance, may also provide tolerance to more than one stress.

Cost decreasing traits may include traits that provide increased herbicide tolerance, male sterility system, increased intrinsic yield, increased nutrient use efficiency e.g., nitrogen use efficiency, increased cold tolerance, increased disease resistance, increased drought tolerance, and increased insect resistance. These traits may decrease cost of production by any where from 14.2% to 0.1% or more (see Table 1).

TABLE 1
Estimated decrease in cost that can be realized by utilizing cost
decreasing traits in producing 6,000,000 units with 86 usable units/acre.

| | Decrease in Cost |
|---|---|
| Traits | (% of production cost) |
| Herbicide tolerance | 14.2% |
| Male Sterility System | 10.8% |
| Intrinsic Yield (10% Improvement) | 3.7% |
| Nitrogen Use Efficiency (10% Yield | 3.7% |
| Improvement) | |
| Cold Tolerance | 2.6% |
| Disease Resistance | 0.7% |
| Drought tolerance | 0.5% |
| Insect Resistance | 0.1% |

Provision of an herbicide tolerance trait in a parent in combination with use of a corresponding herbicide can be used to manage weeds, thereby reducing the utilization of resources by weeds and decreasing the need for inputs such as nutrients and water. This trait can also enhance yield as more resources will be available for growing the hybrid parents. Further, it has been found that providing an herbicide tolerance trait, for example a glyphosate tolerance trait, on a female parent may reduce the production failure rate, i.e. the estimated chance of not producing pure seed, to 0.06% to 10% as measured by trait purity in a seed lot, for example by a measured glyphosate susceptibility rate equal to or more than 2%. This compares to a possible failure rate of 36.1% if the glyphosate tolerant trait is provided on the male parent. An increase in production failure rate means an incremental rise in production acres to reproduce units not meeting the susceptibility tolerance criteria thereby increasing cost of hybrid seed production. By providing an herbicide tolerance trait, such as glyphosate tolerance, on a female parent, a decrease in cost of about 14.2% can be realized resulting in enhanced profitability (Table 1).

One or more herbicide tolerance traits can be provided to control weeds more effectively and to reduce the risk of developing herbicide resistance weeds in a field. For example, herbicide tolerance traits providing tolerance to glyphosate, glufosinate, dicamba, or 2,4-D can be provided. When more than one herbicide tolerant trait is provided then it may be provided on the male and/or the female parent. When a herbicide tolerance trait is provided as a part of a male sterility system (described below), the herbicide tolerance trait may also be provided on the male parent, since the application of the herbicide may render the pollen on a female parent non-viable. In order to provide viable pollen from a male parent, the male parent preferably has a corresponding herbicide tolerance trait to survive the application of the herbicide in the production system. Examples of proteins responsible for herbicide tolerance are exhibited in Tables 2A and 2B.

Various methods can be used to prevent self pollination of the female parent. In conventional plant breeding schemes, at the time of flowering, the tassels of all female parents are typically removed. The detasseling can be achieved manually or by machine. This technique, while effective, is extremely labor intensive and greatly increases the overall cost of hybrid seed production. Alternatively, conventional nuclear or cytoplasmic male sterility systems may be used. The present invention uses biotechnological and chemical male sterility systems (MSS) as a cost decreasing trait. By using such methods a decrease in cost of about 10.8% can be realized resulting in enhanced profitability (Table 1). Examples of such methods can be found in documents listed in Tables 2A and 2B which are also incorporated herein by reference.

By providing a yield trait on a female parent, the number of usable units harvested per acre can be increased. An increase in usable units/per acre means a proportional decrease in the number of acres needed to realize a given unit yield target. For example, a 10% intrinsic yield gain can result in a 10% decrease in the number of acres required to produce hybrid corn. By providing an intrinsic yield trait on a female parent, a decrease in cost of about 3.7% can be realized resulting in enhanced profitability (Table 1). A yield trait may increase yield by improving biomass, grain yield, number of seeds, germination, and high density growth of plants. Examples of proteins responsible for yield traits are exhibited in Tables 2A and 2B to 9.

Some traits that are able to provide tolerance to abiotic stresses such as nutrient deficiency are also cost decreasing traits by allowing reduced use of nutrient inputs, such as nitrogen, or by increasing yield when a given level of nutrient inputs (e.g. fertilizer) is applied. This may subsequently reduce the requirement for production acreage. By providing a trait for nitrogen use efficiency on a female parent a decrease in cost of about 3.7% can be realized resulting in enhanced profitability (Table 1). Examples of proteins responsible for nutrient use efficiency traits are exhibited in Tables 2A and 2B to 9.

Other stress tolerance traits such as a cold tolerance trait, for instance a cold germination tolerance trait, can result in cold tolerance. Assuming a 1″ improvement in standard deviation of seed spacing in final stand of female parents, a 7% increase in yield can be realized, thereby enabling reduced requirement of production acreage and a decrease in cost of about 2.6% resulting in enhanced profitability (Table 1). The cold tolerance trait can be provided on the male and/or the female to optimize the production cost decrease. Examples of proteins responsible for cold stress tolerance are exhibited in Tables 2A and 2B to 9.

A disease resistance trait may be provided on a male parent because the impact of a disease resistance trait on decreasing production cost is typically low. Assuming 99% of total acres is sprayed with fungicides with one application per acre per year at an application cost of $15.10, providing a trait for disease resistance can result in a cost decrease of about 0.7% resulting in enhanced profitability (Table 1). The disease resistance trait may be provided on the female parent in certain situations, for example, if the female is made male sterile by detasseling. Detasseling may further cause wounding. Thus, it may be beneficial to protect the female from any infection through wounds left after detasseling by providing a resistance trait against, for example, fungal diseases such as gray leaf spot or rust diseases, that can seriously harm a corn plant. Disease resistance traits, such as those effective against Helminthosporium carbonum or common rust, may be of further benefit in decreasing cost in a production system. Examples of proteins responsible for disease resistance are exhibited in Tables 2A and 2B to 9

Provision of another trait for combating abiotic stresses such as lack of water can also decrease cost of producing hybrid seed by allowing reduced use of water on irrigated land or increasing yield on dry land. For example, in a 6,000,000 unit production plan at 86 usable units/acre, assuming use of 70% irrigated acres with an irrigation cost of $8.00/acre/inch and the water requirement of 23″/year, with a 10% reduction in irrigation cost and 5% increase in yield on dry land, a producer could realize a decrease in cost of 0.5% in hybrid seed production. This trait can be provided on the male or the female or on both given its low impact on decreasing cost of production (see Table 1). Examples of proteins responsible for drought tolerance are exhibited in Tables 2A and 2B to 9.

With the use of an insect resistance trait in a parent, no foliar or soil insecticidal treatments may be required. For example, in a 6,000,000 unit production plan at 86 usable units/acre, assuming 26.4% of the acres will be sprayed for 1st generation European corn borer, with the female parent representing 77.3% of the total acres, a custom application cost of $5.35/acre, a chemical cost of $6.56/acre, and an application rate of 4 oz/acre, provision of a trait against protection for European corn borer could reduce chemical applications by 80-90% resulting in decrease in total production cost of about 0.1% (see Table 1), as well as environmental benefits. Provision of traits providing protection against several insects such as root worms, spider mites, grasshoppers, Western bean cutworm or other cutworms, or earworms could decrease cost of production by 0.75%. Such protection against several insects can be obtained by combining novel and chimeric genes and/or RNAi methods. One or more insect resistance traits can be provided on the female or the male parent or both. Examples of proteins responsible for insect resistance are exhibited in Tables 2A and 2B to 9.

The nucleic acids encoding proteins that confer insect resistance can be derived from a number of organisms that include, but are not limited to, Bacillus thuringiensis, Xenorhabdus sp., or Photorhabdus sp. For example, transgenic plants which express one or more B. thuringiensis proteins toxic to the same insect species or multiple insect species can be produced in order to allow for resistance management, which may delay the onset of resistance in a population of an otherwise susceptible insect species to one or more of the insecticidal nucleic acids expressed within the transgenic plant. Alternatively, expression of a B. thuringiensis insecticidal protein toxic to a particular target insect pest along with a different proteinaceous agent toxic to the same insect pest but which confers toxicity by a means different from that exhibited by the B. thuringiensis toxin is desirable. Such other different proteinaceous agents may comprise any of Cry insecticidal proteins, Cyt insecticidal proteins, insecticidal proteins from Xenorhabdus sp. or Photorhabdus sp., B. thuringiensis vegetative insecticidal proteins, and the like. Examples of such proteins encoded by insect toxin genes includes, but are not limited to, ET29, TIC809, TIC810, TIC127, TIC128, TIC812 and ET37 (WO 07/027776), TIC807, AXMI-027, AXMI-036, and AXMI-038 (WO 06/107761), AXMI-018, AXMI-020, and AXMI-021 (WO 06/083891), AXMI-010 (WO 05/038032), AXMI-003 (WO 05/021585), AXMI-008 (US 2004/0250311), AXMI-006 (US 2004/0216186), AXMI-007 (US 2004/0210965), AXMI-009 (US 2004/0210964), AXMI-014 (US 2004/0197917), AXMI-004 (US 2004/0197916), AXMI-028 and AXMI-029 (WO 06/119457) and AXMI-007, AXMI-008, AXMI-0080rf2, AXMI-009, AXMI-014 and AXMI-004 (WO 04/074462). All of the foregoing references are incorporated herein in their entirety.

Proteins conferring insect resistance are preferably toxic against coleopteran insect pests that comprises of coleopteran families consisting of Chrysomelidae, Cucujidae, Scarabaeidae, Trogositidae, Tenebrionidae, Curculionidae, Elateridae and Bruchidae. The exemplary coleopteran insects in the family Chrysomelidae may include those that are from the genus Diabrotica including D. virgifera (WCR), D. undecimpunctata (SCR), D. barberi (NCR), D. virgifera zeae (MCR), D. balteata (BZR), and Brazilian Corn Rootworm complex (BCR) consisting of D. viridula and D. speciosa.

A protein conferring insect resistance may also be toxic against hemipteran insect pests that may be selected from the group of hemipteran suborders consisting of Auchenorrhyncha (e.g., cicadas, spittlebugs, hoppers), Sternorrhyncha (e.g., aphids, whiteflies, scales), Heteroptera (e.g., true bugs including Lygus) and Coleorrhyncha. The hemipteran insects can be from the suborder Heteroptera. Exemplary hemipteran insects in the suborder Heteroptera may include those that are from the genus Lygus including Lygus hesperus (western tarnished plant bug), Lygus lineoloris (tarnished plant bug) and Lygus elisus (pale western legume bug).

A protein conferring insect resistance may also be toxic against a Lepidopteran insect pest such as European corn borer (Ostrinia nubilalis), Southwestern corn borer (Diatraea grandiosella), Sugarcane borer (Diatraea saccharalis), Corn earworm (Helicoverpa zea), Fall armyworm (Spodoptera frugiperda), Black cutworm (Agrotis ipsilon) and Western bean cutworm (Loxagrotis albiocosta).

A protein conferring insect resistance can be encoded by one or more genes encoding toxins to nematodes which attack crops. Some exemplary nematode species affecting corn are the corn cyst nematode (Heterodera zeae), the Root knot nematode (Meloidogyne spp.), and the sting nematode (Belonolaimus longicaudatus).

The decrease in cost of production associated with provision of certain traits described above could even be higher for traits where reduced material inputs such as nutrient, fungicide, water, and insecticide would also results in reduced costs related to equipment, labor, fuel, and personnel safety.

In addition to cost decreasing transgenic traits, end user transgenic traits may be added to the female and/or male parent. These traits are considered neutral in terms of enhancing profitability of a hybrid corn production system to a producer. However, these traits will be of benefit to the end users, such as farmers and processors, of hybrid seed. Such end users traits include feed quality, food quality, processing, pharmaceutical, and industrial traits. Example of proteins responsible for end user traits are exhibited in Tables 10A and 10B.

In one embodiment, the method of the present invention further comprises providing an end user transgenic trait on the female and/or the male parent. An end user trait may be defined for this purpose as a trait that requires identity preservation by the end users. Examples of these traits are also given in Tables 10A and 10B. The end user trait may be selected from the group consisting of enhanced amino acid content, enhanced protein content, modified or enhanced fatty acid composition, enhanced oil content, enhanced carbohydrate content, and a combination thereof. Examples of these and other end users traits are also given in Tables 10A and 10B.

The present invention also discloses a hybrid corn seed produced by the method of the present invention.

In another embodiment the traits in the hybrid seed may consist of one or more herbicide tolerance traits, three or more insect resistance traits, and an enhanced amino acid content trait.

The traits in the hybrid seed may consist of one or more of the herbicide tolerance traits, two or more of the insect resistance traits, the enhanced amino acid content trait, and the drought tolerance trait. The traits in the hybrid seed may also consist of one or more of the herbicide tolerance traits, two or more of the insect resistance traits, the enhanced amino acid content trait, the drought tolerance trait, and the male sterility system trait. Alternatively, the traits in the hybrid seed may consist of one or more herbicide tolerance traits, one two or more insect resistance traits, an enhanced amino acid content trait, a drought tolerance trait, a male sterility system trait, and an intrinsic yield trait.

Nucleic acid sequences encoding proteins that confer cost decreasing traits or end-user traits are operably linked to various expression elements to create one or more expression units. These expression units generally comprise in 5′ to 3′ direction: a promoter (usually with one or more enhancers), a nucleic acid encoding a trait of interest, and a 3′ untranslated region. Other expression elements such as a 5′UTRs, organelle transit peptide sequences, and introns may be added to facilitate expression of the trait. Also, instead of using a nucleic acid encoding a trait, one may alternatively provide a nucleic acid sequence for transcription of an RNA molecule for instance via an RNAi-mediated approach in order to manipulate the expression of an endogenous or heterologous gene. Such methods are well in the art.

In another embodiment the traits in the hybrid seed may consist of one or more herbicide tolerance traits, two or more insect resistance traits, an enhanced amino acid content trait, a drought tolerance trait, a male sterility system trait, an intrinsic yield trait, and an enhanced oil content trait.

In another embodiment the traits in the hybrid seed consist of one or more herbicide tolerance traits, one two or more insect resistance traits, one or more enhanced amino acid content trait, one or more drought tolerance traits, a male sterility system trait, an intrinsic yield trait, a nutrient use efficiency trait, an enhanced oil content trait, an enhanced protein content trait, and a cold tolerance trait.

In another embodiment the traits in the hybrid seed may consist of one or more herbicide tolerance traits, two or more insect resistance traits, one or more enhanced amino acid content trait, one or more drought tolerance trait, a male sterility system trait, an intrinsic yield trait, a nutrient use efficiency trait, an enhanced oil content trait, an enhanced protein content trait, and a cold tolerance trait.

Nucleic acids for proteins disclosed in the present invention can be expressed in plant cells by operably linking them to a promoter functional in plants, preferably in monocots, such as corn. Tissue specific and/or inducible promoters may be utilized for appropriate expression of a nucleic acid for a particular trait in a specific tissue or under a particular condition. Examples describing such promoters include U.S. Pat. No. 6,437,217 (maize RS81 promoter), U.S. Pat. No. 5,641,876 (rice actin promoter), U.S. Pat. No. 6,426,446 (maize RS324 promoter), U.S. Pat. No. 6,429,362 (maize PR-1 promoter), U.S. Pat. No. 6,232,526 (maize A3 promoter), U.S. Pat. No. 6,177,611 (constitutive maize promoters), U.S. Pat. Nos. 5,322,938, 5,352,605, 5,359,142 and 5,530,196 (35S promoter), U.S. Pat. No. 6,433,252 (maize L3 oleosin promoter), U.S. Pat. No. 6,429,357 (rice actin 2 promoter as well as a rice actin 2 intron), U.S. Pat. No. 5,837,848 (root specific promoter), U.S. Pat. No. 6,294,714 (light inducible promoters), U.S. Pat. No. 6,140,078 (salt inducible promoters), U.S. Pat. No. 6,252,138 (pathogen inducible promoters), U.S. Pat. No. 6,175,060 (phosphorus deficiency inducible promoters), U.S. Pat. No. 6,635,806 (gamma-coixin promoter), and U.S. patent application publication US 2004-0216189 (maize chloroplast aldolase promoter). Additional promoters that may find use are a nopaline synthase (NOS) promoter (Ebert, et al., 1987, Proc. Natl. Acad. Sci. USA, 84:5745-5749), the octopine synthase (OCS) promoter (which is carried on tumor-inducing plasmids of Agrobacterium tumefaciens), the caulimovirus promoters such as the cauliflower mosaic virus (CaMV) 19S promoter (Lawton, et al., 1987 Plant Mol. Biol. 9: 315-324), the CaMV 35S promoter (Odell, et al., 1985, Nature, 313:810-812), the figwort mosaic virus 35S-promoter (Walker, et al., 1987, Proc. Natl. Acad. Sci. USA, 84:6624), the sucrose synthase promoter (Yang, et al., 1990, Proc. Natl. Acad. Sci. USA, 87:4144-4148), the R gene complex promoter (Chandler, et al., 1989 Plant Cell, 1:1175-1183), and the chlorophyll a/b binding protein gene promoter, etc. In the present invention, CaMV35S with enhancer sequences (U.S. Pat. Nos. 5,322,938; 5,352,605; 5,359,142; and 5,530,196), FMV35S (U.S. Pat. Nos. 6,051,753; 5,378,619), PC1SV (U.S. Pat. No. 5,850,019), Os.Act1 (U.S. Pat. No. 5,641,876), maize globulin 1 promoter (U.S. Pat. No. 6,329,574; US20050132437), maize ubiquitin promoter (US20060037095), rice cytosolic those phosphate isomerase promoter (OsTPI; U.S. Pat. No. 7,132,528), rice actin 15 gene promoter (OsAct15; US Patent Application Publication 20060162010), wheat peroxidase promoter (US2006007013), corn B-32 protein promoter (Hartings et al. 1990, Plant Mol. Biol. 14:1031-1040), wheat peroxidase promoter (GenBank Accession X53675 S54871), and AGRtu.nos promoter (GenBank Accession V00087; Depicker, et al, 1982; Bevan, et al., 1983) may be of particular benefit. In some instances, e.g., OsTPI and OsAct 15, a promoter may include a 5′UTR and/or a first intron. A chimeric promoter may be useful in some instances e.g., a chimera of actin and 35S enhancer and promoter (e.g., see US 2005-0283856).

The 3′ untranslated sequence/region (3′UTR), 3′ transcription termination region, or polyadenylation region is understood to mean a DNA molecule linked to and located downstream in the direction of transcription of a structural polynucleotide molecule responsible for a trait and includes polynucleotides that provide a polyadenylation signal and other regulatory signals capable of affecting transcription, mRNA processing or gene expression. The polyadenylation signal functions in plants to cause the addition of polyadenylate nucleotides to the 3′ end of the mRNA precursor. The polyadenylation sequence can be derived from the natural gene, from a variety of plant genes, or from T-DNA genes. Examples of these include polyadenylation molecules from a Pisum sativum RbcS2 gene (Ps.RbcS2-E9; Coruzzi, et al., 1984, EMBO J., 3:1671-1679) and AGRtu.nos (Rojiyaa, et al., 1987, Genbank Accession E01312). In the present invention, 3′ UTR from the following genes, AGRtu.nos (Rojiyaa, et al., 1987, Genbank Accession E01312), maize globulin 1 (Belanger and Kriz, Genetics, 129:863-872, 1991; US20050132437), E6 (Accession #U30508), ORF25 from Agrobacterium tumefaciens (Barker et al., 1983, Plant Mol. Biol. 2:335-350; US20050039226), and TaHsp17 (wheat low molecular weight heat shock protein gene; GenBank Accession #X13431), and CaMV.35S may be of particular benefit.

A 5′ UTR that functions as a translation leader sequence is a genetic element located between the promoter sequence and the coding sequence. The translation leader sequence is present in the fully processed mRNA upstream of the translation start sequence. The translation leader sequence may affect processing of the primary transcript to mRNA, mRNA stability or translation efficiency. Examples of translation leader sequences include maize and petunia heat shock protein leaders (U.S. Pat. No. 5,362,865), plant virus coat protein leaders, plant rubisco leaders, GmHsp leader (U.S. Pat. No. 5,659,122), PhDnaK leader (U.S. Pat. No. 5,362,865), AtAnt1 leader, TEV leader (Carrington and Freed, Journal of Virology, 64:1590-1597, 1990), and AGRtunos leader (GenBank Accession V00087; Bevan, et al., 1983, Nature, 304:184-187) among others. In the present invention, 5′ UTRs that may in particular find benefit are from the following genes, GmHsp (U.S. Pat. No. 5,659,122), PhDnaK (U.S. Pat. No. 5,362,865), AtAnt1, TEV (Carrington and Freed, 1990, J. Virology 64:1590-1597), wheat major chlorophyll a/b-binding protein (Lamppa et al., 1985, Mol. Cell. Biol. 5, 1370; Genbank accession M10144), AtAnt1 (US Patent Application 20060236420), OsAct1 (U.S. Pat. No. 5,641,876), OsTPI (U.S. Pat. No. 7,132,528), OsAct15 (US Publication No. 20060162010), and AGRtunos (GenBank Accession V00087; Bevan et al., 1983).

In some embodiments, a protein product of a nucleic acid responsible for a particular trait is targeted to an organelle for proper functioning. For example, targeting of a protein to chloroplast is achieved by using a chloroplast transit peptide sequence. These sequences can be isolated or synthesized from amino acid or nucleic acid sequences of nuclear encoded but chloroplast targeted genes such as small subunit (RbcS2) of ribulose-1,5,-bisphosphate carboxylase, ferredoxin, ferredoxin oxidoreductase, the light-harvesting complex protein I and protein II, and thioredoxin F proteins. Other examples of chloroplast targeting sequences include the maize cab-m7 signal sequence (Becker, et al., 1992, Plant Mol. Biol. 20:49; PCT WO 97/41228), the pea glutathione reductase signal sequence (Creissen, et al., 1992, Plant J., 2(1):129-131; PCT WO 97/41228), and the CTP of the Nicotiana tabacum ribulose 1,5-bisphosphate carboxylase small subunit chloroplast transit peptide (NtSSU-CTP) (Mazur, et al., 1985, Nucleic Acids Res., 13:2373-2386). In the present invention, AtRbcS4 (CTP1; U.S. Pat. No. 5,728,925), Petunia hybrida EPSPS CTP (della-Cioppa, et al., 1986), AtShkG (CTP2; Klee, et al., 1987), AtShkGZm (CTP2 synthetic; see SEQ ID NO:14 of WO04009761), and PsRbcS (Coruzzi, et al., 1984) may be used, among others. Other examples of CTPs suitable for the present invention can also be found in SEQ ID NOs:1-22 of this application and in Behrens et al. (Science 316:1185-1189, 2007).

The nucleic acids for traits described herein can be targeted to other organelles such as mitochondria for proper functionality. This can be achieved by adding pre-sequences to a nucleic acid of interest. The nucleic acids can also be targeted to both chloroplast and mitochondria by a dual-targeting peptide to make use of organellar biochemistry more effectively. Such pre-sequence elements are known to those skilled in the art. For example, mitochondrial pre-sequences are described in Silva Filho et al., Plant Mol. Biol. 30:769-780 (1996). Nucleic acid sequences that encode dual-targeting peptide sequences can be identified from the nucleic acids coding for the following proteins which are known be targeted to both chloroplasts and mitochondria: Zn-MP (Moberg et al., Plant J. 36:616-628, 2003), gluthathione reductase (Rudhe et al., J. Mol. Biol. 324:577-585, 2002; Creissen et al., Plant J. 8:167-175, 1995) and histidyl-tRNA synthetase (Akashi et al., FEBS Lett. 431:39-44, 1998).

The term “intron” refers to a polynucleotide molecule that may be isolated or identified from the intervening sequence of a genomic copy of a gene and may be defined generally as a region spliced out during mRNA processing prior to translation. Alternately, introns may be synthetically produced. Introns may themselves contain sub-elements such as cis-elements or enhancer domains that effect the transcription of operably linked genes. A “plant intron” is a native or non-native intron that is functional in plant cells. A plant intron may be used as a regulatory element for modulating expression of an operably linked gene or genes. A polynucleotide molecule sequence in a transformation construct may comprise introns. The introns may be heterologous with respect to the transcribable polynucleotide molecule sequence. Examples of introns useful in the present invention include the corn actin intron and the corn HSP70 intron (U.S. Pat. No. 5,859,347), and rice TPI intron (OsTPI; U.S. Pat. No. 7,132,528).

Duplication of any genetic element across various expression units is avoided due to trait silencing or related effects. Duplicated elements across various expression units are used only when they do not interfere with each other or do not result into silencing of a trait.

Methods are known in the art for assembling and introducing constructs into a cell in such a manner that the nucleic acid molecule for a trait is transcribed into a functional mRNA molecule that is translated and expressed as a protein product. For the practice of the present invention, conventional compositions and methods for preparing and using constructs and host cells are well known to one skilled in the art, see for example, Molecular Cloning: A Laboratory Manual, 3rd edition Volumes 1, 2, and 3 (2000) J. F. Sambrook, D. W. Russell, and N. Irwin, Cold Spring Harbor Laboratory Press. Methods for making transformation constructs particularly suited to plant transformation include, without limitation, those described in U.S. Pat. Nos. 4,971,908, 4,940,835, 4,769,061 and 4,757,011, all of which are herein incorporated by reference in their entirety. These types of vectors have also been reviewed (Rodriguez, et al., Vectors: A Survey of Molecular Cloning Vectors and Their Uses, Butterworths, Boston, 1988; Glick, et al., Methods in Plant Molecular Biology and Biotechnology, CRC Press, Boca Raton, Fla., 1993).

The expression units may be provided between one or more T-DNA borders on a transformation construct designed for Agrobacterium-mediated transformation. The transformation constructs permit the integration of the expression unit between the T-DNA borders into the genome of a plant cell. The constructs may also contain plasmid backbone DNA segments that provide replication function and antibiotic selection in bacterial cells, for example, an Escherichia coli origin of replication such as ori322, a broad host range origin of replication such as oriV or oriRi, and a coding region for a selectable marker such as Spec/Strp that encodes for Tn7 aminoglycoside adenyltransferase (aadA) conferring resistance to spectinomycin or streptomycin, or a gentamicin (Gm, Gent) selectable marker gene. For plant transformation, the host bacterial strain is often Agrobacterium tumefaciens ABI, C58, LBA4404, EHA101, and EHA105 carrying a plasmid having a transfer function for the expression unit. Other strains known to those skilled in the art of plant transformation can function in the present invention.

The traits of the present invention are introduced into inbreds by transformation methods known to those skilled in the art of plant tissue culture and transformation. Any of the techniques known in the art for introducing expression units into plants may be used in accordance with the invention. Examples of such methods include electroporation as illustrated in U.S. Pat. No. 5,384,253; microprojectile bombardment as illustrated in U.S. Pat. Nos. 5,015,580; 5,550,318; 5,538,880; 6,160,208; 6,399,861; and 6,403,865; protoplast transformation as illustrated in U.S. Pat. No. 5,508,184; and Agrobacterium-mediated transformation as illustrated in U.S. Pat. Nos. 5,635,055; 5,824,877; 5,591,616; 5,981,840; and 6,384,301. Preferably, inbreds of corn are transformed by the method disclosed in U.S. Pat. Nos. 5,981,840, 7,060,876, 5,591,616, or WO9506722, US2004244075 and other methods known in the art of corn transformation.

After effecting delivery of expression units to recipient cells, the next steps generally concern identifying the transformed cells for further culturing and plant regeneration. In order to improve the ability to identify transformants, one may desire to employ a selectable or screenable marker gene with a transformation construct prepared in accordance with the invention. In this case, one would then generally assay the potentially transformed cell population by exposing the cells to a selective agent or agents, or one would screen the cells for the desired marker gene trait. Examples of various selectable or screenable markers are disclosed in Miki and McHugh, 2004 (“Selectable marker genes in transgenic plants: applications, alternatives and biosafety”, Journal of Biotechnology, 107:193-232).

Cells that survive exposure to the selective agent, or cells that have been scored positive in a screening assay, may be cultured in media that supports regeneration of plants. In an exemplary embodiment, any suitable plant tissue culture media, for example, MS and N6 media may be modified by including further substances such as growth regulators. Tissue may be maintained on a basic media with growth regulators until sufficient tissue is available to begin plant regeneration efforts, or following repeated rounds of manual selection, until the morphology of the tissue is suitable for regeneration, then transferred to media conducive to shoot formation. Cultures are transferred periodically until sufficient shoot formation had occurred. Once shoots are formed, they are transferred to media conducive to root formation. Once sufficient roots are formed, plants can be transferred to soil for further growth and maturity.

To confirm the presence of the DNA for a transgenic trait in the regenerating plants, a variety of assays may be performed. Such assays include, for example, “molecular biological” assays, such as Southern and northern blotting and PCR™; “biochemical” assays, such as for detecting the presence of a protein product, e.g., by immunological means (ELISAs and western blots) or by enzymatic function; plant part assays, such as leaf or root assays; and also, by analyzing the phenotype of the whole regenerated plant.

Once a transgene for a trait has been introduced into a plant, that gene can be introduced into any plant sexually compatible with the first plant by crossing, without the need for ever directly transforming the second plant. Therefore, as used herein the term “progeny” denotes the offspring of any generation of a parent plant prepared in accordance with the present invention. A “transgenic plant” may thus be of any generation.

In one embodiment of the present invention, the identified cost decreasing traits and/or end-user traits are introduced into the germplasm of the female or the male parent as required either by direct transformation of elite inbreds or by first transforming an easily transformable inbred and then introducing the trait to elite germplasm by breeding into an elite inbred. The traits are introduced individually in individual inbreds. The traits present in individual inbreds are then combined to obtain required traits in a particular female or male inbred.

In another embodiment of the present invention, more than one traits are introduced into an inbred by repeatedly transforming the same inbred with a new trait provided on a transformation construct.

In another embodiment of the present invention, more than one trait is introduced into an inbred by providing more than one trait on a DNA construct used for transformation. For example, two traits may be provided on the same DNA construct and inserted into one locus thereby saving one locus which can be used for inserting different one or more traits.

In another embodiment of the present invention, more than one trait is introduced into an inbred by providing more than one trait on mini-chromosomes, for example, of the type described in the U.S. Pat. No. 7,235,716, U.S. Pat. No. 7,227,057, U.S. Pat. No. 7,226,782, U.S. Pat. No. 7,193,128, U.S. Pat. No. 6,649,347, US20050268359A1, all of which are incorporated herein by reference.

In another embodiment of the present invention, a combination of methods described above is applied to introduce more than one trait in an inbred. Further, inbreds with more than one trait can be crossed with at least one other inbred. In another aspect, an inbred may be crossed with at least two inbreds, non-limiting examples of which may include three-way, four-way, or multi-way crosses known to those skilled in the art of plant breeding.

As used herein, the term “inbred” means a line that has been bred for genetic homogeneity. Without limitation, examples of breeding methods to derive inbreds include pedigree breeding, recurrent selection, single-seed descent, backcrossing, and doubled haploids.

As used herein, the term “hybrid” means a progeny of mating between at least two genetically dissimilar parents. Without limitation, examples of mating schemes include single crosses, modified single cross, double modified single cross, three-way cross, modified three-way cross, and double cross, wherein at least one parent in a modified cross is the progeny of a cross between sister lines.

As used herein, the term “tester” means a line used in a test cross with another line wherein the tester and the lines tested are from different germplasm pools. A tester may be isogenic or nonisogenic.

Herein, “germplasm” includes breeding germplasm, breeding populations, collection of elite inbred lines, populations of random mating individuals, biparental crosses, heterotic groups, and apomictic germplasm.

It is known in the art that maize germplasm can be divided into a number of distinct heterotic groupings. A key goal of hybrid breeding programs is to maximize yield via complementary crosses. Crosses from distinct germplasm pools that result in a yield advantage constitute heterotic groups. The identification of heterotic groups facilitates informed crosses for a yield advantage. During inbred line development, advanced inbred lines are crossed with different tester lines in order to determine how the inbred line performs in hybrid combinations. The effect of a single cross reflects the specific combining ability (SCA) and the effect of the inbred in multiple crosses with different testers (typically in multiple locations) reflects the general combining ability (GCA). The heterotic groups may be used as female or male parents.

Apomictic germplasm can be produced by introducing certain genes such as those disclosed in these published patents and patent applications which are incorporated herein by reference: U.S. Pat. No. 5,710,367, U.S. Pat. No. 5,811,636, U.S. Pat. No. 6,750,376, U.S. Pat. No. 7,148,402, US2002069433A1, US2003082813A1, US2004016022A1, US2004098760A1, US2004103452A1, US2004148667A1, US2004168216A1, US2004168217A1, US2004216193A1, US2005155111A1, and US2005262595A1. Apomixis is a form of reproduction that produces seeds without the need for fertilization to take place. Progeny are consequently clones of the mother plant.

An “elite line” is any line that has resulted from breeding and selection for superior agronomic performance. Examples of elite lines suitable for use in the present invention are provided in Tables 11 and 12.

Descriptions of breeding methods that are commonly used for different traits and crops can be found in one of several reference books (Allard, “Principles of Plant Breeding,” John Wiley & Sons, NY, U. of CA, Davis, CA, 50-98, 1960; Simmonds, “Principles of crop improvement,” Longman, Inc., NY, 369-399, 1979; Sneep and Hendriksen, “Plant breeding perspectives,” Wageningen (ed), Center for Agricultural Publishing and Documentation, 1979; Fehr, In: Soybeans: Improvement, Production and Uses, 2nd Edition, Monograph., 16:249, 1987; Fehr, “Principles of variety development,” Theory and Technique, (Vol 1) and Crop Species Soybean (Vol 2), Iowa State Univ., Macmillan Publ. Co., NY, 360-376, 1987). The production of new elite corn hybrids requires the development and selection of elite inbred lines, the crossing of these lines and selection of superior hybrid crosses. Crosses for the production of the seed include traditional single crosses, three-way crosses, and double crosses or four-way cross. A single cross means the first generation of a cross between two inbred lines (preferably from two different heterotic groups), an inbred line and a foundation back cross, or of two foundation back crosses. A three-way cross means the first generation of a cross between a foundation single cross as one parent and an inbred line or a foundation backcross as the other parent. A double cross or four-way cross means the first generation of a cross between two foundation single crosses. The hybrid seed can be produced by manual crosses between selected male-fertile parents or by using male sterility systems. Additional data on parental lines, as well as the phenotype of the hybrid, influence the breeder's decision whether to continue with the specific hybrid cross. Details on hybrid crop breeding can be found in Bernardo, Breeding for Quantitative Traits in Plants, Stemma Press, Woodbury, Minn., 2002.

In general, two distinct breeding stages are used for commercial development of the female and male inbred containing a transgenic trait. The first stage involves evaluating and selecting a superior transgenic event, while the second stage involves integrating the selected transgenic event in a commercial germplasm.

First a transformation construct responsible for a trait is introduced into the genome via a transformation method. Numerous independent transformants (events) are usually generated for each construct. These events are evaluated to select those with superior performance. The event evaluation process is based on several criteria including 1) transgene expression/efficacy of the trait, 2) molecular characterization of the trait, 3) segregation of the trait, 4) agronomics of the developed event, and 5) stability of the transgenic trait expression. Evaluation of large population of independent events and more thorough evaluation result in the greater chance of success.

Events showing an appropriate level of gene expression or inhibition (via RNAi-mediated approaches) that corresponds with a desired phenotype (efficacy) are selected for further use by evaluating the event for insertion site, transgene copy number, intactness of the transgene, zygosity of the transgene, level of inbreeding associated with a genotype, genetic background, and growth response in various expected environmental conditions.

Events demonstrating, for instance, a clean single intact insert are found by conducting molecular assays for copy number, insert number, insert complexity, presence of the vector backbone, and by development and use of event-specific nucleic acid detection assays and are used for further breeding and development.

Segregation of a trait may be followed to identify transgenic events that follow a single-locus segregation pattern. A direct approach is to evaluate the segregation of the trait. An indirect approach may assess selectable marker segregation (if genetically linked to the transgenic trait). Agronomic performance of a transgenic inbred may vary due to somaclonal variation introduced during tissue culture process, insertional effects, homozygosity of a transgene, level of inbreeding, and genetic background. In advanced generations, agronomic performance is evaluated in several genetic backgrounds in replicated trials to identify the best gene and germplasm combination. For hybrid crops such as corn, agronomic trials are conducted in both inbred and hybrid background.

Transgene event instability over generations may be caused by transgene inactivation due to multiple transgene copies, zygosity level, highly methylated insertion sites, or level of stress. Thus, stability of transgenic trait expression may be ascertained by testing in different generations, environments, and in different genetic backgrounds. Events that show transgenic trait silencing are discarded. Events performing appropriately in a given germplasm background are selected for further development.

Generally, events with a single intact insert that is inherited as a single dominant gene and follow Mendelian segregation ratios are used in commercial breeding strategies such as backcrossing and forward breeding.

In a preferred embodiment, backcrossing is used to recover the genotype of an elite inbred with an additional transgenic trait. In each backcross generation, plants that contain the transgene are identified and crossed to the elite recurrent parent. Several backcross generations with selection for recurrent parent phenotype are generally used by commercial breeders to recover the genotype of the elite parent with the additional transgenic trait. During backcrossing the transgene is kept in a hemizygous state. Therefore, at the end of the backcrossing, the plants are self- or sib-pollinated to fix the transgene in a homozygous state. The number of backcross generations can be reduced by marker-assisted backcrossing (MABC). The MABC method uses molecular markers to identify plants that are most similar to the recurrent parent in each backcross generation. With the use of MABC and appropriate population size, it is possible to identify plants that have recovered over 98% of the recurrent parent genome after only two or three backcross generations. By eliminating several generations of backcrossing, it is often possible to bring a commercial transgenic product to market one year earlier than a product produced by conventional backcrossing.

Forward breeding is any breeding method that has the goal of developing a transgenic variety, inbred line, or hybrid that is genotypically different, and superior, to the parents used to develop the improved genotype. When forward breeding a transgenic crop, selection pressure for the efficacy of the transgene is usually applied during each generation of the breeding program. Additionally, it is usually advantageous to fix the transgene in a homozygous state during the breeding process as soon as possible to uncover potential agronomic problems caused by unfavorable transgene x genotype interactions.

After integrating the transgenic traits into a commercial germplasm, the final inbreds and hybrids are tested in multiple locations. Testing typically includes yield trials in trait neutral environments as well as typical environments of the target markets. If the new transgenic line has been derived from backcrossing, it is usually tested for equivalency by comparing it to the non-transgenic version in all environments.

Several types of genetic markers are known to those skilled in the art and can be used to expedite breeding programs. These genetic markers may include Restriction Fragment Length Polymorphisms (RFLP), Amplified Fragment Length Polymorphisms (AFLP), Simple Sequence Repeats (SSR), Single Nucleotide Polymorphisms (SNP), Insertion/Deletion Polymorphisms (Indels), Variable Number Tandem Repeats (VNTR), and Random Amplified Polymorphic DNA (RAPD).

Doubled-haploid breeding technology can be used to expedite the development of parental lines for crossing as known to those skilled in the art. The development of parental lines can be further enhanced by combining doubled-haploid breeding technologies with high-throughput, non-destructive seed sampling technologies. For example, U.S. Patent Application Publication US2006 0046264 (filed Aug. 26, 2005) and U.S. Patent Application Publication US2007 0204366 (filed Mar. 2, 2007), which are incorporated herein by reference in their entirety, disclose apparatuses and systems for the automated sampling of seeds as well as methods of sampling, testing and bulking seeds.

In another aspect of the present invention, only those transgenic events are selected for further development in which the nucleic acids encoding for cost decreasing traits and/or end user traits are inserted and linked to genomic regions (defined as haplotypes) that are found to provide additional benefits to the crop plant. The transgene and the haplotype comprise a T-type genomic region. Methods for using haplotypes and T-type genomic regions for enhancing breeding are disclosed in U.S. Application Publication 2006 0282911.

The present invention also provides for parts of the plants of the present invention. Plant parts, without limitation, include seed, endosperm, ovule and pollen. In a preferred embodiment of the present invention, the plant part is a seed. The invention also includes and provides transformed plant cells which comprise a nucleic acid molecule of the present invention.

TABLE 2A
Examples of gene/protein molecules for cost decreasing traits suitable
for use in the present invention. All references are incorporated by reference.

| Trait | Gene/protein | Reference |
|---|---|---|
| Herbicide | 5-enolpyruvylshikimate-3-phosphate | U.S. Pat. No. 5,627,061, |
| tolerance | synthases | U.S. Pat. No. 5,633,435, |
| | | U.S. Pat. No. 6,040,497, |
| | | U.S. Pat. No. 5,094,945, |
| | | US20060143727, and |
| | | WO04009761 |
| | glyphosate oxidoreductase (GOX) | U.S. Pat. No. 5,463,175 |
| | glyphosate decarboxylase | WO05003362 and |
| | | US Patent application |
| | | 20040177399 |
| | glyphosate-N-acetyl transferase | US Patent publications |
| | (GAT) | 20030083480, |
| | | 20060200874 |
| | dicamba monooxygenase | US Patent applications |
| | | 20030115626, |
| | | 20030135879 |
| | phosphinothricin acetyltransferase | U.S. Pat. No. 5,646,024, |
| | (bar) | U.S. Pat. No. 5,561,236, |
| | | EP 275,957; |
| | | U.S. Pat. No. 5,276,268; |
| | | U.S. Pat. No. 5,637,489; |
| | | U.S. Pat. No. 5,273,894 |
| | 2,2-dichloropropionic acid | WO9927116 |
| | dehalogenase | |
| | acetohydroxyacid synthase or | U.S. Pat. No. 6225105; U.S. Pat. No. 5767366, |
| | acetolactate synthase | U.S. Pat. No. 4,761,373; |
| | | U.S. Pat. No. 5,633,437; U.S. Pat. No. 6,613,963; |
| | | U.S. Pat. No. 5013659; U.S. Pat. No. 5141870; |
| | | U.S. Pat. No. 5378824; U.S. Pat. No. 5605011 |
| | haloarylnitrilase (Bxn) | U.S. Pat. No. 4,810,648 |
| | acetyl-coenzyme A carboxylase | U.S. Pat. No. 6,414,222 |
| | dihydropteroate synthase (sul I) | U.S. Pat. No. 5597717; |
| | | U.S. Pat. No. 5633444; |
| | | U.S. Pat. No. 5719046 |
| | 32 kD photosystem II polypeptide | Hirschberg et al., 1983, |
| | (psbA) | Science, 222:1346-1349 |
| | anthranilate synthase | U.S. Pat. No. 4,581,847 |
| | phytoene desaturase (crtI) | JP06343473 |
| | hydroxy-phenyl pyruvate dioxygenase | U.S. Pat. No. 6268549 |
| | protoporphyrinogen oxidase I (protox) | U.S. Pat. No. 5,939,602 |
| | aryloxyalkanoate dioxygenase (AAD- | WO05107437; |
| | 1, AAD-12) | WO07053482 |
| Male/female | Several | US20050150013 |
| sterility | Glyphosate/EPSPS | U.S. Pat. No. 6,762,344 |
| system | Male sterility gene linked to herbicide | U.S. Pat. No. 6,646,186 |
| | resistant gene | |
| | Acetylated toxins/deacetylase | U.S. Pat. No. 6,384,304 |
| | Antisense to an essential gene in | U.S. Pat. No. 6,255,564 |
| | pollen formation | |
| | DNAase or endonuclease/restorer | U.S. Pat. No. 6,046,382 |
| | protein | |
| | Ribonuclease/barnase | U.S. Pat. No. 5,633,441 |
| Intrinsic yield | glycolate oxidase or glycolate | US2006009598 |
| | dehydrogenase, glyoxylate | |
| | carboligase, tartronic semialdehyde | |
| | reductase | |
| | eukaryotic initiation Factor 5A; | US20050235378 |
| | deoxyhypusine synthase | |
| | zinc finger protein | US20060048239 |
| | methionine aminopeptidase | US20060037106 |
| | several | US20060037106 |
| | 2,4-D dioxygenase | US20060030488 |
| | serine carboxypeptidase | US20060085872 |
| | several | USRE38,446; 6,716,474; |
| | | 6,663,906; 6,476,295; |
| | | 6,441,277; 6,423,828; |
| | | 6,399,330; 6,372,211; |
| | | 6,235,971; 6,222,098; |
| | | 5,716,837; 6,723,897; |
| | | 6,518,488 |
| Nitrogen use | fungal nitrate reductases, mutant | US20050044585 |
| efficiency | nitrate reductases lacking post- | |
| | translational regulation, glutamate | |
| | synthetase-1, | |
| | glutamate dehydrogenase, | |
| | aminotransferases, nitrate transporters | |
| | (high affinity and low affinities), | |
| | ammonia transporters and amino acid | |
| | transporters | |
| | glutamate dehydrogenase | US20060090219 |
| | cytosolic glutamine synthetase; root- | EP0722494 |
| | specific glutamine synthetase. | |
| | several | WO05103270 |
| | glutamate 2-oxoglutarate | U.S. Pat. No. 6864405 |
| | aminotransferase | |
| Abiotic Stress | succinate semialdehyde | US20060075522 |
| tolerance | dehydrogenase | |
| including | several | WO06032708 |
| cold, heat, | several | US20060008874 |
| drought | transcription factor | US20060162027 |
| Disease | CYP93C (cytochrome P450) | U.S. Pat. No. 7038113 |
| resistance | several | U.S. Pat. Nos. 7038113; |
| | | 6,653,280; 6,573,361; |
| | | 6,506,962; 6,316,407; |
| | | 6,215,048; 5,516,671; |
| | | 5,773,696; 6,121,436; |
| | | 6,316,407; 6,506,962; |
| | | 6,617,496; 6,608,241; |
| | | 6,015,940; 6,013,864; |
| | | 5,850,023; 5,304,730); |
| | | 6,228,992; 5,516,671 |
| Insect | Cry1Aa, Cry1Ab, Cry1Ac, Cry1Ba, | U.S. Pat. Nos. 6,809,078; |
| resistance | Cry1Bb, Cry1Ca, Cry2Aa, Cry2Ab, | 6,713,063; 6,686,452; |
| | Cry3A, Cry3B, Cry3C, Cry9, Cry34 | 6,657,046; 6,645,497; |
| | and Cry35 (PS149B1), ET33, ET34, | 6,642,030; 6,639,054; |
| | ET29, TIC 809, TIC810, TIC900, | 6,620,988; 6,593,293; |
| | TIC901, TIC1201, TIC407, TIC417, | 6,555,655; 6,538,109; |
| | PS149B1, VIP1, VIP2, VIP3 and | 6,537,756; 6,521,442; |
| | VIP3A, Cry1A.105, RNA for gene | 6,501,009; 6,468,523; |
| | suppression targeting an insect gene, | 6,326,351; 6,313,378; |
| | DV49, | 6,284,949; 6,281,016; |
| | | 6,248,536; 6,242,241; |
| | | 6,221,649; 6,177,615; |
| | | 6,156,573; 6,153,814; |
| | | 6,110,464; 6,093,695; |
| | | 6,063,756; 6,063,597; |
| | | 6,023,013; 5,959,091; |
| | | 5,942,664; 5,942,658, |
| | | 5,880,275; 5,763,245; |
| | | 5,763,241; 6,291,156; |
| | | 6,486,157; |
| | | 6,429,360;7,244,820; |
| | | US2005/0210545; |
| | | US2007/022897; |
| | | WO07/027776; |
| | | US20060021087; |
| | | WO07027776A2 |

TABLE 2B
Examples of gene/protein molecules for cost decreasing traits
suitable for use in the present invention are disclosed in the
following references which are incorporated by reference.
Indicated SEQ ID NOs. represents use in preferred embodiments.

| Gene/Protein Name | SEQ ID NO | Reference |
|---|---|---|
| Herbicide Tolerance | | |
| Aryloxyalkanoate dioxygenase | SEQ ID NO: 9 | US20040031072A1 |
| (AAD-1) | | |
| Cytochrome P-450 dependent | | US20040214272A1 |
| monooxygenase | | |
| Cytochrome P-450 | SEQ ID NO: 2 | US20040172684A1 |
| monooxygenase | | |
| G1073 transcription factor | SEQ ID NO: 11 | US20040034888A1 |
| Hap3 protein | SEQ ID NO: 10 | EP0733112B1 |
| Hap3 transcription factor | SEQ ID NO: 4 | US20050086718A1 |
| MuA | SEQ ID NOS: 1, 2 | WO05107437A2 |
| Several | | WO05033319A2 |
| | SEQ ID NOS: 1 | US20040214272A1 |
| | through 142, 842 | |
| | SEQ ID NOS: 1 | EP1005272B1 |
| | through 369, 326 | |
| | SEQ ID NOS: 1 | US20030041357A1 |
| | through 63, 128 | |
| | SEQ ID NOS: 1 | WO9516041A1 |
| | through 36, 564 | |
| | SEQ ID NOS: 1 | U.S. Pat. No. 5,859,325 |
| | through 184, 663 | |
| | SEQ ID NO: 1 | U.S. Pat. No. 6,222,096 |
| Male/female Sterility | | |
| Aintegumenta (ANT) | SEQ ID NOS: 1, 2, 4, 5 | US20050132445A1 |
| Aintegumenta (ANT) | SEQ ID NO: 1, 2 | U.S. Pat. No. 6,559,357 |
| Enzyme of chalcone | | U.S. Pat. No. 6,005,167 |
| biosynthesis pathway | | |
| Female fertility gene | | U.S. Pat. No. 6,297,426 |
| Flavonone-3-hydroxylase | | U.S. Pat. No. 5,432,068 |
| Gene product for | | U.S. Pat. No. 5,859,341 |
| microsporogenesis | | |
| Gibberellin-insensitive gene | SEQ ID NOS. 1-4 or | U.S. Pat. No. 7,129,398 |
| | 16 | |
| GS I glutamine synthetase | SEQ ID NOS: 1 to 12 | WO06125678A2 |
| Male fertility gene | | U.S. Pat. No. 5,478,369 |
| Male fertility restorer gene | | WO9313649A1 |
| Ms26 gene | SEQ ID NOS: 1, 3, 7, | US20060288440A1 |
| | 17 | |
| Ms26 gene | SEQ ID NOS: 1, 3, 7, | US20060015968A1 |
| | 17 | |
| MS45 | SEQ ID NOS: 1, 2 | US20050246796A1 |
| MS45 | SEQ ID NOS: 1, 2 | WO05059121A2 |
| NPG1 | SEQ ID NOS: 1, 7, 9, | US20030061635A1 |
| | 10 | |
| RAFTIN | | WO04013168A2 |
| Restorer gene | | U.S. Pat. No. 6,683,230 |
| Rfo restorer gene | | WO05002324A2 |
| RNA-binding protein | | US20020062499A1 |
| SBMu200 | SEQ ID NOS: 1, 2, 3, | US20020083483A1 |
| | 4, 7, 8 | |
| SBMu200 gene | SEQ ID NOS: 1, 2, 3, | US20030182689A1 |
| | 4, 7, 8 | |
| | SEQ ID NOS: 1-8, 13-26 | US20060085867A1 |
| | SEQ ID NOS: 1-8, 13-26 | WO06036736A2 |
| | SEQ ID NOS: 1, 3, 7 | US20020183274A1 |
| | SEQ ID NOS: 1, 3, 7 | WO02063021A2 |
| | SEQ ID NOS: 1, 2 | U.S. Pat. No. 5,824,524 |
| | | WO9613588A1 |
| | | WO0106845A1 |
| | | WO03057848A2 |
| | | U.S. Pat. No. 6,407,311 |
| Intrinsic Yield | | |
| GATA transcription factor | SEQ ID NO: 1 | US20060179512A1 |
| Cdc25 | | US20030172404A1 |
| CDK2aAt | | WO9954489A1 |
| MsPRP2 | | WO9953016A2 |
| Stilbene synthase | | WO9927114A1 |
| Aldose reductase homologue | | WO9925852A1 |
| Yeast SLC1-1 or SLC1 | SEQ ID NO: 1, 3 | WO9638573A1 |
| Fructosyltransferase | | WO9414970A1 |
| AP2 transcription factor | SEQ ID NO: 18 | WO06069201A2 |
| Beta-glucosidase 1 (AtBG1) | SEQ ID NO: 3 | WO06068442A1 |
| WEE1 | | WO06063963A1 |
| Isopentenyl transferase | SEQ ID NOS: 1, 3, 5, | US20060064786A1 |
| | 7, 8, 10, 11, 13, 14, 16, | |
| | 17, 19-22, 24, 26, 28, | |
| | 40, 42, 44, 45, 47, 48, | |
| | 50, 51, 53, 55, 56, 58, | |
| | 60, 62, 64, 65, 69, 70-74, | |
| | 76 | |
| Cytokinin oxidase | SEQ ID NOS: 4, 5, 7, | WO05097824A2: |
| | 8, 10, 11, 51, 52 | |
| | SEQ ID NOS: 1-18 | WO04104162A2 |
| Metallothionein | SEQ ID NOS: 1-2 | WO04090142A2 |
| Isopentenyl transferase | SEQ ID NO: 1 | WO04074442A2 |
| | SEQ ID NOS: 1-111 | WO04061080A2 |
| | (odd numbered | |
| | sequences only) | |
| | SEQ ID NOS: 1-63 | WO03048319A2 |
| | (odd numbered | |
| | sequences only) | |
| Serine acetyl transferase and | | WO0001833A1 |
| cysteine-gamma-synthase | | |
| | SEQ ID NOS: 1, 2, 3 | WO04087755A2 |
| Several | | US20060143740A1 |
| | SEQ ID NOS: 1, 14-18 | US20050235382A1 |
| Transcription factor | SEQ ID NOS: 1, 3, 5, | US20040128712A1 |
| | 7, 9, 11, 13, 15, 17 | |
| | SEQ ID NOS: 1-368 | US20030233670A1 |
| | SEQ ID NOS: 2, 3, 4, 5 | US20030140381A1 |
| Bax inhibitor-1 (BI-1) | SEQ ID NOS: 2, 3, 4 | US20030009785A1 |
| Ste2O protein | SEQ ID NO: 2 | WO07003660A1 |
| Polypeptide having two WRKY | SEQ ID NOS: 2, 51 | WO07003409A2 |
| domain | | |
| Manganese-containing | | US20070006349A1 |
| superoxide dismutase | | |
| | SEQ ID NOS: 80, 90, | US20070006346A1 |
| | 92, 98, 109, 103 | |
| Prolamin box binding factor | SEQ ID NO: 29 | U.S. Pat. No. 7,157,623 |
| (PBF) | | |
| FAD2 | | WO06133441A2 |
| Leucine rich repeat receptor like | SEQ ID NOS: 9, 10, | WO06131547A1 |
| protein | 13, 14 | |
| | SEQ ID NOS: 80, 90, | US20070006346A1 |
| | 92, 98, 109, 103 | |
| | SEQ ID NOS: 41, 42, | WO06105277A2 |
| | 44, 45, 47, 48, 50, 51, | |
| | 53, 54, 56, 57, 59, 60, | |
| | 62, 63, 65, 66, 68, 69, | |
| | 71, 72, 74, 75, 94-95, | |
| | 97, 98, 100, 101, 103, | |
| | 105, 107, 109, 111, | |
| | 113, 115, 117, 119 | |
| Cyclin D3 (CYCD3) | SEQ ID NO: 2 | WO06100112A1 |
| Synovial sarcoma translocation | SEQ ID NO: 2 | WO06079655A2 |
| (SYT) | | |
| Several | | US20060168696A1 |
| Type I DnaJ-like | | WO06067236A1 |
| Translation elongation factor | | WO06067232A2 |
| | SEQ ID NOS: 1, 2, 3 | US20060107348A1 |
| Na+-K+ cotransporter protein | SEQ ID NO: 4 | WO06045829A1 |
| (HKT) | | |
| Glutamic acid dehydrogenase | | US20060090219A1 |
| | SEQ ID NO: 4-98 | WO06039449A1 |
| Lectin-Like Receptor Kinase 1 | SEQ ID NO: 1, 2 | WO06038891A1 |
| (Oslrk1) | | |
| Succinate semialdehyde | SEQ ID NOS: 270-538 | US20060075522A1 |
| dehydrogenase | | |
| Several | | US20060057724A1 |
| 2xC2H2 zinc finger protein | | US20060048239A1 |
| RNA-binding protein | | WO06018432A2 |
| | SEQ ID NO: 2, 107, | WO06013010A2 |
| | 125, 129, 137 | |
| | SEQ ID NOS: 270-538 | EP1621632A1 |
| SnRK2 (SNF1 related protein | | WO06008271A1 |
| kinase) | | |
| Cytokinin oxidase | SEQ ID NOS: 4, 5, 7, | US20060021082A1 |
| | 8, 10, 11, 51, 52 | |
| RLK827 | SEQ ID NO: 33 | WO06005771A1 |
| YIPPEE-like polypeptide | | WO06005751A1 |
| | SEQ ID NOS: 1-237 | US20060010516A1 |
| Cytokinin | SEQ ID NO: 49 | WO05123926A1 |
| oxidase/dehydrogenase | | |
| DP polypeptide | SEQ ID NOS: 1, 3, 12, | WO05117568A1 |
| | 14, 16, 18, 20, 22 | |
| Vacuolar pyrophosphatase | | US20050262598A1 |
| Aryloxyalkanoate dioxygenase | SEQ ID NO: 9 | WO05107437A2 |
| (AAD-1) | | |
| Cytokinin oxidase | SEQ ID NOS: 4, 5, 7, | WO05097824A2 |
| | 8, 10, 11, 51, 52 | |
| NAP1-like protein | SEQ ID NOS: 1, 2 | WO05094562A1 |
| S-phase specific ribosomal | SEQ ID NOS: 1, 2 | WO05093077A1 |
| protein (S3A) | | |
| S-phase specific ribosomal | SEQ ID NOS: 1, 2 | EP1580275A1 |
| protein (S3A) | | |
| Cyclin D3 (CYCD3) | SEQ ID NOS: 1, 2 | WO05085452A1 |
| D-type Cyclin Dependent | SEQ ID NOS: 1, 2 | WO05083094A2 |
| Kinase (CDKD) | | |
| GA 2-oxidase | | EP1084250B1 |
| GRUBX protein | SEQ ID NO: 2 | WO05059147A2 |
| Hypersensitive response elicitor | | EP1012255B1 |
| Seedy1 | SEQ ID NOS: 1, 5, 7 | WO05049646A2 |
| Dof1 | | US20050114925A1 |
| E2F transcription factor | SEQ ID NOS: 2, 4, 20 | US20050059154A1 |
| Beta-1,3 glucan synthase | SEQ ID NOS: 1, 2 | US20050044590A1 |
| Hap3 transcription factor | SEQ ID NOS: 8, 9, 10 | US20050022266A1 |
| RB3 | SEQ ID NOS: 1, 2 | US20050010975A1 |
| Cis-prenyltransferase | SEQ ID NOS: 2, 4, 6, | WO04106531A1 |
| | 16 | |
| Sugar-metabolizing enzyme | SEQ ID NOS: 10, 20 | WO04099403A1 |
| Carbonic anhydrase, ACCase, | | US20040133944A1 |
| LPAT, DGAT, oleosin | | |
| | SEQ ID NOS: 1-4 | WO04035797A2 |
| Leucine Rich Repeat receptor- | SEQ ID NOS: 1, 2, 3, | US20040067506A1 |
| like kinase | 18 | |
| Invertase inhibitor | SEQ ID NO: 1, 2 | U.S. Pat. No. 6,713,666 |
| | SEQ ID NOS: 1, 19, | US20040060079A1 |
| | 21, 23, 25, 27, 29, 31, | |
| | 33, 35, 37 | |
| Puroindoline | | WO04022759A2 |
| Sucrose phosphate synthase | SEQ ID NOS: 1-5 | USRE38446 |
| (SPS) | | |
| AGB1 | SEQ ID NO: 1 | WO04003146A2 |
| MYB transcription factor | SEQ ID NOS: 13-27 | US20040006797A1 |
| Glutamine synthetase | | EP0714237B2 |
| ABI4-like | SEQ ID NOS: 2, 4, 5 | US20030140381A1 |
| | SEQ ID NOS: 1-63 | WO03048319A2 |
| | (odd numbered | |
| | sequences only) | |
| Cyclin dependent kinase C | SEQ ID NO: 2 | WO03027299A2 |
| (CDKC) kinase | | |
| | SEQ ID NOS: 1-36 | WO03027249A2 |
| E2F transcription factor | SEQ ID NO: 2, 20 | WO03025185A1 |
| Plant transcription factors | SEQ ID NOS: 860, | WO03013227A2 |
| | 802, 240, 274, 558, 24, | |
| | 1120, 44, 460, 286, | |
| | 120, 130, 134, 698, | |
| | 832, 580, 612, 48 | |
| Adenylate kinase (ADK) | SEQ ID NOS: 1, 2 | WO02097101A1 |
| Vacuolar pyrophosphatase | | US20020178464A1 |
| | SEQ ID NOS: 2, 4, 6, | WO02083901A2 |
| | 9, 12, 15, 17, 20, 23, | |
| | 26, 29, 31, 33, 35, 37, | |
| | 39, 41, 43, 45, 47, 49, | |
| | 51, 52, 55, 57, 59, 61, | |
| | 63, 65, 67, 70, 73.75, | |
| | 77, 79, 81, 83, 85, 87, | |
| | 89, 91, 93, 95, 97, 99, | |
| | 101, 103 | |
| Leucine Rich Repeat receptor- | SEQ ID NOS: 1, 2, 3, | WO0246439A2 |
| like kinase | 18 | |
| Phytochrome A (PA) | | WO0246425A1 |
| Glutamate dehydrogenase | | US20020069430A1 |
| Protoporphyrinogen oxidase | | US20020042932A1 |
| Vacuolar pyrophosphatase | | WO0215674A1 |
| Phospholipid binding protein | SEQ ID NOS: 2, 3, 5, | WO0200697A2 |
| | 6, 42 | |
| Cdc2-related kinase | SEQ ID NOS: 2 to 6, | WO0196579A1 |
| | 28 to 36 | |
| Ramosa 1 | SEQ ID NOS: 1, 2, 3, | WO0190343A2 |
| | 5, 6 | |
| Invertase | SEQ ID NOS: 2, 5, 8, | US20010044941A1 |
| | 11, 14, 17, 20, 23, 26, | |
| | 29, 32, 35, 38, 41, 44, | |
| | 47, 50, or 53, 1, 3, 4, 6, | |
| | 7, 9, 10, 12, 13, 15, 16, | |
| | 18, 19, 21, 22, 24, 25, | |
| | 27, 28, 30, 31, 33, 34, | |
| | 36, 37, 39, 40, 42, 43, | |
| | 45, 46, 48, 49, 51, 52, | |
| | 54 | |
| Invertase inhibitors | SEQ ID NOS: 2, 5, 8, | WO0158939A2 |
| | 11, 14, 17, 20, 23, 26, | |
| | 29, 32, 35, 38, 41, 44, | |
| | 47, 50, 53, 1, 3, 4, 6, 7, | |
| | 9, 10, 12, 13, 15, 16, | |
| | 18, 19, 21, 22, 24, 25, | |
| | 27, 28, 30, 31, 33, 34, | |
| | 36, 37, 39, 40, 42, 43, | |
| | 45, 46, 48, 49, 51, 52, | |
| | 54 | |
| Vacuolar pyrophosphatase | | WO0133945A1 |
| Protoporphyrinogen oxidase | | WO0126458A2 |
| Cell division enhancing protein | | WO0123594A2 |
| Sucrose phosphate synthase | | WO0117333A1 |
| Endogenous negative regulator | | WO0102574A1 |
| of systemic acquired resistance | | |
| (SAR) | | |
| Cell cycle interacting protein | | WO0056905A2 |
| Glycogen synthase | | WO0055331A1 |
| DP | | WO0047614A1 |
| Receptor-like protein kinase | | WO0004761A1 |
| Gibberellin 2-oxidase | | WO9966029A2 |
| Glutamate dehydrogenase | | U.S. Pat. No. 5,998,700 |
| Steroid receptor, Bin1 | | WO9859039A1 |
| Glutamine synthetase inhibitor | | EP0714237B1 |
| Hypersensitive response elicitor | | WO9832844A1 |
| polypeptide | | |
| Starch branching enzyme (SBE) | | WO9722703A2 |
| SUC2 invertase | | U.S. Pat. No. 5,436,394 |
| Trehalose synthase | SEQ ID NOS: 2, 4, 82 | WO9317093A2 |
| Isopentenyl transferase | | WO9307272A1 |
| Galactinol synthase | SEQ ID NOS: 5, 6 | WO9302196A1 |
| | SEQ ID NOS: 1 | US20040034888A1 |
| | through 36, 564 | |
| | SEQ ID NOS: 1 | US20040031072A1 |
| | through 142, 842 | |
| Sucrose phosphorylase | SEQ ID NOS: 5 | U.S. Pat. No. 5,716,837 |
| ABI4-like | SEQ ID NOS: 2, 3, 4, 5 | U.S. Pat. No. 7,176,027 |
| Transcription factor | SEQ ID NOS: 1796, | US20070022495A1 |
| | 1995 | |
| Sucrose phosphorylase | SEQ ID NO: 6 | USRE39114 |
| Phytochrome | SEQ ID NOS: 240 | US20060041961A1 |
| | through 390, 392 | |
| | through 478 | |
| Methionine aminopeptidase | SEQ ID NOS: 1 to 4 | US20060037106A1 |
| (MAP protein) | | |
| Puroindoline | | US20050246792A1 |
| S3A protein | SEQ ID NOS: 1, 2 | US20050210544A1 |
| Cytokinin oxidase | SEQ ID NOS: 1, 3, 5, | US20050150012A1 |
| | 7, 9, 11, 25, 26, 27, 28 | |
| | to 31, 33, 34 | |
| Cytokinin-modulating gene | SEQ ID NOS: 1, 3, 4, | US20040237147A1 |
| | 7, 18 | |
| | SEQ ID NOS: 1 | US20040216190A1 |
| | through 11088 | |
| | SEQ ID NOS: 1 | US20040214272A1 |
| | through 369, 326 | |
| Methionine aminopeptidase | SEQ ID NOS: 1, 2, 3, 4 | WO04070027A2 |
| (MAP protein) | | |
| 14-3-3 proteins | SEQ ID NOS: 1 | WO04053055A2 |
| | through 736 | |
| Cytokinin oxidase | SEQ ID NOS: 27, 1, 3, | US20040031073A1 |
| | 5, 7, 9, 11, 25, 26, 28 | |
| | to 31, 33, 34 | |
| | SEQ ID NOS: 1 | US20030233675A1 |
| | through 47374 | |
| Acid invertase and ADPglucose | | US20030217387A1 |
| pyrophosphorylase | | |
| Chymostatin-sensitive serine | | WO04011656A1 |
| protease | | |
| RB3 nucleic acid | SEQ ID NOS: 1, 2 | US20030167505A1 |
| WEE1 | SEQ ID NOS: 1, 2 | US20030041342A1 |
| GDRP | | US20030024003A1 |
| AGL11 | | U.S. Pat. No. 7,179,957 |
| Dominant negative allele for an | | US20070028330A1 |
| endoplasmic reticulum located | | |
| gene | | |
| LMP polynucleotide sequences | SEQ ID NOS: 1-8, 18-28 | WO07016521A2 |
| Cytochrome P-450 | SEQ ID NOS: 2 | EP0733112B1 |
| monooxygenase | | |
| Novel lipid metabolism protein | SEQ ID NOS: 39-42 | WO06133166A2 |
| Putative peroxisomal enoyl | SEQ ID NOS: 1-8 | WO06125756A2 |
| CoA hydratase/isomerase-like | | |
| polypeptide | | |
| Plastid ADP/ATP translocator | SEQ ID NOS: 5, 6, 7 | U.S. Pat. No. 6,891,088 |
| DP | SEQ ID NOS: 1 | US20050076404A1 |
| Dextrinase inhibitor | SEQ ID NOS: 1 | WO04112468A1 |
| Brassica seed coat gene | | US20040250314A |
| Fungal hexokinase | SEQ ID NOS: 2, 6, 8, | WO04101748A2 |
| | 10, 12, 14, 16, 18, 20, | |
| | 22, 24, 26, 28, 30 | |
| Fungal hexokinase | SEQ ID NOS: 2, 6, 8, | US20040237138A1 |
| | 10, 12, 14, 16, 18, 20, | |
| | 22, 24, 26, 28, 30 | |
| | SEQ ID NOS: 1 | US20040214272A1 |
| | through 184, 663 | |
| TOB3 ATPase Domain (TAD) | | WO04087927A1 |
| protein | | |
| Several | | WO04063333A2 |
| | SEQ ID NOS: 1 | US20040123343A1 |
| | through 102, 483 | |
| | | US20040060082A1 |
| | SEQ ID NOS: 1 | US20040034888A1 |
| | through 36, 564 | |
| Cyclin-dependent kinase | SEQ ID NOS: 1, 3, 5 | US20040003433A1 |
| Plant transcription factor | | US20030121070A1 |
| Cyclin D | | US20030041343A1 |
| Lipid metabolism proteins | | WO03014376A2 |
| (LMP) | | |
| Sucrose synthase | SEQ ID NO: 1 | US20020116736A1 |
| | | WO02063020A2 |
| Cytokinin oxidase | SEQ ID NOS: 27, 1, 3, | WO0196580A2 |
| | 5, 7, 9, 11, 25, 26, 28 | |
| | to 31, 33, 34 | |
| Seed storage protein (SSP) | | U.S. Pat. No. 6,326,527 |
| Sucrose phosphorylase | | US20010016953A1 |
| Sucrose phosphorylase | | U.S. Pat. No. 6,222,098 |
| Acid invertase and ADP glucose | | WO0078984A2 |
| pyrophosphorylase | | |
| Amylosucrase | | WO0073422A1 |
| WEE1 | | WO0037645A2 |
| Subtilisin-like serine protease | | WO0022144A2 |
| CycD protein | | WO0017364A2 |
| | | U.S. Pat. No. 6,040,497 |
| Constitutive kinase subunit | SEQ ID NOS: 1, 3 | WO9961619A2 |
| (CKS) | | |
| Cytochrome P-450 dependent | | WO9516041A1 |
| monooxygenase | | |
| | SEQ ID NOS: 1 | US20070044171A1 |
| | through 105, 582 | |
| Amylosucrase | | U.S. Pat. No. 6,566,585 |
| SSE1 protein | SEQ ID NOS: 1-4 | US20030084475A1 |
| | | US20030066105A1 |
| | | EP1090544A4 |
| accD gene | | JP2002335786A2 |
| Isopentenyl transferase | | WO0242440A2 |
| Desaturase gene | | WO0011012A1 |
| Transcription factor | | WO9937794A1 |
| Protein kinase, glucosidase and | SEQ ID NO: 1 | WO9805760A2 |
| phosphate transporter | | |
| Maize promoter RS81 | SEQ ID NO: 4 | U.S. Pat. No. 6,232,526 |
| Maize promoter RS81 | SEQ ID NO: 1 | U.S. Pat. No. 6,207,879 |
| Fructose-1,6-bisphosphate | SEQ ID NO: 2 | U.S. Pat. No. 7,151,204 |
| (F16BP) aldolase | | |
| RCAN5 | SEQ ID NOS: 12, 13 | U.S. Pat. No. 7,126,044 |
| | SEQ ID NOS: 4 | U.S. Pat. No. 7,122,720 |
| O-methyltransferase homolog | | U.S. Pat. No. 7,122,718 |
| Maize promoter RS81 | SEQ ID NOS: 4 | U.S. Pat. No. 6,583,338 |
| Hypersensitive response elicitor | SEQ ID NOS: 23 | U.S. Pat. No. 6,583,107 |
| protein | | |
| p-coumarate Co-enzyme A | SEQ ID NO: 1 | U.S. Pat. No. 6,455,762 |
| ligase | | |
| p34cdc2 | | U.S. Pat. No. 6,087,175 |
| Transcription factor | SEQ ID NO: 1796 | US20070022495A1 |
| | SEQ ID NOS: 1-4 | US20060212966A1 |
| | SEQ ID NOS: 1-64 | US20040219675A1 |
| Homeobox protein domain | | US20060179511A1 |
| family | | |
| NUE | | |
| 5-(enolpyruvyl) shikimate-3- | | U.S. Pat. No. 5,792,930 |
| phosphate synthase | | |
| a translation initiation factor | | US20050108791 |
| E1F-4F | | |
| 5-(enolpyruvyl) shikimate-3- | | U.S. Pat. No. 5,633,435 |
| phosphate synthase | | |
| Acetyl coenzyme A carboxylase | | U.S. Pat. No. 5,290,696 |
| Acetyl coenzyme A carboxylase | | U.S. Pat. No. 5,162,602 |
| Acetylcoenzyme A carboxylase | | U.S. Pat. No. 5,428,001 |
| Corynebacterium glutamicum | | WO0100843A2 |
| Asparagine synthase | | |
| Asparagine synthetase | | U.S. Pat. No. 5,256,558 |
| AP2 transcription factor | 18 | WO06069201A2 |
| Sucrose synthase | | WO9803637A1 |
| Brassica turgor gene-26 | SEQ ID No: 1 | US20050015828A1 |
| Cdc25 protein | | US20030172404A1 |
| Complement of dicarboxylic | | U.S. Pat. No. 5,077,209 |
| acid transport gene | | |
| DNA encoding NADP-GDH | SEQ ID NOS: 1, 3, 18, | EP0859849B1 |
| alpha and beta sub-unit | 19, 23, 25 | |
| Asparagine synthetase | | U.S. Pat. No. 5,955,651 |
| flavohemoglobin | SEQ ID NOS: 1, 2, 260 | WO06121757A2 |
| G482 subclade of the non- | | WO04076638A2 |
| LEC1-like clade of proteins of | | |
| the L1L-related CCAAT | | |
| transcription factor | | |
| GATA transcription factor | SEQ ID NO: 1 | US20060179512A1 |
| Glucosamine-6-phosphate | SEQ ID NO: 1 | WO9835047A1 |
| dearninase | | |
| Glutamate 2-oxoglutarate | | U.S. Pat. No. 6,864,405 |
| aminotransferase | | |
| glutamic acid dehydrogenase | | US20060090219A1 |
| Glycine-specific sym gene | | U.S. Pat. No. 4,818,696 |
| Homeobox protein domain | | US20060179511A1 |
| family | | |
| hypersensitivity related plant | | U.S. Pat. No. 5,550,228 |
| gene (hsr203J gene of SEQ ID | | |
| No. 1) | | |
| lutamine synthetase | | WO0009726A1 |
| nifL or nifL-like gene and | | WO9305154A1 |
| functional nifA(-like) | | |
| phosphoenol pyruvate | | US20030177520A1 |
| carboxylase | | |
| phytochrome | | EP1586652A1 |
| promintron sequence (I) of the | | EP1002868A1 |
| rolA gene | | |
| Protoporphyrinogen oxidase | | U.S. Pat. No. 6,084,155 |
| Protoporphyrinogen oxidase | | U.S. Pat. No. 5,939,602 |
| Protoporphyrinogen oxidase | | U.S. Pat. No. 5,767,373 |
| recombinant polynucleotide | | WO0215675A1 |
| root-specific glutamine | | U.S. Pat. No. 6,107,547 |
| synthetase | | |
| Several | | U.S. Pat. No. 5,767,374 |
| Transcription factor | SEQ ID NOS: 1796, | US20070022495A1 |
| | 1995 | |
| Zea mays brittle2 gene | SEQ ID NOS: 1 | US20060272045A1 |
| | through 36 | |
| | Tables 1-5 | WO05118820A2 |
| | SEQ ID NOS: 2, 4, 6, | WO05103270A2 |
| | 8, 10, 51, 64, 66, 68, 92, | |
| | 94, 219 | |
| | SEQ ID NOS: 1 | |
| | through 368 | |
| | SEQ ID NO: 1 | WO0022138A1 |
| | SEQ ID NOS: 1-4 | WO06102343A2 |
| | SEQ ID NOS: 1-4 | US20060212966A1 |
| | SEQ ID NOS: 2, 4-18 | WO06065552A2 |
| | SEQ ID NOS: 2, 4-18 | US20060137034A1 |
| | SEQ ID NOS: 1-4 | WO06102343A2 |
| | SEQ ID NOS: 1-4 | US20060212966A1 |
| | SEQ ID NOS: 11-13 | WO06130127A2 |
| | SEQ ID NOS: 2, 4, 6, | WO05103270A2 |
| | 8, 10, 51, 64, 66, 68, 92, | |
| | 94, 219 | |
| | | US20030233670A1 |
| | SEQ ID NOS: 859, | US20030226173A1 |
| | 801, 239, 273, 557, 23, | |
| | 1119, 43, 459, 285, | |
| | 119, 129, 133, 697, | |
| | 831, 579, 611, 47 | |
| | SEQ ID NOS: 1, 3, 7, | WO9712983A1 |
| | 8, 9, 10, 11, 12, 13, 14, | |
| | 15, 16, 17, 18, 19, 20, | |
| | 21, 22, 23, 25 | |
| | | US20070033671A1 |
| | | WO04031349A2 |
| | | WO06004955A2 |
| | | US20060294622A1 |
| | | US20060200878A1 |
| | SEQ ID NOS: 8, 15, | WO05003338A1 |
| | 24, 25, 32, 40, 48, 52, | |
| | 54 | |
| | SEQ ID NOS: 1, 14-18 | US20050235382A1 |
| | SEQ ID NOS: 1 | US20040216190A1 |
| | through 11088 | |
| | | WO06076423A2 |
| | | US20030188330A1 |
| Abiotic Stress tolerance | | |
| 9-cis-epoxycarotenoid | 10, 11, 12, 14 to 16, | WO03020015A2 |
| dioxygenase (NCED) | 26, 28, 30, 32, 34 | |
| Arabidopsis CtpA (C terminal | | US20060179511A1 |
| processing protease) | | |
| Cold binding factors | | US20030233680 |
| ABC Transporter Gene | | WO02057406A2 |
| ABI4-like | 2, 3 | U.S. Pat. No. 7,176,027 |
| AKIP1 nucleic acid | | WO04013295A2 |
| Aldose reductase homologue | 1 or 3 | WO9925852A1 |
| AP2 transcription factor | 18 | WO06069201A2 |
| AP2/EREBP domain-containing | 12, 3, 14, 15, 16 | US20040143098A1 |
| transcription factor | | |
| ASR and A22, CYS, LEA, | 16, 18 to 21, 22, 24 to | WO02090547A1 |
| DHN, PKABA | 28, 29, 31 to 34, 64, 5, | |
| | 37 to 48, 68, 70, 35, | |
| | 56, 58, 60 to 63, 74, 76 | |
| ATPase subunit c isoform 2 | 1 | EP1206560B1 |
| Bacterial phytochrome protein | 240 through 390, 392 | US20060041961A1 |
| | through 478 | |
| Bax inhibitor-1 (BI-1) | 2, 3, 4 | US20030009785A1 |
| Bax Inhibitor-1 protein | 2, 6, 8, 10, 12, 14, 16, | WO04081217A2 |
| | 18, 20, 22, 24, 26, 28, | |
| | 30, 32, 38 | |
| Bax Inhibitor-1 protein | | US20060064775A1 |
| Beta-glucosidase 1 (AtBG1) | 3 | WO06068442A1 |
| BOS1 gene | 1, 2, 3 | US20050120410A1 |
| BOS1 nucleic acid | 1, 2, 3, 4 | WO03076597A2 |
| B-type cyclin dependent kinase | 2, 4, 6, 9-13 | WO05024029A2 |
| Calcium dependent protein | | WO9826045A1 |
| kinase (CDPK) | | |
| Calcium-dependent plant | 2 | WO05113777A1 |
| protein kinase | | |
| Cap binding protein 80 (CBP80) | 1, 2 | US20040096861A1 |
| CaPF1 | 1 | WO04099415A1 |
| CDK2aAt | | WO9954489 |
| Chloroplast elongation factor | 1 to 6 | US20030044972A1 |
| EF-Tu | | |
| Class-2 non-symbiotic | 1 | WO04087755A2 |
| hemoglobin | | |
| Coix regulatory sequence | 8, 18, 19 | U.S. Pat. No. 6,635,806 |
| Cold shock protein | 5-65 (odd number | WO05033318A2 |
| | sequences only) | |
| Cold shock protein | 4-92 (even number | US20050097640A1 |
| | sequences only) | |
| CRWAQ81 | 1, 2 | US20050097633A1 |
| CRYO | | US20060200879A1 |
| CRYO protein | 2, 4, 6, 8, 10 | WO04090141A2 |
| Cytokinin modulating | | WO0063401A1 |
| Cytokinin oxidase | 4, 5, 7, 8, 10, 11, 51, 52 | US20060021082A1 |
| Delta-6-desaturase | | WO9306712A1 |
| EhHOG | 1, 2 | WO06027779A1 |
| Fatty acid hydroxylase gene | 1, 4, 5 | EP1185669B1 |
| Flu | | WO02052013A2 |
| Fructose-1,6-bisphosphate | 2 | U.S. Pat. No. 7,151,204 |
| (F16BP) aldolase | | |
| Fructosyltransferase | | U.S. Pat. No. 6,025,542 |
| G1073 clade of transcription | 64 | US20050097638A1 |
| factor polypeptide | | |
| G1792 clade of transcription | 63 | WO06033708A |
| factos | | |
| G47 clade of transcription factor | | WO05047516A2: |
| G482 subclade of the non- | 87, 2907, 2908, 2909, | WO04076638A2 |
| LEC1-like clade of protein | 2910, 2911, 2912, | |
| | 2913, 2914 | |
| G482 subclade of the non- | 4 | US20050086718A1 |
| LEC1-like clade of protein | | |
| G867 clade of transcription | 55 | WO05038034A2 |
| factor | | |
| Glycine Rich Protein (GRP) | 1 | U.S. Pat. No. 6,747,189 |
| promoter | | |
| Hypersensitive response elicitor | 5, 11, 15, 17, 21, 23, 25 | US20050246799A1 |
| protein | | |
| Iron binding protein (IBP) | 1, 2 | US20010039670A1 |
| Iron binding protein (IBP) | 1, 2 | U.S. Pat. No. 6,563,019 |
| Isopentenyl transferase | 1, 3, 5, 7, 8, 10, 11, 13, | US20060064786A1 |
| | 14, 16, 17, 19-22, 24, | |
| | 26, 28, 40, 42, 44, 45, | |
| | 47, 48, 50, 51, 53, 55, | |
| | 56, 58, 60, 62, 64, 65, | |
| | 69, 70-74, 76 | |
| KAT1 | | U.S. Pat. No. 6,635,803 |
| Lipid Metabolism Protein | 1 to 12 | WO06053743A2 |
| Lycopene cyclase | | WO03066866A2 |
| Manganese-containing | | US20070006349A1 |
| superoxide dismutase gene | | |
| Mannitol-1-phosphate | | U.S. Pat. No. 5,780,709 |
| dehydrogenase | | |
| Metallothionein | 1, 2 | WO04090142A2 |
| Methyltransferase | | WO0011142A2 |
| Mitogen-activated protein | 1, 3 | US20040209325A1 |
| kinase-5 (MAPK5) | | |
| MnSOD gene | | WO04053136A1 |
| MsPRP2 | | WO9953016 |
| MuA | 1, 2 | U.S. Pat. No. 6,222,096 |
| Ovule development protein 2 | 2 | US20050257289A1 |
| (ODP2) | | |
| Oxidoreductase stress-related | 1, 3, 5, 7, 9, 11, 13, 15, | US20060064784A1 |
| protein (ORSRP) | 17, 19, 21, 23, 25, 27, | |
| | 129, 31, 33, 35, 37, 39, | |
| | 41, 43, 45, 47, 49 | |
| ParG gene | 1, 2, 3, 4, 15, 16, 23 | WO04090140A2 |
| parp2 | 5-12, 18-20 | WO06045633A1 |
| Plant mitogen-activated protein | 2 | U.S. Pat. No. 6,376,747 |
| kinase kinase (MAPKK or | | |
| MEK) | | |
| Plant protease | | US20050251885A1 |
| Poly (ADP-ribose) | 1, 2, 16 | US20060185038A1 |
| glycohydrolases (ParG) | | |
| ROB5 | 1 | US20060122375A1 |
| Serine acetyl transferase and | | WO0001833A1 |
| cysteine-gamma-synthase | | |
| Several | | US20060143740A1 |
| Several | | WO05118823A2 |
| SFR2 nucleic acid | 1, 3, 5, 7, 9, 11, 13 | WO03091441A1 |
| SLC1-1 | 1, 3 | U.S. Pat. No. 6,051,755 |
| Stilbene synthase | | WO9927114 |
| Stress related protein (SRP) | | US20060137043A1 |
| Stress-related polypeptides | | WO04061080A2 |
| Stress-related polypeptides | | US20060235215A1 |
| STS8 | 1 | U.S. Pat. No. 6,072,103 |
| Transcription factor | | WO04031349A2 |
| Transcription factor | 1, 3 | US20060026716A1 |
| Transcription factor | 1, 3, 5, 7, 9, 11, 13, 15, | US20040128712A1 |
| | 17 | |
| Transcription factor gene | 1, 2 | WO04087923A1 |
| CaPIF1 | | |
| Transcriptional factors ABP2, | 1, 3, 5 | US20060021091A1 |
| ABP4 and ABP9 | | |
| Trehalose-6-phosphate | | US20040123350A1 |
| synthetase (TPS) | | |
| Trehalose-6-phosphate | | WO04000008A1 |
| synthetase and trehalose-6- | | |
| phosphate phosphatase | | |
| Vacuolar pyrophosphatase | | US20050262598A1 |
| WEE1 | | WO06063963A1 |
| Y11414 gene | | US20050204431A1 |
| | 1 to 9214 | WO03008540A2 |
| | 1-195 | WO05021723A2 |
| | 1 to 18 | WO04104162A2 |
| | 1-111 (odd number | WO04061080A2 |
| | sequences only) | |
| | 18-34 | WO04046357A1 |
| | 1 | WO04043145A2 |
| | 1-63 (odd number | WO03048319A2 |
| | sequences only) | |
| | 1-4131, 8263-8353, | |
| | 8445-8829, 17505-17506 | |
| | 1-155, 157-229, 230-232, | WO0216655A2 |
| | 234-557, 559-572, | |
| | 574-605, 607-634, 636-634, | |
| | 636-786, 788-812, | |
| | 814-1261, 1699-1725, | |
| | 1727-1865, 1867-1917, | |
| | 1919-1927, 1929-1969, | |
| | 2227-2427, 2428-2585 | |
| | 2n, where n = 1-509 | US20060272060A1 |
| | 1, 2 | WO05071083A1 |
| | 1, 2 | US20060253938A1 |
| | 1, 4, 11 | US20060168692A1 |
| | 1, 2, 3, 4 | US20060162027A1 |
| | 1-18, 93-98, 247-252 | US20060123516A1 |
| | 18-34, 35-51, 52-68 | US20060107344A1 |
| | 1 to 8 | US20060075523A1 |
| | 1, 14-18 | US20050235382A1 |
| | 2, 4, 6, 8, 10, 12, 14, | US20050235377A1 |
| | 16, 18, 20, 22, 24, 26, | |
| | 27, 29, 31, 33, 35, 37, | |
| | 39, 41, 43, 45, 47, 51 | |
| | 1-195 | US20050097639A1 |
| | 1-63 (odd number | US20040219675A1 |
| | sequences only) | |
| | 1, 2 | US20040098764A1 |
| | 1-368 | US20030233670A1 |
| | | EP1185669B1 |
| | 1 through 36, 564 | US20040034888A1 |
| | 1 through 142, 842 | US20040031072A1 |
| | 1 to 3549 | US20060236419A1 |
| | 1 through 117, 596 | US20040181830A1 |
| | 1, 2, 3, 14, 15, 16 | WO02079245A2 |
| | 1 through 11088 | US20040216190A1 |
| | 1 through 184, 663 | US20040214272A1 |
| | 1 through 31, 564 | US20040172684A1 |
| | 1 through 102, 483 | US20040123343A1 |
| | 2, 4 | US20030188330A1 |
| | 1 to 4 | US20060212966A1 |
| | 1 to 64 | US20040219675A1 |
| | 1 through 184, 664 | US20040214272A1 |
| Disease resistance | | |
| 1-deoxy-D-xylulose 5- | 1, 2 | US20020108148A1 |
| phosphate reductoisomerase | | |
| 5′ regulatory region of emb5 | | U.S. Pat. No. 7,078,234 |
| gene | | |
| 5C9 gene encoding a maize | 1 | U.S. Pat. No. 6,617,498 |
| patatin-like polypeptide | | |
| Activated disease resistance 1 | | WO0220791A1 |
| (ADR1) | | |
| Albicidin detoxification enzyme | 1, 2, 3 | U.S. Pat. No. 6,388,175 |
| Antifungal protein | 1 to 127 | US20030028920A1 |
| Antisense RNA (aRNA) | | U.S. Pat. No. 5,316,930 |
| complementary to the mRNA of | | |
| an alfalfa mosaic virus | | |
| APZ transcription factor | 17, 18 | U.S. Pat. No. 6,664,446 |
| AtPtpl | | WO06081301A2 |
| Avirulence gene (avrPphB, | | US20020029392A1 |
| avrPphC, avrRpm1, avrPto, | | |
| avrRpt2, avr9, avr2, avrB, | | |
| avrPpiB, avrPmaA, avrPpiA, | | |
| avrPgyB, avrRps4, avrBs2, | | |
| avrPphE, avrC, avrD, and | | |
| avrRxv) | | |
| AvrRxv avirulence gene | | U.S. Pat. No. 6,586,657 |
| AvrRxv gene | | U.S. Pat. No. 6,476,292 |
| Basal endosperm transfer cell | 8 | U.S. Pat. No. 7,119,251 |
| layer (BELT) specific gene | | |
| Basal endosperm transfer cell | 1 to 6 | US20040003427A1 |
| layer (BELT) specific gene | | |
| Beta glucosidase | SEQ ID NOS: 1, 2, 3, 4 | U.S. Pat. No. 6,433,249 |
| Blec promoter--gene | 1 | U.S. Pat. No. 5,646,333 |
| BOS1 gene | 1, 2, 3 | US20050120410A1 |
| Calcium dependent protein | | US20030167516A1 |
| kinase (CDPK) | | |
| Cecropin B peptide Shiva-1 | | U.S. Pat. No. 7,151,203 |
| Class II O-methyltransferase | 1, 13, 14 | US20040029167A1 |
| (COMT II) gene | | |
| Coix regulatory sequence | 8, 18, 19 | U.S. Pat. No. 6,635,806 |
| Constitutive disease resistance 1 | 1, 2 | US20020108145A1 |
| (CDR1) polypeptide | | |
| Constitutive disease | 1 | U.S. Pat. No. 6,316,697 |
| resistance(CDR1) gene | | |
| Cysteine proteinase | 1, 3, 5, 7, 9, 11, 13, 15, | US20050102717A1 |
| | 17, 19, 21, 23, 25, 27, | |
| | 29, 31, 33, 35, 37, 39, | |
| | 41, 43, 45, 47, 49, 51, | |
| | 53, 55, 57, 59, 61, 63, | |
| | 65, 67, 69, 71, 73, 75 | |
| Cytochrome P-450 | 1 to 16 | U.S. Pat. No. 5,882,851 |
| monooxygenase | | |
| Disease resistance factor | 1, 3, 5, 7, 9, 11, 13, 15, | US20050102715A1 |
| | 17, 19, 21, 23, 25, 27, | |
| | 29, 31, 33, 35 | |
| Disease resistance gene (R) or | | US20020035739A1 |
| elicitor | | |
| Disease-resistance regulatory | 2, 14 | U.S. Pat. No. 6,100,451 |
| element | | |
| DNA coding for drosomicin | 1, 3 | U.S. Pat. No. 6,465,719 |
| DNA for drosomicin | 4, 6 | US20030167519A1 |
| Elicitin | | US20030040102A1 |
| Endochitinase, stilbene synthase | 1, 2, 3 | US20060288446A1 |
| and superoxide dismutase | | |
| Endogenous negative regulator | | WO0102574A1 |
| polypeptide of systemic | | |
| acquired resistance (SAR) | | |
| Fructose-1,6-bisphosphate | 2 | U.S. Pat. No. 7,151,204 |
| (F16BP) aldolase | | |
| Fus6 polypeptide | 100, 101 | U.S. Pat. No. 6,891,085 |
| Fusion gene | | U.S. Pat. No. 6,903,248 |
| G1792 clade of transcription | SEQ ID NO: 63 | WO06033708A |
| factor polypeptides comprising | | |
| an AP2 domain and an EDLL | | |
| domain | | |
| Gene encoding enzyme in the | 1, 2 | U.S. Pat. No. 6,271,439 |
| C-5 porphyrin pathway | | |
| Glucose oxidase gene | 1 | U.S. Pat. No. 6,054,318 |
| Glutamine synthase gene | 1 to 5 | US20040148651A1 |
| Glycine Rich Protein (GRP) | 1 | U.S. Pat. No. 6,747,189 |
| promoter | | |
| Histone deacetylase | 2, 4, 6, 8, 10, 12, 14, | U.S. Pat. No. 6,479,629 |
| | 16, 18 | |
| Histone deacetylase | 1, 3, 5, 7, 9, 11, 13, 15, | U.S. Pat. No. 6,287,843 |
| | 17 | |
| Histone deacetylase | 1 to 18 | US20020022256A1 |
| Hm1 | 2 | U.S. Pat. No. 5,589,611 |
| Hm2 polynucleotide | 1 | U.S. Pat. No. 6,486,302 |
| Hm2 polynucleotide | 1, 2 | U.S. Pat. No. 6,211,440 |
| Human-derived lysozyme | | U.S. Pat. No. 5,767,377 |
| protein | | |
| Hypersensitive response elicitor | 1, 3, 5, 7, 9 | U.S. Pat. No. 5,776,889 |
| polypeptide or protein | | |
| Hypersensitive response elicitor | 1, 3, 5, 7, 9 | U.S. Pat. No. 5,650,387 |
| polypeptide or protein | | |
| Hypersensitive response elicitor | 1, 2, 3, 4 | US20050120409A1 |
| polypeptide or protein | | |
| Hypersensitive response elicitor | 23, 169 to 403, 210 to | U.S. Pat. No. 6,858,707 |
| protein | 403, 267 to 403 | |
| Hypersensitive response elicitor | 1 to 4 | U.S. Pat. No. 6,855,683 |
| protein | | |
| Hypersensitive response elicitor | 23 | U.S. Pat. No. 6,583,107 |
| protein | | |
| Hypersensitive response elicitor | | U.S. Pat. No. 6,333,302 |
| protein | | |
| Hypersensitive response elicitor | 5, 11, 15, 17, 21, 23, 25 | US20050246799A1 |
| protein | | |
| Hypersensitive response elicitor | 1 to 4 | US20040006789A1 |
| protein | | |
| Hypersensitive response elicitor | 23 | US20030182683A1 |
| protein | | |
| Hypersensitive response elicitor | | US20020116733A1 |
| protein | | |
| Hypersensitive response elicitor | 1, 2 | US20020066122A1 |
| protein | | |
| Hypersensitive response elicitor | | US20020062500A1 |
| protein | | |
| Hypersensitive response elicitor | 23 | US20010011380A1 |
| protein | | |
| Hypersensitivity related plant | SEQ ID NOS: 1 | U.S. Pat. No. 5,550,228 |
| gene | | |
| Hypersensitivity related plant | 1, 2 | U.S. Pat. No. 7,109,397 |
| gene | | |
| Inducible pathogenesis-related | 1 | U.S. Pat. No. 6,841,720 |
| protein gene promoter | | |
| Isochorismate synthase | | US20030051273A1 |
| Isochorismate synthase | | WO0206447A2 |
| Isochorismate synthase | | U.S. Pat. No. 7,070,772 |
| Isoflavone 3′-hydroxylase | 1, 2, 5, 6 | US20050172354A1 |
| Isoflavone O-methyltransferase | 1 | U.S. Pat. No. 6,878,859 |
| LOL1 polypeptide | 1 | US20050055738A1 |
| Mag1 nucleic acid | 1 | US20050166287A1 |
| Maize promoter RS81 | SEQ ID NOS: 4 | U.S. Pat. No. 6,232,526 |
| Maize promoter RS81 | 1 | U.S. Pat. No. 6,207,879 |
| Maize promoter RS81 | 4 | U.S. Pat. No. 6,583,338 |
| Maize promoter RS81 | 1 | U.S. Pat. No. 6,437,217 |
| Maize promoter RS81 | 1 | U.S. Pat. No. 6,207,879 |
| Maize proteinase inhibitor | 1, 2 | US20030033632A1 |
| Maize Rar1-interactor nucleic | 1, 2, 3 | US20030167504A1 |
| acid | | |
| Maize RS324 promoter | 1 | U.S. Pat. No. 6,194,636 |
| Mitogen-activated protein | | EP1078985A2 |
| kinase kinase (MAPKK) | | |
| MLA polypeptide | | US20030192074A1 |
| Mlo gene | 1, 2 | U.S. Pat. No. 6,791,007 |
| Mlo gene | 3, 7, 11, 15 | U.S. Pat. No. 6,576,814 |
| Mlo gene | 1, 2 | U.S. Pat. No. 6,211,433 |
| Mlo homolog polypeptide | 31, 32 | U.S. Pat. No. 6,846,972 |
| Mlo protein | 11, 12 | U.S. Pat. No. 7,183,457 |
| Mlo-like polypeptide | 22, 23 | U.S. Pat. No. 6,680,427 |
| MuA | 1, 2 | U.S. Pat. No. 6,222,096 |
| myb1 gene | 2 | U.S. Pat. No. 5,939,601 |
| Myrosinase | 1 | US20040133936A1 |
| NIM1 gene | 1, 3, 5, 7, 15, 17, 19, | US20030159171A1 |
| | 29, 31, 33, 35, 37, 39, | |
| | 41, 43, 45, 47, 49, 51, | |
| | 53, 55, 57, 61, 63, 65, | |
| | 67, 69, 71, 73 | |
| NIM1 gene | 2, 3, | US20020152499A1 |
| NIM1 gene | 1, 7, 9, 11, 13, 15, 17, | US20050132438A1 |
| | 19 | |
| NIM1 gene (SAR gene) | 2 | U.S. Pat. No. 6,091,004 |
| NIM1 gene (SAR gene) | 2, 8 | U.S. Pat. No. 5,986,082 |
| NPR1 polynucleotide | 1, 3 | U.S. Pat. No. 6,504,084 |
| NPR1 polypeptide | 5 | US20020170094A1 |
| NPR1 polypeptide | 5 | U.S. Pat. No. 6,713,665 |
| NRC1 protein | 2, 4 | US20060206958A1 |
| N-tr protein | 3, 5 | U.S. Pat. No. 6,630,618 |
| Nucleotide Binding Site- | 1, 2, 3, 4, 9, 10 | |
| Leucine-Rich Repeat Proteins | | |
| (NBS-LRRP) | | |
| Nucleotide Binding Site- | 1, 3, 9 | US20040237137A1 |
| Leucine-Rich Repeat Proteins | | |
| (NBS-LRRP) | | |
| p34cdc2 | | U.S. Pat. No. 6,087,175 |
| Papaya ringspot virus coat | 2, 4, 6, 8, 10 | U.S. Pat. No. 7,071,377 |
| protein | | |
| Papaya ringspot virus coat | 1 to 20 | US20030172397A1 |
| protein | | |
| Pathogenesis related (PR-1) | 6 to 11, 14, 15 | US20010025380A1 |
| protein | | |
| Pathogenesis-related (PR1) | 1 to 12 | US20020166146A1 |
| protein | | |
| p-coumarate Co-enzyme A | 1 | U.S. Pat. No. 6,455,762 |
| ligase | | |
| Pi2-like disease resistance | 3, 7, 11 | US20040210957A1 |
| nucleic acid | | |
| Plant mitogen-activated protein | 2 | U.S. Pat. No. 6,376,747 |
| kinase kinase (MAPKK or | | |
| MEK) | | |
| Plant-defense-associated protein | | U.S. Pat. No. 5,530,187 |
| PR-1 gene | | U.S. Pat. No. 6,582,961 |
| Protein kinase gene | 1, 2, 3 | U.S. Pat. No. 5,648,599 |
| Rac genes | SEQ ID NOS: 1, 3, 5, | U.S. Pat. No. 6,555,732 |
| | 7, 9, 15, 17, 19, 21, 23, | |
| | 25, 27, 29, 31, 33 | |
| Rac-like gene | 1, 3, 5, 7, 9, 15, 17, 19, | U.S. Pat. No. 6,555,732 |
| | 21, 23, 25, 27, 29, 31, | |
| | 33 | |
| RAR1 | 1 to 4 | US20030140375A1 |
| RAR1 gene | 1, 2 | U.S. Pat. No. 7,098,378 |
| RAR1 gene | 1 | US20050160491A1 |
| RAR1 gene | 1 | US20050097637A1 |
| Rar1-interactor nucleic acid | 2, 3, 7, 9 | US20050177892A1 |
| RB nucleic acid | 4, 7 | US20050204419A1 |
| Receptor for hypersensitive | 4, 5, 10, 11, 14, 15, 16, | US20030177526A1 |
| response elicitor protein | 17, 20, 21, 22, 23, 24, | |
| | 25 | |
| Receptors for hypersensitive | 1 to 5 | US20020007501A1 |
| response elicitors | | |
| Replicase | 4 | U.S. Pat. No. 5,596,132 |
| Resistance gene | 3, 5 | US20020004944A1 |
| Resistance gene homologue | 1 to 36 | US20020108140A1 |
| (RGH) | | |
| RHIZOC3 nucleic acid | 15 | US20050188440A1 |
| Rice ankyrin repeat gene 1 | 2 | US20030126637A1 |
| (RANK1) | | |
| Rice retrotransposon Tos17 | 1, 2 | U.S. Pat. No. 7,067,717 |
| gene | | |
| Rice sucrose transporter gene | 1 | U.S. Pat. No. 7,186,821 |
| Rps2 DNA | 1, 2 | U.S. Pat. No. 6,127,607 |
| Rps2 DNA | | U.S. Pat. No. 7,179,601 |
| S-adenosyl-L- | 1, 2, 3 | US20030064895A1 |
| methionine:jasmonic acid | | |
| carboxyl methyltransferase | | |
| Salicylic acid-binding protein 2 | 1, 2 | U.S. Pat. No. 7,169,966 |
| (SABP2) gene | | |
| Salicylic acid-binding protein 2 | 1 | US20050034196A1 |
| (SABP2) gene | | |
| SCLBr protein (Mlo protein) | 1, 3, 5, 7, 9, 11, 13, 18, | USRE38252 |
| | 19 | |
| Serine-glyoxylate | 1, 2, 39, 40 | US20030172396A1 |
| aminotransferase (SGT) or an | | |
| alanine-glyoxylate | | |
| aminotransferase | | |
| Soybean calmodulin isoform | 3, 4 | U.S. Pat. No. 6,284,952 |
| (SCaM5) gene | | |
| Stearoyl-ACP desaturase | 1, 2, 3 | US20040049807A1 |
| Steroid receptor, Bin1 | | WO9859039A1 |
| STS8 | 1 | U.S. Pat. No. 6,072,103 |
| Subtilisin-like serine protease | SEQ ID NOS: 1, 2, 7-12 | WO0022144A2 |
| Systemic acquired resistance | 1, 3 | US20020073447A1 |
| gene | | |
| Thionin gene | | U.S. Pat. No. 6,187,995 |
| Tomato aspermy virus 2b gene | 1 | U.S. Pat. No. 6,207,882 |
| Tomato Cf-5 gene | 1, 2, 3, 4 | U.S. Pat. No. 6,225,532 |
| Tos17 gene | 1, 2 | US20040003428A1 |
| Transcription factor | SEQ ID NOS: 1, 3, 5, 7, | US20040128712A1 |
| | 9, 11, 13, 15, 17 | |
| Uubiquitin ligase | 1, 2 | US20020022258A1 |
| Viral replicase | 3, 4 | U.S. Pat. No. 5,945,581 |
| WIPK enzyme | 1 | U.S. Pat. No. 6,765,128 |
| Xa21 encoding RRK | 1, 2, 3, 4 | U.S. Pat. No. 5,977,434 |
| polypeptide | | |
| Xanthomonas hypersensitive | 1, 2 | U.S. Pat. No. 6,960,705 |
| response (XcpHR) elicitor | | |
| protein | | |
| | 1, 68 | U.S. Pat. No. 6,479,731 |
| | SEQ ID NOS: 1-63 | WO03048319A2 |
| | (odd numbered | |
| | sequences only) | |
| | SEQ ID NOS: 1, 14-18 | US20050235382A1 |
| | SEQ ID NOS: 1-368 | US20030233670A1 |
| | SEQ ID NOS: | US20040034888A1 |
| | 1 through 36, 564 | |
| | SEQ ID NOS: 1 | US20040214272A1 |
| | through 369, 326 | |
| | SEQ ID NOS: 1 | US20040123343A1 |
| | through 204,966 | |
| | SEQ ID NOS: 1 | US20040034888A1 |
| | through 36,564 | |
| | SEQ ID NOS: 1-3549 | US20060236419A1 |
| | SEQ ID NOS: 1 | US20040214272A1 |
| | through 184,663 | |
| | SEQ ID NOS: 1 | US20040172684A1 |
| | through 31,564 | |
| | SEQ ID NOS: 1 | US20040123343A1 |
| | through 102,483 | |
| | | U.S. Pat. No. 7,138,278 |
| | 1, 2, 3 | U.S. Pat. No. 6,646,183 |
| | 1 | U.S. Pat. No. 6,608,240 |
| | | U.S. Pat. No. 5,981,843 |
| | 1 to 420 | US20060253924A1 |
| | 1 to 4 | US20060212966A1 |
| | 1 to 4, 49 to 52 | US20060143734A1 |
| | 1, 2, 5-51 | US20060080749A1 |
| | 1, 2 | US20060041955A1 |
| | 100, 102, 104, 106, | US20050166286A1 |
| | 108, 110 | |
| | 1 to 64 | US20040219675A1 |
| | 1 through 184,664 | US20040214272A1 |
| | 1 through 102,483 | US20040123343A1 |
| | 1 to 12 | US20040049804A1 |
| | 1 through 36,564 | US20040034888A1 |
| | 1 through 375 | US20040006787A1 |
| | 1 | US20030226172A1 |
| | 1, 2 | US20030167515A1 |
| | | US20030152975A1 |
| | 1, 2, 3 | US20030101483A1 |
| | 34, 35, 36 | US20030088887A1 |
| | 10, 11, 12 | US20030084478A1 |
| | 2N, where N = 1-56 | US20030046723A1 |
| | 1 to 4 | US20020166143A1 |
| | 1 to 12 | US20020144307A1 |
| | 1, 2, 3 | US20020056152A1 |
| Insect resistance | | |
| Bx1 gene | SEQ ID NOS: 1 | U.S. Pat. No. 6,331,660 |
| Delta-endotoxin gene fragment | | WO9823641A1 |
| Glucose oxidase gene | SEQ ID NO: 1 | U.S. Pat. No. 6,054,318 |
| Hypersensitive response elicitor | SEQ ID NO: 23 | U.S. Pat. No. 6,583,107 |
| protein | | |
| Hypersensitive response elicitor | SEQ ID NOS: 5, 11, | US20050246799A1 |
| protein | 15, 17, 21, 23, 25 | |
| insecticidal protein | | EP0359472B1 |
| MuA | SEQ ID NOS: 1, 2 | U.S. Pat. No. 6,222,096 |
| Proteinase inhibitor | | WO9215690A1 |
| | SEQ ID NOS: 1 | US20040034888A1 |
| | through 36,564 | |
| | SEQ ID NOS: 1 | US20040216190A1 |
| | through 11088 | |
| | | WO06073727A2 |

TABLE 3
Examples of traits and proteins suitable for use in the present invention*

| | | | | | | | Protein |
|---|---|---|---|---|---|---|---|
| | | | | | | | SEQ |
| Trait | | | | | | Protein | ID |
| CK | CS | | | | | (NM_128336) hypothetical protein [Arabidopsis thaliana] | 240 |
| CK | | | | | | (NM_120565) expressed protein [Arabidopsis thaliana] dbj\|BAB08987.1 | 242 |
| CS | | | | | | (NM_126342) predicted by genefinder and genscan [Arabidopsis thaliana] | 244 |
| CS | | | | | | | 246 |
| CS | | | | | | (NM_111270) expressed protein [Arabidopsis thaliana] gb\|AAF05858.1 | 247 |
| CS | | | | | | expressed protein [Arabidopsis thaliana] gi\|11280688\|pir\|\|T45643 hypothetical protein | 248 |
| CS | | | | | | AF488576_1 (AF488576) putative bHLH transcription factor [Arabidopsis thaliana] | 249 |
| CS | | | | | | (AB013396) eukaryotic initiation factor 4, eIF4-like protein [Arabidopsis thaliana] | 251 |
| CS | | | | | | (AF197940) SAM:phospho-ethanolamine N-methyltransferase [Arabidopsis thaliana] g | 252 |
| CS | | | | | | (NM_106587) expressed protein [Arabidopsis thaliana] | 253 |
| CS | | | | | | (NM_127488) putative small heat shock protein [Arabidopsis thaliana] | 254 |
| CS | | | | | | (NM_128284) expressed protein [Arabidopsis thaliana] | 255 |
| CS | | | | | | (NM_126137) putative protein [Arabidopsis thaliana] | 256 |
| CS | | | | | | (AF370340) putative mitochondrial dicarboxylate carrier protein [Arabidopsis thaliana] | 257 |
| CS | | | | | | (NM_118393) HSP associated protein like [Arabidopsis thaliana] | 259 |
| CS | | | | | | (NM_118226) putative protein [Arabidopsis thaliana] | 261 |
| CS | | | | | | (NM_106009) MAP kinase, putative [Arabidopsis thaliana] | 263 |
| CS | | | | | | (NM_118860) GH3 like protein [Arabidopsis thaliana] | 264 |
| CS | | | | | | GAST1-related protein [Arabidopsis thaliana] | 266 |
| CS | HS | | | | | (NM_120567) expressed protein [Arabidopsis thaliana] | 273 |
| CK | HS | PEG | PP | SS | | (NC_001139) gamma-aminobutyrate (GABA) transaminase (4-aminobutyrate | 474 |
| | | | | | | aminotransferase); Uga1p [Saccharomyces cerevisiae] | |
| CS | PP | HS | | | | (NM_119632) putative protein [Arabidopsis thaliana] | 293 |
| CS | DS | HS | PP | PEG | | (NM_122954) glutamate-ammonia ligase (EC 6.3.1.2) precursor, chloroplast (clone | 357 |
| | | | | | | lambdaAtgs\|1) (pir\|\|S18600) [Arabidopsis thaliana] | |
| CS | LN | SS | PP | | | trichome differentiation protein GL1 - Arabidopsis thaliana | 376 |
| DS | | | | | | (NM_129846) putative cytochrome P450 [Arabidopsis thaliana] | 398 |
| DS | | | | | | homeotic protein agamous - Arabidopsis thaliana | 399 |
| DS | | | | | | (AF083220) proliferating cellular nuclear antigen [Arabidopsis thaliana] | 400 |
| DS | | | | | | (NM_126126) cyclin D3-like protein [Arabidopsis thaliana] | 401 |
| DS | | | | | | (NM_129769) unknown protein [Arabidopsis thaliana] | 402 |
| DS | | | | | | (NM_106341) Tub family protein, putative [Arabidopsis thaliana] | 404 |
| DS | | | | | | (NM_119505) 2-dehydro-3-deoxyphosphoheptonate aldolase [Arabidopsis thaliana] | 406 |
| DS | | | | | | L-ascorbate peroxidase (EC 1.11.1.11) precursor - Arabidopsis thaliana (fragment) | 407 |
| DS | | | | | | (NM_118837) putative protein [Arabidopsis thaliana] | 408 |
| DS | | | | | | pollen specific protein-related [Arabidopsis thaliana] | 409 |
| DS | | | | | | (AF013628) reversibly glycosylated polypeptide-2 [Arabidopsis thaliana] | 410 |
| DS | | | | | | (NM_119926) putative protein [Arabidopsis thaliana] | 411 |
| DS | | | | | | (AL161566) putative protein [Arabidopsis thaliana] | 413 |
| DS | | | | | | expressed protein [Arabidopsis thaliana] gi\|7485996\|pir\|\|T00711 | 414 |
| DS | | | | | | O64644\|SP18_ARATH Probable Sin3 associated polypeptide [Arabidopsis thaliana] | 415 |
| DS | | | | | | (NM_102795) putative GTP-binding protein [Arabidopsis thaliana] | 416 |
| DS | | | | | | (NC_003272) hypothetical protein [Nostoc sp. PCC 7120] | 417 |
| DS | | | | | | (NM_106032) ethylene-insensitive3-like3 (EIL3) [Arabidopsis thaliana] | 418 |
| DS | | | | | | AAF26152.1\|AC008261_9 (AC008261) putative homeobox-leucine zipper protein, | 420 |
| | | | | | | HAT7 [Arabidopsis thaliana] | |
| DS | | | | | | (NM_105503) putative transcription factor [Arabidopsis thaliana] | 421 |
| DS | | | | | | (NM_120281) putative homeodomain protein [Arabidopsis thaliana] | 422 |
| DS | | | | | | (NM_116132) putative protein [Arabidopsis thaliana] | 423 |
| DS | PP | HS | | | | (NM_124341) amino acid permease 6 (emb\|CAA65051.1) [Arabidopsis thaliana] | 290 |
| DS | LL | | | | | AAM66940.1\|(AY088617) membrane-associated salt-inducible protein like | 307 |
| | | | | | | [Arabidopsis thaliana] | |
| DS | PP | HS | | | | (NM_111011) expressed protein [Arabidopsis thaliana] | 369 |
| DS | LN | PEG | PP | CK | CS | bacteriophytochrome [Pseudomonas fluorescens] | 241 |
| HS | | | | | | (NM_118746) uncharacterized protein [Arabidopsis thaliana] | 268 |
| HS | | | | | | (NM_111186) expressed protein [Arabidopsis thaliana]] | 269 |
| HS | | | | | | (NM_120571) expressed protein [Arabidopsis thaliana] gb\|AAF61902.1\|AF208051_1 | 270 |
| | | | | | | (AF208051) small heat shock-like protein | |
| HS | | | | | | (NM_118912) putative protein [Arabidopsis thaliana] pir\|\|T05878 isp4 protein homolog | 271 |
| | | | | | | T29A15.220 | |
| HS | | | | | | (NM_101391) expressed protein [Arabidopsis thaliana] | 272 |
| HS | | | | | | (NM_121852) putative protein [Arabidopsis thaliana] | 274 |
| HS | | | | | | probable serine/threonine-specific protein kinase (EC 2.7.1.—) F17I5.140-Arabidopsis | 275 |
| | | | | | | thaliana emb\|CAA19877.1\|(AL031032) protein kinase-like protein [Arabidopsis | |
| | | | | | | thaliana] | |
| HS | | | | | | (AB024028) 20S proteasome beta subunit; multicatalytic endopeptidase [Arabidopsis | 278 |
| | | | | | | thaliana] | |
| HS | | | | | | small zinc finger-related protein [Arabidopsis thaliana] | 279 |
| | | | | | | gi\|12230183\|sp\|Q9XGY4\|IM08_ARATH Mitochondrial import inner membrane | |
| | | | | | | translocase subunit Tim8 | |
| HS | | | | | | AAB01678.1\|(U27590) Fe(II) transport protein [Arabidopsis thaliana] | 280 |
| HS | | | | | | (AY062804) A6 anther-specific protein [Arabidopsis thaliana] | 281 |
| HS | | | | | | (AF370326) putative 2-nitropropane dioxygenase [Arabidopsis thaliana] | 283 |
| HS | | | | | | (NM_104489) SAR DNA binding protein, putative [Arabidopsis thaliana] | 284 |
| | | | | | | gb\|AAF02835.1\|AC009894_6 (AC009894) nucleolar protein [Arabidopsis thaliana] | |
| | | | | | | gb\|AAG40838.1\|AF302492_1 (AF302492) NOP56-like protein [Arabidopsis thaliana] | |
| HS | | | | | | (AY085451) putative 3-isopropylmalate dehydrogenase [Arabidopsis thaliana] | 286 |
| HS | | | | | | (NM_102700) zinc finger protein, putative [Arabidopsis thaliana] | 287 |
| | | | | | | gb\|AAG51745.1\|AC068667_24 (AC068667) zinc finger protein, putative; 86473-88078 | |
| | | | | | | [Arabidopsis thaliana] | |
| HS | | | | | | (NM_124313) xylosidase [Arabidopsis thaliana] | 288 |
| HS | | | | | | (NM_118221) putative protein [Arabidopsis thaliana] | 291 |
| HS | | | | | | (AF488587) putative bHLH transcription factor [Arabidopsis thaliana] | 292 |
| HS | | | | | | (NM_111700) putative non-phototropic hypocotyl [Arabidopsis thaliana] | 359 |
| HS | CK | | | | | (NM_129646) expressed protein [Arabidopsis thaliana] pir\|\|T00747 RING-H2 finger | 276 |
| | | | | | | protein RHC1a | |
| HS | CS | | | | | (NM_119735) hypothetical protein [Arabidopsis thaliana] | 277 |
| HS | PEG | | | | | (AP001305) contains similarity to RNA polymerase transcriptional regulation | 351 |
| | | | | | | mediator~gene_id: MHC9.3 [Arabidopsis thaliana] | |
| HS | SP | PP | LN | | | (NM_128328) putative cytochrome P450 [Arabidopsis thaliana] | 373 |
| HS | PP | | | | | (NM_121408) putative protein [Arabidopsis thaliana] | 395 |
| HS | PP | SS | | | | (NM_101737) hypothetical protein [Arabidopsis thaliana] | 473 |
| LL | | | | | | (NM_123481) purine permease-like protein [Arabidopsis thaliana] dbj\|BAB09718.1\| | 295 |
| | | | | | | (AB010072) purine permease-like protein [Arabidopsis thaliana] | |
| LL | | | | | | (NM_127337) putative senescence-associated protein 12 [Arabidopsis thaliana] | 296 |
| LL | | | | | | (AF014824) histone deacetylase [Arabidopsis thaliana] | 297 |
| LL | | | | | | (NM_111472) expressed protein [Arabidopsis thaliana] | 299 |
| LL | | | | | | expressed protein [Arabidopsis thaliana] gi\|25408990\|pir\|\| | 300 |
| LL | | | | | | (NM_111160) unknown protein [Arabidopsis thaliana] | 301 |
| LL | | | | | | (NM_103835) expressed protein [Arabidopsis thaliana] | 302 |
| LL | | | | | | (NM_100550) sugar kinase, putative [Arabidopsis thaliana] | 303 |
| LL | | | | | | AAD17313.1\|(AF123310) NAC domain protein NAM [Arabidopsis thaliana] | 304 |
| LL | | | | | | (NM_114694) putative protein [Arabidopsis thaliana] | 305 |
| LL | | | | | | (NM_112070) expressed protein [Arabidopsis thaliana] | 306 |
| LL | | | | | | (NM_100045) polyphosphoinositide binding protein, putative [Arabidopsis thaliana] | 308 |
| LL | | | | | | (NM_121863) putative protein [Arabidopsis thaliana] gb\|AAG35778.1\|AF280057_1 | 309 |
| | | | | | | (AF280057) tonneau 2 [Arabidopsis thaliana] | |
| LL | | | | | | (NM_105311) calmodulin-related protein [Arabidopsis thaliana] | 310 |
| LL | | | | | | (NM_125674) 1-deoxy-D-xylulose 5-phosphate reductoisomerase (DXR) [Arabidopsis | 311 |
| | | | | | | thaliana] | |
| LL | | | | | | HMG1/2-like protein (SB11 protein) gi\|99914\|pir\|\|S22309 high mobility group protein | 313 |
| | | | | | | HMG-1-soybean gi\|18645\|emb\|CAA41200.1\|HMG-1 like protein gene [Glycine max] | |
| LL | | | | | | (AP001072) hypothetical protein [Oryza sativa (japonica cultivar-group)] | 314 |
| LL | | | | | | (NM_129374) expressed protein [Arabidopsis thaliana] | 427 |
| LL | CS | | | | | (NM_102942) expressed protein [Arabidopsis thaliana] | 262 |
| LL | HS | | | | | (NM_111953) omega-3 fatty acid desaturase, chloroplast precursor [Arabidopsis | 282 |
| | | | | | | thaliana] | |
| LL | SP | CS | | | | AAC13593.1\|(AF058914) contains similarity to Arabidopsis thaliana DNA-damage- | 434 |
| | | | | | | repair/tolerance resistance protein DRT111 (SW: P42698) | |
| LL | PP | SS | | | | (AC098693) Hypothetical protein [Oryza sativa (japonica cultivar-group)] | 477 |
| LL | LN | SS | | | | (NC_001143) Interacts with and may be a positive regulator of GLC7 which encodes | 478 |
| | | | | | | type1 protein phosphatase; DSs22p [Saccharomyces cerevisiae] | |
| LN | | | | | | (NM_118524) UDPglucose 4-epimerase-like protein [Arabidopsis thaliana] | 315 |
| LN | | | | | | (U80186) pyruvate dehydrogenase E1 beta subunit [Arabidopsis thaliana] | 316 |
| LN | | | | | | (NM_112338) expressed protein [Arabidopsis thaliana] dbj\|BAB02642.1\|(AP002061) | 317 |
| | | | | | | MtN3-like protein | |
| LN | | | | | | (NM_123987) ornithine aminotransferase [Arabidopsis thaliana] | 318 |
| LN | | | | | | (NM_117530) expressed protein [Arabidopsis thaliana] | 319 |
| LN | | | | | | (NM_128763) putative alanine acetyl transferase [Arabidopsis thaliana] | 320 |
| | | | | | | gb\|AAD15401.1 | |
| LN | | | | | | (NM_105480) expressed protein [Arabidopsis thaliana] sp\|Q9M647\|IAR1_ARATH IAA- | 321 |
| | | | | | | alanine resistance protein 1 | |
| LN | | | | | | AF327534_1 (AF327534) putative adenosine triphosphatase [Arabidopsis thaliana] | 322 |
| LN | | | | | | (NM_117486) Expressed protein [Arabidopsis thaliana] gb\|AAK68800.1\|(AY042860) | 323 |
| | | | | | | Unknown protein [Arabidopsis thaliana] | |
| LN | | | | | | (NM_127222) actin depolymerizing factor 5 [Arabidopsis thaliana] | 324 |
| LN | | | | | | nuclear envelope membrane protein-like - Arabidopsis thaliana | 325 |
| LN | | | | | | (NM_124151) farnesyl diphosphate synthase precursor (gb\|AAB49290.1) [Arabidopsis | 327 |
| | | | | | | thaliana] | |
| LN | | | | | | (NM_129758) putative C2H2-type zinc finger protein [Arabidopsis thaliana] | 328 |
| LN | | | | | | (NM_115995) putative DNA-binding protein [Arabidopsis thaliana] | 329 |
| LN | | | | | | (NM_103617) Cyclin, putative [Arabidopsis thaliana] | 331 |
| LN | | | | | | (NM_111813) putative protein kinase [Arabidopsis thaliana] | 332 |
| LN | | | | | | (NM_112485) putative L-asparaginase [Arabidopsis thaliana] | 333 |
| LN | | | | | | (NM_105394) expressed protein [Arabidopsis thaliana] | 334 |
| LN | | | | | | DNA-directed RNA polymerase subunit-related [Arabidopsis thaliana] | 335 |
| | | | | | | gi\|25313101\|pir\|\|A85078 | |
| LN | | | | | | (AP002524) hypothetical protein~similar to Drosophila melanogaster chromosome 3L, | 336 |
| | | | | | | CG10171 gene product [Oryza sativa (japonica cultivar-group)] | |
| LN | | | | | | (NM_121850) AP2-domain DNA-binding protein-like [Arabidopsis thaliana] | 337 |
| LN | | | | | | (NM_124046) bHLH protein-like [Arabidopsis thaliana] | 338 |
| LN | | | | | | (NM_102409) expressed protein [Arabidopsis thaliana] | 339 |
| LN | | | | | | (NM_113143) expressed protein [Arabidopsis thaliana] dbj\|BAB01784.1\|(AB022215) | 340 |
| | | | | | | hydroxyproline-rich glycoprotein [Arabidopsis thaliana] | |
| LN | | | | | | (AF239956) unknown [Hevea brasiliensis] | 341 |
| LN | | | | | | (AP001313) kinetechore (Skp1p-like) protein-like [Arabidopsis thaliana] | 342 |
| LN | | | | | | (NM_100157) ribosomal protein L19, putative [Arabidopsis thaliana] | 343 |
| LN | | | | | | (NM_104101) expressed protein [Arabidopsis thaliana] | 344 |
| LN | | | | | | (NM_102496) expressed protein [Arabidopsis thaliana] | 364 |
| PEG | | | | | | (NM_120445) protein kinase-like protein [Arabidopsis thaliana] | 345 |
| PEG | | | | | | expressed protein [Arabidopsis thaliana] gi\|12322743\|gb\|AAG51367.1\|AC012562_28 | 348 |
| PEG | | | | | | (AC012329) unknown protein; 50647-51606 [Arabidopsis thaliana] | 349 |
| PEG | | | | | | (NM_118352) putative protein [Arabidopsis thaliana] | 350 |
| PEG | | | | | | (NM_101382) expressed protein [Arabidopsis thaliana] gb\|AAD39643.1\|AC007591_8 | 352 |
| | | | | | | (AC007591) Contains a PF\|00175 Oxidoreductase FAD/NADH-binding domain. | |
| PEG | | | | | | (NM_100349) putative K+ channel, beta subunit [Arabidopsis thaliana] | 353 |
| PEG | | | | | | glycine-rich protein [Arabidopsis thaliana] gi\|12597766\|gb\|AAG60079.1\|AC013288_13 | 355 |
| PEG | | | | | | (NM_111769) expressed protein [Arabidopsis thaliana] | 356 |
| PEG | CS | | | | | (NM_125688) Dof zinc finger protein-like [Arabidopsis thaliana] | 346 |
| PEG | HS | | | | | (NM_125077) nucleosome assembly protein [Arabidopsis thaliana] | 347 |
| PEG | SP | HS | | | | (AB036735) allyl alcohol dehydrogenase [Nicotiana tabacum] | 435 |
| PP | | | | | | (NM_111700) putative non-phototropic hypocotyl [Arabidopsis thaliana] | 359 |
| PP | | | | | | dynein light chain-related [Arabidopsis thaliana] gi\|25405535\|pir\|\|E96562 | 360 |
| PP | | | | | | (AP003255) contains ESTs AU100655(C11462), C26007(C11462)~similar to | 361 |
| | | | | | | Arabidopsis thaliana chromosome 3, F24B22.150~unknown protein [Oryza sativa | |
| | | | | | | (japonica cultivar-group)] | |
| PP | | | | | | (AB010875) PHR1 [Arabidopsis thaliana] | 362 |
| PP | | | | | | (NM_112829) putative tyrosine phosphatase [Arabidopsis thaliana] | 363 |
| PP | | | | | | (NM_102496) expressed protein [Arabidopsis thaliana] | 364 |
| PP | | | | | | (NM_112272) expressed protein [Arabidopsis thaliana] | 365 |
| PP | | | | | | (NM_120465) Terminal flower1 (TFL1) [Arabidopsis thaliana] | 375 |
| PP | | | | | | AF488577_1 (AF488577) putative bHLH transcription factor [Arabidopsis thaliana] | 377 |
| PP | | | | | | arabinogalactan-protein [Arabidopsis thaliana] | 378 |
| PP | | | | | | (NM_111831) expressed protein [Arabidopsis thaliana] | 379 |
| PP | | | | | | (NM_129655) unknown protein [Arabidopsis thaliana] | 380 |
| PP | | | | | | (NM_126399) expressed protein [Arabidopsis thaliana] | 381 |
| PP | | | | | | (NM_100303) putative beta-ketoacyl-CoA synthase [Arabidopsis thaliana] pir\|\|T00951 | 382 |
| | | | | | | probable 3-oxoacyl-[acyl-carrier-protein] synthase (EC 2.3.1.41) F20D22.1 | |
| PP | | | | | | (NM_120596) putative protein [Arabidopsis thaliana] | 383 |
| PP | | | | | | (NM_125629) ripening-related protein-like [Arabidopsis thaliana] | 384 |
| PP | | | | | | (NM_120069) cysteine proteinase RD19A [Arabidopsis thaliana] | 385 |
| PP | | | | | | (NM_103632) expressed protein [Arabidopsis thaliana] | 386 |
| PP | | | | | | (NM_104392) expressed protein [Arabidopsis thaliana] | 387 |
| PP | | | | | | (NM_113306) PHD-finger protein, putative [Arabidopsis thaliana] | 388 |
| PP | | | | | | (NM_104559) integral membrane protein, putative [Arabidopsis thaliana] | 389 |
| PP | | | | | | (NM_125746) putative protein [Arabidopsis thaliana] | 392 |
| PP | | | | | | (NM_118332) alcohol dehydrogenase like protein [Arabidopsis thaliana] | 393 |
| PP | | | | | | (NM_122138) Ruv DNA-helicase-like protein [Arabidopsis thaliana] | 394 |
| PP | | | | | | (NC_001134) Amino acid transport protein for valine, leucine, isoleucine, and tyrosine; | 396 |
| | | | | | | Tat1p [Saccharomyces cerevisiae] | |
| PP | | | | | | LATE EMBRYOGENESIS ABUNDANT PROTEIN D-34 (LEA D-34) | 397 |
| PP | HS | | | | | (NM_124576) sorbitol dehydrogenase-like protein [Arabidopsis thaliana] | 368 |
| PP | LN | | | | | (NM_122624) RING-H2 zinc finger protein-like [Arabidopsis thaliana] | 371 |
| PP | PEG | | | | | (AB018114) RING finger protein-like [Arabidopsis thaliana] | 374 |
| PP | CS | | | | | (NM_115079) expressed protein [Arabidopsis thaliana] | 366 |
| PP | CS | | | | | auxin-regulated protein [Arabidopsis thaliana] gi\|30681325\|ref\|NP_849354.1 | 245 |
| PP | CS | | | | | (AC005990) Similar to OBP32pep protein gb\|U37698 from Arabidopsis thaliana | 250 |
| PP | CS | | | | | (NM_115730) transcriptional coactivator-like protein [Arabidopsis thaliana] | 260 |
| PP | CS | | | | | (NM_117229) phospholipid hydroperoxide glutathione peroxidase [Arabidopsis | 265 |
| | | | | | | thaliana] | |
| PP | CS | | | | | (NC_001136) phosphotyrosine-specific protein phosphatase; Ptp1p [Saccharomyces | 267 |
| | | | | | | cerevisiae] | |
| PP | HS | | | | | (AF374475) hypersensitive-induced response protein [Oryza sativa] | 294 |
| PP | SP | | | | | (NM_100509) expressed protein [Arabidopsis thaliana] | 461 |
| PP | PEG | | | | | (NM_118411) predicted protein [Arabidopsis thaliana] | 354 |
| PP | PEG | | | | | (NM_127582) expressed protein [Arabidopsis thaliana] | 358 |
| PP | LN | | | | | SKP1 family [Arabidopsis thaliana] gi\|9759236\|dbj\|BAB09760.1\|contains similarity to | 326 |
| | | | | | | elongin C~gene_id: MNC17.5 [Arabidopsis thaliana] gi\|15028385\|gb\|AAK76669.1\| | |
| | | | | | | putative elongin protein] | |
| PP | LN | | | | | (NM_100065) expressed protein [Arabidopsis thaliana] | 330 |
| PP | SP | LN | | | | glycine-rich RNA binding protein 7 [Arabidopsis thaliana] | 438 |
| PP | SS | HS | | | | (NM_100335) expressed protein [Arabidopsis thaliana] gb\|AAB80630.1\|(AC002376) | 285 |
| | | | | | | Strong similarity to Triticum ABA induced membrane protein (gb\|U80037) | |
| PP | HS | CS | | | | (NM_103926) sterol delta7 reductase [Arabidopsis thaliana] sp | 367 |
| PP | LL | LN | | | | (NM_117178) 98b like protein [Arabidopsis thaliana] p | 370 |
| PP | LL | SS | HS | CS | | (NM_111486) putative dual-specificity protein phosphatase [Arabidopsis thaliana] | 298 |
| SP | | | | | | (NM_104444) expressed protein [Arabidopsis thaliana] | 425 |
| SP | | | | | | Protein farnesyltransferase alpha subunit (CAAX farnesyltransferase alpha subunit) | 426 |
| | | | | | | (RAS proteins prenyltransferase alpha) (FTase-alpha)[Arabidopsis thaliana] | |
| SP | | | | | | (NM_129374) expressed protein [Arabidopsis thaliana] | 427 |
| SP | | | | | | (NM_101474) expressed protein [Arabidopsis thaliana] | 428 |
| SP | | | | | | (NM_103124) expressed protein [Arabidopsis thaliana] | 429 |
| SP | | | | | | (NM_116570) putative chloroplast protein import component [Arabidopsis thaliana] | 430 |
| SP | | | | | | (AC004667) expressed protein [Arabidopsis thaliana] gb\|AAM62820.1\|(AY085599) | 431 |
| | | | | | | zinc finger protein Glo3-like [Arabidopsis thaliana] | |
| SP | | | | | | (NM_104934) similar to flavin-containing monooxygenase (sp\|P36366); similar to | 432 |
| | | | | | | ESTs gb\|R30018, gb\|H36886, gb\|N37822, and gb\|T88100 [Arabidopsis thaliana] | |
| SP | | | | | | (NM_111084) expressed protein [Arabidopsis thaliana] | 433 |
| SP | | | | | | (NM_130339) expressed protein [Arabidopsis thaliana] | 436 |
| SP | | | | | | (NM_106509) expressed protein [Arabidopsis thaliana] | 440 |
| SP | | | | | | (NM_123934) GDSL-motif lipase/hydrolase-like protein [Arabidopsis thaliana] | 441 |
| SP | | | | | | (NM_128881) expressed protein [Arabidopsis thaliana] | 442 |
| SP | | | | | | (NM_111674) expressed protein [Arabidopsis thaliana] | 443 |
| SP | | | | | | (NM_115274) peptide transport-like protein [Arabidopsis thaliana] | 444 |
| SP | | | | | | NP_534027.1\|(NC_003305) 3-oxoacyl-(acyl-carrier-protein) reductase [Agrobacterium | 445 |
| | | | | | | tumefaciens str. C58 (U. Washington)] | |
| SP | | | | | | (NM_116899) nodulin-like protein [Arabidopsis thaliana] | 446 |
| SP | | | | | | (NM_120537) putative protein [Arabidopsis thaliana] | 447 |
| SP | | | | | | (NM_100079) expressed protein [Arabidopsis thaliana] | 448 |
| SP | | | | | | (NM_106680) putative sulfate transporter [Arabidopsis thaliana] | 449 |
| SP | | | | | | (NM_114633) putative protein [Arabidopsis thaliana] | 450 |
| SP | | | | | | (NM_114248) glutathione transferase-like protein [Arabidopsis thaliana] | 451 |
| SP | | | | | | (NM_115620) AP2 transcription factor-like protein [Arabidopsis thaliana] | 452 |
| SP | | | | | | (NM_102603) ethylene-responsive element binding factor, putative [Arabidopsis | 453 |
| | | | | | | thaliana] | |
| SP | | | | | | (NM_114527) scarecrow-like protein [Arabidopsis thaliana] | 454 |
| SP | | | | | | (AC000107) F17F8.3 [Arabidopsis thaliana] | 455 |
| SP | | | | | | (AC012188) Contains similarity to MYB-Related Protein B from Gallus gallus g | 456 |
| | | | | | | [Arabidopsis thaliana] | |
| SP | | | | | | (NM_122484) GATA transcription factor-like [Arabidopsis thaliana] | 457 |
| SP | | | | | | (NM_125879) expressed protein [Arabidopsis thaliana] | 458 |
| SP | | | | | | (NM_121609) UVB-resistance protein-like [Arabidopsis thaliana] | 459 |
| SP | | | | | | AAM62510.1\|(AY085278) homeodomain protein BELL1, putative [Arabidopsis | 460 |
| | | | | | | thaliana] | |
| SP | | | | | | (NM_129533) expressed protein [Arabidopsis thaliana] | 462 |
| SP | DS | | | | | (NM_101226) aminoalcoholphosphotransferase [Arabidopsis thaliana] | 403 |
| SP | DS | | | | | (NM_100757) superoxidase dismutase [Arabidopsis thaliana] | 405 |
| SP | DS | | | | | BAA97512.1\|(AB026634) 3′(2′),5′-bisphosphate nucleotidase protein-like protein | 412 |
| | | | | | | [Arabidopsis thaliana] | |
| SP | DS | | | | | (NM_118107) putative protein [Arabidopsis thaliana] | 419 |
| SP | HS | | | | | (NM_111278) NAM-like protein (no apical meristem) [Arabidopsis thaliana] | 289 |
| SP | LL | | | | | (NM_130339) expressed protein [Arabidopsis thaliana] | 436 |
| SP | LL | | | | | (NM_129353) expressed protein [Arabidopsis thaliana] | 437 |
| SP | PP | SS | | | | (NM_130274) putative protein kinase [Arabidopsis thaliana] pir\|\|T02181 protein kinase | 439 |
| | | | | | | homolog F14M4.11 | |
| SP | SS | PEG | | | | (NM_122447) cyclin 3a [Arabidopsis thaliana] gb\|AAC98445.1\|(AC006258) cyclin 3a | 424 |
| | | | | | | [Arabidopsis thaliana] | |
| SS | | | | | | (NM_106142) myb-related transcription activator, putative [Arabidopsis thaliana] | 463 |
| SS | | | | | | hypothetical protein F9G14.50 - Arabidopsis thaliana | 464 |
| SS | | | | | | AAD18098.1\|(AC006416) Identical to gb\|Y10557 g5bf gene from Arabidopsis thaliana | 465 |
| | | | | | | putative RNA-binding protein [Arabidopsis thaliana] | |
| SS | | | | | | (NM_105902) expressed protein [Arabidopsis thaliana] | 466 |
| SS | | | | | | Cytochrome P450 71B2 dbj\|BAA28537.1\|(D78605) cytochrome P450 | 467 |
| | | | | | | monooxygenase [Arabidopsis thaliana] | |
| SS | | | | | | (NM_114908) expressed protein [Arabidopsis thaliana] | 468 |
| SS | | | | | | (AB047808) proteasel (pfpl)-like protein [Arabidopsis thaliana] | 469 |
| SS | | | | | | (NM_121002) inorganic pyrophosphatase-like protein [Arabidopsis thaliana] | 470 |
| SS | | | | | | (NM_129704) calmodulin-like protein [Arabidopsis thaliana] | 471 |
| SS | | | | | | (NM_101236) unknown protein [Arabidopsis thaliana] | 472 |
| SS | | | | | | trypsin inhibitor-related [Arabidopsis thaliana] gi\|3287862\|sp\|O22867\|ITI5_ARATH | 475 |
| SS | | | | | | (NM_112176) DnaJ protein, putative [Arabidopsis thaliana] | 476 |
| SS | CK | | | | | (AB017071) zinc finger protein-like; Ser/Thr protein kinase-like protein [Arabidopsis | 243 |
| | | | | | | thaliana] | |
| SS | CS | | | | | (NM_116915) hypothetical protein [Arabidopsis thaliana] emb\|CAB77971.1 | 258 |
| SS | LL | | | | | (NM_100475) expressed protein [Arabidopsis thaliana] | 312 |
| SS | PP | | | | | (NM_127194) putative homeodomain transcription factor [Arabidopsis thaliana] | 390 |
| SS | PP | LN | | | | (NM_128415) putative AP2 domain transcription factor [Arabidopsis thaliana] | 372 |

*“CK” indicates cold tolerance improvement identified under a cold shock tolerance screen; “CS” indicates cold tolerance improvement identified by a cold germination tolerance screen; “DS” indicates drought tolerance improvement identified by a soil drought stress tolerance screen; “PEG” indicates osmotic stress tolerance improvement identified by a PEG induced osmotic stress tolerance screen; “HS” indicates heat stress tolerance improvement identified by a heat stress tolerance screen; “SS” indicates high salinity stress tolerance improvement identified by a salt stress tolerance screen; “LN” indicates nitrogen use efficiency improvement identified by a limited nitrogen tolerance screen; “LL” indicates attenuated shade avoidance response identified by a shade tolerance screen under a low light condition; “PP” indicates improved growth and development at early stages identified by an early plant growth and development screen; “SP” indicates improved growth and development at late stages identified by a late plant growth and development screen as described in US 20060041961, and incorporated herein by reference. Some proteins may provide more than one traits. For example, proteins of SEQ ID NOs 240 and 474.

TABLE 4
Examples of traits and proteins suitable for use in the present invention.*

| | | | | | | Protein | |
|---|---|---|---|---|---|---|---|
| Traits | | | | | | SEQ ID | Protein |
| CK | SS | | | | | | ref\|NP_191546.1\|expressed protein [Arabidopsis thaliana] pir\|\|T47811 hypothetical |
| | | | | | | | protein F24G16.140 - Arabidopsis thaliana emb\|CAB75806.1\|putative protein |
| | | | | | | | [Arabidopsis thaliana] |
| CK | | | | | | 271 | ref\|NP_173239.1\|zinc finger (C3HC4-type RING finger) family protein [Arabidopsis |
| | | | | | | | thaliana] |
| CK | | | | | | 272 | ref\|NP_565870.1\|expressed protein [Arabidopsis thaliana] |
| CK | CS | CK | HS | PP | | 273 | ref\|NP_850953.1\|MADS-box protein (AGL9) [Arabidopsis thaliana] gb\|AAM65812.1\| |
| | | | | | | | putative floral homeotic protein, AGL9 [Arabidopsis thaliana] |
| CK | | | | | | 274 | pir\|\|H96827protein F20B17.12 [imported] - Arabidopsis thaliana gb\|AAF68121.1\| |
| | | | | | | | F20B17.12 [Arabidopsis thaliana] |
| CK | | | | | | 275 | ref\|NP_565174.1\|14-3-3 protein GF14 pi (GRF13) [Arabidopsis thaliana] |
| CK | | | | | | 276 | ref\|NP_180770.1\|ovate protein-related [Arabidopsis thaliana] pir\|\|A84729 hypothetical |
| | | | | | | | protein At2g32100 [imported] - Arabidopsis thaliana |
| CK | | | | | | 277 | ref\|NP_200093.1\|ornithine cyclodeaminase/mu-crystallin family protein [Arabidopsis |
| | | | | | | | thaliana] dbj\|BAB10429.1\|unnamed protein product [Arabidopsis thaliana] |
| CK | | | | | | 278 | ref\|NP_180115.1\|2-oxoglutarate-dependent dioxygenase, putative [Arabidopsis thaliana] |
| | | | | | | | pir\|\|E84648 probable dioxygenase [imported] |
| CK | | | | | | 279 | ref\|NP_186854.1\|potassium transporter (KUP3) [Arabidopsis thaliana] |
| CK | | | | | | 280 | ref\|NP_179547.1\|cytidine deaminase (CDD)/cytidine aminohydrolase [Arabidopsis |
| | | | | | | | thaliana] |
| CK | | | | | | 281 | ref\|NP_566315.1\|ABC1 family protein [Arabidopsis thaliana] |
| CK | | | | | | 282 | ref\|NP_195575.1\|26S proteasome regulatory subunit S5A (RPN10) [Arabidopsis |
| | | | | | | | thaliana] sp\|P55034\|PDS4_ARATH 26S proteasome non-ATPase regulatory subunit 4 |
| | | | | | | | (26S proteasome regulatory subunit S5A) (Multiubiquitin chain binding protein) |
| | | | | | | | pir\|\|T05691 multiubiquitin chain-binding protein MBP1 - Arabidopsis thaliana |
| CK | | | | | | 283 | ref\|NP_568693.1\|expressed protein [Arabidopsis thaliana] |
| CK | | | | | | 284 | ref\|NP_194797.1\|MA3 domain-containing protein [Arabidopsis thaliana] pir\|\|A85359 |
| | | | | | | | translation initiation factor-like protein [imported] - Arabidopsis thaliana |
| | | | | | | | emb\|CAB52441.1\|translation initiation factor-like protein [Arabidopsis thaliana] |
| | | | | | | | emb\|CAB79786.1\|translation initiation factor-like protein [Arabidopsis thaliana] |
| CK | PP | | | | | 285 | ref\|NP_931714.1\|Fructose-1,6-bisphosphatase (D-fructose-1,6-bisphosphate 1- |
| | | | | | | | phosphohydrolase) (FBPase) [Photorhabdus luminescens subsp. laumondii TTO1] |
| CK | | | | | | 286 | ref\|NP_389241.1\|similar to aspartate aminotransferase [Bacillus subtilis] |
| | | | | | | | sp\|O31665\|MTNE_BACSU Transaminase mtnE pir\|\|F69863 probable transaminase (EC |
| | | | | | | | 2.6.1.—) ykrV [similarity] - Bacillus subtilis emb\|CAB13231.1\|ykrV [Bacillus subtilis subsp. |
| | | | | | | | subtilis str. 168] |
| CK | SP | | | | | 287 | ref\|NP_200760.1\|zinc transporter (ZIP2) [Arabidopsis thaliana] sp\|Q9LTH9\|ZIP2_ARATH |
| | | | | | | | Zinc transporter 2 precursor (ZRT/IRT-like protein 2) |
| CK | LL | PEG | | | | 382 | ref\|NP_195030.1\|glutaredoxin family protein [Arabidopsis thaliana] pir\|\|T05312 |
| | | | | | | | hypothetical protein F26P21.160 - Arabidopsis thaliana |
| CK | LL | | | | | 383 | ref\|NP_567140.1\|expressed protein [Arabidopsis thaliana] |
| CK | LN | | | | | 389 | ref\|NP_186983.2\|short-chain dehydrogenase/reductase (DSR) family protein |
| | | | | | | | [Arabidopsis thaliana] gb\|AAM83244.1\|AT3g03330/T21P5_25 [Arabidopsis thaliana] |
| | | | | | | | gb\|AAN18119.1\|At3g03330/T21P5_25 [Arabidopsis thaliana] |
| CK | LN | | | | | 398 | ref\|NP_194188.1\|mitochondrial substrate carrier family protein [Arabidopsis thaliana] |
| | | | | | | | pir\|\|T05577 uncoupling protein homolog F22K18.230 - Arabidopsis thaliana |
| CK | PEG | | | | | 402 | ref\|NP_567196.1\|auxin-responsive family protein [Arabidopsis thaliana] pir\|\|T01558 |
| | | | | | | | auxin-induced protein homolog A_TM018A10.6 |
| CK | PEG | | | | | 403 | ref\|NP_636070.1\|conserved hypothetical protein [Xanthomonas campestris pv. |
| | | | | | | | campestris str. ATCC 33913] |
| CK | PEG | | | | | 404 | ref\|NP_182075.1\|cytochrome P450, putative [Arabidopsis thaliana] pir\|\|T00864 |
| | | | | | | | cytochrome P450 homolog F17K2.4 - Arabidopsis thaliana gb\|AAC06153.1\|putative |
| | | | | | | | cytochrome P450 [Arabidopsis thaliana] gb\|AAO43566.1\|At2g45510 [Arabidopsis |
| | | | | | | | thaliana] |
| CK | PP | SP | | | | 432 | ref\|NP_189259.1\|cytochrome P450 family protein [Arabidopsis thaliana] dbj\|BAB02451.1\| |
| | | | | | | | cytochrome P450 [Arabidopsis thaliana] gb\|AAL90915.1\|AT3g26280/MTC11_19 |
| | | | | | | | [Arabidopsis thaliana] gb\|AAN31105.1\|At3g26280/MTC11_19 [Arabidopsis thaliana] |
| CK | PP | SP | | | | 433 | ref\|NP_175816.1\|transcription initiation factor IID (TFIID) 31 kDa subunit (TAFII-31) |
| | | | | | | | family protein [Arabidopsis thaliana] pir\|\|E96582 hypothetical protein F15I1.24 [imported] - |
| | | | | | | | Arabidopsis thaliana gb\|AAD25788.1\|Similar to gb\|U21858 transcription initiation factor |
| | | | | | | | TFIID 31 KD subunit (TAFII32) from Homo sapiens. [Arabidopsis thaliana] |
| | | | | | | | gb\|AAF65406.1\|putative TATA binding protein associated factor 21 kDa subunit |
| | | | | | | | [Arabidopsis thaliana] gb\|AAM65438.1\|transcriptional activation factor TAFII32, putative |
| | | | | | | | [Arabidopsis thaliana] gb\|AAR28026.1\|TAF9 [Arabidopsis thaliana] |
| CK | PEG | CS | PP | PEG | | 438 | ref\|NP_012776.1\|Vacuolar transporter, exports large neutral amino acids from the |
| | | | | | | | vacuole; member of a family of seven S. cerevisiae genes (AVT1-7) related to vesicular |
| | | | | | | | GABA-glycine transporters; Avt3p [Saccharomyces cerevisiae] |
| | | | | | | | sp\|P36062\|YKO6_YEAST Hypothetical 75.5 kDa protein in DSH1-CIM5/YTA3 intergenic |
| | | | | | | | region pir\|\|S37976 hypothetical protein YKL146w-yeast (Saccharomyces cerevisiae) |
| | | | | | | | emb\|CAA81988.1\|unnamed protein product [Saccharomyces cerevisiae] |
| CK | PP | SP | SS | | | 443 | ref\|NP_174236.1\|auxin-responsive family protein [Arabidopsis thaliana] pir\|\|A86417 |
| | | | | | | | probable auxin-induced protein, 45653-45228 |
| CK | PEG | SS | | | | 510 | ref\|NP_012965.1\|general amino acid permease; Gap1p [Saccharomyces cerevisiae] |
| | | | | | | | sp\|P19145\|GAP1_YEAST General amino-acid permease GAP1 pir\|\|S38111 amino acid |
| | | | | | | | transport protein GAP1 [validated]-yeast (Saccharomyces cerevisiae) |
| | | | | | | | emb\|CAA82113.1\|GAP1 [Saccharomyces cerevisiae] |
| CS | | | | | | 288 | ref\|NP_565053.1\|SNF7 family protein [Arabidopsis thaliana] pir\|\|G96755 developmental |
| | | | | | | | protein homolog DG1118 [imported] - Arabidopsis thaliana |
| CS | | | | | | 289 | ref\|NP_013836.1\|Hypothetical ORF; Ymr118cp [Saccharomyces cerevisiae] |
| | | | | | | | sp\|Q04487\|YM07_YEAST Putative succinate dehydrogenase cytochrome B subunit, |
| | | | | | | | mitochondrial precursor pir\|\|S54580 probable membrane protein YMR118c-yeast |
| | | | | | | | (Saccharomyces cerevisiae) emb\|CAA89756.1\|unknown [Saccharomyces cerevisiae] |
| | | | | | | | gb\|AAS56294.1\|YMR118C [Saccharomyces cerevisiae] |
| CS | | | | | | 290 | ref\|NP_566531.1\|expressed protein [Arabidopsis thaliana] |
| CS | PP | | | | | 291 | pir\|\|E84768hypothetical protein At2g35430 [imported] - Arabidopsis thaliana |
| | | | | | | | gb\|AAC36178.1\|hypothetical protein [Arabidopsis thaliana] |
| CS | | | | | | 292 | ref\|NP_191966.2\|malate oxidoreductase, putative [Arabidopsis thaliana] gb\|AAM91599.1\| |
| | | | | | | | putative malate oxidoreductase [Arabidopsis thaliana] |
| CS | LL | PP | | | | 293 | ref\|NP_566667.1\|transcription factor jumonji (jmjC) domain-containing protein |
| | | | | | | | [Arabidopsis thaliana] dbj\|BAB02489.1\|unnamed protein product [Arabidopsis thaliana] |
| | | | | | | | gb\|AAM61040.1\|unknown [Arabidopsis thaliana] |
| CS | | | | | | 294 | sp\|Q9STL0\|C71N_ARATHCytochrome P450 71A23 pir\|\|T06712 probable cytochrome |
| | | | | | | | P450 T29H11.180 - Arabidopsis thaliana emb\|CAB41168.1\|cytochrome p450 like |
| | | | | | | | protein [Arabidopsis thaliana] |
| CS | SP | | | | | 295 | ref\|NP_192100.1\|DC1 domain-containing protein [Arabidopsis thaliana] pir\|\|E85024 |
| | | | | | | | probable CHP-rich zinc finger protein [imported] - Arabidopsis thaliana gb\|AAD22653.1\| |
| | | | | | | | putative CHP-rich zinc finger protein |
| CS | | | | | | 296 | ref\|NP_013583.1\|High-affinity inorganic phosphate (Pi) transporter and low-affinity |
| | | | | | | | manganese transporter; regulated by Pho4p and Spt7p; mutation confers resistance to |
| | | | | | | | arsenate; exit from the ER during maturation requires Pho86p; Pho84p [Saccharomyces |
| | | | | | | | cerevisiae] sp\|P25297\|PH84_YEAST Inorganic phosphate transporter PHO84 |
| | | | | | | | pir\|\|S54061 probable inorganic phosphate transport protein PHO84-yeast |
| | | | | | | | (Saccharomyces cerevisiae) emb\|CAA89157.1\|Pho84p [Saccharomyces cerevisiae] |
| CS | | | | | | 297 | ref\|NP_010071.1\|GABA-specific transport protein; Uga4p [Saccharomyces cerevisiae] |
| | | | | | | | sp\|P32837\|UGA4_YEAST GABA-specific permease (GABA-specific transport protein) |
| | | | | | | | pir\|\|S30253 GABA transport protein-yeast |
| CS | | | | | | 298 | ref\|NP_012036.1\|Subunit of the anaphase-promoting complex/cyclosome (APC/C), |
| | | | | | | | which is a ubiquitin-protein ligase required for degradation of anaphase inhibitors, |
| | | | | | | | including mitotic cyclins, during the metaphase/anaphase transition; Cdc23p |
| | | | | | | | [Saccharomyces cerevisiae] sp\|P16522\|CC23_YEAST Cell division control protein 23 |
| CS | PP | | | | | 299 | ref\|NP_440881.1\|fructokinase [Synechocystis sp. PCC 6803] pir\|\|S77227 fructokinase |
| | | | | | | | (EC 2.7.1.4) - Synechocystis sp. (strain PCC 6803) |
| CS | LL | PP | PEG | | | 300 | ref\|NP_389366.1\|similar to glutaminase [Bacillus subtilis] sp\|O07637\|GLS1_BACSU |
| | | | | | | | Probable glutaminase ylaM pir\|\|C69873 glutaminase homolog ylaM-Bacillus subtilis |
| | | | | | | | emb\|CAB09718.1\|ylaM [Bacillus subtilis] emb\|CAB13356.1\|ylaM [Bacillus subtilis |
| | | | | | | | subsp. subtilis str. 168] |
| CS | PP | | | | | 301 | ref\|NP_355719.1\|AGR_C_5067p [Agrobacterium tumefaciens str. C58] ref\|NP_533456.1\| |
| | | | | | | | 3-isopropylmalate dehydrogenase [Agrobacterium tumefaciens str. C58] |
| | | | | | | | sp\|P24404\|LEU3_AGRT5 3-isopropylmalate dehydrogenase (Beta-IPM dehydrogenase) |
| | | | | | | | (IMDH) (3-IPM-DH) |
| CS | DS | PP | | | | 302 | ref\|NP_388498.1\|similar to fructokinase [Bacillus subtilis] sp\|O34768\|YDJE_BACSU |
| | | | | | | | Hypothetical sugar kinase ydjE pir\|\|A69789 fructokinase homolog ydjE - Bacillus subtilis |
| | | | | | | | dbj\|BAA22760.1\|sugar transport protein [Bacillus subtilis] emb\|CAB12436.1\|ydjE |
| | | | | | | | [Bacillus subtilis subsp. subtilis str. 168] |
| CS | PP | | | | | 303 | ref\|NP_193383.1\|cysteine protease inhibitor family protein/cystatin family protein |
| | | | | | | | [Arabidopsis thaliana] pir\|\|H71431 hypothetical protein - Arabidopsis thaliana |
| | | | | | | | emb\|CAB10426.1\|cysteine proteinase inhibitor like protein [Arabidopsis thaliana] |
| CS | | | | | | 304 | ref\|ZP_00084307.1\|COG1012: NAD-dependent aldehyde dehydrogenases |
| | | | | | | | [Pseudomonas fluorescens PfO-1] |
| CS | | | | | | 305 | ref\|NP_418028.1\|alpha-amylase [Escherichia coli K12] sp\|P25718\|AMY1_ECOLI Alpha- |
| | | | | | | | amylase precursor (1,4-alpha-D-glucan glucanohydrolase) pir\|\|S23807 alpha-amylase |
| | | | | | | | (EC 3.2.1.1) precursor, periplasmic - Escherichia coli (strain K-12) |
| CS | CK | HS | | | | 306 | ref\|NP_441721.1\|unknown protein [Synechocystis sp. PCC 6803] |
| CS | LL | | | | | 307 | ref\|NP_707335.1\|glyceraldehyde-3-phosphate dehydrogenase A [Shigella flexneri 2a str. |
| | | | | | | | 301] |
| CS | | | | | | 308 | ref\|NP_566402.1\|U-box domain-containing protein [Arabidopsis thaliana] |
| CS | | | | | | 309 | ref\|NP_565676.1\|armadillo/beta-catenin repeat family protein/U-box domain-containing |
| | | | | | | | protein [Arabidopsis thaliana] |
| CS | HS | | | | | 310 | ref\|NP_176847.1\|cell division protein kinase, putative [Arabidopsis thaliana] |
| CS | | | | | | 311 | ref\|NP_181907.1\|mitogen-activated protein kinase, putative/MAPK, putative (MPK6) |
| | | | | | | | [Arabidopsis thaliana |
| CS | | | | | | 312 | ref\|NP_180094.2\|protein kinase family protein [Arabidopsis thaliana] |
| CS | HS | SP | | | | 313 | ref\|NP_176132.1\|amino acid permease I (AAP1) [Arabidopsis thaliana] pir\|\|A48187 |
| | | | | | | | amino acid transport protein I - Arabidopsis thaliana |
| CS | PP | | | | | 314 | ref\|ZP_00024043.1\|COG0252: L-asparaginase/archaeal Glu-tRNAGln amidotransferase |
| | | | | | | | subunit D [Ralstonia metallidurans] |
| CS | DS | | | | | 315 | ref\|NP_974481.1\|kelch repeat-containing F-box family protein [Arabidopsis thaliana] |
| CS | DS | LL | LN | | | 316 | ref\|NP_171627.1\|cytochrome P450, putative [Arabidopsis thaliana] |
| CS | HS | | | | | 353 | ref\|NP_563782.1\|expressed protein [Arabidopsis thaliana] |
| CS | HS | | | | | 360 | ref\|NP_196201.2\|phosphate translocator-related [Arabidopsis thaliana] |
| CS | HS | PP | | | | 361 | gb\|AAF23363.1\|CAGL2 [Cucumis sativus] |
| CS | LL | PEG | | | | 373 | ref\|NP_199519.1\|casein kinase II beta chain, putative [Arabidopsis thaliana] |
| | | | | | | | sp\|P40228\|KC2B_ARATH Casein kinase II beta chain (CK II) pir\|\|S47967 casein kinase |
| | | | | | | | II (EC 2.7.1.—) beta chain CKB1 |
| CS | HS | PP | | | | 434 | ref\|NP_177792.1\|expressed protein [Arabidopsis thaliana] |
| CS | CK | PP | | | | 435 | ref\|ZP_00027327.1\|COG1012: NAD-dependent aldehyde dehydrogenases [Burkholderia |
| | | | | | | | fungorum] |
| CS | PEG | CK | PP | PEG | | 439 | ref\|NP_564120.1\|catalase 3 (SEN2) [Arabidopsis thaliana] |
| CS | CK | HS | LL | PP | SS | 444 | ref\|NP_011602.1\|Cytosolic catalase T, has a role in protection from oxidative damage by |
| | | | | | | | hydrogen peroxide; Ctt1p [Saccharomyces cerevisiae] sp\|P06115\|CATT_YEAST |
| | | | | | | | Catalase T pir\|\|CSBYT catalase (EC 1.11.1.6) T-yeast (Saccharomyces cerevisiae) |
| | | | | | | | emb\|CAA97090.1\|CTT1 [Saccharomyces cerevisiae] |
| CS | PP | | | | | 459 | ref\|NP_568066.1\|expressed protein [Arabidopsis thaliana] |
| CS | PP | | | | | 461 | ref\|NP_171806.1\|expressed protein [Arabidopsis thaliana] pir\|\|E86161 F10O3.11 protein - |
| | | | | | | | Arabidopsis thaliana gb\|AAD25802.1\|Belongs to the PF\|01027 Uncharacterized protein |
| | | | | | | | family UPF0005 with 7 transmembrane domains. [Arabidopsis thaliana] gb\|AAR24681.1\| |
| | | | | | | | At1g03070 [Arabidopsis thaliana] |
| CS | PP | | | | | 477 | ref\|NP_931461.1\|Phenylacetaldehyde dehydrogenase (PAD) [Photorhabdus |
| | | | | | | | luminescens subsp. laumondii TTO1] |
| CS | HS | SS | | | | 504 | ref\|NP_175431.1\|branched-chain amino acid aminotransferase 6/branched-chain amino |
| | | | | | | | acid transaminase 6 (BCAT6) [Arabidopsis thaliana] s |
| CS | SS | HS | LN | PP | | 505 | ref\|NP_193115.1\|auxin-responsive protein, putative [Arabidopsis thaliana] pir\|\|T05248 |
| | | | | | | | probable auxin-induced protein F18A5.180 - Arabidopsis thaliana emb\|CAB36843.1\| |
| | | | | | | | SAUR-AC-like protein (small auxin up RNA) [Arabidopsis thaliana] emb\|CAB78421.1\| |
| | | | | | | | SAUR-AC-like protein (small auxin up RNA) [Arabidopsis thaliana] |
| CS | PP | SS | | | | 507 | ref\|NP_441729.1\|unknown protein [Synechocystis sp. PCC 6803] |
| CS | SS | | | | | 514 | gb\|AAO45179.1\|transcription factor Myb1 [Malus xiaojinensis] |
| DS | | | | | | 317 | ref\|ZP_00022985.1\|COG0538: Isocitrate dehydrogenases [Ralstonia metallidurans] |
| DS | PEG | | | | | 318 | ref\|NP_850277.2\|CCAAT-box binding transcription factor, putative [Arabidopsis thaliana] |
| DS | | | | | | 319 | ref\|NP_187154.1\|sodium proton exchanger, putative (NHX2) [Arabidopsis thaliana]] |
| DS | | | | | | 320 | ref\|NP_192555.1\|homeobox protein knotted-1 like 1 (KNAT1) [Arabidopsis thaliana] |
| | | | | | | | sp\|P46639\|HKL1_ARATH Homeobox protein knotted-1 like 1 (KNAT1) pir\|\|D85080 |
| | | | | | | | KNAT1 homeobox-like protein [imported]- Arabidopsis thaliana g |
| DS | | | | | | 321 | ref\|NP_194642.1\|hexokinase 1 (HXK1) [Arabidopsis thaliana] sp\|Q42525\|HXK1_ARATH |
| | | | | | | | Hexokinase 1 pir\|\|S71205 hexokinase (EC 2.7.1.1) 1 - Arabidopsis thaliana |
| DS | | | | | | 322 | ref\|NP_190238.1\|epsin N-terminal homology (ENTH) domain-containing protein/clathrin |
| | | | | | | | assembly protein-related [Arabidopsis thaliana] |
| DS | PP | | | | | 323 | ref\|NP_180632.1\|serine/threonine protein kinase, putative [Arabidopsis thaliana] |
| | | | | | | | pir\|\|B84712 probable protein kinase [imported] |
| DS | | | | | | 324 | ref\|NP_177749.1\|hypothetical protein [Arabidopsis thaliana] gb\|AAF17642.1\|T23E18.15 |
| | | | | | | | [Arabidopsis thaliana] |
| DS | | | | | | 325 | ref\|NP_567716.1\|expressed protein [Arabidopsis thaliana] |
| DS | | | | | | 326 | ref\|NP_197527.1\|expressed protein [Arabidopsis thaliana] |
| DS | | | | | | 327 | ref\|NP_566090.1\|expressed protein [Arabidopsis thaliana] |
| DS | | | | | | 328 | ref\|NP_197755.1\|nodulin MtN3 family protein [Arabidopsis thaliana] pir\|\|T51837 MTN3 |
| | | | | | | | homolog [imported]- Arabidopsis thaliana gb\|AAC64192.1\|MTN3 homolog [Arabidopsis |
| | | | | | | | thaliana] |
| DS | | | | | | 329 | ref\|NP_192875.1\|zinc finger (C3HC4-type RING finger) family protein (RHA1b) |
| | | | | | | | [Arabidopsis thaliana] pir\|\|T13027 RING-H2 finger protein RHA1b - Arabidopsis thaliana |
| | | | | | | | emb\|CAB51420.1\|RING-H2 finger protein RHA1b [Arabidopsis thaliana] |
| DS | PP | | | | | 330 | ref\|NP_849549.1\|zinc finger protein (LDS1) [Arabidopsis thaliana] |
| DS | | | | | | 331 | ref\|NP_010143.1\|plasma membrane glucose sensor; Rgt2p [Saccharomyces cerevisiae] |
| | | | | | | | sp\|Q12300\|RGT2_YEAST High-affinity glucose transporter RGT2 pir\|\|S67684 probable |
| | | | | | | | membrane protein YDL138w-yeast (Saccharomyces cerevisiae) emb\|CAA65621.1\| |
| | | | | | | | RGT2 [Saccharomyces cerevisiae] emb\|CAA98711.1\|RGT2 [Saccharomyces |
| | | | | | | | cerevisiae] |
| DS | | | | | | 332 | ref\|NP_194205.1\|protein kinase (AFC2) [Arabidopsis thaliana] sp\|P51567\|AFC2_ARATH |
| | | | | | | | Protein kinase |
| DS | | | | | | 333 | ref\|NP_638775.1\|conserved hypothetical protein [Xanthomonas campestris pv. |
| | | | | | | | campestris str. ATCC 33913] |
| DS | PP | SP | | | | 334 | ref\|NP_566180.1\|integral membrane family protein [Arabidopsis thaliana] |
| | | | | | | | gb\|AAK73991.1\|AT3g02690/F16B3_32 [Arabidopsis thaliana] |
| DS | | | | | | 335 | ref\|NP_196953.1\|no apical meristem (NAM) family protein [Arabidopsis thaliana] |
| | | | | | | | pir\|\|T48622 hypothetical protein F18O22.280 - Arabidopsis thaliana emb\|CAB87788.1\| |
| | | | | | | | putative protein [Arabidopsis thaliana] |
| DS | | | | | | 336 | ref\|NP_173010.1\|cyclin, putative [Arabidopsis thaliana] |
| DS | | | | | | 337 | ref\|NP_568508.2\|bZIP transcription factor family protein [Arabidopsis thaliana] |
| DS | | | | | | 338 | ref\|NP_181899.1\|acyl-[acyl-carrier-protein] desaturase/stearoyl-ACP desaturase (SSI2) |
| | | | | | | | [Arabidopsis thaliana] pir\|\|E84869 stearoyl-ACP desaturase [imported] - Arabidopsis |
| | | | | | | | thaliana gb\|AAB64035.1\|stearoyl-ACP desaturase [Arabidopsis thaliana] |
| | | | | | | | gb\|AAK85232.1\|stearoyl ACP desaturase [Arabidopsis thaliana] |
| DS | | | | | | 339 | emb\|CAB75429.1\|oligouridylate binding protein [Nicotiana plumbaginifolia] |
| DS | | | | | | 340 | ref\|NP_192588.1\|mitogen-activated protein kinase, putative [Arabidopsis thaliana] |
| | | | | | | | pir\|\|T01835 serine/threonine-specific protein kinase ARA.KIN homolog T15F16.3 - |
| | | | | | | | Arabidopsis thaliana |
| DS | | | | | | 341 | gb\|AAD56659.1\|malate dehydrogenase [Glycine max] |
| DS | | | | | | 342 | gb\|AAM62553.1\|snap25a [Arabidopsis thaliana] |
| DS | | | | | | 343 | sp\|P38604\|ERG7_YEASTLanosterol synthase (Oxidosqualene--lanosterol cyclase) (2,3- |
| | | | | | | | epoxysqualene--lanosterol cyclase) (OSC) gb\|AAA64377.1\|2,3-oxidosqualene-lanosterol |
| | | | | | | | cyclase |
| DS | | | | | | 344 | ref\|NP_683594.1\|NPR1/NIM1-interacting protein 2 (NIMIN-2) [Arabidopsis thaliana] |
| | | | | | | | dbj\|BAB01050.1\|unnamed protein product [Arabidopsis thaliana] emb\|CAC19845.1\| |
| | | | | | | | NIMIN-2 protein [Arabidopsis thaliana] |
| DS | | | | | | 345 | ref\|NP_200108.1\|expressed protein [Arabidopsis thaliana] |
| DS | | | | | | 346 | ref\|NP_200558.1\|expressed protein [Arabidopsis thaliana] |
| DS | SS | | | | | 347 | ref\|NP_014004.1\|Carboxy-terminal domain (CTD) phosphatase, essential for |
| | | | | | | | dephosphorylation of the repeated C-terminal domain of the RNA polymerase II large |
| | | | | | | | subunit (Rpo21p); Fcp1p [Saccharomyces cerevisiae] sp\|Q03254\|FCP1_YEAST RNA |
| | | | | | | | polymerase II subunit A C-terminal domain phosphatase (CTD phosphatase FCP1) |
| | | | | | | | pir\|\|S54584 hypothetical protein YMR277w-yeast (Saccharomyces cerevisiae) |
| | | | | | | | emb\|CAA89775.1\|unknown [Saccharomyces cerevisiae] |
| DS | | | | | | 348 | sp\|Q00618\|BET4_YEASTGeranylgeranyl transferase type II alpha subunit (Type II |
| | | | | | | | protein geranyl-geranyltransferase alpha subunit) (GGTase-II-alpha) (PGGT) |
| | | | | | | | (YPT1/SEC4 proteins geranylgeranyltransferase alpha subunit) pir\|\|S48301 |
| | | | | | | | geranylgeranyl transferase (EC 2.5.1.—) alpha chain-yeast (Saccharomyces cerevisiae) |
| DS | | | | | | 349 | ref\|NP_567780.1\|pfkB-type carbohydrate kinase family protein [Arabidopsis thaliana] |
| DS | | | | | | 350 | ref\|NP_197938.2\|zinc finger (C3HC4-type RING finger) family protein [Arabidopsis |
| | | | | | | | thaliana] |
| DS | LN | | | | | 351 | ref\|NP_182037.1\|zinc finger (C2H2 type) family protein [Arabidopsis thaliana] pir\|\|F84886 |
| | | | | | | | probable C2H2-type zinc finger protein [imported] - Arabidopsis thaliana gb\|AAD32825.1\| |
| | | | | | | | putative C2H2-type zinc finger protein [Arabidopsis thaliana] gb\|AAL69471.1\| |
| | | | | | | | At2g45120/T14P1.7 [Arabidopsis thaliana] |
| DS | LL | | | | | 374 | ref\|NP_196763.1\|17.6 kDa class II heat shock protein (HSP17.6-CII) [Arabidopsis |
| | | | | | | | thaliana] |
| DS | LN | | | | | 390 | gb\|AAF24061.1\|v-SNARE AtVTI1a [Arabidopsis thaliana] |
| DS | LL | LN | | | | 397 | ref\|NP_566542.1\|mitotic phosphoprotein N′ end (MPPN) family protein [Arabidopsis |
| | | | | | | | thaliana] |
| DS | PP | SP | | | | 454 | ref\|NP_196916.1\|nodulin family protein [Arabidopsis thaliana] |
| HS | CS | PEG | | | | 352 | ref\|NP_390450.1\|similar to 6-phosphogluconate dehydrogenase (pentose phosphate) |
| | | | | | | | [Bacillus subtilis] sp\|P54448\|YQEC_BACSU Hypothetical protein yqeC pir\|\|G69950 6- |
| | | | | | | | phosphogluconate dehydrogenase (pentose) homolog yqeC - Bacillus subtilis |
| | | | | | | | dbj\|BAA12439.1\|YqeC [Bacillus subtilis] emb\|CAB14514.1\|yqeC [Bacillus subtilis subsp. |
| | | | | | | | subtilis str. 168] |
| HS | | | | | | 354 | ref\|NP_564592.1\|F-box family protein [Arabidopsis thaliana] |
| HS | | | | | | 355 | ref\|NP_177373.1\|trypsin and protease inhibitor family protein/Kunitz family protein |
| | | | | | | | [Arabidopsis thaliana] pir\|\|F96746 probable drought induced protein |
| HS | | | | | | 356 | ref\|NP_567287.1\|vesicle-associated membrane family protein/VAMP family protein |
| | | | | | | | [Arabidopsis thaliana] gb\|AAM19836.1\|AT4g05060/T32N4_12 [Arabidopsis thaliana] |
| | | | | | | | gb\|AAN28781.1\|At4g05060/T32N4_12 [Arabidopsis thaliana] |
| HS | | | | | | 357 | ref\|NP_201424.1\|expressed protein [Arabidopsis thaliana] |
| HS | SS | | | | | 358 | gb\|AAN65180.1\|mitogen-activated protein kinase 4 [Petroselinum crispum] |
| HS | | | | | | 359 | pir\|\|T09700MADS-box protein-alfalfa (fragment) gb\|AAB51377.1\|MADS-box protein |
| | | | | | | | [Medicago sativa] |
| HS | | | | | | 362 | ref\|NP_188469.1\|no apical meristem (NAM) family protein [Arabidopsis thaliana] |
| | | | | | | | dbj\|BAB01106.1\|unnamed protein product [Arabidopsis thaliana] |
| HS | CS | | SS | | | 363 | ref\|NP_564504.1\|protein phosphatase 2C-related/PP2C-related [Arabidopsis thaliana] |
| HS | | | | | | 364 | ref\|NP_200357.2\|protease inhibitor/seed storage/lipid transfer protein (LTP) family |
| | | | | | | | protein [Arabidopsis thaliana] dbj\|BAC43554.1\|unknown protein [Arabidopsis thaliana] |
| HS | PP | | | | | 365 | ref\|NP_194791.1\|expressed protein [Arabidopsis thaliana] |
| HS | | | | | | 366 | ref\|NP_196433.1\|serine/threonine protein kinase, putative [Arabidopsis thaliana] |
| HS | | | | | | 367 | ref\|NP_565804.1\|expressed protein [Arabidopsis thaliana] |
| HS | CK | | | | | 368 | ref\|NP_195218.1\|1-phosphatidylinositol phosphodiesterase-related [Arabidopsis thaliana] |
| | | | | | | | pir\|\|T10238 hypothetical protein T11I11.160 - Arabidopsis thaliana e |
| HS | | | | | | 369 | ref\|NP_194120.1\|expressed protein [Arabidopsis thaliana] pir\|\|T08908 hypothetical |
| | | | | | | | protein T32A16.60 - Arabidopsis thaliana |
| HS | LL | PEG | | | | 370 | ref\|NP_197088.1\|zinc finger protein CONSTANS (CO) [Arabidopsis thaliana] |
| | | | | | | | sp\|Q39057\|CONS_ARATH Zinc finger protein CONSTANS pir\|\|A56133 constans protein- |
| | | | | | | | Arabidopsis thaliana emb\|CAA64407.1\|CONSTANS protein [Arabidopsis thaliana] |
| | | | | | | | eONSTANS protein [Arabidopsis thaliana] |
| HS | | | | | | 371 | ref\|NP_790208.1\|glutamine synthetase, type I [Pseudomonas syringae pv. tomato str. |
| | | | | | | | DC3000] gb\|AAO53903.1\|glutamine synthetase, type I [Pseudomonas syringae pv. |
| | | | | | | | tomato str. DC3000] |
| HS | | | | | | 372 | ref\|NP_242490.1\|L-asparaginase [Bacillus halodurans C-125] |
| HS | PEG | CK | | | | 409 | ref\|NP_011651.1\|20S proteasome beta-type subunit; the only nonessential 20S subunit; |
| | | | | | | | Pre9p [Saccharomyces cerevisiae] sp\|P23638\|PSA4_YEAST Proteasome component |
| | | | | | | | Y13 (Macropain subunit Y13) (Proteinase YSCE subunit 13) (Multicatalytic |
| | | | | | | | endopeptidase complex subunit Y13) |
| HS | PEG | | | | | 410 | ref\|NP_197599.1\|molybdopterin biosynthesis CNX1 protein/molybdenum cofactor |
| | | | | | | | biosynthesis enzyme CNX1 (CNX1) [Arabidopsis thaliana] |
| HS | PEG | | | | | 412 | ref\|NP_354433.1\|AGR_C_2631p [Agrobacterium tumefaciens str. C58] |
| | | | | | | | sp\|Q8UFH1\|ENO_AGRT5 Enolase (2-phosphoglycerate dehydratase) (2-phospho-D- |
| | | | | | | | glycerate hydro-lyase) |
| HS | DS | PP | | | | 437 | ref\|NP_928322.1\|5-carboxymethyl-2-hydroxymuconate semialdehyde dehydrogenase |
| | | | | | | | [Photorhabdus luminescens subsp. laumondii TTO1] |
| HS | PP | | | | | 466 | ref\|NP_171725.1\|no apical meristem (NAM) family protein [Arabidopsis thaliana] |
| HS | PP | | | | | 479 | ref\|NP_193147.1\|COP9 signalosome subunit, putative/CSN subunit, putative (CSN8) |
| | | | | | | | [Arabidopsis thaliana] sp\|P43255\|COP9_ARATH COP9 protein (FUSCA protein FUS7) |
| HS | PP | SS | | | | 509 | ref\|NP_311823.1\|fructose-bisphosphate aldolase class II [Escherichia coli O157:H7] r |
| HS | SS | | | | | 512 | pir\|\|T07150G-box binding factor 2A-soybean (fragment) gb\|AAB00097.1\|G-box binding |
| | | | | | | | factor |
| HS | SS | | | | | 533 | ref\|NP_442855.1\|unknown protein [Synechocystis sp. PCC 6803] |
| LL | LN | | | | | 375 | ref\|NP_172174.1\|ovate family protein [Arabidopsis thaliana] |
| LL | | | | | | 376 | ref\|NP_564336.1\|double-stranded DNA-binding family protein [Arabidopsis thaliana] |
| | | | | | | | pir\|\|A86422 hypothetical protein F1N18.11 [imported] - Arabidopsis thaliana |
| | | | | | | | gb\|AAG10611.1\|Similar to apoptosis related protein 19 [Arabidopsis thaliana] g |
| LL | | | | | | 377 | ref\|NP_173175.1\|MADS-box protein (AGL100) [Arabidopsis thaliana] pir\|\|E86309 |
| | | | | | | | T13M22.2 protein - Arabidopsis thaliana gb\|AAF79890.1\|Contains similarity to MADS- |
| | | | | | | | box protein from Cucumis sativus gb\|AF035438. It contains a SRF-type transcription |
| | | | | | | | factor (DNA-binding and dimerisation domain) P |
| LL | | | | | | 378 | ref\|NP_176202.1\|two-component responsive regulator/response regulator 3 (ARR3) |
| | | | | | | | [Arabidopsis thaliana] pir\|\|T50853 response regulator 3 [imported] - Arabidopsis thaliana |
| | | | | | | | dbj\|BAA34725.1\|response regulator 3 [Arabidopsis thaliana] gb\|AAD39333.1\|response |
| | | | | | | | regulator 3 [Arabidopsis thaliana] |
| LL | | | | | | 379 | ref\|NP_174152.3\|Dof-type zinc finger domain-containing protein [Arabidopsis thaliana] |
| LL | | | | | | 380 | ref\|NP_187378.1\|transcriptional activator, putative [Arabidopsis thaliana] gb\|AAF20224.1\| |
| | | | | | | | unknown protein [Arabidopsis thaliana] |
| LL | | | | | | 381 | ref\|NP_200562.1\|xyloglucan:xyloglucosyl transferase, putative/xyloglucan |
| | | | | | | | endotransglycosylase, putative/endo-xyloglucan transferase, putative [Arabidopsis |
| | | | | | | | thaliana] |
| LL | | | | | | 384 | ref\|NP_442075.1\|triosephosphate isomerase [Synechocystis sp. PCC 6803] |
| LL | | | | | | 385 | prf\|\|1908224Anucleotide translocator |
| LL | | | | | | 386 | ref\|NP_565172.1\|protein phosphatase 2C, putative/PP2C, putative [Arabidopsis |
| | | | | | | | thaliana] |
| LL | | | | | | 387 | ref\|NP_190087.1\|serine carboxypeptidase III, putative [Arabidopsis thaliana] pir\|\|T48977 |
| | | | | | | | carboxypeptidase-like protein |
| LL | DS | | | | | 388 | ref\|NP_194879.1\|expressed protein [Arabidopsis thaliana] |
| LL | LN | | | | | 400 | ref\|NP_172434.1\|Ras-related GTP-binding protein, putative [Arabidopsis thaliana] |
| | | | | | | | sp\|O04486\|RB1C_ARATH Ras-related protein Rab11C pir\|\|A86230 hypothetical protein |
| | | | | | | | [imported]- Arabidopsis thaliana gb\|AAB60720.1\|Strong similarity to A. thaliana ara-2 |
| | | | | | | | (gb\|ATHARA2). |
| LL | PP | | | | | 468 | ref\|NP_197366.1\|zinc finger (C3HC4-type RING finger) family protein [Arabidopsis |
| | | | | | | | thaliana] |
| LL | SS | | | | | 535 | ref\|NP_441937.1\|unknown protein [Synechocystis sp. PCC 6803] |
| LN | PEG | | | | | 391 | ref\|NP_182059.1\|leucine-rich repeat transmembrane protein kinase, putative |
| | | | | | | | [Arabidopsis thaliana] pir\|\|D84889 probable receptor-like protein kinase |
| LN | SS | | | | | 392 | |
| LN | | | | | | 393 | ref\|NP_195761.1\|stress-responsive protein, putative [Arabidopsis thaliana] |
| | | | | | | | sp\|Q8L940\|PXL3_ARATH Probable pyridoxin biosynthesis PDX1-like protein 3 |
| | | | | | | | pir\|\|T48163 pyridoxine biosynthesis protein-like - Arabidopsis thaliana |
| LN | | | | | | 394 | ref\|NP_179812.1\|inositol-3-phosphate synthase isozyme 2/myo-inositol-1-phosphate |
| | | | | | | | synthase 2/MI-1-P synthase 2/IPS 2 [Arabidopsis thaliana]]] |
| LN | | | | | | 395 | gb\|AAK19619.1\|GHMYB9 [Gossypium hirsutum] |
| LN | | | | | | 396 | ref\|NP_171800.1\|phototropic-responsive NPH3 family protein [Arabidopsis thaliana] |
| | | | | | | | pir\|\|G86160 protein F10O3.17 [imported]- Arabidopsis thaliana gb\|AAD25808.1\| |
| | | | | | | | F10O3.17 [Arabidopsis thaliana] |
| LN | | | | | | 399 | ref\|NP_172003.1\|protein kinase family protein [Arabidopsis thaliana] pir\|\|C86185 |
| | | | | | | | hypothetical protein [imported]- Arabidopsis thaliana gb\|AAC97990.1\|Strong similarity to |
| | | | | | | | Dsor1 protein kinase gb\|D13782 from Drosophila melanogaster. [Arabidopsis thaliana] |
| | | | | | | | gb\|AAL36395.1\|putative NPK1-related MAP kinase [Arabidopsis thaliana]] |
| LN | | | | | | 401 | ref\|NP_195750.1\|phosphatidylethanolamine-binding family protein [Arabidopsis thaliana] |
| | | | | | | | pir\|\|T48152 hypothetical protein T10O8.10 - Arabidopsis thaliana e |
| PEG | CS | SP | PEG | | | 405 | ref\|NP_196421.1\|expressed protein [Arabidopsis thaliana] sp\|Q9DS80\|OM05_ARATH |
| | | | | | | | Mitochondrial import receptor subunit TOM5 homolog (Translocase of outer membrane 5 kDa |
| | | | | | | | subunit homolog) |
| PEG | CS | | | | | 406 | ref\|NP_180926.1\|protein phosphatase 2C, putative/PP2C, putative [Arabidopsis |
| | | | | | | | thaliana] p |
| PEG | CK | HS | SS | | | 407 | ref\|NP_564151.1\|expressed protein [Arabidopsis thaliana] |
| PEG | HS | | | | | 408 | ref\|NP_199275.1\|cytochrome P450 family protein [Arabidopsis thaliana] dbj\|BAA98115.1\| |
| | | | | | | | flavonoid 3′,5′-hydroxylase-like; cytochrome P450 [Arabidopsis thaliana] |
| PEG | HS | SS | PEG | | | 411 | ref\|NP_014896.1\|Nat5p [Saccharomyces cerevisiae] pir\|\|S67150 hypothetical protein |
| | | | | | | | YOR253w-yeast (Saccharomyces cerevisiae) emb\|CAA99475.1\|unnamed protein |
| | | | | | | | product [Saccharomyces cerevisiae] gb\|AAS56076.1\|YOR253W [Saccharomyces |
| | | | | | | | cerevisiae] |
| PEG | CS | | | | | 413 | ref\|NP_195242.1\|O-methyltransferase family 2 protein [Arabidopsis thaliana] pir\|\|T04963 |
| | | | | | | | catechol O-methyltransferase homolog T12J5.30 - Arabidopsis thaliana |
| PEG | CK | | | | | 414 | ref\|NP_177064.1\|basic helix-loop-helix (bHLH) family protein [Arabidopsis thaliana] |
| | | | | | | | pir\|\|E96714 probable DNA-binding protein T6L1.19 [imported] - Arabidopsis thaliana |
| PEG | | | | | | 415 | dbj\|BAA77395.1\|SLL2-S9-protein [Brassica rapa] |
| PEG | CK | | | | | 416 | ref\|NP_175222.1\|E2F transcription factor-2 (E2F2)/transcription factor E2Fc (E2Fc) |
| | | | | | | | [Arabidopsis thaliana] gb\|AAG17609.1\|E2F transcription factor-2 E2F2 [Arabidopsis |
| | | | | | | | thaliana] |
| PEG | | | | | | 417 | ref\|NP_177523.1\|Ssu72-like family protein [Arabidopsis thaliana] pir\|\|F96765 unknown |
| | | | | | | | protein |
| PEG | | | | | | 418 | ref\|NP_564043.1\|expressed protein [Arabidopsis thaliana] |
| PEG | HS | PP | | | | 419 | ref\|NP_200327.1\|small ubiquitin-like modifier 2 (SUMO) [Arabidopsis thaliana] |
| | | | | | | | dbj\|BAB08585.1\|ubiquitin-like protein SMT3-like [Arabidopsis thaliana] |
| PEG | | | | | | 420 | gb\|AAO65311.1\|MADS affecting flowering 3 variant II [Arabidopsis thaliana] |
| PEG | | | | | | 421 | ref\|NP_567637.1\|methionine sulfoxide reductase domain-containing protein/SeIR |
| | | | | | | | domain-containing protein [Arabidopsis thaliana] |
| PEG | PP | PEG | | | | 422 | ref\|NP_191556.1\|methylenetetrahydrofolate reductase 1 (MTHFR1) [Arabidopsis |
| | | | | | | | thaliana] pir\|\|T47821 methylenetetrahydrofolate reductase MTHFR1- |
| PEG | | | | | | 423 | ref\|NP_569028.1\|expressed protein [Arabidopsis thaliana] |
| PEG | | | | | | 424 | ref\|NP_010276.1\|subunit of the Anaphase Promoting Complex; all known APC subunits |
| | | | | | | | co-immunoprecipitate with epitope-tagged Apc11p; Apc11p [Saccharomyces cerevisiae] |
| | | | | | | | pir\|\|S52511 hypothetical protein YDL008w-yeast (Saccharomyces cerevisiae) |
| | | | | | | | emb\|CAA88351.1\|unknown [Saccharomyces cerevisiae] emb\|CAA98564.1\|APC11 |
| | | | | | | | [Saccharomyces cerevisiae] |
| PEG | | | | | | 425 | ref\|NP_199487.1\|human Rev interacting-like family protein/hRIP family protein |
| | | | | | | | [Arabidopsis thaliana] dbj\|BAB08919.1\|zinc finger protein Glo3-like [Arabidopsis |
| | | | | | | | thaliana] |
| PEG | SP | | | | | 426 | ref\|NP_189282.1\|octicosapeptide/Phox/Bem1p (PB1) domain-containing protein |
| | | | | | | | [Arabidopsis thaliana] ref\|NP_850632.2\| |
| PEG | | | | | | 427 | ref\|NP_927823.1\|maltodextrin phosphorylase [Photorhabdus luminescens subsp. |
| | | | | | | | laumondii TTO1] |
| PEG | PP | PEG | | | | 428 | ref\|NP_241405.1\|NADP-dependent aldehyde dehydrogenase [Bacillus halodurans C- |
| | | | | | | | 125] |
| PEG | | | | | | 429 | ref\|ZP_00007935.1\|COG1850: Ribulose 1,5-bisphosphate carboxylase, large subunit |
| | | | | | | | [Rhodobacter sphaeroides] |
| PEG | PP | PEG | | | | 430 | ref\|NP_197727.1\|GRAM domain-containing protein/ABA-responsive protein-related |
| | | | | | | | [Arabidopsis thaliana] |
| PEG | CK | PP | PEG | | | 431 | ref\|NP_564994.1\|ubiquitin-conjugating enzyme family protein [Arabidopsis thaliana] |
| PEG | PP | SS | | | | 440 | ref\|NP_012367.1\|Histone methyltransferase with a role in transcriptional elongation, |
| | | | | | | | methylates a lysine residue of histone H3; associates with the C-terminal domain of |
| | | | | | | | Rpo21p; histone methylation activity is regulated by phosphorylation status of Rpo21p; |
| | | | | | | | Set2p [Saccharomyces cerevisiae] sp\|P46995\|SET2_YEAST SET domain protein 2 |
| PEG | PP | PEG | | | | 441 | ref\|NP_015436.1\|kinase required for late nuclear division; Dbf20p [Saccharomyces |
| | | | | | | | cerevisiae] sp\|P32328\|DBFB_YEAST Serine/threonine-protein kinase DBF20 |
| | | | | | | | pir\|\|S59776 protein kinase DBF20 (EC 2.7.1.—)-yeast (Saccharomyces cerevisiae) |
| PEG | DS | HS | PP | SS | | 442 | ref\|NP_535240.1\|succinate semialdehyde dehydrogenase [Agrobacterium tumefaciens |
| | | | | | | | str. C58] pir\|\|AF3142 succinate semialdehyde dehydrogenase gabD [imported]- |
| | | | | | | | Agrobacterium tumefaciens (strain C58, Dupont) |
| PEG | SS | HS | PP | | | 445 | ref\|NP_186975.1\|UTP--glucose-1-phosphate uridylyltransferase, putative/UDP-glucose |
| | | | | | | | pyrophosphorylase, putative/UGPase, putative [Arabidopsis thaliana] |
| PEG | PP | SS | | | | 446 | ref\|NP_181451.1\|protein kinase family protein [Arabidopsis thaliana] pir\|\|T02584 |
| | | | | | | | probable protein kinase |
| PEG | PP | SS | | | | 447 | ref\|NP_946297.1\|glyceraldehyde-3-phosphate dehydrogenase(GAPDH) |
| | | | | | | | [Rhodopseudomonas palustris CGA009] |
| PEG | LL | SS | | | | 511 | ref\|NP_011623.1\|role in DNA replication during S phase; Clb6p [Saccharomyces |
| | | | | | | | cerevisiae] sp\|P32943\|CGS6_YEAST S-PHASE ENTRY CYCLIN 6 pir\|\|S64417 cyclin B6- |
| | | | | | | | yeast (Saccharomyces cerevisiae) emb\|CAA97113.1\|CLB6 [Saccharomyces |
| | | | | | | | cerevisiae] |
| PP | HS | SS | | | | 436 | ref\|NP_568342.1\|rubredoxin family protein [Arabidopsis thaliana] dbj\|BAB10504.1\| |
| | | | | | | | gene_id:MKP11.2~unknown protein [Arabidopsis thaliana] |
| PP | SS | | | | | 448 | ref\|NP_188303.1\|protein phosphatase 2C, putative/PP2C, putative [Arabidopsis |
| | | | | | | | thaliana] |
| PP | SP | SS | | | | 449 | ref\|NP_011962.1\|Low-affinity glucose transporter of the major facilitator superfamily, |
| | | | | | | | expression is induced by Hxk2p in the presence of glucose and repressed by Rgt1p |
| | | | | | | | when glucose is limiting; Hxt1p [Saccharomyces cerevisiae] |
| PP | SS | | | | | 450 | ref\|NP_356098.1\|AGR_L_619p [Agrobacterium tumefaciens str. C58] pir\|\|A98170 |
| | | | | | | | hypothetical protein AGR_L_619 [imported] - Agrobacterium tumefaciens (strain C58, |
| | | | | | | | Cereon) gb\|AAK88883.1\|AGR_L_619p [Agrobacterium tumefaciens str. C58 (Cereon)] |
| PP | | | | | | 451 | ref\|NP_356568.1\|AGR_L_1560p [Agrobacterium tumefaciens str. C58] ref\|NP_534561.1\| |
| | | | | | | | glucose-1-phosphate adenylyltransferase [Agrobacterium tumefaciens str. C58] |
| | | | | | | | sp\|Q8U8L5\|GLGC_AGRT5 Glucose-1-phosphate adenylyltransferase (ADP-glucose |
| | | | | | | | synthase) (ADP-glucose pyrophosphorylase) (ADPGlc PPase) |
| PP | | | | | | 452 | ref\|NP_568731.1\|squamosa promoter-binding protein, putative [Arabidopsis thaliana] |
| PP | | | | | | 453 | ref\|NP_568102.1\|short-chain dehydrogenase/reductase (DSR) family protein |
| | | | | | | | [Arabidopsis thaliana] |
| PP | | | | | | 455 | ref\|NP_178317.2\|zinc finger (C2H2 type) family protein [Arabidopsis thaliana] |
| PP | | | | | | 456 | ref\|NP_188242.1\|F-box family protein [Arabidopsis thaliana] dbj\|BAB01261.1\|unnamed |
| | | | | | | | protein product [Arabidopsis thaliana] |
| PP | SP | | | | | 457 | ref\|NP_195254.1\|zinc finger (C2H2 type) family protein [Arabidopsis thaliana] |
| PP | | | | | | 458 | ref\|NP_568751.1\|polyadenylate-binding protein, putative/PABP, putative [Arabidopsis |
| | | | | | | | thaliana] |
| PP | SP | | | | | 460 | ref\|NP_195290.1\|isocitrate dehydrogenase, putative/NAD+ isocitrate dehydrogenase, |
| | | | | | | | putative [Arabidopsis thaliana] |
| PP | | | | | | 462 | ref\|NP_179604.1\|26S protease regulatory complex subunit 4, putative [Arabidopsis |
| | | | | | | | thaliana] pir\|\|E84585 26S proteasome subunit 4 [imported] - Arabidopsis thaliana |
| PP | | | | | | 463 | ref\|NP_191550.1\|expressed protein [Arabidopsis thaliana] |
| PP | | | | | | 464 | ref\|NP_172127.1\|shaggy-related protein kinase iota/ASK-iota (ASK9) (GSK1) |
| | | | | | | | [Arabidopsis thaliana] (EC 2.7.1.—) |
| PP | | | | | | 465 | ref\|NP_566544.1\|phosphotransfer family protein [Arabidopsis thaliana] gb\|AAB63642.1\| |
| | | | | | | | hypothetical protein [Arabidopsis thaliana] dbj\|BAA94763.1\|HPt phosphotransmitter |
| | | | | | | | [Arabidopsis thaliana] |
| PP | SP | | | | | 467 | emb\|CAD29662.1\|putative auxin response factor 23 [Arabidopsis thaliana] |
| PP | PEG | | | | | 469 | ref\|NP_200597.1\|anthranilate synthase beta subunit, putative [Arabidopsis thaliana] |
| PP | | | | | | 470 | ref\|NP_014177.1\|functionally related to TFIIB, affects start site selection in vivo; Ssu72p |
| | | | | | | | [Saccharomyces cerevisiae] sp\|P53538\|SS72_YEAST SSU72 PROTEIN pir\|\|S63180 |
| | | | | | | | hypothetical protein YNL222w-yeast (Saccharomyces cerevisiae) gb\|AAA86497.1\| |
| | | | | | | | Ssu72p [Saccharomyces cerevisiae] emb\|CAA96125.1\|SSU72 [Saccharomyces |
| | | | | | | | cerevisiae] prf\|\|2211396A SSU72 protein |
| PP | | | | | | 471 | ref\|NP_567238.2\|AAA-type ATPase family protein [Arabidopsis thaliana] |
| PP | PEG | | | | | 472 | ref\|NP_015049.1\|S-adenosylMethionine Permease; Sam3p [Saccharomyces cerevisiae] |
| | | | | | | | pir\|\|S65307 probable membrane protein YPL274w-yeast (Saccharomyces cerevisiae) |
| | | | | | | | emb\|CAA98011.1\|unnamed protein product [Saccharomyces cerevisiae] |
| PP | | | | | | 473 | ref\|NP_010944.1\|One of three possible beta-subunits of the Snf1 kinase complex, allows |
| | | | | | | | nuclear localization of the Snf1 kinase complex in the presence of a nonfermentable |
| | | | | | | | carbon source; contains glycogen-binding domain; Gal83p [Saccharomyces cerevisiae] |
| | | | | | | | sp\|Q04739\|GA83_YEAST Glucose repression protein GAL83 (SPM1 protein) |
| PP | | | | | | 474 | ref\|NP_441738.1\|fructose 1,6-bisphosphatase [Synechocystis sp. PCC 6803] |
| | | | | | | | sp\|P74324\|F16P_SYNY3 Fructose-1,6-bisphosphatase (D-fructose-1,6-bisphosphate 1- |
| | | | | | | | phosphohydrolase) (FBPase) |
| PP | | | | | | 475 | ref\|ZP_00087347.1\|COG1012: NAD-dependent aldehyde dehydrogenases |
| | | | | | | | [Pseudomonas fluorescens PfO-1] |
| PP | SS | | | | | 476 | ref\|NP_385551.1\|PYRUVATE DEHYDROGENASE ALPHA2 SUBUNIT PROTEIN |
| | | | | | | | [Sinorhizobium meliloti 1021] sp\|Q9R9N5\|ODPA_RHIME Pyruvate dehydrogenase E1 |
| | | | | | | | component, alpha subunit |
| PP | SS | | | | | 478 | ref\|NP_440132.1\|transaldolase [Synechocystis sp. PCC 6803] sp\|P72797\|TAL_SYNY3 |
| | | | | | | | Transaldolase pir\|\|S74660 transaldolase (EC 2.2.1.2) talB- |
| PP | SS | | | | | 529 | pir\|\|A96787protein F10A5.6 [imported] - Arabidopsis thaliana gb\|AAF87108.1\|F10A5.6 |
| | | | | | | | [Arabidopsis thaliana] |
| PP | SS | | | | | 534 | ref\|NP_389461.1\|similar to ribulose-5-phosphate 3-epimerase [Bacillus subtilis] |
| | | | | | | | sp\|O34557\|RPE_BACSU Ribulose-phosphate 3-epimerase (Pentose-5-phosphate 3- |
| | | | | | | | epimerase) (PPE) (R5P3E) |
| SP | | | | | | 480 | ref\|NP_567894.1\|expressed protein [Arabidopsis thaliana] |
| SP | | | | | | 481 | ref\|NP_563885.1\|expressed protein [Arabidopsis thaliana] |
| SP | | | | | | 482 | ref\|NP_197993.1\|PHD finger family protein [Arabidopsis thaliana] gb\|AAM64729.1\| |
| | | | | | | | nucleic acid binding protein-like [Arabidopsis thaliana] |
| SP | | | | | | 483 | ref\|NP_564171.1\|basic helix-loop-helix (bHLH) family protein [Arabidopsis thaliana] |
| SP | | | | | | 484 | ref\|NP_564133.1\|transporter-related [Arabidopsis thaliana] pir\|\|G86343 hypothetical |
| | | | | | | | protein T22I11.10 |
| SP | | | | | | 485 | ref\|NP_188710.1\|fertilization-independent endosperm protein (FIE) [Arabidopsis thaliana] |
| | | | | | | | sp\|Q9LT47\|FIE_ARATH Polycomb group protein] |
| SP | | | | | | 486 | ref\|NP_182182.2\|Dof zinc finger protein DAG2/Dof affecting germination 2 (DAG2) |
| | | | | | | | [Arabidopsis thaliana] |
| SP | | | | | | 487 | ref\|NP_565249.1\|phospholipid/glycerol acyltransferase family protein [Arabidopsis |
| | | | | | | | thaliana] |
| SP | | | | | | 488 | ref\|NP_198006.1\|hexose transporter, putative [Arabidopsis thaliana] pir\|\|T01853 |
| | | | | | | | probable hexose transport protein F9D12.17 - Arabidopsis thaliana gb\|AAC26243.1\| |
| | | | | | | | contains similarity to sugar transporters (Pfam: sugar_tr.hmm, score: 395.39) |
| SP | | | | | | 489 | ref\|NP_567693.1\|Dof-type zinc finger domain-containing protein [Arabidopsis thaliana] |
| | | | | | | | gb\|AAB63618.1\|zinc finger protein isolog [Arabidopsis thaliana] |
| SP | | | | | | 490 | ref\|NP_564354.1\|early-responsive to dehydration stress protein (ERD4) [Arabidopsis |
| | | | | | | | thaliana] pir\|\|H86427 unknown protein [imported] - Arabidopsis thaliana |
| SP | HS | | | | | 491 | ref\|NP_198936.1\|MADS-box family protein [Arabidopsis thaliana] dbj\|BAB09722.1\| |
| | | | | | | | unnamed protein product [Arabidopsis thaliana] |
| SP | | | | | | 492 | ref\|NP_173589.1\|SWIRM domain-containing protein/DNA-binding family protein |
| | | | | | | | [Arabidopsis thaliana] pir\|\|D86350 F8K7.13 protein - Arabidopsis thaliana |
| | | | | | | | gb\|AAD41423.1\|Contains similarity to gb\|AF033823 moira protein from Drosophila |
| | | | | | | | melanogaster and contains a PF\|00249 Myb-like DNA-binding domain. |
| SP | PP | | | | | 493 | ref\|NP_193702.1\|zinc finger (C3HC4-type RING finger) family protein [Arabidopsis |
| | | | | | | | thaliana] |
| SP | | | | | | 494 | ref\|NP_568256.1\|conserved oligomeric Golgi complex component-related/COG |
| | | | | | | | complex component-related [Arabidopsis thaliana] |
| SP | | | | | | 495 | ref\|NP_198387.1\|lectin protein kinase family protein [Arabidopsis thaliana] |
| SP | | | | | | 496 | ref\|NP_187953.1\|transcription initiation factor IID-1 (TFIID-1)/TATA-box factor 1/TATA |
| | | | | | | | sequence-binding protein 1 (TBP1) [Arabidopsis thaliana] |
| SP | | | | | | 497 | ref\|NP_177577.1\|zinc finger (C3HC4-type RING finger) family protein [Arabidopsis |
| | | | | | | | thaliana] pir\|\|D96772 probable RING zinc finger protein |
| SP | | | | | | 498 | gb\|AAF73257.1\|MAP kinase PsMAPK2 [Pisum sativum] |
| SP | | | | | | 499 | sp\|P48001\|HKL4_ARATHHomeobox protein knotted-1 like 4 (KNAT4) pir\|\|T51795 |
| | | | | | | | HOMEOBOX PROTEIN KNOTTED-1 LIKE 4 (KNAT4) - Arabidopsis thaliana |
| | | | | | | | emb\|CAC03454.1\|HOMEOBOX PROTEIN KNOTTED-1 LIKE 4 (KNAT4) [Arabidopsis |
| | | | | | | | thaliana] |
| SP | | | | | | 500 | ref\|NP_172008.1\|ent-kaurenoic acid hydroxylase (KAO1)/cytochrome P450 88A3, |
| | | | | | | | putative (CYP88A3) [Arabidopsis thaliana] sp\|O23051\|C883_ARATH Cytochrome P450 |
| | | | | | | | 88A3 pir\|\|H86185 hypothetical protein [imported] - Arabidopsis thaliana gb\|AAB71462.1\| |
| | | | | | | | Similar to Zea DWARF3 (gb\|U32579). [Arabidopsis thaliana] |
| SP | | | | | | 501 | ref\|NP_566061.1\|expressed protein [Arabidopsis thaliana] |
| SP | | | | | | 502 | pir\|\|D84423probable WD-40-repeat protein [imported] - Arabidopsis thaliana |
| | | | | | | | gb\|AAD14533.1\|putative stress protein [Arabidopsis thaliana] |
| SP | | | | | | 503 | ref\|NP_566585.1\|cyclic nucleotide-binding transporter 1/CNBT1 (CNGC20) |
| | | | | | | | [Arabidopsis thaliana] sp\|Q9LD37\|CG20_ARATH Probable cyclic nucleotide-gated ion |
| | | | | | | | channel 20, chloroplast precursor (Cyclic nucleotide-binding transporter 1) |
| SP | SS | | | | | 537 | gb\|AAO17183.1\|Orf17 [Photorhabdus luminescens] |
| SS | CS | DS | | | | 506 | ref\|NP_199800.1\|chloride channel protein (CLC-c) [Arabidopsis thaliana] |
| | | | | | | | sp\|Q96282\|CLCC_ARATH Chloride channel protein CLC-c (AtCLC-c) |
| SS | CS | HS | | | | 508 | ref\|NP_011665.1\|Hypothetical ORF; Ygr149wp [Saccharomyces cerevisiae] |
| | | | | | | | sp\|P48236\|YG3L_YEAST HYPOTHETICAL 51.6 KDA PROTEIN IN RPL24B-RSR1 |
| | | | | | | | INTERGENIC REGION pir\|\|S60440 probable membrane protein YGR149w-yeast |
| | | | | | | | (Saccharomyces cerevisiae) |
| SS | | | | | | 513 | ref\|NP_175691.1\|2-oxoglutarate-dependent dioxygenase, putative [Arabidopsis thaliana] |
| | | | | | | | pir\|\|D96569 probable oxidoreductase, |
| SS | PP | SP | | | | 515 | ref\|NP_193499.1\|casein kinase II beta chain, putative [Arabidopsis thaliana] |
| | | | | | | | sp\|P40229\|KC2C_ARATH Casein kinase II beta′ chain (CK II) pir\|\|S47968 casein kinase |
| | | | | | | | II (EC 2.7.1.—) beta chain CKB2 - Arabidopsis thaliana |
| SS | | | | | | 516 | ref\|NP_849990.1\|K+ efflux antiporter, putative (KEA4) [Arabidopsis thaliana] |
| SS | | | | | | 517 | ref\|NP_197992.1\|mitochondrial substrate carrier family protein [Arabidopsis thaliana] |
| SS | | | | | | 518 | emb\|CAA67968.1\|MADS4 protein [Betula pendula] |
| SS | | | | | | 519 | ref\|NP_014257.1\|belongs to a ubiquitous family of cytoplasmic membrane proteins that |
| | | | | | | | transport only ammonium (NH(4)(+) + NH(3)).; Mep2p [Saccharomyces cerevisiae] |
| | | | | | | | sp\|P41948\|MEP2_YEAST Ammonium transporter MEP2 pir\|\|S51089 ammonium |
| | | | | | | | transport protein MEP2-yeast (Saccharomyces cerevisiae) emb\|CAA58587.1\| |
| | | | | | | | ammonium transporter [Saccharomyces cerevisiae] emb\|CAA86884.1\|NH3 permease |
| | | | | | | | [Saccharomyces cerevisiae] emb\|CAA96025.1\|MEP2 [Saccharomyces cerevisiae] |
| SS | | | | | | 520 | ref\|NP_182083.1\|protein kinase family protein [Arabidopsis thaliana] |
| SS | CS | PP | | | | 521 | ref\|NP_391447.1\|UTP-glucose-1-phosphate uridylyltransferase [Bacillus subtilis] |
| | | | | | | | sp\|Q05852\|GTAB_BACSU UTP--glucose-1-phosphate uridylyltransferase (UDP-glucose |
| | | | | | | | pyrophosphorylase) (UDPGP) (Alpha-D-glucosyl-1-phosphate uridylyltransferase) |
| | | | | | | | (Uridine diphosphoglucose pyrophosphorylase) (General stress protein 33) (GSP33) p |
| SS | | | | | | 522 | ref\|NP_565553.1\|extra-large guanine nucleotide binding protein/G-protein (XLG) |
| | | | | | | | [Arabidopsis thaliana] pir\|\|T51593 GTP-binding regulatory protein extra-large [validated] |
| SS | | | | | | 523 | ref\|NP_009855.1\|Na+/Pi cotransporter, active in early growth phase; similar to phosphate |
| | | | | | | | transporters of Neurospora crassa; transcription regulated by inorganic phosphate |
| | | | | | | | concentrations and Pho4p; Pho89p [Saccharomyces cerevisiae] |
| | | | | | | | sp\|P38361\|YB8I_YEAST PUTATIVE PHOSPHATE-REPRESSIBLE PHOSPHATE |
| | | | | | | | PERMEASE YBR29C pir\|\|S46178 phosphate-repressible phosphate permease homolog |
| | | | | | | | YBR296c-yeast (Saccharomyces cerevisiae) emb\|CAA85261.1\|unnamed protein |
| | | | | | | | product [Saccharomyces cerevisiae] |
| SS | | | | | | 524 | ref\|NP_390639.1\|adenine phosphoribosyltransferase [Bacillus subtilis] |
| | | | | | | | sp\|O34443\|APT_BACSU Adenine phosphoribosyltransferase (APRT) pir\|\|B69587 |
| | | | | | | | adenine phosphoribosyltransferase (EC 2.4.2.7)- |
| SS | SP | | | | | 525 | ref\|NP_563659.1\|glycosyl hydrolase family 3 protein [Arabidopsis thaliana] |
| | | | | | | | gb\|AAK56255.1\|At1g02640/T14P4_11 [Arabidopsis thaliana] |
| SS | | | | | | 526 | ref\|NP_354584.1\|AGR_C_2921p [Agrobacterium tumefaciens str. C58] pir\|\|H97551 |
| | | | | | | | probable aminotransferase aatc [imported] - Agrobacterium tumefaciens (strain C58, |
| | | | | | | | Cereon) gb\|AAK87369.1\|AGR_C_2921p [Agrobacterium tumefaciens str. C58 (Cereon)] |
| SS | PP | | | | | 527 | ref\|NP_568226.1\|histidinol-phosphate aminotransferase, putative [Arabidopsis thaliana] |
| SS | | | | | | 528 | ref\|NP_201393.1\|U-box domain-containing protein [Arabidopsis thaliana] |
| | | | | | | | dbj\|BAB11139.1\|unnamed protein product [Arabidopsis thaliana] |
| SS | | | | | | 530 | ref\|NP_188576.1\|cinnamyl-alcohol dehydrogenase (CAD) [Arabidopsis thaliana] |
| | | | | | | | sp\|P48523\|CAD1_ARATH Cinnamyl-alcohol dehydrogenase (CAD) emb\|CAA83508.1\| |
| | | | | | | | cinnamyl alcohol dehydrogenase [Arabidopsis thaliana] |
| SS | | | | | | 531 | ref\|NP_388687.1\|acetoin dehydrogenase E1 component (TPP-dependent alpha subunit) |
| | | | | | | | [Bacillus subtilis] pir\|\|D69581 acetoin dehydrogenase E1 component (TPP-dependent |
| | | | | | | | alpha subuni) acoA - Bacillus subtilis |
| SS | | | | | | 532 | ref\|NP_242141.1\|uridine kinase [Bacillus halodurans C-125] sp\|Q9KDD8\|URK_BACHD |
| | | | | | | | Uridine kinase (Uridine monophosphokinase) (Cytidine monophosphokinase) p |
| SS | | | | | | 536 | ref\|NP_415984.1\|cryptic nitrate reductase 2 beta subunit [Escherichia coli K12] |
| | | | | | | | sp\|P19318\|NARY_ECOLI Respiratory nitrate reductase 2 beta chain pir\|\|F64899 nitrate |
| | | | | | | | reductase (EC 1.7.99.4) 2 beta chain - Escherichia coli (strain K-12) dbj\|BAA15104.1\| |
| | | | | | | | NarY protein [Escherichia coli] |
| SS | | | | | | 538 | ref\|NP_388315.1\|similar to pyruvate oxidase [Bacillus subtilis] pir\|\|G69769 pyruvate |
| | | | | | | | oxidase homolog ydaP -I Bacillus subtilis dbj\|BAA19271.1\|SIMILAR TO PYRUVATE |
| | | | | | | | OXIDASE AND ACETOLACTATE SYNTHASE. [Bacillus subtilis] emb\|CAB12241.1\| |
| | | | | | | | ydaP [Bacillus subtilis subsp. subtilis str. 168] |

*“CK” indicates cold tolerance improvement identified under a cold shock tolerance screen; “CS” indicates cold tolerance improvement identified by a cold germination tolerance screen; “DS” indicates drought tolerance improvement identified by a soil drought stress tolerance screen; “PEG” indicates osmotic stress tolerance improvement identified by a PEG induced osmotic stress tolerance screen; “HS” indicates heat stress tolerance improvement identified by a heat stress tolerance screen; “SS” indicates high salinity stress tolerance improvement identified by a salt stress tolerance screen; “LN” indicates nitrogen use efficiency improvement identified by a limited nitrogen tolerance screen; “LL” indicates attenuated shade avoidance response identified by a shade tolerance screen under a low light condition; “PP” indicates improved growth and development at early stages identified by an early plant growth and development screen; “SP” indicates improved growth and development at late stages identified by a late plant growth and development screen as described in US 20060075522, and incorporated herein by reference. Some proteins may provide more than one traits. For example, proteins of SEQ ID NOs 273 and 285.

TABLE 5
Examples of traits and proteins suitable for use in present invention.*

| | | Nucleic | |
|---|---|---|---|
| | | Acid | Protein |
| Trait | Protein | SEQ ID | SEQ ID |
| Apomixis | Curly Leaf - variant 1 | 12 | 626 |
| Apomixis | Curly Leaf - variant 2 | 13 | 627 |
| Carbon and/or nitrogen | E. coli glnB | 8 | 622 |
| metabolism | | | |
| Carbon and/or nitrogen | Anabaena SPP | 43 | 656 |
| metabolism | | | |
| Carbon and/or nitrogen | Corn SPP | 44 | 657 |
| metabolism | | | |
| Carbon and/or nitrogen | UDPgpp | 45 | 658 |
| metabolism | | | |
| Carbon and/or nitrogen | Synechocystis Sucrose phosphate synthase | 60 | 673 |
| metabolism | | | |
| Carbon and/or nitrogen | maize PPDK | 85 | 696 |
| metabolism | | | |
| Carbon and/or nitrogen | ZmSPS2-2 | 133 | 742 |
| metabolism | | | |
| Carbon and/or nitrogen | 700072387_FLI - ZmSPS3-1 | 223 | 831 |
| metabolism | | | |
| Carbon and/or nitrogen | GATE90 - ZmSPS2-3 | 224 | 832 |
| metabolism | | | |
| Carbon and/or nitrogen | Nostoc sp. PCC 7120 glnB | 228 | 836 |
| metabolism | | | |
| Carbon and/or nitrogen | Brassica P-II | 229 | 837 |
| metabolism | | | |
| Carbon and/or nitrogen | Nitrosomonas europaea dual function SBPase/FBPase- | 260 | 868 |
| metabolism | | | |
| Carbon and/or nitrogen | Nostoc sp. PCC 7120 GlpX protein | 264 | 872 |
| metabolism | | | |
| Carbon and/or nitrogen | Nostoc punctiforme strain ATCC 29133 GlpX protein-NOS1c0617 | 265 | 873 |
| metabolism | | | |
| Carbon and/or nitrogen | Anabaena SPS C154 | 266 | 874 |
| metabolism | | | |
| Carbon and/or nitrogen | Anabaena SPS C287 | 267 | 875 |
| metabolism | | | |
| Carbon and/or nitrogen | AtSUC1 | 286 | 893 |
| metabolism | | | |
| Carbon and/or nitrogen | Arabidopsis SUT2 | 287 | 894 |
| metabolism | | | |
| Carbon and/or nitrogen | Arabidopsis SUT4 | 288 | 895 |
| metabolism | | | |
| Carbon and/or nitrogen | OsSUT1 - D87819 | 289 | 896 |
| metabolism | | | |
| Carbon and/or nitrogen | OsSUT3 - AB071809 | 290 | 897 |
| metabolism | | | |
| Carbon and/or nitrogen | AtSUC2 | 299 | 906 |
| metabolism | | | |
| Carbon and/or nitrogen | corn duf6 2 | 303 | 910 |
| metabolism | | | |
| Carbon and/or nitrogen | corn duf6 3 | 304 | 911 |
| metabolism | | | |
| Carbon and/or nitrogen | corn duf6 | 305 | 912 |
| metabolism | | | |
| Carbon and/or nitrogen | corn duf6 6 | 306 | 913 |
| metabolism | | | |
| Carbon and/or nitrogen | corn duf6 11 | 307 | 914 |
| metabolism | | | |
| Carbon and/or nitrogen | corn duf6 12 | 308 | 915 |
| metabolism | | | |
| Carbon and/or nitrogen | corn duf6 13 | 309 | 916 |
| metabolism | | | |
| Carbon and/or nitrogen | corn duf6 14 | 310 | 917 |
| metabolism | | | |
| Carbon and/or nitrogen | corn duf6 15 | 311 | 918 |
| metabolism | | | |
| Carbon and/or nitrogen | corn duf6 16 | 312 | 919 |
| metabolism | | | |
| Carbon and/or nitrogen | corn duf6 17 | 313 | 920 |
| metabolism | | | |
| Carbon and/or nitrogen | corn duf6 18 | 314 | 921 |
| metabolism | | | |
| Carbon and/or nitrogen | corn duf6 19 | 315 | 922 |
| metabolism | | | |
| Carbon and/or nitrogen | corn duf6 20 | 316 | 923 |
| metabolism | | | |
| Carbon and/or nitrogen | soy duf6 1 | 317 | 924 |
| metabolism | | | |
| Carbon and/or nitrogen | soy duf6 2 | 318 | 925 |
| metabolism | | | |
| Carbon and/or nitrogen | soy duf6 3 | 319 | 926 |
| metabolism | | | |
| Carbon and/or nitrogen | soy duf6 4 | 320 | 927 |
| metabolism | | | |
| Carbon and/or nitrogen | soy duf6 5 | 321 | 928 |
| metabolism | | | |
| Carbon and/or nitrogen | soy duf6 6 | 322 | 929 |
| metabolism | | | |
| Carbon and/or nitrogen | soy duf6 7 | 323 | 930 |
| metabolism | | | |
| Carbon and/or nitrogen | soy duf6 8 | 324 | 931 |
| metabolism | | | |
| Carbon and/or nitrogen | soy duf6 9 | 325 | 932 |
| metabolism | | | |
| Carbon and/or nitrogen | soy duf6 10 | 326 | 933 |
| metabolism | | | |
| Carbon and/or nitrogen | soy duf6 11 | 327 | 934 |
| metabolism | | | |
| Carbon and/or nitrogen | soy duf6 12 | 328 | 935 |
| metabolism | | | |
| Carbon and/or nitrogen | soy duf6 13 | 329 | 936 |
| metabolism | | | |
| Carbon and/or nitrogen | soy duf6 15 | 330 | 937 |
| metabolism | | | |
| Carbon and/or nitrogen | corn SPS2-4 | 442 | 1049 |
| metabolism | | | |
| Carbon and/or nitrogen | corn sucrose export defective 1 (sdx1) - AF302187 | 534 | 1134 |
| metabolism | | | |
| Carbon and/or nitrogen | rice sdx1 delta ctp | 535 | 1135 |
| metabolism | | | |
| Carbon and/or nitrogen | Arabidopsis sucrose export defective 1-AF302188 | 536 | 1136 |
| metabolism | | | |
| Carbon and/or nitrogen | Nostoc sp. PCC 7120 sdx1-like - 17134979 | 537 | 1137 |
| metabolism | | | |
| Carbon and/or nitrogen | Synechocystis sp. PCC 6803 sdx1-like - 1652844 | 538 | 1138 |
| metabolism | | | |
| Carbon and/or nitrogen | Nostoc punctiforme sdx1-like | 539 | 1139 |
| metabolism | | | |
| Carbon and/or nitrogen | yeast VHT1 - YGR065C | 557 | 1157 |
| metabolism | | | |
| Carbon and/or nitrogen | Arabidopsis Suc5 - AJ252133 | 558 | 1158 |
| metabolism | | | |
| Carbon and/or nitrogen | rice putative phosphate translocator - AAK21346 | 591 | 1191 |
| metabolism | | | |
| Carbon and/or nitrogen | rice glucose-6-phosphate/phosphate-tranlocator-13486660 | 592 | 1192 |
| metabolism | | | |
| Carbon and/or nitrogen | corn glucose-6-phosphate/phosphate-tranlocator- | 593 | 1193 |
| metabolism | | | |
| Carbon and/or nitrogen | rice putative sugar transporter - AF416867 | 594 | 1194 |
| metabolism | | | |
| Cell division | rice cycD2 | 9 | 348 |
| Cell division | rice GCR1 | 11 | 350 |
| Cell division | corn Knotted1 | 12 | 351 |
| Cell division | corn cycD2.1 | 32 | 371 |
| Cell division | rice cyclin 2 | 35 | 374 |
| Cell division | rice cycC | 36 | 375 |
| Cell division | rice cycB2 | 37 | 376 |
| Cell division | rice cycA1 | 38 | 377 |
| Cell division | rice cycB5 | 39 | 378 |
| Cell division | corn cycA1 | 44 | 383 |
| Cell division | corn cycA2 | 45 | 384 |
| Cell division | corn cycB2 | 46 | 385 |
| Cell division | corn cycB5 | 47 | 386 |
| Cell division | LIB3279-180-C9_FLI - maize cyclin III | 48 | 387 |
| Cell division | corn cycB4 | 49 | 388 |
| Cell division | corn cycD3.2 | 50 | 389 |
| Cell division | corn cycDx.1 | 51 | 390 |
| Cell division | corn cycD1.1 | 52 | 391 |
| Cell division | retinoblastoma-related protein 1 | 70 | 409 |
| Cell division | C1 protein | 71 | 410 |
| Cell division | corn cycB3 | 75 | 414 |
| Cell division | rice cyclin H | 77 | 416 |
| Cell division | rice cdc2+/CDC28-related protein kinase | 78 | 417 |
| Cell division | Cdk-activating kinase 1 | 79 | 418 |
| Cell division | corn cycD3.1 | 89 | 428 |
| Cell division | corn cycD1.2 | 93 | 432 |
| Cell division | corn cycD1.3 | 94 | 433 |
| Cell division | Receiver domain (RR3-like) 7 | 100 | 439 |
| Cell division | Receiver domain (ARR2-like) 1 | 101 | 440 |
| Cell division | Receiver domain (TOC1-like) 2 | 102 | 441 |
| Cell division | Receiver domain (TOC1-like) 3 | 103 | 442 |
| Cell division | Receiver domain (ARR2-like) 4 | 104 | 443 |
| Cell division | Receiver domain (RR11-like) 5 | 105 | 444 |
| Cell division | Receiver domain (RR3-like) 6 | 106 | 445 |
| Cell division | Receiver domain (RR3-like) 8 | 107 | 446 |
| Cell division | Receiver domain 9 | 108 | 447 |
| Cell division | ZmRR2 | 109 | 448 |
| Cell division | Receiver domain (TOC1-like) 10 | 110 | 449 |
| Cell division | soy E2F-like | 195 | 534 |
| Cell division | nuclear matrix constituent protein | 196 | 535 |
| Cell division | OsE2F1 | 197 | 536 |
| Cell division | corn GCR1 | 198 | 537 |
| Cell division | menage a trois-like | 208 | 547 |
| Cell division | CDC28-related protein kinase | 278 | 617 |
| Cold tolerance | maize cellulose synthase (eskimo 2) | 1 | 340 |
| Cold tolerance | rice AOX1b (alternative oxidase) | 14 | 353 |
| Cold tolerance | Emericella nidulans alxA | 15 | 354 |
| Cold tolerance | soy omega-3 fatty acid desaturase | 20 | 359 |
| Cold tolerance | AtFAD7 | 21 | 360 |
| Cold tolerance | AtFAD8 | 22 | 361 |
| Cold tolerance | corn LEA3 | 147 | 486 |
| Cold tolerance | soy LEA protein | 168 | 507 |
| Cold tolerance | dehydrin-like protein | 169 | 508 |
| Cold tolerance | dehydrin 3 | 170 | 509 |
| Cold tolerance | probable lipase | 171 | 510 |
| Cold tolerance | yeast GRE1 | 172 | 511 |
| Cold tolerance | yeast STF2 | 173 | 512 |
| Cold tolerance | yeast SIP18 | 174 | 513 |
| Cold tolerance | yeast YBM6 | 175 | 514 |
| Cold tolerance | yeast HSP12 | 176 | 515 |
| Cold tolerance | S52650 - Synechocystis desB | 186 | 525 |
| Disease resistance | corn RNase PH | 155 | 494 |
| Disease resistance | SKI2 | 156 | 495 |
| Disease resistance | SKI3 | 157 | 496 |
| Disease resistance | SKI4 | 158 | 497 |
| Disease resistance | SKI6 | 159 | 498 |
| Disease resistance | SKI7 | 160 | 499 |
| Disease resistance | rice SKI7-like | 161 | 500 |
| Disease resistance | wheat G28-like | 203 | 542 |
| Disease resistance | SKI4-like protein | 231 | 570 |
| Disease resistance | putative 3 exoribonuclease | 232 | 571 |
| Disease resistance | corn ELI17-like protein 1 | 225 | 833 |
| Disease resistance | soy ELI17-like protein 1 | 226 | 834 |
| Disease resistance (cercosporin | PDR5 | 76 | 415 |
| tolerance) | | | |
| DNA integration | corn H2A.F/Z 1 | 241 | 849 |
| DNA integration | corn H2A 1 | 242 | 850 |
| DNA integration | corn H2A 3 | 243 | 851 |
| DNA integration | corn H2A 7 | 244 | 852 |
| Ethylene signaling | ethylene response sensor | 16 | 630 |
| Flower development | corn unknown protein | 17 | 356 |
| Flower development | corn SVP-like | 40 | 379 |
| Flower development | corn SVP-like | 41 | 380 |
| Flower development | soy SVP-like | 42 | 381 |
| Flower development | soy jointless-like | 43 | 382 |
| Flower development | ZmSE001-like | 213 | 552 |
| Flower development | wheat SVP-like 1 | 326 | 665 |
| Flower development | corn SVP-like 3 | 327 | 666 |
| Flower development | corn SVP-like 5 | 328 | 667 |
| Flower development | casein kinase II alpha subunit | 14 | 628 |
| Flower development | yeast TFS1 - X62105 | 131 | 740 |
| Flower development | yeast YLR179C-AAB67472 | 132 | 741 |
| Flower development | rice FPF1-like 1 | 134 | 743 |
| Flower development | rice FPF1-like 3 | 135 | 744 |
| Flower development | corn FPF1-like 1 | 136 | 745 |
| Flower development | corn FPF1-like 6 | 137 | 746 |
| Flower development | corn FLC-like 3 | 138 | 747 |
| Flower development | corn FLC-like 9 | 139 | 748 |
| Flower development | MADS affecting flowering 1-AAK37527 | 153 | 762 |
| Flower development | G1760 - Q9SZJ6 | 154 | 763 |
| Flower development | G2010 - CAB56582 | 155 | 764 |
| Flower development | corn FLT-like 1 | 158 | 767 |
| Flower development | corn FLT-like 2 | 159 | 768 |
| Flower development | corn FLT-like 3 | 160 | 769 |
| Flower development | rice leafy - AB005620 | 161 | 770 |
| Flower development | corn HD1-like 1 | 162 | 771 |
| Flower development | rice ANT-like | 263 | 871 |
| Flower development | yeast casein kinase II alpha - CAA86916 | 358 | 965 |
| Flower development | rice casein kinase alpha subunit - AAL34126 | 359 | 966 |
| Flower development | soy casein kinase alpha | 360 | 967 |
| Flower development | rice casein kinase II beta subunit - AAG60201 | 361 | 968 |
| Flower development | yeast CKB1 - Z72541 | 362 | 969 |
| Flower development | corn casein kinase beta 1 | 363 | 970 |
| Flower development | corn casein kinase beta 2 | 364 | 971 |
| Flower development | Arabidopsis LFY | 529 | 1129 |
| Glyphosphate tolerance | AnFPPS (farnesyl-pyrophosphate synthetase) | 90 | 429 |
| Glyphosphate tolerance | OsFPPS | 91 | 430 |
| Glyphosphate tolerance | 700331819_FLI - corn FPPS 2 | 92 | 431 |
| Heat tolerance/Water use | HSF1 | 84 | 423 |
| efficiency | | | |
| Heat tolerance/Water use | Zm HSP101 | 85 | 424 |
| efficiency | | | |
| Heat tolerance/Water use | E. coli clpB | 86 | 425 |
| efficiency | | | |
| Heat tolerance/Water use | Synechocystis clpB | 87 | 426 |
| efficiency | | | |
| Heat tolerance/Water use | Xylella clpB | 88 | 427 |
| efficiency | | | |
| Heat tolerance/Water use | maize p23 | 130 | 469 |
| efficiency | | | |
| Heat tolerance/Water use | maize cyclophilin | 131 | 470 |
| efficiency | | | |
| Heat tolerance/Water use | yeast SIT1 | 132 | 471 |
| efficiency | | | |
| Heat tolerance/Water use | yeast CNS1 | 133 | 472 |
| efficiency | | | |
| Heat tolerance/Water use | soy HSF | 148 | 487 |
| efficiency | | | |
| Heat tolerance/Water use | soy HSF | 149 | 488 |
| efficiency | | | |
| Heat tolerance/Water use | rice p23 co-chaperone | 220 | 559 |
| efficiency | | | |
| Heat tolerance/Water use | corn p23 co-chaperone | 221 | 560 |
| efficiency | | | |
| Heat tolerance/Water use | rice p23 co-chaperone | 222 | 561 |
| efficiency | | | |
| Heat tolerance/Water use | corn p23 co-chaperone | 223 | 562 |
| efficiency | | | |
| Heat tolerance/Water use | rice cns1-like | 320 | 659 |
| efficiency | | | |
| Heat tolerance/Water use | corn HCH1-like 1 | 321 | 660 |
| efficiency | | | |
| Heat tolerance/Water use | corn HOP-like 1 | 322 | 661 |
| efficiency | | | |
| Heat tolerance/Water use | corn HOP-like 2 | 323 | 662 |
| efficiency | | | |
| Heat tolerance/Water use | rice CHIP-like 1 | 324 | 663 |
| efficiency | | | |
| Heat tolerance/Water use | corn CHIP-like 2 | 325 | 664 |
| efficiency | | | |
| Homologous recombination | sRAD54 - with NLS | 57 | 396 |
| Homologous recombination | T4 endonuclease VII (gp49) - with NLS | 58 | 397 |
| Iron uptake | wheat nicotianamine aminotransferase | 102 | 711 |
| Iron uptake | corn nicotianamine synthase 1 | 103 | 712 |
| Iron uptake | corn ys1-like1 | 104 | 713 |
| Isoprenoid biosynthesis | soy 1-deoxy-D-xylulose-5-phosphate reductoisomerase- | 231 | 839 |
| Isoprenoid biosynthesis | Synechocystis 1-deoxy-D-xylulose-5-phosphate reductoisomerase-D64000 | 232 | 840 |
| Isoprenoid biosynthesis | Agrobacterium 1-deoxy-D-xylulose 5-phosphate reductoisomerase- | 233 | 841 |
| | AAK88334 | | |
| Isoprenoid biosynthesis | corn 1-deoxy-D-xylulose-5-phosphate synthase- | 234 | 842 |
| Isoprenoid biosynthesis | Agrobacterium 1-deoxy-D-xylulose-5-phosphate synthase-AAK86554 | 235 | 843 |
| Isoprenoid biosynthesis | Xylella 1-deoxyxylulose-5-phosphate synthase-AAF85048 | 236 | 844 |
| Light response | rice phyA with corn phyC intron 1 | 23 | 362 |
| Light response | sorghum phyA with corn phyC intron 1 | 24 | 363 |
| Light response | rice phyB with corn phyC intron 1 | 25 | 364 |
| Light response | sorghum phyB with corn phyC intron 1 | 26 | 365 |
| Light response | rice phyC with corn phyC intron 1 | 27 | 366 |
| Light response | sorghum phyC with corn phyC intron 1 | 28 | 367 |
| Light response | rice PF1 | 29 | 368 |
| Light response | rice GT2 | 30 | 369 |
| Light response | Synechocystis biliverdin reductase | 31 | 370 |
| Light response | corn nph1 | 33 | 372 |
| Light response | corn cp NDK - 700479629 | 54 | 393 |
| Light response | corn NDK - LIB3597020Q1K6C3 | 55 | 394 |
| Light response | corn NDK - 700241377 | 56 | 395 |
| Light response | corn NDPK - fC-zmemLIB3957015Q1K6H6 | 59 | 398 |
| Light response | PAS domain kinase | 98 | 437 |
| Light response | corn HY5-like | 111 | 450 |
| Light response | scarecrow 1 (PAT1-like) | 112 | 451 |
| Light response | scarecrow 2 | 113 | 452 |
| Light response | scarecrow 6 | 207 | 546 |
| Light response | gibberellin response modulator | 215 | 554 |
| Light response | scarecrow-like protein | 216 | 555 |
| Light response | helix-loop-helix protein (PIF3-like) | 230 | 569 |
| Light response | scarecrow 17 | 240 | 579 |
| Light response | PAT1-like scarecrow 9 | 277 | 616 |
| Light response | GATE5 - corn SPA1-like 1 | 311 | 650 |
| Light response | corn PIF3-like | 312 | 651 |
| Light response | soy Athb-2-like 1 | 313 | 652 |
| Light response | corn SUB1-like 1 | 314 | 653 |
| Light response | soy GH3 protein | 315 | 654 |
| Light response | soy homeobox-leucine zipper protein homolog h1- | 195 | 803 |
| Light response | soy zinc finger protein | 196 | 804 |
| Light response | soy TGACG-motif-binding protein STF2 | 197 | 805 |
| Light response | corn putative zinc finger protein | 198 | 806 |
| Light response | soy AP2 domain transcription factor | 199 | 807 |
| Light response | soy pseudo-response regulator | 200 | 808 |
| Light response | soy CONSTANS-like B-box zinc finger protein | 201 | 809 |
| Light response | soy RPT2-like | 202 | 810 |
| Light response | corn myb-related protein | 203 | 811 |
| Light response | Arabidopsis salt-tolerance protein | 204 | 812 |
| Light response | soy AP2 domain transcription factor | 205 | 813 |
| Light response | soy late elongated hypocotyl | 206 | 814 |
| Light response | soy zinc finger protein | 207 | 815 |
| Light response | soy putative protein kinase | 208 | 816 |
| Light response | soy auxin-responsive GH3 protein | 209 | 817 |
| Light response | corn ripening-related protein | 210 | 818 |
| Light response | corn beta-expansin 4 | 211 | 819 |
| Light response | corn hypothetical protein | 212 | 820 |
| Light response | corn unknown protein | 213 | 821 |
| Light response | corn unknown protein | 214 | 822 |
| Light response | corn unknown protein | 215 | 823 |
| Light response | soy unknown protein | 216 | 824 |
| Light response | corn unknown protein | 217 | 825 |
| Light response | rice Lls1 (CAO) - AF284781 | 218 | 826 |
| Light response | G557 (HY5) | 268 | 876 |
| Light response | Aspergillis phytochrome | 291 | 898 |
| Light response | corn spa1-like | 541 | 1141 |
| Light response | corn mt NDK - LIB189022Q1E1E9 | 53 | 392 |
| Metabolite transport | H+ transporting ATPase | 279 | 618 |
| Metabolite transport | cation-transporting ATPase | 280 | 619 |
| Metabolite transport | yeast DRS2 (ALA1-like) - L01795 | 281 | 620 |
| Metabolite transport | S. pombe ALA1-like-CAA21897 | 282 | 621 |
| Metabolite transport | rice ALA1-like 1 - BAA89544 | 283 | 622 |
| Nitric oxide signaling | yeast flavohemoglobin - mitochondrial | 72 | 411 |
| Nitric oxide signaling | X58872 - E. coli hmp1 | 227 | 835 |
| Nitric oxide signaling | yeast YDL168W/SFA1 - NP_010113 | 583 | 1183 |
| Nitric oxide signaling | E. coli adhC - AE000142 | 584 | 1184 |
| Nitric oxide signaling | Nostoc sp. PCC 7120 glutathione dependent formaldehyde dehydrogenase- | 585 | 1185 |
| | BAB74509 | | |
| Nitric oxide signaling | rice glutathione dependent formaldehyde dehydrogenase-U77637 | 586 | 1186 |
| Nitric oxide signaling | corn glutathione-dependent formaldehyde dehydrogenase-Y11029 | 587 | 1187 |
| Nitric oxide signaling | corn corn glutathione-dependent formaldehyde dehydrogenase 2- | 588 | 1188 |
| Nitric oxide signaling | corn corn glutathione-dependent formaldehyde dehydrogenase 3- | 589 | 1189 |
| Nitric oxide signaling | soy glutathione dependent formaldehyde dehydrogenase- | 590 | 1190 |
| Nitrogen assimilation | TOR1 | 60 | 399 |
| Nitrogen assimilation | 14-3-3-like protein 2 | 115 | 454 |
| Nitrogen assimilation | 14-3-3-like protein D | 116 | 455 |
| Nitrogen assimilation | 14-3-3 protein 1 | 117 | 456 |
| Nitrogen assimilation | Rice FAP1-like protein | 118 | 457 |
| Nitrogen assimilation | rice TAP42-like | 119 | 458 |
| Nitrogen assimilation | BMH1 | 120 | 459 |
| Nitrogen assimilation | GF14-c protein | 233 | 572 |
| Nitrogen assimilation | 14-3-3-like protein | 234 | 573 |
| Nitrogen assimilation | rice elF-(iso)4F | 235 | 574 |
| Nitrogen assimilation | rice elF-4F | 236 | 575 |
| Nitrogen assimilation | sorghum elF-(iso)4F | 237 | 576 |
| Nitrogen assimilation | sorghum elF-4F | 238 | 577 |
| Nitrogen assimilation | rice FIP37-like | 239 | 578 |
| Nitrogen metabolism | Synechocystis cobA w cp transit peptide | 162 | 501 |
| Nitrogen metabolism | Xylella tetrapyrrole methylase with transit peptide | 163 | 502 |
| Nitrogen metabolism | maize uroporphyrinogen III methyltransferase | 164 | 503 |
| Nitrogen transport | corn AAP6-like | 16 | 355 |
| Nitrogen transport | sorghum proline permease | 204 | 543 |
| Nitrogen transport | rice AA transporter | 205 | 544 |
| Nitrogen transport | rice amino acid transporter-like protein | 295 | 634 |
| Nitrogen transport | corn amino acid permease | 296 | 635 |
| Nitrogen transport | rice proline transport protein | 297 | 636 |
| Nitrogen uptake/Seed | CHL1 | 80 | 419 |
| development | | | |
| Nitrogen uptake/Seed | NTR1 | 81 | 420 |
| development | | | |
| Pest tolerance (Insect resistance) | oryzacystatin | 209 | 548 |
| Pest tolerance (Insect resistance) | Similar to cysteine proteinase inhibitor | 210 | 549 |
| Pest tolerance (Insect resistance) | cysteine proteinase inhibitor | 211 | 550 |
| Phosphate uptake | RNAse S | 134 | 473 |
| Phosphate uptake | maize ecto-apyrase | 135 | 474 |
| Phosphate uptake | PHO5 | 136 | 475 |
| Phosphate uptake | high affinity phosphate translocator | 137 | 476 |
| Phosphate uptake | high affinity phosphate translocator | 138 | 477 |
| Phosphate uptake | Xylella citrate synthase | 139 | 478 |
| Phosphate uptake | E. coli citrate synthase | 140 | 479 |
| Phosphate uptake | rice citrate synthase | 141 | 480 |
| Phosphate uptake | citrate synthase | 142 | 481 |
| Phosphate uptake | citrate synthase | 143 | 482 |
| Phosphate uptake | putative purple acid phosphatase precursor | 224 | 563 |
| Phosphate uptake | acid phosphatase type 5 | 225 | 564 |
| Phosphate uptake | aleurone ribonuclease | 226 | 565 |
| Phosphate uptake | putative ribonuclease | 227 | 566 |
| Phosphate uptake | S-like RNase | 228 | 567 |
| Phosphate uptake | ribonuclease | 229 | 568 |
| Photosynthesis | thylakoid carbonic anhydrase, cah3 | 151 | 490 |
| Photosynthesis | thylakoid carbonic anhydrase, ecaA | 152 | 491 |
| Photosynthesis | Chlamydomonas reinhardtii envelope protein LIP-36G1 | 153 | 492 |
| Photosynthesis | psbO transit peptide::Synechococcus sp. PCC 7942 ictB | 154 | 493 |
| Photosynthesis/Carbon | rice chloroplastic sedoheptulose-1,7-bisphosphatase- | 284 | 623 |
| partitioning | | | |
| Photosynthesis/Carbon | rice cytosolic fructose-1,6-bisphosphatase | 285 | 624 |
| partitioning | | | |
| Photosynthesis/Carbon | Wheat sedoheptulose-1,7-bisphosphatase | 286 | 625 |
| partitioning | | | |
| Photosynthesis/Carbon | sedoheptulose-1,7-bisphosphatase | 287 | 626 |
| partitioning | | | |
| Plant growth and development | corn GRF1-like protein | 18 | 357 |
| Plant growth and development | rice GRF1 | 19 | 358 |
| Plant growth and development | corn elF-5A | 61 | 400 |
| Plant growth and development | yeast elF-5A | 62 | 401 |
| Plant growth and development | yeast deoxyhypusine synthase | 63 | 402 |
| Plant growth and development | yeast L5 | 64 | 403 |
| Plant growth and development | yeast ornithine decarboxylase | 65 | 404 |
| Plant growth and development | rice exportin 4-like | 66 | 405 |
| Plant growth and development | yeast S-adenosylmethionine decarboxylase | 67 | 406 |
| Plant growth and development | corn S-adenosylmethionine decarboxylase 1 | 68 | 407 |
| Plant growth and development | corn S-adenosylmethionine decarboxylase 2 | 69 | 408 |
| Plant growth and development | deoxyhypusine synthase | 150 | 489 |
| Plant growth and development | corn TIR1-like | 179 | 518 |
| Plant growth and development | F-box protein | 182 | 521 |
| Plant growth and development | F-box protein | 183 | 522 |
| Plant growth and development | cytochrome P450-like protein | 188 | 527 |
| Plant growth and development | cytochrome P450 | 189 | 528 |
| Plant growth and development | cytochrome P450-like | 190 | 529 |
| Plant growth and development | CYP90 protein | 191 | 530 |
| Plant growth and development | cytochrome P450 DWARF3 | 192 | 531 |
| Plant growth and development | cytochrome P450 | 193 | 532 |
| Plant growth and development | rice receptor protein kinase | 194 | 533 |
| Plant growth and development | Zm-GRF1 (GA responsive factor) | 212 | 551 |
| Plant growth and development | deoxyhypusine synthase | 214 | 553 |
| Plant growth and development | maize tubby-like | 273 | 612 |
| Plant growth and development | maize tubby-like | 274 | 613 |
| Plant growth and development | soy phantastica | 288 | 627 |
| Plant growth and development | soy phantastica 2 | 289 | 628 |
| Plant growth and development | maize rough sheath 1 | 290 | 629 |
| Plant growth and development | soy Ig3-like 1 | 291 | 630 |
| Plant growth and development | soy rough sheath1-like 1 | 292 | 631 |
| Plant growth and development | soy G559-like | 293 | 632 |
| Plant growth and development | soy G1635-like 1 | 294 | 633 |
| Plant growth and development | G1073 | 22 | 1215 |
| Plant growth and development | G1411 | 23 | 636 |
| Plant growth and development | G1449 | 24 | 637 |
| Plant growth and development | G1635 | 25 | 638 |
| Plant growth and development | G559 | 28 | 641 |
| Plant growth and development | G865 | 29 | 642 |
| Plant growth and development | 700151210_FLI - corn L5 a | 31 | 644 |
| Plant growth and development | LIB3957-023-C10_FLI - corn C-type cyclin | 32 | 645 |
| Plant growth and development | corn pescadillo-like 1 | 37 | 650 |
| Plant growth and development | yeast pescadillo homologue - Z72888 | 38 | 651 |
| Plant growth and development | soy GA2-oxidase | 39 | 652 |
| Plant growth and development | soy U-box protein 1 | 46 | 659 |
| Plant growth and development | soy U-box protein 2 | 47 | 660 |
| Plant growth and development | soy U-box protein 3 | 48 | 661 |
| Plant growth and development | corn U-box protein 3 | 49 | 662 |
| Plant growth and development | corn U-box protein 4 | 50 | 663 |
| Plant growth and development | soy RING finger protein 1 [BCRA1] | 58 | 671 |
| Plant growth and development | yeast HAL3-Z28297 | 61 | 674 |
| Plant growth and development | yeast GLC8-P41818 | 62 | 675 |
| Plant growth and development | corn HAL3-like 1-LIB3060-046-G12_FLI | 63 | 676 |
| Plant growth and development | corn HAL3-like 2- | 64 | 677 |
| Plant growth and development | corn GLC8-like 2 | 65 | 678 |
| Plant growth and development | corn GLC8-like 1 | 66 | 679 |
| Plant growth and development | corn RING finger protein 25 | 74 | 685 |
| Plant growth and development | Xylella SAG13-like - E82748 | 88 | 699 |
| Plant growth and development | corn SAG13-like 1 | 89 | 700 |
| Plant growth and development | soy SAG13-like 1 | 90 | 701 |
| Plant growth and development | soy SAG13-like 2 | 91 | 702 |
| Plant growth and development | Nostoc punctiforme SAG13-like | 92 | 703 |
| Plant growth and development | corn clavata3-like | 93 | 704 |
| Plant growth and development | corn clavata3-like | 94 | 705 |
| Plant growth and development | wheat clv3-like | 95 | 1218 |
| Plant growth and development | corn clv3-like 2 | 96 | 706 |
| Plant growth and development | corn ESR2 | 97 | 1219 |
| Plant growth and development | rice glutamate decarboxylase | 105 | 714 |
| Plant growth and development | rice GA2 oxidase | 106 | 715 |
| Plant growth and development | corn mlo-like 1 | 107 | 716 |
| Plant growth and development | corn G alpha 1 | 108 | 717 |
| Plant growth and development | corn G beta 2 | 109 | 718 |
| Plant growth and development | corn g gamma-like 1 | 110 | 719 |
| Plant growth and development | rice tubby 1 | 111 | 720 |
| Plant growth and development | maize tubby 3 | 112 | 721 |
| Plant growth and development | maize tubby 4 | 113 | 722 |
| Plant growth and development | corn tubby 5 | 114 | 723 |
| Plant growth and development | maize tubby 6 | 115 | 724 |
| Plant growth and development | maize tubby 7 | 116 | 725 |
| Plant growth and development | corn tubby 9 | 117 | 726 |
| Plant growth and development | rice LRR.F-box protein 1 | 118 | 727 |
| Plant growth and development | corn LRR.F-box protein 7 | 119 | 728 |
| Plant growth and development | rice G1449-like 1 | 123 | 732 |
| Plant growth and development | corn adenylate transporter 1 | 128 | 737 |
| Plant growth and development | Xylella adenylate transporter - XF1738 | 129 | 738 |
| Plant growth and development | yeast AAC2 - Z35791 | 130 | 739 |
| Plant growth and development | SAG13 - AAF09487 | 156 | 765 |
| Plant growth and development | Rice SAG13-like | 157 | 766 |
| Plant growth and development | rice plastidic ATP/ADP-transporter - BAB40979 | 163 | 772 |
| Plant growth and development | rice L5 a | 164 | 773 |
| Plant growth and development | corn L5 | 165 | 774 |
| Plant growth and development | corn L5-like | 166 | 775 |
| Plant growth and development | Agrobacterium ornithine decarboxylase | 167 | 776 |
| Plant growth and development | corn IAP-like RING finger protein 1 | 168 | 777 |
| Plant growth and development | yeast BIR1 | 169 | 1220 |
| Plant growth and development | corn BAX inhibitor 1-like 1 | 170 | 778 |
| Plant growth and development | corn Bax inhibitor 1-like 2 | 171 | 779 |
| Plant growth and development | corn Bax inhibitor 1-like 3 | 172 | 780 |
| Plant growth and development | corn caspase-like 1 | 173 | 781 |
| Plant growth and development | corn caspase-like 2 | 174 | 782 |
| Plant growth and development | corn caspase-like 3 | 175 | 783 |
| Plant growth and development | yeast YOR197w | 176 | 784 |
| Plant growth and development | soy Lls1-like | 177 | 785 |
| Plant growth and development | Synechocystis Rieske iron-sulfur protein | 178 | 786 |
| Plant growth and development | Arabidopsis AGL15 | 179 | 787 |
| Plant growth and development | yeast OST2 | 180 | 788 |
| Plant growth and development | corn DAD1 | 181 | 789 |
| Plant growth and development | corn pirin | 182 | 790 |
| Plant growth and development | G896 | 219 | 827 |
| Plant growth and development | G1435 | 220 | 828 |
| Plant growth and development | LIB4074-003-H1_FLI - corn G1435-like | 221 | 829 |
| Plant growth and development | G1496 | 222 | 830 |
| Plant growth and development | yeast DBF2 - P22204 | 254 | 862 |
| Plant growth and development | yeast DBF20 - P32328 | 255 | 863 |
| Plant growth and development | Arabidopsis agl8 - Q38876 | 256 | 864 |
| Plant growth and development | corn AGL8-like B | 257 | 865 |
| Plant growth and development | corn PHR1-like 1 | 258 | 866 |
| Plant growth and development | corn PHR1-like 2 | 259 | 867 |
| Plant growth and development | G189 | 269 | 877 |
| Plant growth and development | G736 | 270 | 878 |
| Plant growth and development | G1073 | 271 | 1221 |
| Plant growth and development | G671 | 272 | 879 |
| Plant growth and development | G247 | 273 | 880 |
| Plant growth and development | G1384 | 274 | 881 |
| Plant growth and development | G779 | 275 | 882 |
| Plant growth and development | G1795 | 276 | 883 |
| Plant growth and development | G977 | 277 | 884 |
| Plant growth and development | G568 | 278 | 885 |
| Plant growth and development | G1269 | 279 | 886 |
| Plant growth and development | G1050 | 280 | 887 |
| Plant growth and development | phytosulfokine 4 - 13399211 | 282 | 889 |
| Plant growth and development | rice phytosulfokine 2 - 13399209 | 283 | 890 |
| Plant growth and development | rice phytosulfokine-alpha - 11907498 | 284 | 891 |
| Plant growth and development | rice phytosulphokine SH27A - 3201971 | 285 | 892 |
| Plant growth and development | corn histidine phosphotransfer protein 1 | 292 | 899 |
| Plant growth and development | corn histidine phosphotransfer protein 2 | 293 | 900 |
| Plant growth and development | soy histidine phosphotransfer protein 1 | 294 | 901 |
| Plant growth and development | YPD1 - Z74283 | 295 | 902 |
| Plant growth and development | rice ethylene receptor - AF013979 | 296 | 903 |
| Plant growth and development | rice ethylene responsive factor - AAK70909 | 297 | 904 |
| Plant growth and development | CYP79F1 (supershoot) - AF370512 | 298 | 905 |
| Plant growth and development | rice G alpha XL - BAA93022 | 357 | 964 |
| Plant growth and development | corn RING finger 100 | 373 | 980 |
| Plant growth and development | corn RING finger 101 | 374 | 981 |
| Plant growth and development | corn RING finger 103 | 375 | 982 |
| Plant growth and development | corn RING finger 102 | 376 | 983 |
| Plant growth and development | corn RING finger 104 | 377 | 984 |
| Plant growth and development | corn RING finger 105 | 378 | 985 |
| Plant growth and development | corn RING finger 106 | 379 | 986 |
| Plant growth and development | corn RING finger 108 | 380 | 987 |
| Plant growth and development | corn RING finger 109 | 381 | 988 |
| Plant growth and development | corn RING finger 110 [ReMembR-H2 protein JR702]- | 382 | 989 |
| Plant growth and development | corn RING finger 111 | 383 | 990 |
| Plant growth and development | corn RING finger 112 | 384 | 991 |
| Plant growth and development | corn RING finger 113 | 385 | 992 |
| Plant growth and development | corn RING finger 114 | 386 | 993 |
| Plant growth and development | corn RING finger 115 | 387 | 994 |
| Plant growth and development | corn RING finger 116 | 388 | 995 |
| Plant growth and development | corn Skp1-like 118 [UIP2] | 389 | 996 |
| Plant growth and development | corn Skp1-like 119 | 390 | 997 |
| Plant growth and development | corn Skp1-like 120 [UIP2] | 391 | 998 |
| Plant growth and development | corn F-box 123 | 392 | 999 |
| Plant growth and development | corn F-box 124 [TIR1] | 393 | 1000 |
| Plant growth and development | corn F-box 125 [TIR1] | 394 | 1001 |
| Plant growth and development | corn F-box 126 | 395 | 1002 |
| Plant growth and development | corn RING finger 128 [response regulator 6] | 396 | 1003 |
| Plant growth and development | corn RING finger 132 | 397 | 1004 |
| Plant growth and development | corn RING finger 133 | 398 | 1005 |
| Plant growth and development | corn RING finger 136 | 400 | 1007 |
| Plant growth and development | corn RING finger 138 | 401 | 1008 |
| Plant growth and development | corn F-box 141 | 402 | 1009 |
| Plant growth and development | corn F-box 142 | 403 | 1010 |
| Plant growth and development | corn F-box 143 | 404 | 1011 |
| Plant growth and development | corn F-box 144 | 405 | 1012 |
| Plant growth and development | corn F-box 145 | 406 | 1013 |
| Plant growth and development | corn RING finger 147 | 407 | 1014 |
| Plant growth and development | corn RING finger 149 [anaphase promoting complex subunit 11]- | 408 | 1015 |
| Plant growth and development | corn RING finger 151 [ROC1] | 409 | 1016 |
| Plant growth and development | corn U-box 153 | 410 | 1017 |
| Plant growth and development | corn RING finger 154 [VIP2] | 411 | 1018 |
| Plant growth and development | corn RING finger 155 [VIP2] | 412 | 1019 |
| Plant growth and development | rice U-box 100 | 413 | 1020 |
| Plant growth and development | corn RING finger protein 157 | 414 | 1021 |
| Plant growth and development | corn F-box 159 [FKF1-like] | 415 | 1022 |
| Plant growth and development | corn F-box 160 [FKF-like] | 416 | 1023 |
| Plant growth and development | corn RING finger 161 [cellulose synthase] | 417 | 1024 |
| Plant growth and development | corn RING finger 162 [cellulose synthase] | 418 | 1025 |
| Plant growth and development | corn RING finger 163 | 419 | 1026 |
| Plant growth and development | corn RING finger 164 | 420 | 1027 |
| Plant growth and development | corn RING finger 165 | 421 | 1028 |
| Plant growth and development | corn F-box 166 | 422 | 1029 |
| Plant growth and development | corn RING finger 167 [seven in absentia-like] | 423 | 1030 |
| Plant growth and development | corn F-box 168 | 424 | 1031 |
| Plant growth and development | corn RING finger 169 [S-ribonuclease binding protein] | 425 | 1032 |
| Plant growth and development | corn F-box 170 | 426 | 1033 |
| Plant growth and development | corn F-box 172 | 427 | 1034 |
| Plant growth and development | corn RING finger 174 | 428 | 1035 |
| Plant growth and development | corn RING 175 | 429 | 1036 |
| Plant growth and development | corn Cullin 176 | 430 | 1037 |
| Plant growth and development | corn RING finger 177 | 431 | 1038 |
| Plant growth and development | corn RING finger 178 | 432 | 1039 |
| Plant growth and development | corn RING finger 179 [alpha-galactosidase] | 433 | 1040 |
| Plant growth and development | corn RING finger 180 | 434 | 1041 |
| Plant growth and development | corn F-box 181 | 435 | 1042 |
| Plant growth and development | corn RING finger 182 | 436 | 1043 |
| Plant growth and development | corn RING finger 183 | 437 | 1044 |
| Plant growth and development | corn RING finger 185 | 438 | 1045 |
| Plant growth and development | corn F-box 186 | 439 | 1046 |
| Plant growth and development | corn F-box 187 | 440 | 1047 |
| Plant growth and development | corn RING/U-box 188 | 441 | 1048 |
| Plant growth and development | corn sigma factor 1 | 474 | 1074 |
| Plant growth and development | corn sigma factor 2 [Sig3] | 475 | 1075 |
| Plant growth and development | corn sigma factor 3 [Sig1] | 476 | 1076 |
| Plant growth and development | soy G-gamma subunit DC-terminus | 523 | 1123 |
| Plant growth and development | soy AGL8-like 1 | 524 | 1124 |
| Plant growth and development | soy AGL8-like 3 | 525 | 1125 |
| Plant growth and development | corn Agl8D (LIB5131-001-H1) | 526 | 1126 |
| Plant growth and development | corn Agl8E (LIB5131-001-H2) | 527 | 1127 |
| Plant growth and development | corn Agl8F (LIB5131-001-H3) | 528 | 1128 |
| Plant growth and development | corn lsr | 530 | 1130 |
| Plant growth and development | soy lsr-like 1 | 531 | 1131 |
| Plant growth and development | rice lsr-like 1 | 532 | 1132 |
| Plant growth and development | rice G1073-like 1 | 549 | 1149 |
| Plant growth and development | rice inosine monophosphate dehydrogenase - AAK09225 | 550 | 1150 |
| Plant growth and development | yeast IMP dehydrogenase [Imd2p]- NP_012088 | 551 | 1151 |
| Plant growth and development | E. coli guaB - NP_417003 | 552 | 1152 |
| Plant growth and development | Agrobacterium GuaB - AE007996 | 553 | 1153 |
| Plant growth and development | corn RING finger protein 25 | 556 | 1156 |
| Plant growth and development | Glutamate Decarboxylase | 561 | 1161 |
| Plant growth and development | putative carnitine/acylcarnitine translocase- | 562 | 1162 |
| Plant growth and development | corn cycA-like [GATE80] | 563 | 1163 |
| Plant growth and development | corn cycA-like [GAT81] | 564 | 1164 |
| Plant growth and development | corn E4/E8 binding protein-like [GATE68] | 565 | 1165 |
| Plant growth and development | Arabidopsis G748 | 566 | 1166 |
| Plant growth and development | Arabidopsis NAM (no apical meristem)-like protein- | 567 | 1167 |
| Plant growth and development | soy G1452-like protein | 568 | 1168 |
| Plant growth and development | corn cytochrome P450 | 569 | 1169 |
| Plant growth and development | rice receptor-like protein | 570 | 1170 |
| Plant growth and development | rice receptor-like protein kinase | 571 | 1171 |
| Plant growth and development | rice putative brassinosteroid-insensitive protein | 572 | 1172 |
| Plant growth and development | rice cell elongation protein DIMINUTO | 573 | 1173 |
| Plant growth and development | corn Dwarf1-like 1 | 574 | 1174 |
| Plant growth and development | rice putative sterol-C5(6)-desaturase | 575 | 1175 |
| Plant growth and development | corn sterol-C5(6)-desaturase 2 | 576 | 1176 |
| Plant growth and development | corn sterol-C5(6)-desaturase 1 | 577 | 1177 |
| Plant growth and development | soy sterol-C5(6)-desaturase | 578 | 1178 |
| Plant growth and development | rice BRS1-like protease 4 | 579 | 1179 |
| Plant growth and development | corn BRS1-like protease 1 | 580 | 1180 |
| Plant growth and development | soy FKF1-like protein | 581 | 1181 |
| Plant growth and development | corn FKF-like protein 2 | 582 | 1182 |
| Plant growth and development | soy cycD4-like protein | 595 | 1195 |
| Plant growth and development | rice RPN12 - AB037153 | 596 | 1196 |
| Plant growth and development | YFR052W/RPN12 - NP_116710 | 597 | 1197 |
| Plant growth and development | corn rpn12 | 598 | 1198 |
| Plant growth and development | soy rpn12 | 599 | 1199 |
| Plant growth and development | ZmHK1 | 602 | 1202 |
| Plant growth and development | Arabidopsis CRE1b | 603 | 1203 |
| Plant growth and development | Arabidopsis HK2 | 604 | 1204 |
| Plant growth and development | Arabidopsis HK3 | 605 | 1205 |
| Plant growth and development | rice histidine kinase | 609 | 1209 |
| Plant growth and | G protein b subunit | 114 | 453 |
| development/Stress tolerance | | | |
| Plant growth and | soy mlo-like | 199 | 538 |
| development/Stress tolerance | | | |
| Plant growth and | soy mlo-like | 200 | 539 |
| development/Stress tolerance | | | |
| Plant growth and | rice G alpha 1 | 201 | 540 |
| development/Stress tolerance | | | |
| Plant growth and | soy G-gamma subunit | 202 | 541 |
| development/Stress tolerance | | | |
| Plastid division | corn AAA-ATPase 2 | 13 | 352 |
| Plastid division | corn AAA-ATPase 1 | 339 | 678 |
| Root mass | Arabidopsis RAV2/G9 | 2 | 341 |
| Root mass | rice G9-like 1 | 3 | 342 |
| Root mass | rice G9-like 2 | 4 | 343 |
| Root mass | corn aquaporin RS81 | 8 | 347 |
| Seed development | Zm SET domain 2 | 82 | 421 |
| Seed development | Zm SET domain 1 | 83 | 422 |
| Seed development | Su(var) 3-9-like | 99 | 438 |
| Seed development | nucellin-like protein | 165 | 504 |
| Seed development | nucellin-like protein | 166 | 505 |
| Seed development | nucellin-like protein | 167 | 506 |
| Seed development | SET-domain protein-like | 206 | 545 |
| Seed development | corn histone H4 | 72 | 683 |
| Seed development | high sulfur zein protein precursor | 73 | 684 |
| Seed development | corn nucellin-like protein 1 | 121 | 730 |
| Seed development | rice nucellin-like protein 1 | 122 | 731 |
| Seed development | rice Crinkly4 - AB057787 | 331 | 938 |
| Seed development | rice S-domain receptor-like protein kinase 1 - BAA94516 | 332 | 939 |
| Seed development | rice S-domain receptor-like protein kinase 2 - BAB07906.1 | 333 | 940 |
| Seed development | rice S-domain receptor-like protein kinase 3 - BAB07905.1 | 334 | 941 |
| Seed development | rice S-domain receptor-like protein kinase 4 - BAB07904.1 | 335 | 942 |
| Seed development | rice S-domain receptor-like protein kinase 5 | 336 | 943 |
| Seed development | rice S-receptor kinase PK3 - BAB64641.1 | 337 | 944 |
| Seed development | OsPK10 - L27821 | 338 | 945 |
| Seed development | rice IRE1 - AB031396 | 443 | 1050 |
| Seed development | rice IRE1 N-terminal domain - AB031396 | 444 | 1051 |
| Seed development | rice IRE1 C-terminal domain - AB031396 | 445 | 1052 |
| Seed development | yeast IRE1 - P32361 | 446 | 1053 |
| Seed development | yeast IRE1 N-terminal domain - P32361 | 447 | 1054 |
| Seed development | yeast IRE1 C-terminal domain - P32361 | 448 | 1055 |
| Seed development | corn cytosine deaminase-like | 449 | 1056 |
| Seed development | corn aldose reductase | 450 | 1057 |
| Seed development | soy NADPH dependent mannose 6-phosphate reductase- | 451 | 1058 |
| Seed development | yeast YBL107c - Z35868 | 452 | 1222 |
| Seed development | yeast Ydr210w - NP_010496 | 453 | 1059 |
| Seed development | yeast YDR209c - S61572 | 454 | 1223 |
| Seed development | yeast Soh1 - NP_011388 | 455 | 1060 |
| Seed development | yeast TAD2 - NP_012499 | 456 | 1061 |
| Seed development | yeast Yet1 - NP_012858 | 457 | 1224 |
| Seed development | yeast Ylr162w - NP_013263 | 458 | 1225 |
| Seed development | yeast Ynl010w - NP_014388 | 459 | 1062 |
| Seed development | yeast Ynl124w - NP_014275 | 460 | 1063 |
| Seed development | yeast Ydl124w - NP_010159 | 461 | 1064 |
| Seed development | yeast Ygr039w - NP_011553 | 462 | 1226 |
| Seed development | yeast Usa1 - NP_013683 | 463 | 1227 |
| Seed development | yeast Ynr061c - NP_014459 | 464 | 1228 |
| Seed development | yeast Glutaredoxin - NP_009895 | 465 | 1065 |
| Seed development | soy unknown protein | 466 | 1066 |
| Seed development | soy putative protein | 467 | 1067 |
| Seed development | corn bromodomain protein | 477 | 1077 |
| Seed development | corn homeodomain leucine zipper protein | 478 | 1078 |
| Seed development | corn bZIP protein 3 | 479 | 1079 |
| Seed development | corn bZIP protein 4 [G-box binding factor 1] | 480 | 1080 |
| Seed development | corn remorin like DNA-binding protein 1 | 481 | 1081 |
| Seed development | corn remorin like DNA-binding protein 2 | 482 | 1082 |
| Seed development | corn Gld-Tea protein 2 | 483 | 1083 |
| Seed development | corn homeobox protein 1 | 484 | 1084 |
| Seed development | corn homeobox protein 3 | 485 | 1085 |
| Seed development | corn heat shock transcription factor 1 | 486 | 1086 |
| Seed development | corn heat shock transcription factor 2 | 487 | 1087 |
| Seed development | corn heat shock transcription factor 3 | 488 | 1088 |
| Seed development | corn heat shock transcription factor 4 | 489 | 1089 |
| Seed development | corn IAA-like 1 | 490 | 1090 |
| Seed development | corn IAA-like 4 | 491 | 1091 |
| Seed development | corn MADS box protein 100 | 492 | 1092 |
| Seed development | corn MADS box protein 102 | 493 | 1093 |
| Seed development | corn MADS box protein 103 | 494 | 1094 |
| Seed development | corn MADS box protein 104 | 495 | 1095 |
| Seed development | corn MADS box protein 105 | 496 | 1096 |
| Seed development | corn MADS box protein 108 | 497 | 1097 |
| Seed development | corn MADS box protein 114 | 498 | 1098 |
| Seed development | corn LIM domain protein 1 | 499 | 1099 |
| Seed development | corn LIM domain protein 2 | 500 | 1100 |
| Seed development | corn myb-like DNA binding protein | 501 | 1101 |
| Seed development | corn myb domain protein 1 | 502 | 1102 |
| Seed development | corn myb domain protein 3 | 503 | 1103 |
| Seed development | corn myb domain protein 4 | 504 | 1104 |
| Seed development | corn NAM-like protein | 505 | 1105 |
| Seed development | corn transcriptional co-activator-like protein 1 | 506 | 1106 |
| Seed development | corn LSD1-like protein 1 | 507 | 1107 |
| Seed development | corn GS1-like protein | 508 | 1108 |
| Seed development | corn RING finger 200 | 509 | 1109 |
| Seed development | corn RING finger protein 202 | 510 | 1110 |
| Seed development | corn PGPD14-like protein | 511 | 1111 |
| Seed development | corn Ankyrin protein 1 | 512 | 1112 |
| Seed development | corn zinc finger protein 10 [corn G325-like 3] | 513 | 1113 |
| Seed development | corn scarecrow protein 100 | 514 | 1114 |
| Seed development | corn transcription elongation factor | 515 | 1115 |
| Seed development | corn constans-like protein 10 | 516 | 1116 |
| Seed development | corn copine-like protein 1 | 517 | 1117 |
| Seed development | corn copine-like protein 2 | 518 | 1118 |
| Seed development | corn RING finger protein 201 | 519 | 1119 |
| Seed development | corn zinc finger protein | 520 | 1120 |
| Seed development | corn IAA-like 10 | 521 | 1121 |
| Seed development | corn constans-like 5 | 522 | 1122 |
| Seed development | corn Transcription Factor #1 | 554 | 1154 |
| Seed development | corn Transcription Factor #2 | 555 | 1155 |
| Seed development | maize nitrate transporter like 1 sequence | 606 | 1206 |
| Seed development | rice nitrate transporter like 1 sequence | 607 | 1207 |
| Seed development | rice nitrate transporter like 2 sequence | 608 | 1208 |
| Seed development | Arabidopsis nitrate transporter NTL1 like sequence | 610 | 1210 |
| Seed development | maize nitrate transporter NTL1 like sequence | 611 | 1211 |
| Seed development | maize nitrate transporter NTL1 like 2 sequence | 612 | 1212 |
| Seed development | rice nitrate transporter NTL1 like 1 sequence | 613 | 1213 |
| Seed development | rice nitrate transporter NTL1 like 2 sequence | 614 | 1214 |
| Signal transduction | corn MKP 1 [MAP kinase 4] | 75 | 686 |
| Signal transduction | corn MKP 3 [MAP kinase 5] | 76 | 687 |
| Signal transduction | corn MKP 4 | 77 | 688 |
| Signal transduction | corn MKP 6 | 78 | 689 |
| Signal transduction | corn MKP 7 | 79 | 690 |
| Signal transduction | corn MKP 8 [cdc2 kinase] | 80 | 691 |
| Signal transduction | soy MKP 1 | 81 | 692 |
| Signal transduction | soy MKP 4 | 82 | 693 |
| Stress tolerance | corn hemoglobin 1 | 34 | 373 |
| Stress tolerance | ASH1 | 95 | 434 |
| Stress tolerance | rice ASH1-like1 | 96 | 435 |
| Stress tolerance | rice MtN2-like | 97 | 436 |
| Stress tolerance | maize ferritin 2 | 144 | 483 |
| Stress tolerance | maize ferritin 1 | 145 | 484 |
| Stress tolerance | E. coli cytoplasmic ferritin | 146 | 485 |
| Stress tolerance | yeast glutamate decarboxylase | 187 | 526 |
| Stress tolerance | maize catalase-1 | 241 | 580 |
| Stress tolerance | maize catalase-3 | 242 | 581 |
| Stress tolerance | ascorbate peroxidase | 243 | 582 |
| Stress tolerance | corn GDI | 244 | 583 |
| Stress tolerance | soy GDI | 245 | 584 |
| Stress tolerance | corn rho GDI | 246 | 585 |
| Stress tolerance | basic blue copper protein | 247 | 586 |
| Stress tolerance | plantacyanin | 248 | 587 |
| Stress tolerance | basic blue copper protein | 249 | 588 |
| Stress tolerance | Similar to blue copper protein precursor | 250 | 589 |
| Stress tolerance | lamin | 251 | 590 |
| Stress tolerance | fC-zmfl700551169a-allyl alcohol dehydrogenase | 252 | 591 |
| Stress tolerance | allyl alcohol dehydrogenase | 253 | 592 |
| Stress tolerance | allyl alcohol dehydrogenase | 254 | 593 |
| Stress tolerance | quinone oxidoreductase | 255 | 594 |
| Stress tolerance | E. nidulans cysA - AF029885 | 256 | 595 |
| Stress tolerance | BAA18167 - Synechocystis cysE | 257 | 596 |
| Stress tolerance | Synechocystis thiol-specific antioxidant protein - BAA10136 | 258 | 597 |
| Stress tolerance | yeast TSA2 - NP_010741 | 259 | 598 |
| Stress tolerance | yeast mTPx - Z35825 | 260 | 599 |
| Stress tolerance | yeast TPx III - NP_013210 | 261 | 600 |
| Stress tolerance | soy putative 2-cys peroxiredoxin | 262 | 601 |
| Stress tolerance | soy peroxiredoxin | 263 | 602 |
| Stress tolerance | heat shock protein 26, plastid-localized | 264 | 603 |
| Stress tolerance | heat shock protein | 265 | 604 |
| Stress tolerance | low molecular weight heat shock protein | 266 | 605 |
| Stress tolerance | 18 kDa heat shock protein | 267 | 606 |
| Stress tolerance | heat shock protein 16.9 | 268 | 607 |
| Stress tolerance | HSP21-like protein | 269 | 608 |
| Stress tolerance | Opt1p - NP_012323 | 270 | 609 |
| Stress tolerance | SVCT2-like permease | 271 | 610 |
| Stress tolerance | SVCT2-like permease | 272 | 611 |
| Stress tolerance | soy HMG CoA synthase | 275 | 614 |
| Stress tolerance | yeast HMGS - X96617 | 276 | 615 |
| Stress tolerance | soy isoflavone synthase | 309 | 648 |
| Stress tolerance | soy ttg1-like 2 | 310 | 649 |
| Stress tolerance | yeast YKL091c - Z28091 | 331 | 670 |
| Stress tolerance | corn Ssh1-like protein 1 | 332 | 671 |
| Stress tolerance | corn Ssh1-like protein 3 | 333 | 672 |
| Stress tolerance | corn Ssh1-like protein 4 | 334 | 673 |
| Stress tolerance | soy Ssh1-like protein 2 [ssh2] | 335 | 674 |
| Stress tolerance | Arabidopsis AtHAP3a | 1 | 615 |
| Stress tolerance | corn AtHAP3a-like1 | 2 | 616 |
| Stress tolerance | corn AtHAP3a-like2 | 3 | 617 |
| Stress tolerance | corn AtHAP3a-like3 | 4 | 618 |
| Stress tolerance | Arabidopsis CCA1 | 5 | 619 |
| Stress tolerance | corn hemoglobin 2 | 6 | 620 |
| Stress tolerance | At TOR1-like | 7 | 621 |
| Stress tolerance | G28/BAA32418 | 9 | 623 |
| Stress tolerance | G378 | 10 | 624 |
| Stress tolerance | G464 | 11 | 625 |
| Stress tolerance | Adenosylhomo cysteinase | 15 | 629 |
| Stress tolerance | maize glycine rich protein | 17 | 631 |
| Stress tolerance | wilt-like 1 | 18 | 632 |
| Stress tolerance | wilt-like 2 | 19 | 633 |
| Stress tolerance | wilt-like 3 | 20 | 634 |
| Stress tolerance | wilt-like 4 | 21 | 635 |
| Stress tolerance | G188 | 26 | 639 |
| Stress tolerance | G19 | 27 | 640 |
| Stress tolerance | corn G214 | 30 | 643 |
| Stress tolerance | ASH2 - F17I14_190 (Suppressor of hog1) | 33 | 646 |
| Stress tolerance | soy ASH2-like 1 | 34 | 647 |
| Stress tolerance | soy ASH2-like 3 | 35 | 648 |
| Stress tolerance | soy ASH2-like 4 | 36 | 649 |
| Stress tolerance | corn phospholipase C 1 | 40 | 653 |
| Stress tolerance | corn diacylglycerol kinase 2 | 41 | 654 |
| Stress tolerance | corn Ins(1,4,5)P(3) 5-phosphatase 1 | 42 | 655 |
| Stress tolerance | yeast ado1 | 51 | 664 |
| Stress tolerance | corn adenosine kinase 1 | 52 | 665 |
| Stress tolerance | soy adenosine kinase 2 | 53 | 666 |
| Stress tolerance | Synechocystis hypothetical sugar kinase - BAA10827 | 54 | 667 |
| Stress tolerance | corn adenosylhomocysteinase 1 | 55 | 668 |
| Stress tolerance | yeast S-adenosyl-L-homocysteine hydrolase - NP_010961 | 56 | 669 |
| Stress tolerance | Synechocystis S-adenosylhomocysteine hydrolase-BAA18079 | 57 | 670 |
| Stress tolerance | Aspergillus yA (laccase 1) - X52552 | 59 | 672 |
| Stress tolerance | Synechocystis ssr3189 - BAA17701 | 67 | 1216 |
| Stress tolerance | Synechocystis ssr2315 - BAA17190 | 68 | 1217 |
| Stress tolerance | Agrobacterium aiiA-like protein [attM]- AAD43990 | 69 | 680 |
| Stress tolerance | Xylella aiiA-like protein - XF1361 | 70 | 681 |
| Stress tolerance | Xanthomonas aiiA - like protein | 71 | 682 |
| Stress tolerance | corn ABF2-like 1 | 83 | 694 |
| Stress tolerance | soy ABF2-like 1 | 84 | 695 |
| Stress tolerance | yeast YOR161c - Z75069 | 86 | 697 |
| Stress tolerance | yeast HNM1 - Z72599 | 87 | 698 |
| Stress tolerance | corn violaxanthin de-epoxidase | 98 | 707 |
| Stress tolerance | rice serine acetyltransferase 1 | 99 | 708 |
| Stress tolerance | rice serine acetyltranferase 2 | 100 | 709 |
| Stress tolerance | corn CEO-like protein | 101 | 710 |
| Stress tolerance | cotton ttg1-1 | 120 | 729 |
| Stress tolerance | rice LEA 1 | 124 | 733 |
| Stress tolerance | rice LEA 2 [wsi18] | 125 | 734 |
| Stress tolerance | corn LEA 1 | 126 | 735 |
| Stress tolerance | corn LEA 2 | 127 | 736 |
| Stress tolerance | corn selenium-binding protein | 140 | 749 |
| Stress tolerance | maize glutathione S-transferase IV | 141 | 750 |
| Stress tolerance | calcium-dependent protein kinase | 142 | 751 |
| Stress tolerance | protein kinase CK2 regulatory subunit CK2B3 | 143 | 752 |
| Stress tolerance | 19K zein precursor | 144 | 753 |
| Stress tolerance | corn EREBP/AP2-like transcription factor | 145 | 754 |
| Stress tolerance | G-box binding factor 1 | 146 | 755 |
| Stress tolerance | corn high mobility group protein | 147 | 756 |
| Stress tolerance | corn glycine-rich RNA-binding protein | 148 | 757 |
| Stress tolerance | corn enolase 1 | 149 | 758 |
| Stress tolerance | maize RING finger protein | 150 | 759 |
| Stress tolerance | maize 60S acidic ribosomal protein P0 | 151 | 760 |
| Stress tolerance | corn nucleotide-binding protein | 152 | 761 |
| Stress tolerance | corn betaine-aldehyde dehydrogenase | 183 | 791 |
| Stress tolerance | rice betaine aldehyde dehydrogenase | 184 | 792 |
| Stress tolerance | yeast PEM1/CHO2 | 185 | 793 |
| Stress tolerance | yeast PEM2/OPI3 | 186 | 794 |
| Stress tolerance | corn phosphoethanolamine N-methyltransferase 2 | 187 | 795 |
| Stress tolerance | E. coli betT | 188 | 796 |
| Stress tolerance | Xenorhabdus BetT-like 1 | 189 | 797 |
| Stress tolerance | corn NHX1-like 1 | 190 | 798 |
| Stress tolerance | OsNHX1 - AB021878 | 191 | 799 |
| Stress tolerance | corn vacuolar H+-pyrophosphatase 1 | 192 | 800 |
| Stress tolerance | corn vacuolar H+-pyrophosphatase 2 | 193 | 801 |
| Stress tolerance | corn vacuolar H+-pyrophosphatase 5 | 194 | 802 |
| Stress tolerance | Arabidopsis eskimo 1 | 230 | 838 |
| Stress tolerance | corn HMT 1 (N-methyltransferase) | 237 | 845 |
| Stress tolerance | corn HMT 2 (N-methyltransferase) | 238 | 846 |
| Stress tolerance | E. coli yagD homocysteine S-methyltransferase-Q47690 | 239 | 847 |
| Stress tolerance | yeast Mht1 - NP_013038 | 240 | 848 |
| Stress tolerance | wilt-1256 | 245 | 853 |
| Stress tolerance | corn NAD(P)H quinone oxidoreductase | 246 | 854 |
| Stress tolerance | rice CBP80 - AY017415 | 247 | 855 |
| Stress tolerance | yeast GCR3 - D10224 | 248 | 856 |
| Stress tolerance | corn CBP20 | 249 | 857 |
| Stress tolerance | yeast CBC2 - Z73534 | 250 | 858 |
| Stress tolerance | rice serine decarboxylase - AAG12476 | 251 | 859 |
| Stress tolerance | corn serine decarboxylase | 252 | 860 |
| Stress tolerance | soy serine decarboxylase | 253 | 861 |
| Stress tolerance | rice VP14-like 1 | 261 | 869 |
| Stress tolerance | rice VP14-like 2 | 262 | 870 |
| Stress tolerance | yeast GPA2 | 281 | 888 |
| Stress tolerance | soy G28 like | 300 | 907 |
| Stress tolerance | corn G1792-like 2 | 301 | 908 |
| Stress tolerance | G1792 | 302 | 909 |
| Stress tolerance | soy 14-3-3 22 | 339 | 946 |
| Stress tolerance | soy 14-3-3 22 N-terminus | 340 | 947 |
| Stress tolerance | 14-3-3-like protein N-terminus | 341 | 948 |
| Stress tolerance | sorghum 14-3-3 10 | 342 | 949 |
| Stress tolerance | sorghum 14-3-3 10 N-terminus | 343 | 950 |
| Stress tolerance | rice 14-3-3 15 | 344 | 951 |
| Stress tolerance | rice 14-3-3 15 N-terminus | 345 | 952 |
| Stress tolerance | corn 14-3-3 13 | 346 | 953 |
| Stress tolerance | corn 14-3-3 13 N-terminus | 347 | 954 |
| Stress tolerance | 14-3-3 protein N-terminus | 348 | 955 |
| Stress tolerance | rice 14-3-3 10 | 349 | 956 |
| Stress tolerance | rice 14-3-3 10 N-terminus | 350 | 957 |
| Stress tolerance | soy 14-3-3 21 | 351 | 958 |
| Stress tolerance | soy 14-3-3 21 N-terminus | 352 | 959 |
| Stress tolerance | wheat 14-3-3 10 | 353 | 960 |
| Stress tolerance | wheat 14-3-3 10 N-terminus | 354 | 961 |
| Stress tolerance | corn 14-3-3 17 | 355 | 962 |
| Stress tolerance | corn 14-3-3 17 N-terminus | 356 | 963 |
| Stress tolerance | CAR1-like 1 | 365 | 972 |
| Stress tolerance | corn CAR1-like 3 | 366 | 973 |
| Stress tolerance | corn CAR1-like 4 | 367 | 974 |
| Stress tolerance | corn CAR1-like 5 | 368 | 975 |
| Stress tolerance | yeast hnRNP methyltransferase-CAA53689 | 369 | 976 |
| Stress tolerance | corn arginine N-methyl transferase 1 | 370 | 977 |
| Stress tolerance | corn arginine N-methyl transferase 2 | 371 | 978 |
| Stress tolerance | CAR1 - corn ABA inducible RNA-binding protein | 372 | 979 |
| Stress tolerance | corn ASH1 | 399 | 1006 |
| Stress tolerance | corn CLC1 | 468 | 1068 |
| Stress tolerance | corn CLD1 | 469 | 1069 |
| Stress tolerance | rice Asr1 - AF039573 | 470 | 1070 |
| Stress tolerance | rice CLC1-like 1 - BAB19059 | 471 | 1071 |
| Stress tolerance | corn Asr1-like 1 | 472 | 1072 |
| Stress tolerance | corn Asr1-like 5 | 473 | 1073 |
| Stress tolerance | soy G1792-like | 533 | 1133 |
| Stress tolerance | corn CVY-CIK | 540 | 1140 |
| Stress tolerance | corn calcium dependant protein kinase | 542 | 1142 |
| Stress tolerance | corn unknown protein | 543 | 1143 |
| Stress tolerance | corn putative splicing factor | 544 | 1144 |
| Stress tolerance | rice hydroxyproline-rich glycoprotein | 545 | 1145 |
| Stress tolerance | rice unknown protein - AAK14418 | 546 | 1146 |
| Stress tolerance | corn unknown protein | 547 | 1147 |
| Stress tolerance | corn alpha-amylase | 548 | 1148 |
| Stress tolerance | soy G1792-like 2 | 559 | 1159 |
| Stress tolerance | corn G1792-like 3 | 560 | 1160 |
| Stress tolerance | sorghum TTG1-like | 600 | 1200 |
| Stress tolerance | corn TTG1-like protein 2 | 601 | 1201 |
| Stress tolerance/Disease | corn allene oxide synthase | 177 | 516 |
| resistance | | | |
| Stress tolerance/Disease | corn COI1-like | 178 | 517 |
| resistance | | | |
| Stress tolerance/Disease | corn COI1-like | 180 | 519 |
| resistance | | | |
| Stress tolerance/Disease | COI1-like | 181 | 520 |
| resistance | | | |
| Stress tolerance/Disease | corn 1-aminocyclopropane-1-carboxylate oxidase | 184 | 523 |
| resistance | | | |
| Stress tolerance/Disease | rice 1-aminocyclopropane-1 carboxylate synthase | 185 | 524 |
| resistance | | | |
| Stress tolerance/Disease | corn 12-oxophytodienoate reductase 1 | 316 | 655 |
| resistance | | | |
| Stress tolerance/Disease | corn 12-oxo-phytodienoate reductase-like 3 | 317 | 656 |
| resistance | | | |
| Stress tolerance/Disease | corn 12-oxophytodienoate reductase-like 4 | 318 | 657 |
| resistance | | | |
| Stress tolerance/Disease | corn hydroperoxide lyase | 319 | 658 |
| resistance | | | |
| Stress tolerance/Disease | soy JMT-like protien 1 | 336 | 675 |
| resistance | | | |
| Stress tolerance/Disease | corn JMT-like protein 1 | 337 | 676 |
| resistance | | | |
| Stress tolerance/Disease | corn JMT-like protein 2 | 338 | 677 |
| resistance | | | |
| Sucrose production/transport | invW | 10 | 349 |
| Sucrose transport | corn monosaccharide transporter 1 | 298 | 637 |
| Sucrose transport | soy monosaccharide transporter 3 | 299 | 638 |
| Sucrose transport | corn monosaccharide transporter 3 | 300 | 639 |
| Sucrose transport | soy monosaccharide transporter 1 | 301 | 640 |
| Sucrose transport | corn monosaccharide transporter 6 | 302 | 641 |
| Sucrose transport | corn monosaccharide transporter 4 | 303 | 642 |
| Sucrose transport | soy monosaccharide transporter 2 | 304 | 643 |
| Sucrose transport | soy sucrose transporter | 305 | 644 |
| Sucrose transport | corn sucrose transporter 2 | 306 | 645 |
| Sucrose transport | corn monosaccharide transporter 8 | 307 | 646 |
| Sucrose transport | corn monosaccharide transporter 7 | 308 | 647 |
| Water use efficiency | rice G975 | 5 | 344 |
| Water use efficiency | corn G975 | 6 | 345 |
| Water use efficiency | corn Glossy15 | 7 | 346 |
| Water use efficiency | Arabidopsis G975 | 73 | 412 |
| Water use efficiency | CUT1 | 74 | 413 |
| Yield associated genes | rice chloroplastic fructose-1,6-bisphosphatase | 121 | 460 |
| Yield associated genes | E. coli fructose-1,6-bisphosphatase | 122 | 461 |
| Yield associated genes | Synechocystis fructose-1,6-bisphosphatase F-I | 123 | 462 |
| Yield associated genes | Synechocystis fructose-1,6-bisphosphatase F-II | 124 | 463 |
| Yield associated genes | Yeast RPT5 | 125 | 464 |
| Yield associated genes | Yeast RRP5 | 126 | 465 |
| Yield associated genes | Rice CBP-like gene | 127 | 466 |
| Yield associated genes | rice BAB09754 | 128 | 467 |
| Yield associated genes | LIB3061-001-H7_FLI | 129 | 468 |
| Yield associated genes | ubiquitin-conjugating enzyme-like protein | 217 | 556 |
| Yield associated genes | unknown protein recognized by PF01169 | 218 | 557 |
| Yield associated genes | 26S protease regulatory subunit 6A homolog | 219 | 558 |
| Yield associated genes | fC-zmhuLIB3062-044-Q1-K1-B8 | 329 | 668 |
| Yield associated genes | corn E4/E8 binding protein-like | 330 | 669 |

*These have been disclosed in U.S. Patent Application Publication No. US20030233670 which is incorporated herein by reference. Some proteins may provide more than one traits. For example, proteins of SEQ ID NOs 419 and 423. Intrinsic yield includes traits such as apomixis, carbon and/or nitrogen metabolism, cell division, DNA integration, ethylene signaling, flower development, homologous recombination, light response, photosynthesis, carbon portioning, plant growth and development, plastid division, root mass, seed development, signal transduction, sucrose production/transport. Nutreient use efficiency may include nitrogen metabolism, iron uptake, metabolite transport, nitric oxide signaling, nitrogen assimilation, nitrogen transport, nitrogen uptake, phosphate uptake, root mass. Stress tolerance may include signal transduction. Drought tolerance includes water use efficiency.

TABLE 6
Examples of traits and proteins suitable for use in the present invention. These have been disclosed in PCT Application Pub.
No. WO2007/044043, which is incorporated herein by reference. Some proteins may provide more than one traits. For example,
proteins of SEQ ID NOs 79 and 31. Drought tolerance includes enhanced water use efficiency.

| | | SEG |
|---|---|---|
| Trait | Protein/Gene | ID |
| Enhanced cold tolerance, and enhanced water use efficiency | Arabidopsis CtpA | 37 |
| Enhanced nitrogen use efficiency, enhanced water use | corn OsPK7-like | 79 |
| efficiency, increased yield | | |
| Enhanced cold tolerance | rab7c | 2 |
| Enhanced cold tolerance | rice MADS3 delta MADS-box-L37528 | 7 |
| Enhanced cold tolerance | Arabidopsis homeodomain transcription factor- | 9 |
| Enhanced cold tolerance | Arabidopsis AP2 domain transcription factor | 10 |
| Enhanced cold tolerance | Arabidopsis GATA domain transcription factor | 11 |
| Enhanced cold tolerance | Arabidopsis AT-hook domain transcription factor- | 12 |
| Enhanced cold tolerance | Arabidopsis hypothetical protein [NM_114802] | 15 |
| Enhanced cold tolerance | corn hypothetical protein | 16 |
| Enhanced cold tolerance | Agrobacterium cryptochrome-like protein-AE008050 | 18 |
| Enhanced cold tolerance | soybean MAP kinase 6 like 2 sequence | 30 |
| Enhanced cold tolerance | Arabidopsis protein phosphatase 1A | 36 |
| Enhanced cold tolerance | Corn glyoxalase II isozyme | 27 |
| Enhanced cold tolerance | Corn RNA 3-TERMINAL PHOSPHATE CYCLASE-LIKE | 28 |
| | PROTEIN | |
| Enhanced cold tolerance | rice Di19 like sequence | 29 |
| Enhanced cold tolerance | Corn protein similar to Arabidopsis Probable microsomal signal | 39 |
| | peptidase | |
| Enhanced cold tolerance and enhanced water use efficiency | soybean hsp17.4 like 1 sequence | 33 |
| Enhanced cold tolerance enhanced nitrogen use efficiency | Arabidopsis E2F | 35 |
| Enhanced cold tolerance and enhanced water use efficiency | corn SNF1-like protein 8 | 21 |
| Enhanced cold tolerance and increased yield | NLI Interacting Isoform T1- | 23 |
| Enhanced cold tolerance condition and increased yield | maize synaptobrevin-related sequnece 1- | 24 |
| Enhanced cold tolerance, and enhanced nitrogen use | Ralstonia metallidurans glutamate decarboxylase | 31 |
| efficiency | | |
| Enhanced nitrogen use efficiency | soy Pra2-like protein 2 | 17 |
| Enhanced nitrogen use efficiency | [Oryza sativa] putative aldose reductase | 40 |
| Enhanced nitrogen use efficiency and enhance cold tolerance | corn MADS box protein 110 | 8 |
| Enhanced nitrogen use efficiency and enhanced cold | rice DET1-like-BAB16336 | 13 |
| tolerance | | |
| Enhanced nitrogen use efficiency and increased yield | maize magnesium transporter mrs2-1-like 1 sequence | 25 |
| Enhanced nitrogen use efficiency, enhanced water use | rice SNF1-like protein 9 [OsPK4]-AB011967 | 19 |
| efficiency, increased yield | | |
| Enhanced nitrogen use efficiency, enhanced water use | soy SNF-like protein 2 | 76 |
| efficiency, increased yield | | |
| Enhanced seed protein | lactoylglutathione lyase | 1 |
| Enhanced water use efficiency | soybean G482-like 1 | 14 |
| Enhanced water use efficiency | Corn Protein similar to nodulin MtN3 protein | 26 |
| Enhanced water use efficiency | rice HSF5 like sequence | 32 |
| Enhanced water use efficiency | Corn putative pyrrolidone carboxyl peptidase | 34 |
| Enhanced water use efficiency | CDPK kinase domain | 3 |
| Enhanced water use efficiency and enhanced cold tolerance | SCOF-1 | 4 |
| Enhanced water use efficiency and enhanced nitrogen use | corn SNF1-like protein 3 | 20 |
| efficiency | | |
| Improved cold tolerance | Arabidopsis agl11 delta K-box | 6 |
| Improved growth under cold stress | corn hypothetical protein | 69 |
| Improved growth under cold stress | corn hypothetical protein | 70 |
| Improved growth under cold stress | Arabidopsis hypothetical protein | 73 |
| Improved growth under cold stress | rice G975 like1 | 81 |
| Improved growth under low nitrogen, drought, and/or cold | yeast SNF1-A26030 | 74 |
| stresses | | |
| Increased seed protein | Zea Mays Kinase II (similar to Yeast IKS1 & At MRK1) | 41 |
| Increased seed protein | soy G1928 like 1 | 43 |
| Increased yield | Arabidopsis CtpA | 38 |
| Increased yield | Fructose-1-6-bisphosphatase | 42 |
| Increased yield | Synechocystis sp. 6803 Hik19 | 44 |
| Increased yield | Synechocystis sp. 6803 Hik19 | 45 |
| Increased yield | Arabidopsis NAC domain transcription factor | 46 |
| Increased yield | soybean catalase-like 1 | 48 |
| Increased yield | corn ALG-2 interacting protein | 49 |
| Increased yield | Putative Serine Carboxypeptidase- | 50 |
| Increased yield | Putative Ankyrin Like Protein- | 51 |
| Increased yield | Putative Kinase Like Protein- | 52 |
| Increased yield | Putative Protein- | 53 |
| Increased yield | yeast YPR145W/asn1-U40829 | 54 |
| Increased yield | rice AtHSP17.6A like 1 sequence | 55 |
| Increased yield | yeast YDL123w | 56 |
| Increased yield | rice 12-oxophytodienoate reductase like 1 sequence | 57 |
| Increased yield | soybean MAP kinase 6 like 3 sequence | 58 |
| Increased yield | Arabidopsis GAD1 | 59 |
| Increased yield | soybean hsp17.4 like 4 sequence | 61 |
| Increased yield | maize hsp60 like 4 sequence | 62 |
| Increased yield | soy dsPTP 1 | 63 |
| Increased yield | Yeast GLC3 Glycogen branching enzyme | 64 |
| Increased yield | Arabidopsis unknown protein | 65 |
| Increased yield | beta-D-glucosidase | 66 |
| Increased yield | unknown protein1 | 67 |
| Increased yield | aldehyde oxidase | 68 |
| Increased yield | Arabidopsis cysteine proteinase inhibitor | 71 |
| Increased yield | Arabidopsis cysteine proteinase inhibitor | 72 |
| Increased yield | soy SNF1-like protein 1 | 75 |
| Increased yield | Rice Glyoxalase II | 78 |
| Increased yield | rice phyA with Arabidopsis phyC intron 1 | 80 |
| Increased yield | Corn Phytochrome A | 82 |
| Increased yield | Arabidopsis G1760 | 83 |
| Increased yield and enhanced nitrogen use efficiency | yeast alanine aminotransferase 1-AAB67593 | 47 |
| Increased yield and enhanced water use efficiency | soy G1760 | 77 |
| Increased yield, enhanced cold tolerance and enhanced | Corn Rubisco Activase 2 | 22 |
| nitrogen use efficiency | | |
| Increased yield, enhanced cold tolerance and enhanced | Synechococcus sp. PCC 6301 Delta9 desaturase | 5 |
| water use efficiency | | |

TABLE 7
Examples of traits and proteins suitable for use in the present invention.*

| PEP | | | | | | | |
|---|---|---|---|---|---|---|---|
| SEQ | | | | | | | |
| ID | Proetein | Traits | | | | | |
| 205 | gb\|AAD48981.1\|contains similarity to | HS | | | | | |
| | Solanum lycopersicum (tomato) wound | | | | | | |
| | induced protein | | | | | | |
| 206 | gb\|AAM62490.1\|putative zinc finger | LN | | | | | |
| | protein | | | | | | |
| 207 | ref\|NP_191871.1\|cyclin family protein | HS | | | | | |
| 208 | ref\|NP_566299.1\|GPI transamidase | CK | CS | | | | |
| | component Gpi16 subunit family | | | | | | |
| | protein | | | | | | |
| 209 | ref\|NP_566244.1\|transmembrane | CK | | | | | |
| | protein, putative | | | | | | |
| 210 | emb\|CAB81279.1\|putative protein | HS | PP | PEG | | | |
| 211 | dbj\|BAC43077.1\|unknown protein | PP | SS | HS | | | |
| 212 | ref\|NP_175971.3\|transcription factor- | CK | | | | | |
| | related | | | | | | |
| 213 | gb\|AAM44902.1\|putative catalase | PP | SP | | | | |
| 214 | gb\|AAC97995.1\|Similar to gb\|Z30094 | LN | PP | HS | | | |
| | basic transcripion factor 2, 44 kD | | | | | | |
| | subunit from Homo sapiens. | | | | | | |
| 215 | gb\|AAD55662.1\|Highly similar to non | DS | | | | | |
| | intermediate filament IFA binding | | | | | | |
| | protein | | | | | | |
| 216 | gb\|AAM61703.1\|protein kinase-like | CK | | | | | |
| | protein | | | | | | |
| 217 | emb\|CAB41143.1\|putative peptide | CK | | | | | |
| | transporter | | | | | | |
| 218 | gb\|AAK64127.1\|unknown protein | PEG | | | | | |
| 219 | gb\|AAC23773.1\|putative acetone- | DS | | | | | |
| | cyanohydrin lyase | | | | | | |
| 220 | gb\|AAK93693.1\|putative 3- | CK | PEG | CS | HS | | PP |
| | methyladenine DNA glycosylase | | | | | | |
| 221 | ref\|NP_011658.1\|Btn2p | PP | | | | | |
| | [Saccharomyces cerevisiae] | | | | | | |
| 222 | ref\|NP_178499.2\|MATE efflux family | LN | | | | | |
| | protein | | | | | | |
| 223 | ref\|NP_198887.1\|zinc finger (C2H2 | CS | | | | | |
| | type) family protein | | | | | | |
| 224 | gb\|AAN28776.1\|At3g51780/ORF3 | CK | | | | | |
| 225 | gb\|AAF27024.1\|putative nodulin | CS | | | | | |
| | [Arabidopsis thaliana] | | | | | | |
| | ref\|NP_187169.1\|GDSL-motif | | | | | | |
| | lipase/hydrolase family protein | | | | | | |
| 226 | gb\|AAM51318.1\|unknown protein | CS | | | | | |
| 227 | gb\|AAM64944.1\|betaine aldehyde | PP | | | | | |
| | dehydrogenase, putative | | | | | | |
| 228 | ref\|NP_171616.1\|33 kDa | PEG | SS | | | | |
| | ribonucleoprotein, chloroplast, putative/ | | | | | | |
| | RNA-binding protein cp33, putative | | | | | | |
| 229 | emb\|CAA58159.1\|glutamic-dependent | CK | CS | SS | | | |
| | asparagine synthase | | | | | | |
| 230 | pir\|\|A86468probable zinc finger protein | PP | PEG | | | | |
| 231 | gb\|AAM10965.1\|putative bHLH | CK | | | | | |
| | transcription factor [Arabidopsis | | | | | | |
| | thaliana] | | | | | | |
| 232 | ref\|NP_566566.1\|protein phosphatase | CK | HS | PEG | CS | | |
| | 2C family protein | | | | | | |
| 233 | gb\|AAM65842.1\|putative RING-H2 | PP | HS | | | | |
| | zinc finger protein | | | | | | |
| 234 | gb\|AAV51937.1\|AP2/EREBP | CK | | | | | |
| | transcription factor ERF-2 | | | | | | |
| 235 | emb\|CAA09367.1\|HB2 homeodomain | CK | CS | | | | |
| | protein | | | | | | |
| 236 | gb\|AAD27870.1\|BRH1 RING finger | HS | PP | PEG | | | |
| | protein | | | | | | |
| 237 | gb\|AAM14913.1\|putative malonyl- | HS | | | | | |
| | CoA:Acyl carrier protein transacylase | | | | | | |
| 238 | gb\|AAM13238.1\|putative NADPH- | PP | PEG | | | | |
| | dependent mannose 6-phosphate | | | | | | |
| | reductase | | | | | | |
| 239 | ref\|NP_567300.1\|short-chain | HS | | | | | |
| | dehydrogenase/reductase (SDR) | | | | | | |
| | family protein | | | | | | |
| 240 | gb\|AAS46245.1\|HMG-CoA synthase 2 | CK | HS | SS | | | |
| 241 | gb\|AAM21344.1\|MADS-box protein 4 | SP | | | | | |
| 242 | ref\|NP_196254.1\|ribosomal protein | SS | HS | | | | |
| | S8e family protein | | | | | | |
| 243 | gb\|AAD23719.1\|putative RING zinc | PEG | | | | | |
| | finger protein | | | | | | |
| 244 | pir\|\|T52379zinc finger protein ZPT3-3 | PEG | | | | | |
| 245 | emb\|CAC12995.1\|putative AUX1-like | CK | PEG | CS | | | |
| | permease | | | | | | |
| 246 | gb\|AAM63089.1\|cold-regulated protein | CS | HS | PP | | | |
| | cor15b precursor | | | | | | |
| 247 | pir\|\|D96781cytochrome P450, | PEG | | | | | |
| | probable, 64213-66051 | | | | | | |
| 248 | gb\|AAC77858.1\|putative cytochrome | PP | | | | | |
| | P450 | | | | | | |
| 249 | gb\|AAM65367.1\|phi-1-like protein | LN | LL | | | | |
| 250 | ref\|NP_200258.2\|zinc finger (B-box | DS | | | | | |
| | type) family protein | | | | | | |
| 251 | gb\|AAC36698.1\|protein phosphatase- | PP | CK | | | | |
| | 2C; PP2C | | | | | | |
| 252 | gb\|AAM61425.1\|unknown | CK | PEG | | | | |
| 253 | ref\|NP_174092.1\|glycine-rich protein | HS | PP | | | | |
| 254 | ref\|NP_565909.1\|radical SAM | PEG | | | | | |
| | domain-containing protein | | | | | | |
| 255 | emb\|CAB43520.1\|MAP kinase | CK | CS | | | | |
| 256 | gb\|AAC34217.1\|putative alcohol | CS | | | | | |
| | dehydrogenase | | | | | | |
| 257 | gb\|AAC34486.1\|E3 ubiquitin ligase | CK | LL | CS | | | |
| | SCF complex subunit SKP1/ASK1 | | | | | | |
| | (At19), | | | | | | |
| 258 | dbj\|BAA95756.1\|expansin-like protein | PP | HS | | | | |
| 259 | gb\|AAK28345.1\|receptor-like protein | CK | PP | | | | |
| | kinase 3 | | | | | | |
| 260 | gb\|AAG60115.1\|hypothetical protein | PP | | | | | |
| 261 | gb\|AAM14112.1\|putative | LL | | | | | |
| | ubiquinone/menaquinone biosynthesis | | | | | | |
| | methyltransferase | | | | | | |
| 262 | ref\|NP_176849.1\|nodulin MtN3 family | SS | PEG | | | | |
| | protein | | | | | | |
| 263 | sp\|P53780\|METC_ARATH | PP | SS | | | | |
| | Cystathionine beta-lyase, chloroplast | | | | | | |
| | precursor (CBL) (Beta-cystathionase) | | | | | | |
| | (Cysteine lyase) | | | | | | |
| 264 | gb\|AAM48022.1\|photoassimilate- | SP | DS | | | | |
| | responsive protein PAR-1b-like protein | | | | | | |
| 265 | ref\|NP_173376.1\|very-long-chain | PEG | PP | | | | |
| | fatty acid condensing enzyme, putative | | | | | | |
| 266 | sp\|P20438\|CG12_YEAST G1/S- | DS | | | | | |
| | specific cyclin CLN2 gb\|AAA65725.1\| | | | | | | |
| | cyclin 2 | | | | | | |
| 267 | dbj\|BAA28775.1\|Cdk-activating kinase | LL | | | | | |
| | 1At | | | | | | |
| 268 | ref\|NP_011956.1\|Nucleolar protein | SP | | | | | |
| | involved in the assembly of the large | | | | | | |
| | ribosomal subunit; contains a | | | | | | |
| | sigma(70)-like motif, which is thought | | | | | | |
| | to bind RNA | | | | | | |
| 269 | gb\|AAM51327.1\|putative histidyl-tRNA | CK | HS | CS | | | |
| | synthetase | | | | | | |
| 270 | ref\|NP_116708.1\|20S proteasome | PP | PEG | SS | | | |
| | beta-type subunit | | | | | | |
| 271 | pir\|\|S69027 ammonium transport | LL | DS | | | | |
| | protein MEP3 | | | | | | |
| 272 | sp\|O24543\|AX2E_PHAAU Auxin- | DS | | | | | |
| | induced protein 22E (Indole-3-acetic | | | | | | |
| | acid induced protein ARG14) | | | | | | |
| 273 | dbj\|BAB09771.1\|serine/threonine | LL | | | | | |
| | protein kinase-like protein | | | | | | |
| 274 | emb\|CAE26387.1\|phosphoglycerate | CK | DS | CS | | | |
| | kinase | | | | | | |
| 275 | dbj\|BAB08997.1\|monosaccharide | PP | PEG | | | | |
| | transporter | | | | | | |
| 276 | ref\|NP_010046.1\|Phosphorelay | HS | | | | | |
| | intermediate protein, phosphorylated | | | | | | |
| | by the plasma membrane sensor | | | | | | |
| | Sln1p in response to osmotic stress | | | | | | |
| | and then in turn phosphorylates the | | | | | | |
| | response regulators Ssk1p in the | | | | | | |
| | cytosol and Skn7p in the nucleus | | | | | | |
| 277 | ref\|NP_565524.1\|stress enhanced | DS | LN | | | | |
| | protein 2 (SEP2) | | | | | | |
| 278 | ref\|NP_012797.1\|Required for | SS | | | | | |
| | transcription of rDNA by RNA | | | | | | |
| | Polymerase I; DNA-independent RNA | | | | | | |
| | Polymerase I transcription factor | | | | | | |
| 279 | pir\|\|S69555 myo-inositol transport | HS | SS | PP | | | |
| | protein ITR1-yeast) | | | | | | |
| 280 | pir\|\|T52011ethylene responsive | LL | | | | | |
| | element binding factor 3 | | | | | | |
| 281 | gb\|AAD24392.1\|putative cAMP- | PP | | | | | |
| | dependent protein kinase | | | | | | |
| 282 | ref\|NP_193037.1\|oxidoreductase, | LL | SS | | | | |
| | zinc-binding dehydrogenase family | | | | | | |
| | protein | | | | | | |
| 283 | gb\|AAM44967.1\|putative cinnamyl | HS | | | | | |
| | alcohol dehydrogenase | | | | | | |
| 284 | ref\|NP_568453.1\|alcohol | PEG | | | | | |
| | dehydrogenase, putative [Arabidopsis | | | | | | |
| | thaliana] | | | | | | |
| 285 | gb\|AAM91227.1\|alcohol | PP | | | | | |
| | dehydrogenase | | | | | | |
| 286 | sp\|O04202\|IF35_ARATH Eukaryotic | CS | SS | CK | | | |
| | translation initiation factor 3 subunit 5 | | | | | | |
| | (eIF-3 epsilon) (eIF3 p32 subunit) | | | | | | |
| | (eIF3f) | | | | | | |
| 287 | ref\|NP_196239.1\|RNA-binding | PP | | | | | |
| | protein, putative | | | | | | |
| 288 | dbj\|BAB10987.1\|nuclear cap-binding | PP | | | | | |
| | protein; CBP20 | | | | | | |
| 289 | gb\|AAG48797.1\|putative delta 9 | CS | PEG | | | | |
| | desaturase | | | | | | |
| 290 | gb\|AAG48791.1\|putative GTP-binding | LN | SS | PP | | | |
| | protein RAB11D | | | | | | |
| 291 | gb\|AAP86673.1\|26S proteasome | HS | SS | | | | |
| | subunit RPN12 | | | | | | |
| 292 | dbj\|BAC43525.1\|putative DNA-directed | CK | HS | PP | SS | PEG | CS |
| | RNA polymerase 14 kDa subunit | | | | | | |
| | AtRPAC14 | | | | | | |
| 293 | gb\|AAM63517.1\|probable glutathione | CK | CS | PEG | | PP | |
| | peroxidase At2g31570 | | | | | | |
| 294 | ref\|NP_187617.1\|immunophilin, | SP | CK | | | | |
| | putative/FKBP-type peptidyl-prolyl | | | | | | |
| | cis-trans isomerase, putative | | | | | | |
| 295 | gb\|AAF23189.1\|putative GTP-binding | PP | | | | | |
| | protein (ATFP8) [Arabidopsis thaliana] | | | | | | |
| 296 | ref\|NP_566563.1\|ubiquitin- | HS | PP | SS | | | |
| | conjugating enzyme, putative | | | | | | |
| 297 | gb\|AAM63126.1\|20S proteasome | SS | | | | | |
| | subunit PAC1 | | | | | | |
| 298 | gb\|AAM64996.1\|GTP-binding protein | PP | | | | | |
| | Rab11 | | | | | | |
| 299 | ref\|NP_190336.1\|malate | SS | HS | | | | |
| | dehydrogenase [NAD], chloroplast | | | | | | |
| | (MDH) | | | | | | |
| 300 | emb\|CAB62652.1\|rac-like GTP binding | PEG | | | | | |
| | protein Arac11 | | | | | | |
| 301 | gb\|AAM63631.1\|ubiquitin activating | SS | | | | | |
| | enzyme-like protein | | | | | | |
| 302 | ref\|NP_173453.1\|homeobox-leucine | PP | | | | | |
| | zipper protein-related | | | | | | |
| 303 | ref\|NP_177122.2\|acid phosphatase, | SP | PP | | | | |
| | putative [Arabidopsis thaliana] | | | | | | |
| 304 | gb\|AAO50658.1\|putative C-4 sterol | PP | | | | | |
| | methyloxidase | | | | | | |
| 305 | ref\|NP_565794.1\|hydroxyproline-rich | CS | SS | CK | | | |
| | glycoprotein family protein | | | | | | |
| | [Arabidopsis thaliana] | | | | | | |
| 306 | ref\|NP_385549.1\|PROBABLE | SP | LL | | | | |
| | ENOLASE PROTEIN | | | | | | |
| 307 | pir\|\|A69990 UTP-glucose-1-phosphate | LL | | | | | |
| | uridylyltransferase homolog ytdA | | | | | | |
| 308 | ref\|NP_752427.1\|Pyrroline-5- | LN | | | | | |
| | carboxylate reductase | | | | | | |
| 309 | ref\|ZP_00108826.1\|COG0345: | PP | | | | | |
| | Pyrroline-5-carboxylate reductase | | | | | | |
| 310 | ref\|NP_435872.1\|probable alcohol | PEG | | | | | |
| 311 | pir\|\|H97551 probable | LL | | | | | |
| | aminotransferase aatc | | | | | | |
| 312 | ref\|NP_390984.1\|glycine betaine | CS | CK | | | | |
| | aldehyde dehydrogenase | | | | | | |
| 313 | ref\|NP_243169.1\|UTP-glucose-1- | PP | | | | | |
| | phosphate uridylyltransferas | | | | | | |
| 314 | ref\|ZP_00097007.1\|COG0205: 6- | CS | PP | CK | | | |
| | phosphofructokinase | | | | | | |
| 315 | ref\|NP_929737.1\|UTP--glucose-1- | PEG | SS | | | | |
| | phosphate uridylyltransferase (UDP- | | | | | | |
| | glucose pyrophosphorylase) (UDPGP) | | | | | | |
| 316 | ref\|NP_415448.1\|aspartate | PP | | | | | |
| | aminotransferase | | | | | | |
| 317 | ref\|NP_390975.1\|glucose-1-phosphate | DS | | | | | |
| | adenylyltransferase | | | | | | |
| 318 | ref\|ZP_00266198.1\|COG1012: NAD- | HS | PP | | | | |
| | dependent aldehyde dehydrogenases | | | | | | |
| 319 | ref\|NP_415818.1\|4-aminobutyrate | SS | PP | | | | |
| | aminotransferase | | | | | | |
| 320 | ref\|NP_415133.1\|putative PLP- | PP | | | | | |
| | dependent aminotransferase | | | | | | |
| 321 | ref\|NP_837887.1\|putative | CS | CK | | | | |
| | aminotransferase | | | | | | |
| 322 | ref\|NP_416651.1\|bifunctional: putative | SS | PP | | | | |
| | glutamate synthase (N-terminal); | | | | | | |
| | putative oxidoreductase (C-terminal) | | | | | | |
| 323 | ref\|NP_415214.1\|phosphoglucomutase | SP | PP | | | | |
| 324 | ref\|NP_417544.3\|probable ornithine | CS | CK | | | | |
| | aminotransferase [Escherichia coli | | | | | | |
| | K12] (EC 2.6.1.13) | | | | | | |
| 325 | ref\|ZP_00263253.1\|COG0508: | CK | PP | | | | |
| | Pyruvate/2-oxoglutarate | | | | | | |
| | dehydrogenase complex, | | | | | | |
| | dihydrolipoamide acyltransferase (E2) | | | | | | |
| | component, and related enzymes | | | | | | |
| 326 | ref\|NP_792021.1\|2-oxoglutarate | CK | PP | | | | |
| | dehydrogenase, E2 component, | | | | | | |
| | dihydrolipoamide succinyltransferase | | | | | | |
| 327 | ref\|NP_927918.1\|Transaldolase B | PP | | | | | |
| 328 | ref\|NP_928729.1\|Dihydrolipoamide | LN | PP | | | | |
| | succinyltransferase component of 2- | | | | | | |
| | oxoglutarate dehydrogenase complex | | | | | | |
| | (E2) | | | | | | |
| 329 | ref\|NP_443062.1\|hypothetical protein | CS | PP | HS | | | |
| | slr0607 | | | | | | |
| 330 | ref\|NP_242691.1\|acetoin | LL | | | | | |
| | dehydrogenase E3 component | | | | | | |
| 331 | ref\|NP_243630.1\|dihydrolipoamide | LN | | | | | |
| | dehydrogenase | | | | | | |
| 332 | ref\|NP_389344.1\|dihydrolipoamide | HS | DS | | | | |
| | dehydrogenase E3 subunit of both | | | | | | |
| | pyruvate dehydrogenase and 2- | | | | | | |
| | oxoglutarate dehydrogenase | | | | | | |
| | complexes | | | | | | |
| 333 | ref\|NP_286443.1\|2-oxoglutarate | CS | CK | | | | |
| | dehydrogenase | | | | | | |
| 334 | gb\|AAM91743.1\|putative | LN | | | | | |
| | phosphate/phosphoenolpyruvate | | | | | | |
| | translocator precursor protein | | | | | | |
| 335 | ref\|NP_415825.3\|putative | HS | SS | | | | |
| | polysaccharide hydrolase | | | | | | |
| 336 | ref\|NP_355125.1\|AGR_C_3927p | CK | HS | PP | PEG | CS | SS |
| | [Agrobacterium tumefaciens str. C58] | | | | | | |
| | ref\|NP_532838.1\| | | | | | | |
| | bacteriophytochrome protein | | | | | | |
| 337 | ref\|NP_241476.1\|sulfite reductase | PP | CK | CS | PEG | | |
| | (NADPH) | | | | | | |
| 338 | ref\|NP_836920.1\|nitrate reductase 1, | LN | | | | | |
| | beta subunit | | | | | | |
| 339 | ref\|NP_290659.1\|glucosephosphate | PP | CS | | | | |
| | isomerase | | | | | | |
| 340 | ref\|ZP_00107424.1\|COG0243: | PP | PEG | HS | | | |
| | Anaerobic dehydrogenases, typically | | | | | | |
| | selenocysteine-containing | | | | | | |
| 341 | ref\|NP_790798.1\|glucose-6-phosphate | CK | PP | SP | PEG | CS | |
| | isomerase | | | | | | |
| 342 | ref\|NP_440155.1\|isocitrate | CK | CS | | | | |
| | dehydrogenase (NADP+) | | | | | | |
| 343 | ref\|NP_928049.1\|sulfite reductase | DS | PP | PEG | | | |
| | [NADPH] hemoprotein beta- | | | | | | |
| | component (SIR-HP) | | | | | | |
| 344 | gb\|AAF26964.1\|unknown protein | LN | | | | | |
| 345 | gb\|AAF26105.1\|unknown protein | LN | | | | | |
| 346 | ref\|NP_177343.2\|protease- | CS | | | | | |
| | associated zinc finger (C3HC4-type | | | | | | |
| | RING finger) family protein | | | | | | |
| 347 | ref\|NP_850943.1\|glutamine | CK | HS | CS | | | |
| | amidotransferase-related | | | | | | |
| 348 | dbj\|BAC42398.1\|unknown protein | CK | CS | | | | |
| | [Arabidopsis thaliana] | | | | | | |
| 349 | gb\|AAF27107.1\|Unknown protein | CK | CS | | | | |
| 350 | gb\|AAM20367.1\|putative cyclin protein | PP | | | | | |
| 351 | ref\|NP_849559.1\|WRKY family | CK | CS | | | | |
| | transcription factor [Arabidopsis | | | | | | |
| | thaliana] | | | | | | |
| 352 | ref\|XP_479706.1\|putative | PP | HS | SS | | | |
| | Shwachman-Bodian-Diamond | | | | | | |
| | syndrome protein | | | | | | |
| 353 | ref\|XP_474428.1\|OSJNBa0070M12.6 | CK | PP | | | | |
| 354 | emb\|CAB44664.1\|BETL4 protein | PP | | | | | |
| 355 | ref\|NP_565108.2\|zinc finger (CCCH- | HS | SS | PEG | | | |
| | type) family protein | | | | | | |
| 356 | dbj\|BAD43912.1\|hypothetical protein | CK | PP | | | | |
| 357 | ref\|NP_442123.1\|hypothetical protein | SS | | | | | |
| | slr0013 | | | | | | |
| 358 | ref\|NP_635758.1\|vanillate O- | CS | CK | | | | |
| | demethylase oxygenase subunit | | | | | | |
| 359 | sp\|Q55891\|PCYA_SYNY3 | CK | PP | CS | | | |
| | Phycocyanobilin:ferredoxin | | | | | | |
| | oxidoreductase | | | | | | |
| 360 | ref\|NP_442600.1\|hypothetical protein | SP | CK | | | | |
| | slr0304 | | | | | | |
| 361 | gb\|AAM65293.1\|putative cold- | LN | | | | | |
| | regulated protein ref\|NP_178469.1\| | | | | | | |
| | late embryogenesis abundant domain- | | | | | | |
| | containing protein/LEA domain- | | | | | | |
| | containing protein | | | | | | |
| 362 | sp\|P73690\|Y51L_SYNY3 Ycf51-like | HS | | | | | |
| | protein dbj\|BAA17736.1\| | | | | | | |
| | ORF_ID:sII1702~hypothetical protein | | | | | | |
| 363 | ref\|NP_240950.1\|hypoxanthine- | PEG | | SS | | | |
| | guanine phosphoribosyltransferase | | | | | | |
| 364 | sp\|P23748\|MPIP_YEAST M-phase | CS | LL | PP | CK | HS | SS |
| | inducer phosphatase (Mitosis initiation | | | | | | |
| | protein MIH1) (Mitotic inducer | | | | | | |
| | homolog) | | | | | | |
| 365 | ref\|NP_910584.1\|EST | PP | | | | | |
| | AU082567(S21715) corresponds to a | | | | | | |
| | region of the predicted gene.~Similar | | | | | | |
| | to S. tuberosum ubiquinol cytochrome c | | | | | | |
| | reductase. (X79275) | | | | | | |
| 366 | gb\|AAM64477.1\|ring-box protein-like | HS | SS | | | | |
| 367 | dbj\|BAB92553.1\|DNA cross-link repair | CK | HS | | | | |
| | 1B-like protein | | | | | | |
| 368 | gb\|AAM51272.1\|putative nodulin-26 | LN | | | | | |
| | protein | | | | | | |
| 369 | ref\|XP_462727.1\|putative | LN | | | | | |
| | phenylalkylamine binding protein | | | | | | |
| | sp\|Q9FTZ2\|EBP_ORYSA Probable 3- | | | | | | |
| | beta-hydroxysteroid-delta(8),delta(7)- | | | | | | |
| | isomerase (Cholestenol delta- | | | | | | |
| | isomerase) (Delta8-delta7 sterol | | | | | | |
| | isomerase) (D8-D7 sterol isomerase) | | | | | | |
| | dbj\|BAB92148.1\|putative C-8,7 sterol | | | | | | |
| | isomerase | | | | | | |
| 370 | pir\|\|T00967hypothetical protein | HS | | | | | |
| | At2g26340 | | | | | | |
| 371 | ref\|XP_464932.1\|cytochrome c | LL | PP | | | | |
| | biogenesis protein-like | | | | | | |
| 372 | ref\|XP_450543.1\|unknown protein | CK | LL | CS | PP | SS | |
| 373 | ref\|XP_476851.1\|bifunctional | CK | CS | | | | |
| | phosphopantetheine adenylyl | | | | | | |
| | transferase dephospho CoA kinase- | | | | | | |
| | like protein | | | | | | |
| 374 | dbj\|BAD01556.1\|ERF-like protein | PP | | | | | |
| 375 | ref\|NP_568850.1\|basic helix-loop-helix | PP | | | | | |
| | (bHLH) family protein | | | | | | |
| 376 | ref\|NP_568344.2\|myb family | SP | PEG | | | | |
| | transcription factor | | | | | | |
| 377 | ref\|NP_921812.1\|putative HAM-1-like | LN | | | | | |
| | protein | | | | | | |
| 378 | gb\|AAO22987.1\|MADS-box | LL | SS | | | | |
| | transcription factor CDM104 | | | | | | |
| 379 | emb\|CAB72174.1\|responce reactor 4 | CK | SS | | | | |
| | [Arabidopsis thaliana] | | | | | | |
| 380 | ref\|XP_466611.1\|putative PLRR-4 | CS | CK | | | | |
| | polymorphic leucine-rich repeat protein | | | | | | |
| 381 | gb\|AAK70215.1\|MADS-box protein | CS | CK | PEG | | | |
| 382 | dbj\|BAB91414.1\|E2F-like repressor | CK | PEG | CS | | | |
| | E2L3 | | | | | | |
| 383 | ref\|XP_466302.1\|unknown protein | PEG | | | | | |
| 384 | gb\|AAT76334.1\|putative DNA-directed | HS | | | | | |
| | RNA polymerase II subunit | | | | | | |
| 385 | ref\|NP_922264.1\|unknown protein | LN | | | | | |
| 386 | ref\|XP_481813.1\|transfactor-like | HS | | | | | |
| 387 | dbj\|BAD53026.1\|putative ring finger | LN | | | | | |
| | protein 1 | | | | | | |
| 388 | ref\|NP_850517.1\|transcription factor, | LN | | | | | |
| | putative/zinc finger (C3HC4 type | | | | | | |
| | RING finger) family protein | | | | | | |
| 389 | emb\|CAC84710.1\|aux/IAA protein | PEG | | | | | |
| 390 | ref\|NP_172358.1\|myb family | LL | LN | | | | |
| | transcription factor (MYB60) | | | | | | |
| 391 | ref\|XP_482634.1\|AP2/EREBP | LN | | | | | |
| | transcription factor-like protein | | | | | | |
| 392 | ref\|NP_189337.1\|TCP family | CK | CS | | | | |
| | transcription factor, putative | | | | | | |
| | [Arabidopsis thaliana] | | | | | | |
| 393 | emb\|CAA85320.1\|C-terminal zinc- | HS | PP | PEG | | | |
| | finger | | | | | | |
| 394 | ref\|NP_566010.1\|SET domain- | PP | SS | | | | |
| | containing protein (ASHH3) | | | | | | |
| 395 | gb\|AAM62833.1\|putative zinc finger | PP | PEG | CS | | | |
| | protein | | | | | | |
| 396 | ref\|NP_974448.1\|zinc finger (C3HC4- | LL | LN | | | | |
| | type RING finger) family protein | | | | | | |
| 397 | gb\|AAU06309.1\|MYB transcription | PP | LN | | | | |
| | factor | | | | | | |
| 398 | gb\|AAK84890.1\|TGA-type basic | LN | | | | | |
| | leucine zipper protein TGA2.2 | | | | | | |
| 399 | gb\|AAO45753.1\|RING/C3HC4/PHD | LN | | | | | |
| | zinc finger-like protein | | | | | | |
| 400 | ref\|NP_188743.3\|transducin family | HS | | | | | |
| | protein/WD-40 repeat family protein | | | | | | |
| 401 | ref\|NP_177307.1\|AP2 domain- | CS | LL | LN | | | |
| | containing transcription factor, putative | | | | | | |
| 402 | ref\|XP_463719.1\|P0466H10.27 | PEG | | | | | |
| 403 | ref\|ZP_00357872.1\|COG0160: 4- | SP | CS | | | | |
| | aminobutyrate aminotransferase and | | | | | | |
| | related aminotransferases | | | | | | |
| 404 | ref\|ZP_00357032.1\|COG0696: | PP | SS | PEG | HS | | |
| | Phosphoglyceromutase | | | | | | |
| 405 | ref\|NP_201207.2\|expressed protein | DS | HS | | | | |
| 406 | gb\|AAK44125.1\|unknown protein | DS | CS | | | | |
| 407 | gb\|AAG52129.1\|hypothetical protein; | LL | | | | | |
| | 63994-65574 | | | | | | |
| 408 | gb\|AAM91121.1\|photoreceptor- | CS | PP | | | | |
| | interacting protein-like | | | | | | |

“CK” indicates cold tolerance improvement identified under a cold shock tolerance screen;
“CS” indicates cold tolerance improvement identified by a cold germination tolerance screen;
“DS” indicates drought tolerance improvement identified by a soil drought stress tolerance screen;
“PEG” indicates osmotic stress tolerance improvement identified by a PEG induced osmotic stress tolerance screen;
“HS” indicates heat stress tolerance improvement identified by a heat stress tolerance screen;
“SS” indicates high salinity stress tolerance improvement identified by a salt stress tolerance screen;
“LN” indicates nitrogen use efficiency improvement identified by a limited nitrogen tolerance screen;
“LL” indicates attenuated shade avoidance response identified by a shade tolerance screen under a low light condition;
“PP” indicates improved growth and development at early stages identified by an early plant growth and development screen;
“SP” indicates improved growth and development at late stages identified by a late plant growth and development screen as described in Patent Application Publication WO06076423, and incorporated herein by reference. Some proteins may provide more than one traits. For example, proteins of SEQ ID NOs. 355 and 364.

TABLE 8
Examples of traits and proteins suitable for use in the present invention.*

| PEP | | | | | | |
|---|---|---|---|---|---|---|
| SEQ | | | | | | |
| ID | | | | | | |
| NO | Protein description | Traits | | | | |
| 426 | histone H2A | CK | | | | |
| 427 | ref\|NP_195785.1\|macrophage migration | CK | | | | |
| | inhibitory factor family protein/MIF | | | | | |
| | family protein [Arabidopsis thaliana] | | | | | |
| 428 | ref\|NP_180648.1\|transducin family | CK | | | | |
| | protein/WD-40 repeat family protein | | | | | |
| 429 | gb\|AAN15690.1\|unknown protein | CK | | | | |
| | [Arabidopsis thaliana] | | | | | |
| 430 | ref\|NP_194910.2\|phototropic-responsive | CK | LN | | | |
| | NPH3 family protein | | | | | |
| 431 | ref\|NP_173542.1\|small nuclear | CK | | | | |
| | ribonucleoprotein, putative/snRNP, | | | | | |
| | putative/Sm protein, putative | | | | | |
| 432 | ref\|NP_187673.1\|diadenosine 5′,5′″- | CK | CS | PP | HS | |
| | P1,P4-tetraphosphate hydrolase, putative | | | | | |
| | [Arabidopsis thaliana] | | | | | |
| 433 | ref\|NP_172113.1\|DNA-binding | CK | SS | PEG | | |
| | bromodomain-containing protein | | | | | |
| | Contains similarity to a Ring3 protein | | | | | |
| 434 | gb\|AAC17827.1\|similar to late | CK | PP | | | |
| | embryogenesis abundant proteins | | | | | |
| 435 | ref\|NP_564623.2\|sodium/calcium | CK | | | | |
| | exchanger family protein/calcium- | | | | | |
| | binding EF hand family protein | | | | | |
| 436 | ref\|NP_179396.1\|histone H1-3 (HIS1-3) | CK | | | | |
| 437 | ref\|NP_563973.1\|lactoylglutathione lyase | CK | | | | |
| | family protein/glyoxalase I family | | | | | |
| | protein | | | | | |
| 438 | ref\|NP_176890.1\|F-box family protein | CK | CS | HS | | |
| | [Arabidopsis thaliana] | | | | | |
| 439 | ref\|NP_199024.1\|expressed protein | CK | | | | |
| | [Arabidopsis thaliana] | | | | | |
| 440 | ref\|NP_176371.1\|postsynaptic protein- | CK | HS | SS | | |
| | related [Arabidopsis thaliana] | | | | | |
| 441 | ref\|NP_027420.1\|zinc finger (CCCH- | CK | CS | SS | LL | PEG |
| | type) family protein [Arabidopsis | | | | | |
| | thaliana] | | | | | |
| 442 | ref\|NP_567671.1\|bile acid:sodium | CK | | | | |
| | symporter family protein | | | | | |
| 443 | ref\|NP_850182.1\|PUR alpha-1 protein | CK | PP | HS | SS | |
| | [Arabidopsis thaliana] | | | | | |
| 444 | ref\|NP_567709.1\|26S proteasome | CK | CS | PP | | |
| | regulatory subunit, putative (RPN7) | | | | | |
| 445 | ref\|NP_850094.1\|CBL-interacting | CK | SS | PEG | | |
| | protein kinase 3 (CIPK3) | | | | | |
| 446 | dbj\|BAB02193.1\|cytochrome p450 | CK | PP | | | |
| 447 | emb\|CAB80133.1\|cyclin delta-3 | CK | PP | HS | SS | PEG |
| 448 | gb\|AAP51420.1\|ferric-chelate reductase | CK | | | | |
| | [Arabidopsis thaliana] | | | | | |
| 449 | ref\|NP_563791.1\|protein phosphatase 2C | CK | | | | |
| | family protein/PP2C family protein | | | | | |
| 450 | ref\|NP_197894.1\|cytochrome P450 | CK | DS | | | |
| | family protein [Arabidopsis thaliana] | | | | | |
| 451 | gb\|AAM62660.1\|unknown [Arabidopsis | CK | | | | |
| | thaliana] | | | | | |
| 452 | ref\|NP_193140.1\|selenium-binding | CK | HS | | | |
| | protein, putative [Arabidopsis thaliana] | | | | | |
| 453 | ref\|NP_565411.2\|calcium-dependent | CK | CS | HS | SS | LL |
| | protein kinase isoform 6 (CPK6) | | | | | |
| 454 | ref\|NP_197849.1\|expressed protein | CK | LN | | | |
| | [Arabidopsis thaliana] | | | | | |
| 455 | ref\|NP_174418.1\|photosystem I reaction | CK | PEG | | | |
| | center subunit III family protein | | | | | |
| 456 | ref\|NP_179369.2\|expressed protein | CK | | | | |
| | [Arabidopsis thaliana] | | | | | |
| 457 | ref\|NP_190390.1\|expressed protein | CK | | | | |
| | [Arabidopsis thaliana] | | | | | |
| 458 | ref\|NP_179889.1\|casein kinase II alpha | CK | | | | |
| | chain, putative [Arabidopsis thaliana] | | | | | |
| 459 | ref\|NP_851182.1\|protein kinase family | CK | SS | | | |
| | protein [Arabidopsis thaliana] | | | | | |
| 460 | ref\|NP_742382.1\|4-aminobutyrate | CK | DS | | | |
| | aminotransferase [Pseudomonas putida | | | | | |
| | KT2440] | | | | | |
| 461 | ref\|NP_929593.1\|4-aminobutyrate | CK | | | | |
| | aminotransferase (gamma-amino-N- | | | | | |
| | butyrate transaminase) (GABA | | | | | |
| | transaminase) | | | | | |
| 462 | ref\|NP_931210.1\|glutamate synthase | CK | | | | |
| | [NADPH] small chain (glutamate | | | | | |
| | synthase beta subunit) (NADPH- | | | | | |
| | GOGAT) (GLTS beta chain) | | | | | |
| 463 | ref\|NP_175649.1\|glycosyl hydrolase | CK | PP | | | |
| | family 1 protein/beta-glucosidase, | | | | | |
| | putative a | | | | | |
| 464 | ref\|NP_849681.1\|serine/threonine protein | CK | LL | LN | | |
| | phosphatase 2A (PP2A) 55 kDa | | | | | |
| | regulatory subunit B [Arabidopsis | | | | | |
| | thaliana] | | | | | |
| 465 | ref\|NP_391273.1\|phosphoglycerate | CK | PP | | | |
| | kinase [Bacillus subtilis subsp. subtilis | | | | | |
| | str. 168] | | | | | |
| 466 | gb\|AAF27012.1\|putative SAR DNA- | CK | | | | |
| | binding protein-1 [Arabidopsis thaliana] | | | | | |
| 467 | ref\|NP_181253.2\|transducin family | CK | HS | | | |
| | protein/WD-40 repeat family protein | | | | | |
| 468 | ref\|NP_177237.1\|C2 domain-containing | CK | | | | |
| | protein | | | | | |
| 469 | ref\|NP_193273.1\|cytochrome P450 | CK | PP | | | |
| | family protein [Arabidopsis thaliana] | | | | | |
| 470 | ref\|NP_190771.2\|F-box family protein/ | CK | | | | |
| | WD-40 repeat family protein | | | | | |
| | [Arabidopsis thaliana] | | | | | |
| 471 | ref\|NP_566517.1\|expressed protein | CK | CS | SS | | |
| | [Arabidopsis thaliana] | | | | | |
| 472 | dbj\|BAB09992.1\|serine/threonine protein | CK | SS | LL | | |
| | kinase-like [Arabidopsis thaliana] | | | | | |
| 473 | ref\|NP_568971.1\|leucine-rich repeat | CK | | | | |
| | transmembrane protein kinase, putative | | | | | |
| 474 | gb\|AAG50866.1\|protein kinase, putative | CK | | | | |
| | [Arabidopsis thaliana] | | | | | |
| 475 | ref\|NP_181590.1\|protein kinase family | CK | | | | |
| | protein | | | | | |
| 476 | ref\|NP_564161.1\| | CK | SS | | | |
| | phosphoglycerate/bisphosphoglycerate | | | | | |
| | mutase family protein | | | | | |
| 477 | ref\|NP_386871.1\|PROBABLE | CK | | | | |
| | PHOSPHOGLYCERATE KINASE | | | | | |
| | PROTEIN | | | | | |
| 478 | ref\|NP_795041.1\|glutamine synthetase | CK | CS | | | |
| 479 | ref\|NP_192046.1\|mitogen-activated | CK | SS | PEG | | |
| | protein kinase, putative/MAPK, putative | | | | | |
| | (MPK4) | | | | | |
| 480 | ref\|NP_242595.1\|glutamate synthase | CK | | | | |
| | (small subunit) | | | | | |
| 481 | ref\|NP_391914.1\|ornithine | CK | PP | | | |
| | aminotransferase | | | | | |
| 482 | ref\|NP_564469.1\|WD-40 repeat family | CK | HS | SS | LL | PEG |
| | protein [Arabidopsis thaliana] | | | | | |
| 483 | ref\|NP_181783.1\|protein kinase family | CK | | | | |
| | protein [Arabidopsis thaliana] | | | | | |
| 484 | ref\|NP_188976.1\|casein kinase, putative | CK | CS | PP | SS | PEG |
| | [Arabidopsis thaliana] | | | | | |
| 485 | ref\|NP_176390.1\|mitochondrial | CK | LN | | | |
| | transcription termination factor-related/ | | | | | |
| | mTERF-related [Arabidopsis thaliana] | | | | | |
| 486 | dbj\|BAB10854.1\|unnamed protein | CK | LL | | | |
| | product [Arabidopsis thaliana] | | | | | |
| 487 | ref\|NP_177573.1\|protein kinase, putative | CK | LL | | | |
| | [Arabidopsis thaliana] | | | | | |
| 488 | ref\|NP_174217.1\|CBL-interacting protein | CK | CS | LL | PEG | |
| | kinase 18 (CIPK18) | | | | | |
| 489 | ref\|NP_189420.1\|monodehydroascorbate | CK | PP | LL | | |
| | reductase, putative [Arabidopsis thaliana] | | | | | |
| 490 | gb\|AAL34198.1\|putative glutathione | CK | | | | |
| | peroxidase [Arabidopsis thaliana] | | | | | |
| 491 | ref\|NP_192114.1\|sugar transporter, | CK | | | | |
| | putative [Arabidopsis thaliana] | | | | | |
| 492 | ref\|NP_200733.2\|sugar transporter | CK | | | | |
| | family protein [Arabidopsis thaliana] | | | | | |
| 493 | ref\|NP_922597.1\|unknown protein | CK | SS | LL | | |
| 494 | ref\|NP_916703.1\|putative nuclear RNA | CK | | | | |
| | binding protein A [Oryza sativa | | | | | |
| 495 | ref\|NP_189139.1\|zinc finger (C3HC4- | CK | PP | | | |
| | type RING finger) family protein | | | | | |
| 496 | ref\|ZP_00039591.1\|COG0149: | CK | PEG | | | |
| | Triosephosphate isomerase | | | | | |
| 497 | ref\|XP_473139.1\|OSJNBa0004N05.3 | CK | | | | |
| | [Oryza sativa (japonica cultivar-group)] | | | | | |
| 498 | ref\|ZP_00107950.1\|COG0155: Sulfite | CK | | | | |
| | reductase, beta subunit (hemoprotein) | | | | | |
| | [Nostoc punctiforme PCC 73102] | | | | | |
| 499 | ref\|NP_191594.1\|armadillo/beta-catenin | CK | PEG | | | |
| | repeat family protein/F-box family | | | | | |
| | protein [Arabidopsis thaliana] | | | | | |
| 500 | dbj\|BAB09237.1\|beta-amylase | CK | | | | |
| | [Arabidopsis thaliana] | | | | | |
| 501 | ref\|NP_178065.1\|inosine-5′- | CK | | | | |
| | monophosphate dehydrogenase | | | | | |
| | [Arabidopsis thaliana] | | | | | |
| 502 | gb\|AAV44116.1\|unknown protein [Oryza | CK | | | | |
| | sativa (japonica cultivar-group)] | | | | | |
| 503 | ref\|XP_480615.1\|putative | CK | | | | |
| | aminoimidazolecarboximide | | | | | |
| | ribonucleotide transformylase | | | | | |
| 504 | ref\|NP_199602.1\|respiratory burst | CK | | | | |
| | oxidase protein D (RbohD)/NADPH | | | | | |
| | oxidase | | | | | |
| 505 | dbj\|BAD87004.1\|unknown protein | CK | | | | |
| | [Oryza sativa (japonica cultivar-group)] | | | | | |
| 506 | ref\|NP_171679.1\|protein kinase family | CK | | | | |
| | protein [Arabidopsis thaliana] | | | | | |
| 507 | ref\|NP_172414.1\|ATP-citrate synthase | CK | SS | | | |
| | (ATP-citrate (pro-S-)-lyase/citrate | | | | | |
| | cleavage enzyme), putative | | | | | |
| 508 | ref\|NP_567724.1\|fibrillarin 2 (FIB2) | CK | | | | |
| | [Arabidopsis thaliana] | | | | | |
| 509 | ref\|NP_193573.1\|F-box family protein | CK | LL | PEG | | |
| | [Arabidopsis thaliana] | | | | | |
| 510 | ref\|NP_564267.1\|peptidyl-prolyl cis- | CK | | | | |
| | trans isomerase cyclophilin-type family. | | | | | |
| | protein [Arabidopsis thaliana] | | | | | |
| 511 | ref\|NP_918239.1\|pectate lyase-like | CK | | | | |
| | protein | | | | | |
| 512 | dbj\|BAD54684.1\|unknown protein | CK | | | | |
| | [Oryza sativa (japonica cultivar-group)] | | | | | |
| 513 | ref\|XP_483682.1\|putative auxin induced | CK | LL | | | |
| | protein | | | | | |
| 514 | dbj\|BAD73644.1\|60S ribosomal protein | CK | CS | PEG | | |
| | L18A-like | | | | | |
| 515 | dbj\|BAB84492.1\|nuclear movement | CK | | | | |
| | protein-like | | | | | |
| 516 | ref\|NP_566157.1\|nodulin family protein | CK | | | | |
| | [Arabidopsis thaliana] | | | | | |
| 517 | ref\|NP_186908.1\|delta 7-sterol-C5- | CK | LL | | | |
| | desaturase, putative [Arabidopsis | | | | | |
| | Nostoc punctiformethaliana] | | | | | |
| 518 | ref\|NP_486997.1\|hypothetical protein | CK | PP | | | |
| | alr2957 [Nostoc sp. PCC 7120] | | | | | |
| 519 | ref\|NP_916905.1\|putative glyoxal oxidase | CK | | | | |
| 520 | ref\|NP_565499.1\|expressed protein | CK | SS | | | |
| | [Arabidopsis thaliana] | | | | | |
| 521 | ref\|XP_476805.1\|unknown protein | CK | SS | LN | PEG | |
| 522 | ref\|NP_197956.1\|expressed protein | CK | PP | SS | PEG | |
| 523 | emb\|CAB67657.2\|splicing factor-like | CS | PP | | | |
| | protein [Arabidopsis thaliana] | | | | | |
| | ref\|NP_190918.3\|zinc knuckle (CCHC- | | | | | |
| | type) family protein [Arabidopsis | | | | | |
| | thaliana] | | | | | |
| 524 | ref\|NP_187382.1\|forkhead-associated | CS | PEG | | | |
| | domain-containing protein/FHA | | | | | |
| | domain-containing protein | | | | | |
| 525 | ref\|NP_188451.1\| | CS | PP | HS | LN | |
| | octicosapeptide/Phox/Bem1p (PB1) | | | | | |
| | domain-containing protein [Arabidopsis | | | | | |
| | thaliana] | | | | | |
| 526 | dbj\|BAB09537.1\|unnamed protein | CS | | | | |
| | product [Arabidopsis thaliana] | | | | | |
| 527 | ref\|NP_563769.1\|F-box family protein | CS | PP | HS | SS | |
| 528 | ref\|NP_172880.1\|phytochrome kinase, | CS | | | | |
| | putative [Arabidopsis thaliana] | | | | | |
| 529 | ref\|NP_179646.1\|DNAJ heat shock | CS | PP | SS | | |
| | family protein [Arabidopsis thaliana] | | | | | |
| 530 | ref\|NP_201441.1\|dehydrin (RAB18) | CS | PEG | | | |
| | rab18 protein-Arabidopsis thaliana | | | | | |
| | sp\|P30185\|DHR18_ARATH Dehydrin | | | | | |
| | Rab18 | | | | | |
| 531 | dbj\|BAB70612.1\|anthocyanin-related | CS | SS | | | |
| | membrane protein 1 | | | | | |
| 532 | ref\|NP_849480.1\|expressed protein | CS | PP | PEG | | |
| 533 | ref\|NP_568089.1\|expressed protein | CS | | | | |
| 534 | ref\|NP_174469.1\|histidine biosynthesis | CS | | | | |
| | bifunctional protein (HISIE) [Arabidopsis | | | | | |
| | thaliana] pir\|\|T51812 phosphoribosyl- | | | | | |
| | AMP cyclohydrolase (EC 3.5.4.19) | | | | | |
| 535 | ref\|XP_493889.1\|putative protein kinase | CS | | | | |
| | [Oryza sativa] | | | | | |
| 536 | emb\|CAD92450.1\|amino acid permease 6 | CS | SS | | | |
| | [Brassica napus] | | | | | |
| 537 | sp\|Q12068\|GRE2_YEAST NADPH- | CS | | | | |
| | dependent methylglyoxal reductase | | | | | |
| | GRE2 (Genes de respuesta a estres | | | | | |
| | protein 2) | | | | | |
| 538 | ref\|NP_566612.1\|histone deacetylase | CS | | | | |
| | family protein [Arabidopsis thaliana] | | | | | |
| 539 | ref\|NP_851041.1\|zinc finger (CCCH- | CS | PEG | | | |
| | type) Family protein [Arabidopsis | | | | | |
| | thaliana] | | | | | |
| 540 | ref\|NP_921503.1\|putative polyprotein | CS | PP | | | |
| | [Oryza sativa (japonica cultivar-group)] | | | | | |
| 541 | emb\|CAB80464.1\|cinnamyl-alcohol | CS | | | | |
| | dehydrogenase ELI3-2 [Arabidopsis | | | | | |
| | thaliana] | | | | | |
| 542 | ref\|NP_196086.1\|cytochrome P450, | CS | | | | |
| | putative [Arabidopsis thaliana] | | | | | |
| 543 | ref\|NP_199845.2\|DNA repair protein- | CS | PEG | | | |
| | related [Arabidopsis thaliana] | | | | | |
| 544 | ref\|NP_195917.1\|hydrolase, alpha/beta | CS | PEG | | | |
| | fold family protein [Arabidopsis thaliana] | | | | | |
| 545 | ref\|NP_176968.1\|glycerate | CS | HS | SS | PEG | |
| | dehydrogenase/NADH-dependent | | | | | |
| | hydroxypyruvate reductase | | | | | |
| 546 | ref\|NP_198904.1\|WD-40 repeat family | CS | PEG | | | |
| | protein/zfwd3 protein (ZFWD3) | | | | | |
| | [Arabidopsis thaliana] | | | | | |
| 547 | ref\|NP_567705.1\|ubiquitin-specific | CS | | | | |
| | protease 16, putative (UBP16) | | | | | |
| 548 | sp\|P53845\|YN03_YEAST Hypothetical | CS | | | | |
| | 35.5 kDa protein in PIK1-POL2 | | | | | |
| | intergenic region | | | | | |
| 549 | ref\|NP_013146.1\|Microtubule-associated | CS | | | | |
| | protein (MAP) of the XMAP215/Dis1 | | | | | |
| | family; regulates microtubule dynamics | | | | | |
| | during spindle orientation and metaphase | | | | | |
| | chromosome alignment; interacts with | | | | | |
| | spindle pole body component Spc72p | | | | | |
| | [Saccharomyces cerevisiae] | | | | | |
| 550 | ref\|NP_015058.1\|Dicarboxylic amino | CS | PP | | | |
| | acid permease, mediates high-affinity and | | | | | |
| | high-capacity transport of L-glutamate | | | | | |
| | and L-aspartate; also a transporter for | | | | | |
| | Gln, Asn, Ser, Ala, and Gly | | | | | |
| | [Saccharomyces cerevisiae] | | | | | |
| 551 | sp\|P50947\|YNJ7_YEAST Hypothetical | CS | PEG | | | |
| | 37.0 kDa protein in RAS2-RPS7B | | | | | |
| | intergenic region | | | | | |
| 552 | ref\|NP_009495.1\|Shp1p [Saccharomyces | CS | | | | |
| | cerevisiae] emb\|CAA80789.1\|YBLO515 | | | | | |
| | [Saccharomyces cerevisiae] | | | | | |
| | emb\|CAA84878.1\|SHP1 | | | | | |
| 553 | ref\|NP_197455.1\|expressed protein | CS | PP | | | |
| | [Arabidopsis thaliana] | | | | | |
| 554 | ref\|NP_566453.1\|WD-40 repeat family | CS | | | | |
| | protein [Arabidopsis thaliana] | | | | | |
| 555 | ref\|ZP_00276041.1\|COG1062: Zn- | CS | | | | |
| | dependent alcohol dehydrogenases, class | | | | | |
| | III [Ralstonia metallidurans CH34] | | | | | |
| 556 | ref\|NP_416429.3\|D-cysteine | CS | | | | |
| | desulfhydrase, PLP-dependent enzyme | | | | | |
| | cysteine desulfhydrase, PLP-dependent | | | | | |
| | enzyme | | | | | |
| 557 | gb\|AAK21273.1\|aberrant lateral root | CS | PP | SS | | |
| | formation 5 [Arabidopsis thaliana] | | | | | |
| | ref\|NP_566730.1\|MATE efflux family | | | | | |
| | protein [Arabidopsis thaliana] | | | | | |
| 558 | ref\|NP_175641.1\|protein kinase family | CS | SS | | | |
| | protein/C-type lectin domain-containing | | | | | |
| | protein | | | | | |
| 559 | ref\|NP_564579.1\|expressed protein | CS | SS | | | |
| | [Arabidopsis thaliana] | | | | | |
| 560 | ref\|NP_195770.1\|mitochondrial substrate | CS | LL | | | |
| | carrier family protein [Arabidopsis | | | | | |
| | thaliana] | | | | | |
| 561 | gb\|AAF02870.1\|Hypothetical protein | CS | | | | |
| | [Arabidopsis thaliana] | | | | | |
| 562 | emb\|CAB78986.1\|lectin like protein | CS | SS | | | |
| | [Arabidopsis thaliana] | | | | | |
| 563 | ref\|NP_188127.1\|oxidoreductase, zinc- | CS | | | | |
| | binding dehydrogenase family protein | | | | | |
| 564 | ref\|NP_199669.1\|FK506-binding protein | CS | PEG | | | |
| | 2-2 (FKBP15-2)/immunophilin/ | | | | | |
| | peptidyl-prolyl cis-trans isomerase/ | | | | | |
| | rotamase | | | | | |
| 565 | ref\|NP_195534.1\|expansin, putative | CS | PP | | | |
| | (EXP20) | | | | | |
| 566 | ref\|NP_196975.1\|expressed protein | CS | | | | |
| | [Arabidopsis thaliana] | | | | | |
| 567 | ref\|NP_442351.1\|hypothetical protein | CS | LN | | | |
| | ssl0353 [Synechocystis sp. PCC 6803] | | | | | |
| 568 | ref\|NP_564383.1\|ABC transporter | CS | | | | |
| | family protein [Arabidopsis thaliana] | | | | | |
| 569 | ref\|NP_192879.1\|ARID/BRIGHT DNA- | CS | PP | SS | | |
| | binding domain-containing protein/ | | | | | |
| | ELM2 domain-containing protein/Myb- | | | | | |
| | like DNA-binding domain-containing | | | | | |
| | protein | | | | | |
| 570 | emb\|CAA48415.1\|unnamed protein | CS | | | | |
| | product [synthetic construct] | | | | | |
| 571 | dbj\|BAB03922.1\|glycine betaine | CS | PEG | | | |
| | aldehyde dehydrogenase [Bacillus | | | | | |
| | halodurans C-125] | | | | | |
| 572 | ref\|NP_181434.1\|aquaporin, putative | CS | PP | | | |
| | [Arabidopsis thaliana] | | | | | |
| 573 | ref\|NP_927470.1\|phosphoenolpyruvate | CS | | | | |
| | carboxykinase [ATP] [Photorhabdus | | | | | |
| | luminescens subsp. laumondii TTO1] | | | | | |
| 574 | ref\|NP_564217.1\|lysine and histidine | CS | | | | |
| | specific transporter, putative [Arabidopsis | | | | | |
| | thaliana] | | | | | |
| 575 | ref\|NP_243603.1\|1-pyrroline-5- | CS | | | | |
| | carboxylate dehydrogenase [Bacillus | | | | | |
| | halodurans C-125] | | | | | |
| 576 | ref\|NP_244029.1\|pyruvate kinase | CS | PP | | | |
| | [Bacillus halodurans C-125] | | | | | |
| 577 | dbj\|BAB09466.1\|auxin-induced protein- | CS | | | | |
| | like [Arabidopsis thaliana] | | | | | |
| 578 | ref\|NP_193699.1\|Ras-related GTP- | CS | LN | | | |
| | binding protein, putative [Arabidopsis | | | | | |
| | thaliana] | | | | | |
| 579 | sp\|O22203\|C98A3_ARATHCytochrome | CS | | | | |
| | P450 98A3 | | | | | |
| 580 | ref\|NP_187156.1\|protein kinase family | CS | SS | PEG | | |
| | protein [Arabidopsis thaliana] | | | | | |
| 581 | ref\|NP_195397.1\|transporter-related | CS | PP | SS | | |
| | [Arabidopsis thaliana] | | | | | |
| 582 | ref\|NP_564797.1\|flavin-containing | CS | SS | PEG | | |
| | monooxygenase family protein/FMO | | | | | |
| | family protein | | | | | |
| 583 | gb\|AAM45011.1\|putative protein kinase | CS | | | | |
| | [Arabidopsis thaliana] | | | | | |
| 584 | gb\|AAO15285.1\|Putative | CS | | | | |
| | dihydrodipicolinate reductase-like protein | | | | | |
| | [Oryza sativa (japonica cultivar-group)] | | | | | |
| 585 | ref\|ZP_00099233.1\|COG0036: Pentose- | CS | | | | |
| | 5-phosphate-3-epimerase | | | | | |
| | [Desulfitobacterium hafniense DCB-2] | | | | | |
| 586 | \|YCF3_SYNY3 Photosystem I assembly | CS | | | | |
| | protein ycf3 | | | | | |
| 587 | ref\|NP_191332.2\|protein kinase, | CS | DS | SS | PEG | |
| | putative [Arabidopsis thaliana] | | | | | |
| 588 | gb\|AAD20142.1\|putative poly(A) binding | CS | | | | |
| | protein [Arabidopsis thaliana] | | | | | |
| 589 | gb\|AAD49757.1\|Contains F-box domain | CS | | | | |
| | PF\|00646. [Arabidopsis thaliana] | | | | | |
| 590 | ref\|NP_192170.1\|tryptophan synthase, | CS | | | | |
| | alpha subunit, putative [Arabidopsis | | | | | |
| | thaliana] | | | | | |
| 591 | ref\|NP_198924.1\|glycerophosphoryl | CS | HS | | | |
| | diester phosphodiesterase family protein | | | | | |
| 592 | dbj\|BAD81286.1\|putative dual specificity | CS | | | | |
| | kinase 1 | | | | | |
| 593 | ref\|NP_011933.1\|Ssf1p [Saccharomyces | CS | DS | | | |
| | cerevisiae] sp\|P38789\|SSF1_YEAST | | | | | |
| | Ribosome biogenesis protein SSF1 | | | | | |
| 594 | ref\|XP_464982.1\|lipase class 3-like | CS | | | | |
| | [Oryza sativa (japonica cultivar-group)] | | | | | |
| | dbj\|BAD21500.1\|lipase class 3-like | | | | | |
| | [Oryza sativa (japonica cultivar-group)] | | | | | |
| 595 | gb\|AAB81672.1\|putative protein kinase | CS | HS | PEG | | |
| | [Arabidopsis thaliana] | | | | | |
| 596 | ref\|NP_201503.1\|expressed protein | CS | | | | |
| | [Arabidopsis thaliana] | | | | | |
| 597 | ref\|NP_190376.1\|L-galactono-1,4- | CS | SS | | | |
| | lactone dehydrogenase, putative | | | | | |
| 598 | ref\|NP_564800.1\|expressed protein | CS | | | | |
| | [Arabidopsis thaliana] | | | | | |
| 599 | ref\|XP_467673.1\|putative nicastrin | CS | DS | SS | PEG | |
| | [Oryza sativa (japonica cultivar-group)] | | | | | |
| 600 | ref\|XP_450109.1\|nodulation receptor | CS | PP | SS | | |
| | kinase-like protein | | | | | |
| 601 | ref\|NP_850693.1\|expressed protein | DS | | | | |
| | [Arabidopsis thaliana] | | | | | |
| 602 | ref\|NP_179923.2\|nicotinate | DS | | | | |
| | phosphoribosyltransferase family protein/ | | | | | |
| | NAPRTase family protein [Arabidopsis | | | | | |
| | thaliana] | | | | | |
| 603 | gb\|AAF00630.1\|hypothetical protein | DS | LN | | | |
| | [Arabidopsis thaliana] | | | | | |
| 604 | ref\|NP_566273.1\|heavy-metal-associated | DS | | | | |
| | domain-containing protein | | | | | |
| 605 | ref\|NP_177794.1\|12-oxophytodienoate | DS | | | | |
| | reductase (OPR1) [Arabidopsis thaliana] | | | | | |
| 606 | gb\|AAM45040.1\|putative AtMlo-h1 | DS | | | | |
| | protein [Arabidopsis thaliana] | | | | | |
| 607 | ref\|NP_191438.2\|glycosyl transferase | DS | SS | | | |
| | family 8 protein [Arabidopsis thaliana] | | | | | |
| 608 | ref\|NP_190235.1\|armadillo/beta-catenin | DS | | | | |
| | repeat family protein/U-box domain- | | | | | |
| | containing family protein | | | | | |
| 609 | ref\|NP_193087.1\|ammonium transporter | DS | | | | |
| | 1, member 1 (AMT1.1) | | | | | |
| 610 | ref\|NP_187413.1\|expressed protein | DS | LN | PEG | | |
| | [Arabidopsis thaliana] | | | | | |
| 611 | ref\|NP_198627.1\|ASF1-like anti- | DS | PP | HS | | |
| | silencing family protein [Arabidopsis | | | | | |
| | thaliana] | | | | | |
| 612 | pir\|\|A86318protein F15H18.11 | DS | PP | | | |
| | [imported]-Arabidopsis thaliana | | | | | |
| | gb\|AAF25996.1\|F15H18.11 | | | | | |
| 613 | ref\|NP_440678.1\|hypothetical protein | DS | | | | |
| | slr1900 [Synechocystis sp. PCC 6803] | | | | | |
| 614 | gb\|AAP37785.1\|At4g24520 [Arabidopsis | DS | SS | | | |
| | thaliana] emb\|CAB79362.1\|NADPH- | | | | | |
| | ferrihemoprotein reductase ATR1 | | | | | |
| 615 | gb\|AAC27147.1\|Contains similarity to | DS | LL | PEG | | |
| | ABC transporter gb\|1651790 from | | | | | |
| | Synechocystis sp. | | | | | |
| 616 | ref\|NP_193578.1\|Ras-related GTP- | DS | | | | |
| | binding protein, putative [Arabidopsis | | | | | |
| | thaliana] | | | | | |
| 617 | ref\|NP_565310.1\|expressed protein | DS | PEG | | | |
| | [Arabidopsis thaliana] | | | | | |
| 618 | ref\|NP_199518.1\|protein kinase, putative | DS | | | | |
| | [Arabidopsis thaliana] | | | | | |
| 619 | dbj\|BAB03027.1\|glutamine-fructose-6- | DS | | | | |
| | phosphate transaminase 2 [Arabidopsis | | | | | |
| | thaliana] | | | | | |
| 620 | ref\|NP_194265.2\|EXS family protein/ | DS | | | | |
| | ERD1/XPR1/SYG1 family protein | | | | | |
| | [Arabidopsis thaliana] gb\|AAR99486.1\| | | | | | |
| | PHO1-like protein [Arabidopsis thaliana] | | | | | |
| 621 | ref\|XP_478910.1\|serine/threonine protein | DS | SS | PEG | | |
| | kinase PKPA-like protein [Oryza sativa | | | | | |
| 622 | gb\|AAD49980.1\|Similar to gb\|AF110333 | HS | | | | |
| | PrMC3 protein from Pinus radiata and is | | | | | |
| | a member of PF\|00135 Carboxylesterases | | | | | |
| | family. | | | | | |
| 623 | ref\|NP_178620.1\|glycine-rich protein | HS | SS | | | |
| | (GRP) [Arabidopsis thaliana] | | | | | |
| 624 | ref\|NP_564833.1\|expressed protein | HS | | | | |
| | [Arabidopsis thaliana] | | | | | |
| 625 | ref\|NP_180326.1\|zinc finger (AN1-like) | HS | | | | |
| | family protein [Arabidopsis thaliana] | | | | | |
| 626 | ref\|NP_417147.1\|succinate-semialdehyde | HS | | | | |
| | dehydrogenase I, NADP-dependent | | | | | |
| | [Escherichia coli K12] | | | | | |
| 627 | ref\|NP_176713.1\|cytochrome P450, | HS | PEG | | | |
| | putative [Arabidopsis thaliana] | | | | | |
| 628 | ref\|NP_439997.1\|hypothetical protein | HS | | | | |
| | slr0731 [Synechocystis sp. PCC 6803] | | | | | |
| 629 | gb\|AAG51407.1\|putative cysteine | HS | | | | |
| | synthase; 39489-37437 [Arabidopsis | | | | | |
| | thaliana] | | | | | |
| 630 | gb\|AAO50687.1\|unknown protein | HS | PEG | | | |
| | [Arabidopsis thaliana] | | | | | |
| 631 | ref\|NP_196259.2\|DNAJ heat shock N- | HS | SS | LL | | |
| | terminal domain-containing protein | | | | | |
| 632 | gb\|AAM63172.1\|putative integral | LL | | | | |
| | membrane protein [Arabidopsis thaliana] | | | | | |
| 633 | ref\|NP_440484.1\|formaldehyde | LL | | | | |
| | dehydrogenase (glutathione) | | | | | |
| 634 | ref\|NP_190148.1\|transducin family | LL | | | | |
| | protein/WD-40 repeat family protein | | | | | |
| 635 | ref\|NP_180485.1\|lectin protein kinase, | LL | LN | | | |
| | putative [Arabidopsis thaliana] | | | | | |
| 636 | ref\|NP_190708.1\|expressed protein | LL | LN | | | |
| | [Arabidopsis thaliana] | | | | | |
| 637 | ref\|NP_201402.2\|heterogeneous nuclear | LL | LN | | | |
| | ribonucleoprotein, putative/hnRNP, | | | | | |
| | putative | | | | | |
| 638 | ref\|NP_176819.1\|protein kinase family | LL | | | | |
| | protein [Arabidopsis thaliana] | | | | | |
| 639 | ref\|NP_914663.1\|P0431G06.11 [Oryza | LL | | | | |
| | sativa (japonica cultivar-group)] | | | | | |
| 640 | ref\|NP_662751.1\|6-phosphogluconate | LL | | | | |
| | dehydrogenase, decarboxylating, putative | | | | | |
| | [Chlorobium tepidum TLS] | | | | | |
| 641 | ref\|XP_473078.1\|OSJNBa0014K14.9 | LL | PEG | | | |
| | [Oryza sativa (japonica cultivar-group)] | | | | | |
| 642 | gb\|AAU44517.1\|hypothetical protein | LL | PEG | | | |
| | AT4G22980 [Arabidopsis thaliana] | | | | | |
| 643 | ref\|NP_200586.1\|expressed protein | LL | LN | | | |
| | [Arabidopsis thaliana] | | | | | |
| 644 | ref\|XP_482907.1\|putative glycoprotein 3- | LL | LN | | | |
| | alpha-L-fucosyltransferase | | | | | |
| 645 | emb\|CAB79230.1\|predicted protein | LL | | | | |
| | [Arabidopsis thaliana] | | | | | |
| 646 | ref\|XP_474072.1\|OSJNBb0079B02.14 | LL | LN | | | |
| | [Oryza sativa (japonica cultivar-group)] | | | | | |
| 647 | ref\|NP_244766.1\|acetyl- | LL | | | | |
| | CoA:acetoacetyl-CoA transferase | | | | | |
| | [Bacillus halodurans C-125] | | | | | |
| 648 | ref\|NP_566384.1\|xanthine/uracil | LL | | | | |
| | permease family protein [Arabidopsis | | | | | |
| | thaliana] | | | | | |
| 649 | ref\|NP_201459.1\|expressed protein | LL | | | | |
| | [Arabidopsis thaliana] | | | | | |
| 650 | ref\|NP_201145.1\|adenylate kinase | LN | | | | |
| | [Arabidopsis thaliana] | | | | | |
| 651 | ref\|NP_851136.1\|cytochrome P450 | LN | | | | |
| | family protein [Arabidopsis thaliana] | | | | | |
| 652 | ref\|NP_851196.1\|outward rectifying | LN | | | | |
| | potassium channel (KCO1) [Arabidopsis | | | | | |
| | thaliana] | | | | | |
| 653 | ref\|NP_180901.1\|plastid developmental | LN | | | | |
| | protein DAG, putative [Arabidopsis | | | | | |
| | thaliana] | | | | | |
| 654 | ref\|NP_195967.1\|serine/threonine protein | LN | | | | |
| | phosphatase 2A (PP2A) regulatory | | | | | |
| | subunit B′ (B′alpha) | | | | | |
| 655 | gb\|AAT69222.1\|hypothetical protein | LN | | | | |
| | At2g30900 [Arabidopsis thaliana] | | | | | |
| 656 | ] ref\|NP_191804.1\|expressed protein | LN | | | | |
| | [Arabidopsis thaliana] | | | | | |
| 657 | ref\|NP_190313.1\|phosphoinositide- | LN | | | | |
| | specific phospholipase C family protein | | | | | |
| 658 | gb\|AAN12963.1\|enolase (2-phospho-D- | LN | | | | |
| | glycerate hydroylase) [Arabidopsis | | | | | |
| | thaliana] | | | | | |
| 659 | ref\|NP_566528.1\|expressed protein | LN | | | | |
| | [Arabidopsis thaliana] | | | | | |
| 660 | ref\|NP_566763.1\|auxin-responsive | LN | | | | |
| | family protein [Arabidopsis thaliana] | | | | | |
| 661 | ref\|NP_194252.1\|transporter, putative | LN | | | | |
| | [Arabidopsis thaliana] | | | | | |
| 662 | ref\|NP_182046.1\|auxin-responsive | LN | | | | |
| | protein-related [Arabidopsis thaliana] | | | | | |
| 663 | ref\|NP_192980.1\|trehalose-6-phosphate | LN | | | | |
| | phosphatase, putative | | | | | |
| 664 | ref\|NP_385829.1\|PROBABLE | LN | | | | |
| | AMINOTRANSFERASE PROTEIN | | | | | |
| 665 | ref\|NP_927371.1\|glutathione dependent | LN | | | | |
| | formaldehyde dehydrogenase | | | | | |
| 666 | ref\|NP_565973.1\|LOB domain protein | LN | | | | |
| | 16/lateral organ boundaries domain | | | | | |
| | protein 16 | | | | | |
| 667 | ref\|ZP_00264891.1\|COG1012: NAD- | LN | | | | |
| | dependent aldehyde dehydrogenases | | | | | |
| | [Pseudomonas fluorescens PfO-1] | | | | | |
| 668 | ref\|NP_177763.1\|protein kinase, putative | LN | | | | |
| | [Arabidopsis thaliana] | | | | | |
| 669 | ref\|NP_181639.1\|RNA recognition | LN | | | | |
| | motif (RRM)-containing protein | | | | | |
| 670 | ref\|XP_463875.1\|putative iron- | LN | | | | |
| | phytosiderophore transporter protein | | | | | |
| | yellow stripe 1 | | | | | |
| 671 | ref\|NP_189339.1\|expressed protein | LN | | | | |
| | [Arabidopsis thaliana] | | | | | |
| 672 | dbj\|BAD37240.1\|putative phosphotyrosyl | LN | | | | |
| | phosphatase activator [Oryza sativa | | | | | |
| | (japonica cultivar-group)] | | | | | |
| 673 | ref\|NP_190798.1\|ATP synthase D chain- | LN | | | | |
| | related [Arabidopsis thaliana] | | | | | |
| 674 | ref\|NP_173294.1\|sulfotransferase family | LN | | | | |
| | protein [Arabidopsis thaliana] | | | | | |
| 675 | ref\|NP_194058.2\|protein kinase family | LN | | | | |
| | protein [Arabidopsis thaliana] | | | | | |
| 676 | ref\|NP_177412.1\|cinnamyl-alcohol | LN | | | | |
| | dehydrogenase, putative | | | | | |
| 677 | ref\|NP_851141.1\|RNA recognition motif | LN | | | | |
| | (RRM)-containing protein | | | | | |
| 678 | emb\|CAB82755.1\|protein kinase ATN1- | LN | | | | |
| | like protein [Arabidopsis thaliana] | | | | | |
| 679 | ref\|NP_197288.1\|cation exchanger, | LN | | | | |
| | putative (CAX7) [Arabidopsis thaliana] | | | | | |
| 680 | gb\|AAM14984.1\|high affinity K+ | LN | | | | |
| | transporter (AtKUP1 AtKT1p) | | | | | |
| | [Arabidopsis thaliana] | | | | | |
| 681 | ref\|NP_175713.1\|protein kinase family | LN | | | | |
| | protein [Arabidopsis thaliana] | | | | | |
| 682 | ref\|NP_568809.2\|protein kinase family | LN | | | | |
| | protein [Arabidopsis thaliana] | | | | | |
| 683 | ref\|NP_196762.1\|expressed protein | LN | | | | |
| | [Arabidopsis thaliana] | | | | | |
| 684 | ref\|NP_200323.1\|expressed protein | LN | | | | |
| | [Arabidopsis thaliana] | | | | | |
| 685 | ref\|NP_191519.1\|DNA-directed RNA | LN | PEG | | | |
| | polymerase I, II, and III, putative | | | | | |
| 686 | ref\|NP_193550.1\|outward rectifying | LN | | | | |
| | potassium channel, putative (KCO6) | | | | | |
| 687 | ref\|NP_195093.1\|L-galactose | LN | | | | |
| | dehydrogenase (L-GalDH) [Arabidopsis | | | | | |
| | thaliana] | | | | | |
| 688 | ref\|NP_568016.1\|expressed protein | LN | | | | |
| | [Arabidopsis thaliana] | | | | | |
| 689 | emb\|CAB79384.1\|protein kinase (AFC2) | LN | | | | |
| | [Arabidopsis thaliana] | | | | | |
| 690 | dbj\|BAD87508.1\|putative calcyclin- | LN | | | | |
| | binding protein [Oryza sativa (japonica | | | | | |
| | cultivar-group)] | | | | | |
| 691 | emb\|CAD44270.1\|monomeric G-protein | LN | | | | |
| | [Arabidopsis thaliana] | | | | | |
| 692 | ref\|NP_194256.1\|invertase/pectin | LN | | | | |
| | methylesterase inhibitor family protein | | | | | |
| 693 | ref\|NP_195832.1\|thylakoid membrane | LN | | | | |
| | one helix protein (OHP) [Arabidopsis | | | | | |
| | thaliana] | | | | | |
| 694 | gb\|AAM67563.1\|putative protein | PEG | | | | |
| | transport protein SEC12p [Arabidopsis | | | | | |
| | thaliana] | | | | | |
| 695 | ref\|NP_199045.1\|ubiquitin family | PEG | | | | |
| | protein [Arabidopsis thaliana] | | | | | |
| 696 | ref\|NP_179457.1\|zinc finger (C3HC4- | PEG | | | | |
| | type RING finger) family protein | | | | | |
| 697 | ref\|NP_192355.1\|aspartyl protease | PEG | | | | |
| | family protein [Arabidopsis thaliana] | | | | | |
| 698 | ref\|NP_850347.1\|F-box family protein | PEG | | | | |
| | [Arabidopsis thaliana] | | | | | |
| 699 | gb\|AAT06481.1\|At3g23540 [Arabidopsis | PEG | | | | |
| | thaliana] | | | | | |
| 700 | gb\|AAM66021.1\|plasma membrane | PEG | | | | |
| | intrinsic protein SIMIP [Arabidopsis | | | | | |
| | thaliana] | | | | | |
| 701 | dbj\|BAA07547.1\|phosphoinositide | PEG | | | | |
| | specific phospholipase C [Arabidopsis | | | | | |
| | thaliana] | | | | | |
| 702 | ref\|NP_195154.2\|transducin family | PEG | | | | |
| | protein/WD-40 repeat family protein | | | | | |
| 703 | ref\|NP_188176.1\|phototropic-responsive | PEG | | | | |
| | NPH3 family protein [Arabidopsis | | | | | |
| | thaliana] | | | | | |
| 704 | ref\|NP_189177.1\|formin homology 2 | PEG | | | | |
| | domain-containing protein/FH2 domain- | | | | | |
| | containing protein [Arabidopsis thaliana] | | | | | |
| 705 | ref\|NP_015238.1\|Ydc1p [Saccharomyces | PEG | | | | |
| | cerevisiae] gb\|AAB68212.1\|Lpg21p | | | | | |
| | gb\|AAG22594.1\|alkaline ceramidase | | | | | |
| | Ydc1p | | | | | |
| 706 | ref\|NP_565508.1\|fructose-bisphosphate | PEG | | | | |
| | aldolase, putative [Arabidopsis thaliana] | | | | | |
| 707 | ref\|NP_012795.1\|Pgm1p [Saccharomyces | PEG | | | | |
| | cerevisiae] emb\|CAA50895.1\| | | | | | |
| | phosphoglucomutase | | | | | |
| 708 | ref\|NP_243583.1\|glutaminase [Bacillus | PEG | | | | |
| | halodurans C-125] | | | | | |
| 709 | ref\|NP_193265.1\|cytochrome P450 | PEG | | | | |
| | family protein [Arabidopsis thaliana] | | | | | |
| 710 | sp\|P38970\|HAL5_YEAST | PEG | | | | |
| | Serine/threonine-protein kinase HAL5 | | | | | |
| 711 | ref\|NP_564866.1\|U-box domain- | PEG | | | | |
| | containing protein | | | | | |
| 712 | ref\|NP_391762.1\|aldehyde | PEG | | | | |
| | dehydrogenase [Bacillus subtilis subsp. | | | | | |
| | subtilis str. 168] | | | | | |
| 713 | ref\|NP_928238.1\|glutamate-1- | PEG | | | | |
| | semialdehyde 2,1-aminomutase | | | | | |
| | [Photorhabdus luminescens subsp. | | | | | |
| | laumondii TTO1] | | | | | |
| 714 | ref\|NP_388662.1\|trehalose-6-phosphate | PEG | | | | |
| | hydrolase [Bacillus subtilis subsp. subtilis | | | | | |
| | str. 168] | | | | | |
| 715 | ref\|NP_171654.1\|late embryogenesis | PEG | | | | |
| | abundant protein, putative/LEA protein, | | | | | |
| | putative | | | | | |
| 716 | ref\|NP_193486.1\|Ras-related GTP- | PEG | | | | |
| | binding protein, putative [Arabidopsis | | | | | |
| | thaliana] | | | | | |
| 717 | ref\|NP_563987.1\|expressed protein | PEG | | | | |
| | [Arabidopsis thaliana] gb\|AAF18498.1\| | | | | | |
| | Identical to gb\|Y10291 GAG1 protein | | | | | |
| 718 | gb\|AAU44232.1\|hypothetical protein | PEG | | | | |
| | [Oryza sativa (japonica cultivar-group)] | | | | | |
| 719 | gb\|AAC31235.1\|hypothetical protein | PEG | | | | |
| | [Arabidopsis thaliana] gb\|AAT71954.1\| | | | | | |
| 720 | ref\|NP_441145.1\|hypothetical protein | PEG | | | | |
| | sll1162 [Synechocystis sp. PCC 6803] | | | | | |
| 721 | ref\|NP_484561.1\|fructokinase [Nostoc | PEG | | | | |
| | sp. PCC 7120] | | | | | |
| 722 | ref\|NP_567109.1\|COP9 signalosome | PEG | | | | |
| | complex subunit 1/CSN complex | | | | | |
| | subunit 1 (CSN1)/COP11 protein | | | | | |
| | (COP11)/FUSCA protein (FUS6) | | | | | |
| | [Arabidopsis thaliana] | | | | | |
| 723 | ref\|NP_180431.1\|beta-ketoacyl-CoA | PEG | | | | |
| | synthase family protein [Arabidopsis | | | | | |
| | thaliana] | | | | | |
| 724 | ref\|NP_189034.1\|beta-amylase, putative/ | PEG | | | | |
| | 1,4-alpha-D-glucan maltohydrolase, | | | | | |
| | putative [Arabidopsis thaliana] | | | | | |
| 725 | ref\|NP_201100.1\|expressed protein | PEG | | | | |
| | [Arabidopsis thaliana] | | | | | |
| 726 | ref\|NP_196701.1\|protein kinase-related | PEG | | | | |
| | [Arabidopsis thaliana] | | | | | |
| 727 | emb\|CAB61965.1\|1-aminocyclopropane- | PEG | | | | |
| | 1-carboxylic acid oxidase-like protein | | | | | |
| 728 | dbj\|BAD87231.1\|membrane protein-like | PEG | | | | |
| | [Oryza sativa (japonica cultivar-group)] | | | | | |
| 729 | ref\|XP_466202.1\|putative DNA J domain | PEG | | | | |
| | protein [Oryza sativa (japonica cultivar- | | | | | |
| | group)] | | | | | |
| 730 | ref\|NP_179479.1\|protein kinase family | PEG | | | | |
| | protein [Arabidopsis thaliana] | | | | | |
| 731 | ref\|NP_200428.1\|expressed protein | PEG | | | | |
| | [Arabidopsis thaliana] | | | | | |
| 732 | ref\|NP_178191.1\|major intrinsic family | PEG | | | | |
| | protein/MIP family protein | | | | | |
| 733 | gb\|AAN64166.1\|unknown protein | PEG | | | | |
| | [Arabidopsis thaliana] | | | | | |
| 734 | ref\|NP_636017.1\|phosphomannose | PEG | | | | |
| | isomerase/GDP-mannose | | | | | |
| | pyrophosphorylase | | | | | |
| 735 | dbj\|BAB10630.1\|glucose-6-phosphate | PP | | | | |
| | isomerase, cytosolic [Arabidopsis | | | | | |
| | thaliana] | | | | | |
| 736 | ref\|NP_564835.1\|expressed protein | PP | HS | | | |
| | [Arabidopsis thaliana] | | | | | |
| 737 | ref\|NP_197149.1\|dimethylmenaquinone | PP | PEG | | | |
| | methyltransferase family protein | | | | | |
| 738 | ref\|NP_172592.1\|glucose transporter | PP | | | | |
| | (STP1) [Arabidopsis thaliana] | | | | | |
| 739 | ref\|NP_564017.1\|integral membrane | PP | SS | PEG | | |
| | family protein [Arabidopsis thaliana] | | | | | |
| 740 | ref\|NP_198871.2\|expressed protein | PP | | | | |
| | [Arabidopsis thaliana] | | | | | |
| 741 | ref\|NP_177188.1\|spermidine synthase 2 | PP | SS | PEG | | |
| | (SPDSYN2)/putrescine | | | | | |
| | aminopropyltransferase 2 | | | | | |
| 742 | emb\|CAB41169.1\|cytochrome P450-like | PP | | | | |
| | protein [Arabidopsis thaliana] | | | | | |
| 743 | ref\|NP_173165.1\|translation initiation | PP | | | | |
| | factor IF-2, chloroplast, putative | | | | | |
| | [Arabidopsis thaliana] | | | | | |
| 744 | ref\|NP_181996.1\|casein kinase II beta | PP | LL | LN | PEG | |
| | chain, putative [Arabidopsis thaliana] | | | | | |
| 745 | dbj\|BAB09497.1\|chloroplast nucleoid | PP | | | | |
| | DNA-binding protein-like [Arabidopsis | | | | | |
| | thaliana] ref\|NP_199325.1\|aspartyl | | | | | |
| | protease family protein [Arabidopsis | | | | | |
| | thaliana] | | | | | |
| 746 | ref\|NP_180133.1\|protein phosphatase | PP | SS | | | |
| | 2C, putative/PP2C, putative | | | | | |
| 747 | ref\|NP_195661.1\|cytochrome P450 | PP | | | | |
| | family protein [Arabidopsis thaliana] | | | | | |
| 748 | gb\|AAM63133.1\|delta tonoplast integral | PP | | | | |
| | protein delta-TIP [Arabidopsis thaliana] | | | | | |
| 749 | ref\|NP_795058.1\|phosphoglycerate | PP | | | | |
| | mutase, 2,3-bisphosphoglycerate- | | | | | |
| | independent | | | | | |
| 750 | pir\|\|A95262probable formate | PP | PEG | | | |
| | dehydrogenase (EC 1.2.1.2) alpha chain | | | | | |
| | FdoG [imported]-SinoRhizobium | | | | | |
| | meliloti (strain 1021) magaplasmid | | | | | |
| | pSymA | | | | | |
| 751 | ref\|NP_190945.1\|late embryogenesis | PP | SS | PEG | | |
| | abundant protein-related/LEA protein- | | | | | |
| | related | | | | | |
| 752 | ref\|ZP_00108145.1\|COG1523: Type II | PP | SS | | | |
| | secretory pathway, pullulanase PulA and | | | | | |
| | related glycosidases [Nostoc punctiforme | | | | | |
| | PCC 73102] | | | | | |
| 753 | ref\|NP_564975.2\|CBS domain-containing | PP | | | | |
| | protein [Arabidopsis thaliana] | | | | | |
| 754 | ref\|NP_566716.1\|protein kinase, putative | PP | SS | LL | | |
| | [Arabidopsis thaliana] | | | | | |
| 755 | ref\|NP_566876.3\|protein kinase family | PP | SS | | | |
| | protein [Arabidopsis thaliana] | | | | | |
| 756 | ref\|NP_441848.1\|hypothetical protein | PP | | | | |
| | sll0359 [Synechocystis sp. PCC 6803] | | | | | |
| 757 | emb\|CAB42913.1\|putative protein | PP | | | | |
| | [Arabidopsis thaliana] pir\|\|T08405 | | | | | |
| | hypothetical protein F18B3.120- | | | | | |
| | Arabidopsis thaliana | | | | | |
| 758 | ref\|NP_564042.1\|expressed protein | PP | | | | |
| | [Arabidopsis thaliana] | | | | | |
| 759 | ref\|NP_858035.1\|mercuric reductase | PP | | | | |
| | [uncultured bacterium] | | | | | |
| 760 | ref\|ZP_00356067.1\|COG4992: | PP | SS | | | |
| | Ornithine/acetylornithine | | | | | |
| | aminotransferase [Chloroflexus | | | | | |
| | aurantiacus] | | | | | |
| 761 | ref\|NP_186761.1\|cystathionine gamma- | PP | LL | | | |
| | synthase, chloroplast/O- | | | | | |
| | succinylhomoserine (Thiol)-lyase (CGS) | | | | | |
| 762 | ref\|NP_197266.1\|expressed protein | PP | SS | LL | LN | PEG |
| | [Arabidopsis thaliana] | | | | | |
| 763 | pir\|\|T48630 high affinity nitrate | PP | | | | |
| | transporter-like protein-Arabidopsis | | | | | |
| | thaliana | | | | | |
| 764 | ref\|NP_850901.1\|expressed protein | PP | | | | |
| | [Arabidopsis thaliana] | | | | | |
| 765 | ref\|XP_483810.1\|putative Bet1/Sft1- | PP | PEG | | | |
| | related SNARE (AtBS14a) [Oryza sativa | | | | | |
| 766 | emb\|CAB88286.1\|serine/threonine- | PP | | | | |
| | specific protein kinase-like protein | | | | | |
| 767 | ref\|XP_475647.1\|putative glutaryl-CoA | PP | HS | PEG | | |
| | dehydrogenase | | | | | |
| 768 | ref\|NP_196906.2\|expressed protein | PP | SS | | | |
| | [Arabidopsis thaliana] | | | | | |
| 769 | ref\|XP_467724.1\|unknown protein | PP | PEG | | | |
| | [Oryza sativa (japonica cultivar-group)] | | | | | |
| 770 | gb\|AAF23278.1\|unknown protein | PP | HS | SS | PEG | |
| | [Arabidopsis thaliana] | | | | | |
| 771 | ref\|NP_920131.1\|putative Magnaporthe | PP | | | | |
| | grisea pathogenicity protein | | | | | |
| 772 | ref\|NP_192381.1\|calcium-dependent | SP | HS | PEG | | |
| | protein kinase, putative/CDPK, putative | | | | | |
| 773 | ref\|NP_179791.1\|expressed protein | SP | CK | PP | PEG | |
| | [Arabidopsis thaliana] | | | | | |
| 774 | ref\|NP_177550.1\|sulfotransferase family | SP | | | | |
| | protein [Arabidopsis thaliana] | | | | | |
| 775 | ref\|NP_192830.1\|transcriptional | SP | SS | | | |
| | coactivator p15 (PC4) family protein | | | | | |
| | (KELP) | | | | | |
| 776 | ref\|NP_176726.1\|flowering locus T | SP | SS | PEG | | |
| | protein (FT) [Arabidopsis thaliana] | | | | | |
| 777 | ref\|NP_565844.1\|zinc finger (AN1-like) | SP | LN | | | |
| | family protein [Arabidopsis thaliana] | | | | | |
| 778 | gb\|AAF79653.1\|F5O11.17 [Arabidopsis | SP | LN | | | |
| | thaliana] | | | | | |
| 779 | ref\|NP_196373.1\|glycine-rich protein | SP | CK | | | |
| | (GRP20) [Arabidopsis thaliana] | | | | | |
| 780 | ref\|NP_188020.1\|exopolygalacturonase/ | SP | PP | PEG | | |
| | galacturan 1,4-alpha-galacturonidase/ | | | | | |
| | pectinase | | | | | |
| 781 | ref\|NP_200440.1\|peroxisomal targeting | SP | CK | | | |
| | signal type 1 receptor (PEX5) | | | | | |
| 782 | ref\|NP_194774.1\|glycine-rich protein | SP | CK | | | |
| | [Arabidopsis thaliana] | | | | | |
| 783 | ref\|NP_197608.1\|leucine-rich repeat | SP | DS | PEG | | |
| | protein, putative [Arabidopsis thaliana] | | | | | |
| 784 | ref\|NP_563653.2\|NPR1/NIM1- | SP | SS | LN | | |
| | interacting protein 1 (NIMIN-1) | | | | | |
| | [Arabidopsis thaliana] | | | | | |
| 785 | ref\|NP_173727.1\|C2 domain-containing | SP | CS | PEG | | |
| | protein [Arabidopsis thaliana] | | | | | |
| 786 | ref\|NP_706078.2\|hypoxanthine | SP | SS | PEG | | |
| | phosphoribosyltransferase [Shigella | | | | | |
| | flexneri 2a str. 301] EDL933] | | | | | |
| 787 | ref\|NP_442276.1\|hypothetical protein | SP | SS | | | |
| | slr0630 [Synechocystis sp. PCC 6803] | | | | | |
| 788 | ref\|NP_568786.1\|protein phosphatase | SP | | | | |
| | 2C, putative/PP2C, putative | | | | | |
| 789 | ref\|NP_974249.1\|prephenate | SP | PEG | | | |
| | dehydratase family protein [Arabidopsis | | | | | |
| | thaliana] | | | | | |
| 790 | ref\|NP_174536.1\|plastid developmental | SP | CS | PEG | | |
| | protein DAG, putative | | | | | |
| 791 | dbj\|BAB10767.1\|unnamed protein | SP | SS | | | |
| | product [Arabidopsis thaliana] | | | | | |
| 792 | emb\|CAA48415.1\|unnamed protein | SP | CK | PEG | | |
| | product [synthetic construct] | | | | | |
| 793 | ref\|NP_180570.1\|GCN5-related N- | SP | CS | | | |
| | acetyltransferase (GNAT) family protein | | | | | |
| 794 | ref\|NP_442494.1\|aldehyde | SP | SS | PEG | | |
| | dehydrogenase [Synechocystis sp. PCC | | | | | |
| | 6803] | | | | | |
| 795 | emb\|CAB82954.1\|cytocluome c oxidase | SP | CK | CS | PEG | |
| | subunit 5c-like protein [Arabidopsis | | | | | |
| | thaliana] | | | | | |
| 796 | ref\|NP_175374.2\|expressed protein | SP | CS | HS | PEG | |
| | [Arabidopsis thaliana] | | | | | |
| 797 | ref\|NP_188286.1\|translationally | SP | PP | SS | | |
| | controlled tumor family protein | | | | | |
| | [Arabidopsis thaliana] | | | | | |
| 798 | ref\|NP_194825.1\|CBL-interacting | SP | LN | | | |
| | protein kinase 6 (CIPK6) [Arabidopsis | | | | | |
| | thaliana] | | | | | |
| 799 | gb\|AAD12699.1\|putative ribophorin I | SP | | | | |
| | [Arabidopsis thaliana] | | | | | |
| 800 | ref\|NP_200169.1\|RabGAP/TBC | SS | | | | |
| | domain-containing protein [Arabidopsis | | | | | |
| | thaliana] | | | | | |
| 801 | ref\|NP_189210.2\|myrcene/ocimene | SS | PEG | | | |
| | synthase, putative [Arabidopsis thaliana] | | | | | |
| 802 | ref\|NP_564266.1\|expressed protein | SS | | | | |
| | [Arabidopsis thaliana] | | | | | |
| 803 | ref\|NP_974213.1\|ankyrin repeat family | SS | PEG | | | |
| | protein/regulator of chromosome | | | | | |
| | condensation (RCC1) family protein | | | | | |
| | [Arabidopsis thaliana] | | | | | |
| 804 | ref\|NP_190632.1\|kip-related protein 2 | SS | | | | |
| | (KRP2)/cyclin-dependent kinase | | | | | |
| | inhibitor 2 (ICK2)/cdc2a-interacting | | | | | |
| | protein [Arabidopsis thaliana] | | | | | |
| 805 | ref\|NP_200680.1\|PRLI-interacting | SS | LN | | | |
| | factor, putative [Arabidopsis thaliana] | | | | | |
| 806 | gb\|AAM55306.1\|auxin influx carrier | SS | | | | |
| | protein [Medicago truncatula] | | | | | |
| 807 | gb\|Aref\|NP_190468.1\|AMP-dependent | SS | | | | |
| | synthetase and ligase family protein | | | | | |
| 808 | ref\|NP_564424.1\|PHD finger family | SS | | | | |
| | protein [Arabidopsis thaliana] | | | | | |
| 809 | ref\|NP_194207.1\|expressed protein | SS | LN | | | |
| | [Arabidopsis thaliana] | | | | | |
| 810 | ref\|NP_186938.1\|leucine-rich repeat | SS | PEG | | | |
| | transmembrane protein kinase, putative | | | | | |
| 811 | gb\|AAO22792.1\|putative cytochrome c | SS | PEG | | | |
| | oxidoreductase [Arabidopsis thaliana] | | | | | |
| 812 | ref\|NP_192805.1\|transcription | SS | | | | |
| | elongation factor-related [Arabidopsis | | | | | |
| | thaliana] | | | | | |
| 813 | ref\|NP_568107.1\|pseudo-response | SS | | | | |
| | regulator 7 (APRR7) [Arabidopsis | | | | | |
| | thaliana] sp\|Q93WK5\|APRR7_ARATH | | | | | |
| | Two-component response regulator-like | | | | | |
| | APRR7 (Pseudo-response regulator 7) | | | | | |
| 814 | ref\|NP_442120.1\|ribulose bisphosphate | SS | | | | |
| | carboxylase large subunit | | | | | |
| 815 | ref\|NP_440401.1\|mercuric reductase | SS | | | | |
| | [Synechocystis sp. PCC 6803] | | | | | |
| 816 | gb\|AAN31115.1\|At2g26250/T1D16.11 | SS | | | | |
| | [Arabidopsis thaliana] gb\|AAG60062.1\| | | | | | |
| | putative beta-ketoacyl-CoA synthase | | | | | |
| | FIDDLEHEAD | | | | | |
| 817 | ref\|NP_186798.1\|protein kinase, | SS | | | | |
| | putative [Arabidopsis thaliana] | | | | | |
| 818 | gb\|AAM65778.1\|putative NADPH | SS | | | | |
| | quinone oxidoreductase [Arabidopsis | | | | | |
| | thaliana] | | | | | |
| 819 | ref\|ZP_00107727.1\|COG0166: Glucose- | SS | | | | |
| | 6-phosphate isomerase [Nostoc | | | | | |
| | punctiforme PCC 73102] | | | | | |
| 820 | ref\|NP_388166.1\|hypothetical protein | SS | PEG | | | |
| | BSU02840 [Bacillus subtilis subsp. | | | | | |
| | subtilis str. 168] | | | | | |
| 821 | ref\|NP_194624.1\|Rac-like GTP-binding | SS | | | | |
| | protein (ARAC7) [Arabidopsis thaliana] | | | | | |
| 822 | gb\|AAO24554.1\|At1g61150 [Arabidopsis | SS | PEG | | | |
| | thaliana] | | | | | |
| 823 | ref\|NP_850128.1\|protein kinase family | SS | | | | |
| | protein [Arabidopsis thaliana] | | | | | |
| 824 | ref\|NP_441594.1\|hypothetical protein | SS | LL | | | |
| | slr1273 [Synechocystis sp. PCC 6803] | | | | | |
| 825 | ref\|NP_194073.2\|short-chain | SS | | | | |
| | dehydrogenase/reductase (SDR) family | | | | | |
| | protein | | | | | |
| 826 | ref\|NP_173437.1\|Rac-like GTP-binding | SS | | | | |
| | protein (ARAC4)/Rho-like GTP-binding | | | | | |
| | protein (ROP2) | | | | | |
| 827 | ref\|NP_850393.1\|sugar transporter | SS | | | | |
| | family protein [Arabidopsis thaliana] | | | | | |
| 828 | gb\|AAC04613.1\|arginase [Glycine max] | SS | | | | |
| | pir\|\|T06222 probable arginase (EC | | | | | |
| | 3.5.3.1)-soybean | | | | | |
| | sp\|O49046\|ARGI_SOYBN Arginase | | | | | |
| 829 | gb\|AAR87370.1\|expressed protein [Oryza | SS | | | | |
| | sativa (japonica cultivar-group)] | | | | | |
| 830 | emb\|CAH40838.1\|protein-O- | SS | | | | |
| | fucosyltransferase 1 [Saccharum | | | | | |
| | officinarum] | | | | | |
| 831 | ref\|NP_175261.1\|G protein coupled | SS | | | | |
| | receptor-related [Arabidopsis thaliana] | | | | | |
| 832 | ref\|NP_176128.1\|hypothetical protein | SS | | | | |
| | [Arabidopsis thaliana] | | | | | |
| 833 | ref\|NP_974937.1\|RNA recognition motif | SS | LN | | | |
| | (RRM)-containing protein | | | | | |
| 834 | ref\|NP_180456.1\|mitochondrial import | SS | | | | |
| | inner membrane translocase subunit | | | | | |
| | Tim17/Tim22/Tim23 family protein | | | | | |
| | [Arabidopsis thaliana] | | | | | |
| 835 | ref\|NP_565766.1\|glycolipid transfer | SS | | | | |
| | protein-related [Arabidopsis thaliana] | | | | | |
| 836 | ref\|NP_193484.1\|ubiquitin carboxyl- | SS | | | | |
| | terminal hydrolase | | | | | |
| 837 | ref\|NP_199436.1\|inward rectifying | SS | LN | | | |
| | potassium channel (KAT1) | | | | | |
| 838 | ref\|NP_190844.1\|integral membrane | SS | PEG | | | |
| | Yip1 family protein [Arabidopsis | | | | | |
| | thaliana] | | | | | |
| 839 | ref\|NP_201509.1\|protein kinase family | SS | LN | | | |
| | protein [Arabidopsis thaliana] | | | | | |
| 840 | ref\|NP_200137.1\|arabinogalactan- | SS | | | | |
| | protein, putative (AGP22) [Arabidopsis | | | | | |
| | thaliana] | | | | | |
| 841 | ref\|NP_563862.1\|expressed protein | SS | PEG | | | |
| | [Arabidopsis thaliana] | | | | | |
| 842 | dbj\|BAD28194.1\|putative MFAP1 protein | SS | | | | |
| | [Oryza sativa (japonica cultivar-group)] | | | | | |
| 843 | gb\|AAC19375.1\|submergence induced | SS | | | | |
| | protein 2A [Oryza sativa] | | | | | |
| 844 | gb\|AAO72703.1\|unknown [Oryza sativa | SS | | | | |
| | (japonica cultivar-group)] | | | | | |
| 845 | ref\|NP_187911.1\|transporter-related | SS | PEG | | | |
| | [Arabidopsis thaliana] | | | | | |
| 846 | ref\|NP_599939.1\|detergent sensitivity | SS | LN | | | |
| | rescuer dtsR2 [Corynebacterium | | | | | |
| | glutamicum ATCC 13032] | | | | | |
| 847 | gb\|AAQ14479.1\|putative | SS | | | | |
| | aminotransferase [Oryza sativa] | | | | | |
| 848 | gb\|AAC63962.1\|pyruvate dehydrogenase | SS | | | | |
| | kinase isoform 2; PDK2 [Zea mays] | | | | | |
| 849 | ref\|NP_244081.1\|hypothetical protein | SS | PEG | | | |
| | BH3215 [Bacillus halodurans C-125] | | | | | |
| 850 | ref\|NP_564285.1\|calmodulin-binding | SS | PEG | | | |
| | protein [Arabidopsis thaliana] | | | | | |

“CK” indicates cold tolerance improvement identified under a cold shock tolerance screen;
“CS” indicates cold tolerance improvement identified by a cold germination tolerance screen;
“DS” indicates drought tolerance improvement identified by a soil drought stress tolerance screen;
“PEG” indicates osmotic stress tolerance improvement identified by a PEG induced osmotic stress tolerance screen;
“HS” indicates heat stress tolerance improvement identified by a heat stress tolerance screen;
“SS” indicates high salinity stress tolerance improvement identified by a salt stress tolerance screen;
“LN” indicates nitrogen use efficiency improvement identified by a limited nitrogen tolerance screen;
“LL” indicates attenuated shade avoidance response identified by a shade tolerance screen under a low light condition;
“PP” indicates improved growth and development at early stages identified by an early plant growth and development screen;
“SP” indicates improved growth and development at late stages identified by a late plant growth and development screen as described in U.S. Patent Application Publication No. 2007-0124833, and incorporated herein by reference. Some proteins may provide more than one traits. For example, proteins of SEQ ID NOs. 432 and 447.

TABLE 9
Examples of nucleic acids and proteins for various cost decreasing traits for use in the present invention. These are
described in Patent Application Publication No. WO07027866, and incorporated herein by reference.

| NUC | PEP | |
|---|---|---|
| SEQ | SEQ | |
| ID | ID | |
| NO | NO | Annotation and description of the SEQ ID |
| 1 | 194 | gb\|AAA33498.1\|pyruvate, orthophosphate dikinase |
| 2 | 195 | gb\|AAA33498.1\|pyruvate, orthophosphate dikinase |
| 3 | 196 | ref\|NP_915342.1\|putative calcium-dependent protein kinase [Oryza sativa (japonica cultivar-group)] |
| 4 | 197 | ref\|XP_470045.1\|putative calmodulin-domain protein kinase [Oryza sativa (japonica cultivar-group)] |
| 5 | 198 | gb\|AAU95457.1\|At5g12180 [Arabidopsis thaliana] dbj\|BAB10036.1\|calcium-dependent protein kinase |
| 6 | 199 | dbj\|BAC43300.1\|putative calcium-dependent protein kinase [Arabidopsis thaliana] |
| 7 | 200 | dbj\|BAB92151.1\|putative CBL-interacting protein kinase 2 [Oryza sativa (japonica |
| 8 | 201 | ref\|XP_507586.1\|PREDICTED P0524F03.33 gene product [Oryza sativa (japonica cultivar-group)] |
| | | ref\|XP_482612.1\|putative CCR4-NOT transcription complex, subunit 7 |
| 9 | 202 | emb\|CAA58052.1\|aspaiTagine synthetase [Zea mays] |
| 10 | 203 | sp\|Q9LDE6\|CKX1_ORYSA Probable cytokinin dehydrogenase precursor (Cytokinin oxidase) (CKO) |
| 11 | 204 | ref\|NP_171645.1\|myb family transcription factor [Arabidopsis thaliana] |
| 12 | 205 | emb\|CAE03364.1\|OSJNBb0065L13.7 [Oryza sativa (japonica cultivar-group)] |
| 13 | 206 | dbj\|BAB07329.1\|cold-shock protein [Bacillus halodurans C-125] |
| 14 | 207 | emb\|CAF18741.1\|COLD-SHOCK PROTEIN CSPA [Corynebacterium glutamicum ATCC 13032] |
| 15 | 208 | dbj\|BAD32022.1\|putative transcription factor [Oryza sativa |
| 16 | 209 | gb\|AAG09919.1\|MADS box protein 2 [Zea mays] |
| 17 | 210 | emb\|CAB80652.1\|small GTP-binding protein-like [Arabidopsis thaliana] |
| 18 | 211 | emb\|CAB79187.1\|hypothetical protein [Arabidopsis thaliana] |
| 19 | 212 | ref\|XP_465955.1\|putative nodulin 3 [Oryza sativa (japonica cultivar-group)] |
| 20 | 213 | emb\|CAD37109.2\|OSJNBa0024J22.22 [Oryza sativa (japonica cultivar-group)] |
| 21 | 214 | dbj\|BAD45770.1\|putative Cyt-P450 monooxygenase [Oryza sativa (japonica cultivar-group)] |
| 22 | 215 | gb\|AAP79441.1\|glutamate decarboxylase [Oryza sativa (japonica cultivar-group)] |
| 23 | 216 | dbj\|BAB63676.1\|induced protein MgI1 [Oryza sativa (japonica cultivar-group)] |
| 24 | 217 | dbj\|BAB63676.1\|induced protein MgI1[Oryza sativa (japonica cultivar-group)] |
| 25 | 218 | ref\|NP_195478.2\|cyclin family protein [Arabidopsis thaliana] gb\|AAS49095.1\|At4g37630 [Arabidopsis |
| | | thaliana] |
| 26 | 219 | gb\|AAL99744.1\|pyruvate decarboxylase [Zea mays] |
| 27 | 220 | gb\|AAG43027.1\|aldehyde dehydrogenase [Oryza sativa] |
| 28 | 221 | ref\|XP_464496.1\|ribosomal protein L12-like protein [Oryza sativa (japonica cultivar-group)] |
| 29 | 222 | dbj\|BAD37896.1\|ARG1-like protein [Oryza sativa (japonica cultivar-group)] |
| 30 | 223 | ref\|XP_464309.1\|putative choline-phosphate cytidylyltransferase [Oryza sativa (japonica cultivar-group)] |
| 31 | 224 | gb\|AAV44199.1\|dehydroascorbate reductase [Oryza sativa (japonica cultivar-group)] |
| 32 | 225 | ref\|XP_550011.1\|hypothetical protein [Oryza sativa (japonica cultivar-group)] |
| 33 | 226 | ref\|XP_470303.1\|hypothetical protein [Oryza sativa (japonica cultivar-group)] |
| 34 | 227 | ref\|XP_507362.1\|PREDICTED OSJNBa0077F02.127 gene product [Oryza sativa (japonica cultivar-group)] |
| 35 | 228 | gb\|AAA20093.21Alfin-1 [Medicago saliva] pir∥T09646 probable zinc finger protein-alfalfa (fragment) |
| 36 | 229 | gb\|AAM64652.1\|LAX1/AUX1-like permease [Arabidopsis thaliana] |
| 37 | 230 | emb\|CAB79732.1\|cytokinin oxidase-like protein [Arabidopsis thaliana] |
| 38 | 231 | dbj\|BAA14344.1\|sucrose phosphorylase [Leuconostoc mesenteroides] |
| 12607 | 231 | dbj\|BAA14344.1\|sucrose phosphorylase [Leuconostoc mesenteroides] |
| 39 | 232 | dbj\|BAA87958.1\|CW14 [Arabidopsis thaliana] |
| 40 | 233 | ref\|NP_191207.2\|myosin heavy chain-related [Arabidopsis thaliana] |
| 41 | 234 | dbj\|BAD91881.1\|transcription factor lim1 [Eucalyptus camaldulensis] |
| 42 | 235 | dbj\|BAD91881.1\|transcription factor lim1 [Eucalyptus camaldulensis] |
| 43 | 236 | ref\|NP_563710.1\|AWPM-19-like membrane family protein [Arabidopsis thaliana] |
| 44 | 237 | ref\|NP_568751.1\|polyadenylate-binding protein, putative/PABP, putative [Arabidopsis thaliana] |
| 45 | 238 | ref\|NP_192188.2\|GTP-binding family protein [Arabidopsis thaliana] |
| 46 | 239 | ref\|NP_192188.2\|GTP-binding family protein [Arabidopsis thaliana] |
| 47 | 240 | ref\|NP_196133.3\|transcription elongation factor-related [Arabidopsis thaliana] |
| 48 | 241 | ref\|NP_566493.1\|nodulin MtN3 family protein [Arabidopsis thaliana] |
| 49 | 242 | emb\|CAA42168.1\|L-ascorbate peroxidase [Arabidopsis thaliana] |
| 50 | 243 | emb\|CAB78019.1\|putative phi-1-like phosphate-induced protein [Arabidopsis thaliana] gb\|AAM18526.1\|cell |
| | | cycle-related protein [Arabidopsis thaliana] |
| 51 | 244 | gb\|AAK06866.1\|putative ATPase [Arabidopsis thaliana] ref\|NP_173536.1\|O-methyltransferase, putative |
| | | [Arabidopsis thaliana] |
| 52 | 245 | gb\|AAK06866.1\|putative ATPase [Arabidopsis thaliana] ref\|NP_173536.1\|O-methyltransferase, putative |
| | | [Arabidopsis thaliana] |
| 53 | 246 | ref\|NP_568761.1\|expressed protein [Arabidopsis thaliana] |
| 54 | 247 | ref\|NP_565390.1\|actin-depolymerizing factor 5 (ADF5) [Arabidopsis thaliana] |
| 55 | 248 | ref\|ZP_00272945.1\|COG0538: Isocitrate dehydrogenases [Ralstonia metallidurans CH34] |
| 56 | 249 | ref\|NP_441003.1\|phycocyanin alpha phycocyanobilin lyase; CpcE [Synechocystis sp. PCC 6803] |
| 57 | 250 | ref\|NP_441003.1\|phycocyanin alpha phycocyanobilin lyase; CpcE [Synechocystis sp. PCC 6803] |
| 58 | 251 | ref\|NP_199781.1\|DNA-binding protein-related [Arabidopsis thaliana] |
| 59 | 252 | ref\|NP_200010.1\|sorbitol dehydrogenase, putative/L-iditol 2-dehydrogenase, putative [Arabidopsis thaliana] |
| 60 | 253 | ref\|NP_187478.1\|phototropic-responsive protein, putative [Arabidopsis thaliana] |
| 61 | 254 | emb\|CAB79666.1\|phytochrome-associated protein PAP2 [Arabidopsis thaliana] |
| 62 | 255 | ref\|NP_198423.1\|glycosyl hydrolase family protein 17 [Arabidopsis thaliana] |
| 63 | 256 | emb\|CAC34495.1\|putative strictosidine synthase-like [Arabidopsis thaliana] |
| 64 | 257 | ref\|NP_569050.1\|adenylylsulfate kinase, putative [Arabidopsis thaliana] |
| 65 | 258 | ref\|NP_199253.1\|FAD-binding domain-containing protein [Arabidopsis thaliana] |
| 66 | 259 | gb\|AAD39335.1\|Putative Aldo/keto reductase [Arabidopsis thaliana] |
| 67 | 260 | emb\|CAB80527.1\|multiubiquitin chain binding protein (MBP1) [Arabidopsis thaliana] |
| 68 | 261 | ref\|NP_182075.1\|cytochrome P450, putative [Arabidopsis thaliana] |
| 69 | 262 | ref\|NP_200218.1\|heat shock transcription factor family protein [Arabidopsis thaliana] . |
| 70 | 263 | ref\|NP_192426.1\|basic helix-loop-helix (bHLH) family protein [Arabidopsis thaliana] |
| 71 | 264 | gb\|AAS09998.1\|MY13 transcription factor [Arabidopsis thaliana] |
| 72 | 265 | ref\|NP_195750.1\|phosphatidylethanolamine-binding family protein [Arabidopsis thaliana] |
| 73 | 266 | ref\|NP_190239.1\|fasciclin-like arabinogalactan family protein [Arabidopsis thaliana] |
| 74 | 267 | gb\|AAC49371.1\|RF2 gb\|AAG43988.1\|T cytoplasm male sterility restorer factor 2 [Zea mays] |
| 75 | 268 | dbj\|BAD54414.1\|aldehyde dehydrogenase ALDH2b [Oryzo sativa (japonica cultivar-group)] |
| 76 | 269 | gb\|AAM46894.1\|early drought induced protein [Oryza sativa (indica cultivar-group)] |
| 77 | 270 | ref\|NP_010562.1\|Small plasma membrane protein related to a family of plant polypeptides that are |
| | | overexpressed under high salt concentration or low temperature, not essential for viability, deletion causes |
| | | hyperpolarization of the plasma membrane potential; Pmp3p [Saccharomyces cerevisiae] |
| 78 | 271 | ref\|NP_188922.1\|heat shock transcription factor family protein [Arabidopsis thaliana] |
| 79 | 272 | emb\|CAA45039.1\|heat shock protein 17.6-II [Arabidopsis thaliana] |
| 80 | 273 | dbj\|BAC76332.1\|HAP3 [Oryza sativa (japonica cultivar-group)] |
| 81 | 274 | gb\|AAK95562.1\|1eafy cotyledonl [Zea mays] |
| 82 | 275 | dbj\|BAD32022.1\|putative transcription factor [Oryza sativa (japonica cultivar-group)] |
| 83 | 276 | ref\|NP_914939.1\|putative CCAAT-binding transcription factor subunit A(CBF-A) [Oryza sativa |
| 84 | 277 | ref\|NP_850005.1\|expressed protein [Arabidopsis thaliand |
| 85 | 278 | dbj\|BAC76333.1\|HAP3 [Oryza sativa (japonica cultivar-group)] |
| 86 | 279 | gb\|AAL73485.1\|repressor protein [Oryza sativa] |
| 87 | 280 | gb\|AAL73488.1\|repressor protein [Zea mays] |
| 88 | 281 | gb\|AAB63549.1\|putative protein kinase [Arabidopsis thaliana] |
| 89 | 282 | gb\|AAB63549.1\|putative protein kinase [Arabidopsis thaliana] |
| 90 | 283 | ref\|NP_197112.1\|expressed protein [Arabidopsis thaliana] |
| 91 | 284 | ref\|NP_850005.1\|expressed protein [Arabidopsis thaliana] |
| 92 | 285 | ref\|NP_195009.1\|protein kinase, putative [Arabidopsis thaliana] |
| 93 | 286 | ref\|NP_195009.1\|protein kinase, putative [Arabidopsis thaliana] |
| 94 | 287 | ref\|NP_973478.1\|protein kinase, putative [Arabidopsis thaliana] |
| 95 | 288 | ref\|NP_973478.1\|protein kinase, putative [Arabidopsis thaliana] |
| 96 | 289 | ref\|NP_201267.1\|invertase/pectin methylesterase inhibitor family protein [Arabidopsis thaliana] |
| 97 | 290 | ref\|NP_201267.1\|invertase/pectin methylesterase inhibitor family protein [Arabidopsis thaliana] |
| 98 | 291 | ref\|NP_013662.1\|Hypothetical ORF; Ym1050wp [Saccharomyces cerevisiae] |
| 99 | 292 | ref\|NP_013662.1\|Hypothetical ORF; Ym1050wp [Saccharomyces cerevisiae] |
| 100 | 293 | ref\|NP_567548.1\|pseudo-response regulator 2 (APRR2) (TOC2) [Arabidopsis thaliana] |
| 101 | 294 | ref\|NP_566402.1\|U-box domain-containing protein [Arabidopsis thaliana] |
| 102 | 295 | gb\|AAL47207.1\|HAP3-like transcriptional-activator [Oryza sativa (indica cultivar-group)1 |
| 103 | 296 | gb\|AAB23208.1\|isocitrate lyase, threo-D S-isocitrate glyoxylate-lyase, IL [EC 4.1.3.1] [Brassica napus, |
| | | seedlings, Peptide, 576 aa] |
| 104 | 297 | ref\|XP_478504.1\|putative isocitrate lyase [Oryza sativa (japonica cultivar-group)] |
| 105 | 298 | emb\|CAE03088.2\|OSJNBa0017B10.3 [Oryza sativa (japonica cultivar-group)] |
| 106 | 299 | gb\|AAV63915.1\|hypothetical protein At4g03965 [Arabidopsis thaliana] |
| 107 | 300 | sp\|O48646\|GPX4_ARATH Probable phospholipid hydroperoxide glutathione peroxidase, mitochondrial |
| | | precursor (PHGPx) (AtGPX1) |
| 108 | 301 | ref\|NP_192897.2\|glutathione peroxidase, putative [Arabidopsis thaliana] |
| 109 | 302 | ref\|XP_464932.1\|cytochrome c biogenesis protein-like [Oryza sativa (japonica cultivar-group)] |
| 110 | 303 | gb\|AA042384.1\|putative major intrinsic protein [Arabidopsis thaliana] |
| 111 | 304 | ref\|XP_482812.1\|major intrinsic protein-like [Oryza sativa (japonica cultivar-group)] |
| 112 | 305 | ref\|XP_481321.1\|unlcnown protein [Oryza sativa (japonica cultivar-group)] |
| 113 | 306 | ref\|NP_229483.1\|cold shock protein [Thermotoga maritima MSB8] |
| 114 | 307 | ref\|NP_229670.1\|cold shock protein [Thermotoga maritima MSB8] |
| 115 | 308 | ref\|NP_623328.1\|Cold shock proteins [Thermoanaerobacter tengcongensis MB4] |
| 116 | 309 | ref\|YP_147209.1\|cold shock protein [Geobacillus kaustophilus HTA426] |
| 117 | 310 | ref\|YP_050486.1\|cold shock protein [Erwinia carotovora subsp. atroseptica SCRI1043] |
| 118 | 311 | ref\|XP_450543.1\|unknown protein [Oryza sativa (japonica cultivar-group)] |
| 119 | 312 | ref\|NP_974566.1\|calcineurin B-like protein 1 (CBL1) [Arabidopsis thaliana] |
| 120 | 313 | ref\|NP_849501.1\|phospholipase D delta/PLD delta (PLDDELTA) [Arabidopsis thaliana] |
| 121 | 314 | emb\|CAB80188.1\|arginine decarboxylase SPE2 [Arabidopsis thaliana] |
| 122 | 315 | gb\|AAC17191.1\|spermidine synthase [Saccharomyces cerevisiae] |
| 123 | 316 | spIP502641FMS1_YEAST Polyamine oxidase FMS1 (Fenpropimorph resistance multicopy suppressor 1) |
| 124 | 317 | ref\|NP_567276.1\|amidohydrolase family protein [Arabidopsis thaliana] |
| 125 | 318 | gb\|AAQ06658.1\|apetala2 domain-containing CBF1-like protein [Oryza saliva] |
| 126 | 319 | gb\|AAN76804.1\|DREB-like protein [Zea mays] |
| 127 | 320 | gb\|AAG40345.1\|AT5g17460 [Arabidopsis thaliana] |
| 128 | 321 | ref\|NP_012214.1\|Pho85p cyclin of the Pho80p subfamily, forms a functional kinase complex with Pho85p |
| | | which phosphotylates Mmr1p and is regulated by Pho81p; involved in glycogen metabolism, expression is cell- |
| | | cycle regulated; Pc17p [Saccharomyces cerevisiae] |
| 129 | 322 | ref\|NP_354295.1\|hypothetical protein AGR_C_2368 [Agrobacterium tumefaciens str. C58] |
| 130 | 323 | ref\|NP_187978.1\|seven in absentia (SINA) family protein [Arabidopsis thaliana] |
| 131 | 324 | ref\|NP_187978.1\|seven in absentia (SINA) family protein [Arabidopsis thaliana] |
| 132 | 325 | ref\|NP_198627.1\|ASF1-like anti-silencing family protein [Arabidopsis thaliana] |
| 133 | 326 | dbj\|BAB19648.1\|preprophytosulfokine [Oryza sativa] |
| 134 | 327 | ref\|NP_190653.2\|phototropic-responsive NPH3 family protein [Arabidopsis thalianal |
| 135 | 328 | ref\|NP_173200.1\|ribosomal protein L14 family protein [Arabidopsis thaliana] |
| 136 | 329 | ref\|NP_440132.1\|transaldolase [Synechocystis sp. PCC 6803] |
| 137 | 330 | pirl1S29317 phosphoprotein phosphatase (EC 3.1.3.16) 1-maize gb\|AAA33545.1\|protein phosphatase-1 |
| 138 | 331 | ref\|YP_148593.1\|6-phosphofructokinase (phosphofructokinase) (phosphohexokinase) [Geobacillus kaustophilus |
| | | HTA426] |
| 139 | 332 | ref\|YP_148593.1\|6-phosphofructokinase (phosphofructokinase) (phosphohexokinase) [Geobacillus kaustophilus |
| | | HTA426] |
| 140 | 333 | ref\|NP_974942.1\|F-box family protein [Arabidopsis thaliana] |
| 141 | 334 | ref\|XP_480608.1\|putative gamma-aminobutyrate transaminase subunit precursor isozyme 3 [Oryza sativa |
| | | (japonica cultivar-group)] |
| 142 | 335 | emb\|CAB80281.1\|NAD+ dependent isocitrate dehydrogenase-like protein [Arabidopsis thaliana] |
| 143 | 336 | ref\|NP_176491.1\|AP2 domain-containing transcription factor, putative [Arabidopsis thaliana] |
| 144 | 337 | ref\|NP_918065.1\|putative fatty acid condensing enzyme CUT1 [Oryza sativa (japonica cultivar-group)] |
| 145 | 338 | ref\|NP_565174.1\|14-3-3 protein GF14 pi (GRF13) [Arabidopsis thaliana] |
| 146 | 339 | ref\|NP_565174.1\|14-3-3 protein GF14 pi (GRF13) [Arabidopsis thaliana] |
| 147 | 340 | pirlIT02447 hypothetical protein At2g46000 Arabidopsis thaliana |
| 148 | 341 | gb\|AAM63147.1\|unknown [Arabidopsis thaliana] |
| 149 | 342 | gb\|AAY78681.1\|putative E3 ubiquitin ligase SCF complex subunit SKPl/ASK1 [Arabidopsis thaliana] |
| 150 | 343 | dbj\|BAD43212.1\|putative glutamate/aspartate-binding peptide [Arabidopsis thaliana] |
| 151 | 344 | dbj\|BAD43212.1\|putative glutamate/aspartate-binding peptide [Arabidopsis thaliana] |
| 152 | 345 | ref\|NP_568396.1\|ring-box protein-related [Arabidopsis thaliana] |
| 153 | 346 | emb\|CAB78344,1\|serine/threonine-specific protein kinase MHK [Arabidopsis thaliana] |
| 154 | 347 | emb\|CAB61629.1\|spermidine synthase 1 [Oryza sativa] |
| 155 | 348 | ref\|NP_564556.1\|zinc finger (C3HC4-type RING finger) family protein [Arabidopsis thaliana] |
| 156 | 349 | ref\|NP_564556.1\|zinc finger (C31IC4-type RING finger) family protein [Arabidopsis thaliana] |
| 157 | 350 | ref\|NP_172848.1\|eukaryotic translation initiation factor 5A-1/eIF-5A 1 [Arabidopsis thaliana] , |
| 158 | 351 | ref\|NP_177100.1\|eukaryotic translation initiation factor 5A, putative/eIF-5A, putative [Arabidopsis thaliana] |
| 159 | 352 | emb\|CAB62652.1\|rac-like GTP binding protein Aracll [Arabidopsis thaliana] |
| 160 | 353 | ref\|NP_178062.1\|succinate-semialdehyde dehydrogenase (SSADH1) [Arabidopsis thaliana] |
| 161 | 354 | ref\|NP_849582.1\|expressed protein [Arabidopsis thaliana] |
| 162 | 355 | gb\|AAT93875.1\|unknown protein [Oryza sativa (japonica cultivar-group)] |
| 163 | 356 | ref\|NP_200265.1\|expressed protein [Arabidopsis thaliana] |
| 164 | 357 | ref\|XP_466086.1\|putative multiple stress-responsive zinc-finger protein [Oryza sativa (japonica cultivar-group)] |
| 165 | 358 | ref\|NP_180514.1\|DNA-directed RNA polymerase I(A) and III(C) 14 kDa subunit (RPAC14) [Arabidopsis |
| | | thaliana] |
| 166 | 359 | splQ6UNT2\|RL5_CUCSA 60S ribosomal protein L5 |
| 167 | 360 | ref\|NP_192634.1\|phosphate-responsive protein, putative (EXO) [Arabidopsis thaliana] |
| 168 | 361 | ref\|XP_506910.1\|PREDICTED OSJNBa0057007.4 gene product [Oryza sativa (japonica cultivar-group)] |
| 169 | 362 | emb\|CAC03739.1\|flavin containing polyamine oxidase [Zea mays] |
| 170 | 363 | ref\|XP_466372.1\|cryptochrome la [Oryza sativa (japonica cultivar-group)] |
| 171 | 364 | gb\|AAX96768.1\|expressed protein [Oryza sativa (japonica cultivar-group)] |
| 172 | 365 | ref\|NP_199265.1\|ribose 5-phosphate isomerase-related [Arabidopsis thaliana] |
| 173 | 366 | dbj\|BAB07982.1\|FPF1 protein-like [Oryza sativa (japonica cultivar-group)] |
| 174 | 367 | ref\|XP_323418.1\|hypothetical protein [Neurospora crassa] |
| 175 | 368 | ref\|NP_564152.1\|expressed protein [Arabidopsis thaliana] |
| 176 | 369 | ref\|NP_564354.1\|early-responsive to dehydration stress protein (ERD4) [Arabidopsis thaliana] |
| 177 | 370 | ref\|NP_974167.1\|WD-40 repeat family protein [Arabidopsis thaliana] |
| 178 | 371 | ref\|NP_565168.1\|WD-40 repeat family protein [Arabidopsis thaliana] |
| 179 | 372 | ref\|NP_190318.1\|asparagine synthetase 1 [glutamine-hydrolyzing]/glutamine-dependent asparagine synthetase |
| | | 1 (ASN1) [Arabidopsis thaliana] |
| 180 | 373 | ref\|NP_850664.1\|asparagine synthetase 1 [glutamine-hydrolyzing]/glutamine-dependent asparagine synthetase |
| | | 1 (ASNI) [Arabidopsis thaliana] |
| 181 | 374 | ref\|NP_850663.1\|asparagine synthetase 1 [glutamine-hydrolyzing]/glutamine-dependent asparagine synthetase |
| | | 1 (ASN1) [Arabidopsis thaliana] |
| 182 | 375 | ref\|NP_196821.1\|nodulin MtN3 family protein [Arabidopsis thaliana] |
| 183 | 376 | ref\|NP_179580.1\|malate oxidoreductase, putative [Arabidopsis thaliana] |
| 184 | 377 | ref\|NP_196510.1\|hydroxyproline-rich glycoprotein family protein [Arabidopsis thaliana] |
| 185 | 378 | ref\|NP_196515.1\|hydroxyproline-rich glycoprotein family protein [Arabidopsis thaliana] |
| 186 | 379 | ref\|NP_181807.1\|citrate synthase, glyoxysomal, putative [Arabidopsis thaliana] |
| 187 | 380 | ref\|NP_191179.1\|amino acid transporter family protein [Arabidopsis thaliana] |
| 188 | 381 | ref\|NP_195772.1\|zinc finger (C3HC4-type RING finger) family protein [Arabidopsis thaliana] |
| 189 | 382 | ref\|NP_195772.1\|zinc finger (C3HC4-type RING finger) family protein [Arabidopsis thaliana] |
| 190 | 383 | dref\|NP_201479.1\|expressed protein [Arabidopsis thaliand |
| 191 | 384 | ref\|NP_568905.1\|lipid transfer protein 3 (LTP3) [Arabidopsis thaliana] |
| 192 | 385 | gbIAAM14303.1\|putative homeodomain transcription factor protein ATHB-7 [Arabidopsis thaliana] |
| 193 | 386 | ref\|NP_180896.1\|calcium-binding RD20 protein (RD20) [Arabidopsis thaliana] |

TABLE 10A
Examples of gene/protein molecules for end user traits suitable for
use in the present invention. All references are incorporated by reference.

| Trait | Gene/protein | Reference |
|---|---|---|
| Enhanced | glutamate dehydrogenase | U.S. Pat. No. 6,969,782 |
| amino acid | threonine deaminase | US20050289668 |
| content | dihydrodipicolinic acid | U.S. Pat. No. 5,258,300 |
| | synthase (dap A) | |
| Enhanced | several | US20050055746 |
| protein | | |
| content | | |
| Modified fatty | several | U.S. Pat. No. 6,949,698; |
| acids | | U.S. Pat. Nos. 6,444,876; |
| | | 6,426,447; 6,380,462; |
| | | U.S. Pat. Nos. |
| | | U.S. Pat. Nos. 6,949,698; |
| | | 6,828,475; 6,822,141; |
| | | 6,770,465; 6,706,950; |
| | | 6,660,849; 6,596,538; |
| | | 6,589,767; 6,537,750; |
| | | 6,489,461; 6,459,018 |
| Enhanced oil | several | U.S. Pat. Nos. 6,495,739; |
| content | | 5,608,149; 6,483,008; |
| | | 6,476,295 |
| Carbohydrate | raffinose saccharides | U.S. Pat. No. 6,967,262 |
| production | | |
| Starch | several | U.S. Pat. Nos. 6,951,969; |
| production | | 6,538,181; 6,538,179; |
| | | 6,538,178; 5,750,876; |
| | | 6,476,295 |
| Phytic acid | inositol polyphosphate 2-kinase | WO06029296 |
| reduction | inositol 1,3,4-triphosphate 5/6- | US20050202486 |
| | kinases | |
| Processing | Several | WO05096804; U.S. |
| enzymes | Alpha-amylase | Pat. No. 5,543,576 |
| production | phytase | US20060200877 |
| | | US20030170293 |
| Biopolymers | several | U.S. Pat. Nos. |
| | | USRE37,543; |
| | | 6,228,623; 5,958,745 |
| | | and U.S. Patent |
| | | Publication No. |
| | | US20030028917 |
| Enhanced | several | U.S. Pat. Nos. 6,723,837; |
| nutrition | | 6,653,530; 6,5412,59; |
| | | 5,985,605; 6,171,640 |
| Pharmaceutical | several | U.S. Pat. Nos. 6,812,379; |
| peptides and | | 6,774,283; 6,140,075; |
| secretable | | 6,080,560 |
| peptides | | |
| Improved | sucrose phosphorylase | U.S. Pat. No. 6,476,295 |
| processing trait | | |
| Improved | thioredoxin and/or thioredoxin | U.S. Pat. No. 6,531,648 |
| digestibility | reductase | |

TABLE 10B
Examples of gene/protein molecules for end user traits suitable for use in the present invention are disclosed in the following
references which are incorporated by reference. Indicated SEQ ID NOS. represents use in preferred embodiments.

| | | |
|---|---|---|
| Gene/Protein Name | SEQ ID NO | Reference |

Enhanced protein and/or amino acid content

| | | |
|---|---|---|
| ABI4-like | SEQ ID NOS: 2, 3,4, 5 | U.S. Pat. No. 7,176,027 |
| ABI4-like polypeptide | SEQ ID NOS: 2, 4, 5 | US20030140381A1 |
| Anthranilate synthase | | WO07008546A2 |
| Anthranilate synthase | SEQ ID NOS: 4, 7, 43, 58, 59, 60, 61, 62, 63, 64, 65, 69, | WO02090497A2 |
| | 70, 77, 78, 79, 80, 81, 82 | |
| Anthranilate synthasc | SEQ ID NOS: 12, 16 | U.S. Pat. No. 6,118,047 |
| Anthranilate synthase | SEQ ID NOS: 1, 2, 8, 12, 14, 16 | US20020061570A1 |
| Anthranilate synthase | SEQ ID NOS: 1, 2, 8, 12, 14, 16 | U.S. Pat. No. 6,271,016 |
| Brassica seed coat gene | US20040250314A | |
| Carbonic anhydrase, ACCase, LPAT, | | US20040133944A1 |
| DGAT, oleosin | | |
| Cdc25 phosphoprotein phosphatase | | WO0052171A1 |
| Com accession REN 001 | | WO03056903A2 |
| Cyclin-dependent kinase | SEQ ID NOS: 1, 3, 5 | US20040003433A1 |
| Delta-12 fatty acid desaturase | SEQ ID NOS: 2275-2672, 5959, 5972, 5973, 5977-5990, | US20040016025A1 |
| | 6001, 1598-1885, 5960-5971, 1886-1918, 2554, 1919- | |
| | 2085, 2144-2274, 2086-214 | |
| Delta-15 fatty acid desaturase | | US20040010819A1 |
| Fungal hexokinase | SEQ ID NOS: 2, 6, 8, 10, 12, 14, 16, 18, 20, 22, 24, 26, | US20040237138A1 |
| | 28, 30 | |
| Glutamate dehydrogenase | | US20020069430A1 |
| Myo-inositol kinase | SEQ ID NOS: 1, 3, 4, 35 | US20050289670A1 |
| Plastid-specific transit peptide | SEQ ID NOS: 3, 7, 9 | WO9720941A1 |
| Prolamin box binding factor peptide | | US20030051272A1 |
| Prolamin box binding factor peptide (PBF) | SEQ ID NO: 29 | U.S. Pat. No. 7,157,623 |
| Pyruvate carboxylase | | WO0127258A2 |
| RNA binding activity | SEQ ID NOS: 1, 3, 5,7, 9, 11, 13, 28, 29, 30 | US20050138686A1 |
| Several | | WO04063333A2 |
| SSE1 protein | SEQ ID NOS: 1-14 | US20030084475A1 |
| Sucrose phosphorylase | SEQ ID NO: 6 | USRE39114 |
| Sucrose phosphorylase | SEQ ID NO: 5 | US20030110535A1 |
| Sucrose phosphorylase | | U.S. Pat. No. 6222098 |
| Threonine aldolase | SEQ ID NOS: 1, 3, 5, 7, 9, 11, 13, 15 | WO05047472A2 |
| Zpr10/(22) | | WO9001869A1 |
| | SEQ ID NOS: 1-36 | WO03027249A2 |
| | SEQ ID NOS: 1 through 47374 | US20030233675A1 |
| | SEQ ID NOS: 2, 3, 4, 5 | US20030140381A1 |
| | | US20040060082A1 |
| | | WO04006659A1 |
| | | US20040060082A1 |
| | | WO04006659A1 |

Enhanced Oil Content and/or Modified Fatty Acid Content

| | | |
|---|---|---|
| ABI4-like | SEQ ID NOS: 2, 3, 4, 5 | U.S. Pat. No. 7,176,027 |
| ABI4-like polypeptide | SEQ ID NOS: 2, 4, 5 | US20030140381A1 |
| accD gene | | JP2002335786A2 |
| accD gene | | JP2002335786A2 |
| Acetyl-CoA carboxylase | | WO9967367A1 |
| AGL11 gene | | U.S. Pat. No. 7,179,957 |
| ATCC Accession Number 209181 | | U.S. Pat. No. 5,912,416 |
| Beta-ketoacyl-CoA synthase | SEQ ID NOS: 1-24 | WO06066859A |
| Beta-ketoacyl-CoA synthase | SEQ ID NOS: 1 through 23 (odd number sequences only) | WO06066859A1 |
| Brassica seed coat gene | | US20040250314A |
| Brassica seed coat gene | | US20030208793A1 |
| Brassica seed coat gene | | US20030163843A1 |
| CGPG894 | SEQ ID NOs: 250, 3 | WO06076423 |
| Com accession REN 001 | | WO03056903A2 |
| Delta 12-FAD | SEQ ID NO: 9 | U.S. Pat. No. 7,135,614 |
| Delta 12-FAD | | EP0880312B1 |
| Delta 6-desaturase | SEQ ID NO: 4 | EP0801680B1 |
| Delta 6-desaturase | | WO9621022A2 |
| Delta-12 and delta-15 desaturase | | U.S. Pat. No. 5850026 |
| Delta-12 FAD | SEQ ID NOS: 2275-2672, 5959, 5972, 5973, 5977-5990, | US20040016025A1 |
| | 6001, 1598-1885, 5960-5971, 1886-1918, 2554, 1919- | |
| | 2085, 2144-2274, 2086-214 | |
| Delta-15 FAD | | US20040010819A1 |
| Desaturase gene | | WO0011012A1 |
| Diacylglycerol acyltransferase | SEQ ID NOS: 33, 34, others | WO04011671A2; |
| | | U.S. Pat. No. 6,822,141; |
| | | US 20040107459; |
| | | U.S. Pat. No. 7,135,617; |
| | | U.S. Pat. No. 6,444,876 |
| Fatty acid desaturase 2-like (FAD2-like) | SEQ ID NOS: 5-36 | WO06073787A2 |
| polypeptide | | |
| Fatty acid modifying enzyme | SEQ ID NOS: 1, 3, 19, 23, 29 | US20030126640A1 |
| Fatty acid modifying enzyme | SEQ ID NOS: 1, 3, 19, 23, 29 | US20030024010A1 |
| Fatty acyl hydroxylase | | WO9610075A1 |
| Fertility restorer gene | | EP0853874A1 |
| Hexokinase | SEQ ID NOS: 2, 6, 8, 10, 12, 14, 16, 18, 20, 22, 24, 26, | US20040237138A1 |
| | 28, 30 | |
| HOI001 granule bound starch synthase | SEQ ID NOS: 1, 11, 3 | U.S. Pat. No. 7,179,956 |
| G200 | SEQ ID NOS: 262, 263 | US20070022495 |
| Gamma-tocopherol methyltransferase | | WO9904622A1 |
| Homozygous recessive gene pair fan3fan3 | | EP097337513 1 |
| Isocitrate lyase (ICL) | SEQ ID NO: 2 | US20050257288A1 |
| Lipid metabolism protein | SEQ ID NOS: 1-32 | WO03014376A2 |
| Lipid metabolism protein (LMP) | SEQ ID NOS: 1-31 (odd numbered sequences only) | US20030154512A1 |
| LMP polynucleotide sequences | SEQ ID NOS: 1-8, 18-28 | WO07016521A2 |
| Microsomal delta-15 fatty acid desaturase | | US20060137040A1 |
| MYB transcription factor | SEQ ID NOS: 13-27 | US20040006797A1 |
| Myo-inositol kinase | SEQ ID NOS: 1, 3, 4, 35 | US20050289670A1 |
| oil-associated gene | | WO05024017A1 |
| Ovule development protein 2 (ODP2) | SEQ ID NO: 2 | US20050257289A1 |
| Plastid ADP/ATP translocator | SEQ ID NOS: 5, 6, 7 | U.S. Pat. No. 6,891,088 |
| SLC1-1 | SEQ ID NOS: 1, 3 | U.S. Pat. No. 6,051,755 |
| SSE1 protein | SEQ ID NOS: 1 -14 | US20030084475A1 |
| Stearoyl-ACP desaturase and a delta-12 | | WO9964579A2 |
| desaturase | | |
| Sucrose phosphorylase | SEQ ID NO: 6 | USRE39114 |
| Sucrose phosphorylase | SEQ ID NO: 5 | US20030110535A1 |
| Sucrose phosphorylase | | U.S. Pat. No. 6,222,098 |
| Transcription factor | SEQ ID NOS: 1796, 1995 | US20070022495A1 |
| Transcription factor | | US20030188330A1 |
| Yeast SLC1-1 or SLC1 | SEQ ID NOS: 1, 3 | WO9638573A1 |
| | SEQ ID NOS: 2-252, 270-288 | WO06056468A1 |
| | (even numbered sequences) | US20040060082A1 |
| | | WO04006659A1 |
| | SEQ ID NOS: 1 through 47374 | US20030233675A1 |
| | SEQ ID NOS: 2, 3, 4, 5 | US20030140381A1 |
| | SEQ ID NOS: 1, 2 | US20060107348A1 |
| | | WO06039449A1 |
| | | US20040060082A1 |
| | | WO04006659A1 |
| | SEQ ID NOS: 1, 3, 12 | US20030204871A1 |
| | SEQ ID NOS: 2, 4 | US20030188330A1 |
| | SEQ ID NOS: 1 through 105,582 | US20070044171A1 |
| | | US7173162 |
| | | US20030066105A1 |
| | | EP1090544A4 |
| | | US20020129408A1 |
| | | US20020088020A1 |
| | | EP0813357B1 |
| | | U.S. Pat. No. 5,840,946 |
| | | U.S. Pat. No. 5,545,821 |
| | | U.S. Pat. No. 5,387,758 |
| | | EP0323753B1 |
| | SEQ ID NOS: 1, 2, 4, 6, 8, 10, 12, 71, 14 | US20060143728A1 |
| | | US20040025202A1 |

TABLE 11
Examples of corn inbred lines suitable for use in the present invention.

| Patent | | Patent | | Patent | | Patent | | Patent | |
|---|---|---|---|---|---|---|---|---|---|
| Number | Line | Number | Line | Number | Line | Number | Line | Number | Line |
| 5,731,492 | PH19A | 5,731,500 | CG5NF22 | 5,633,427 | PHHB | 5,567,861 | PHN46 | 5,489,744 | 4P33339 |
| 5,731,493 | PH63B | 5,731,501 | CG4NU15 | 5,633,428 | LH262 | 5,569,813 | ZS0223 | 5,491,286 | PHKM5 |
| 5,811,645 | NP2045 | 5,750,834 | PH80B | 5,633,429 | LH227 | 5,569,816 | phajo | 5,491,293 | LH225 |
| 5,817,914 | NP993 | 5,750,835 | PH47A | 5,639,941 | LH226 | 5,569,817 | PHJJ3 | 5,491,294 | LH185 |
| 5,824,843 | LH290 | 5,750,847 | PH38D | 5,639,942 | LH235 | 5,569,818 | phap8 | 5,491,296 | LH176 |
| 5,824,848 | F361 | 5,750,849 | PH05W | 5,639,943 | LH234 | 5,569,819 | PHPP8 | 5,495,065 | PHW06 |
| 5,824,849 | 171KI3 | 5,750,850 | LH242 | 5,639,946 | PHDP0 | 5,569,820 | ZS1284 | 5,495,067 | LH252 |
| 5,824,852 | NP2013 | 5,750,851 | QH101 | 5,675,066 | PH06N | 5,569,821 | PHT11 | 5,495,068 | LH231 |
| 5,841,015 | PH05G | 5,750,852 | NR109 | 5,684,227 | LH177 | 5,569,822 | phte4 | 5,495,069 | PHTE4 |
| 5,844,116 | PH1W2 | 5,763,743 | PH63A | 5,689,034 | PH24E | 5,569,826 | ZS0114 | 5,506,367 | PHP38 |
| 5,844,117 | PHOGP | 5,763,744 | PH67A | 5,708,189 | PHP38 | 5,576,473 | 7054 | 5,506,368 | PHN82 |
| 5,850,007 | PH1MR | 5,763,746 | PH20A | 5,714,671 | ASG06 | 5,585,533 | ZS0560 | 5,527,986 | PHTD5 |
| 5,850,008 | LH263 | 5,763,752 | ZSO1602 | 5,723,721 | CG00685 | 5,585,534 | ZS0853 | 5,530,181 | 899 |
| 5,850,009 | PH0HC | 5,763,753 | ZS01262 | 5,723,722 | PHND1 | 5,585,539 | ZS1791 | 5,530,184 | PHAP1 |
| 5,850,010 | PH56C | 5,763,754 | ZS01219 | 5,723,723 | PH44A | 5,585,541 | ZS1513 | 5,534,661 | PHKW3 |
| 5,856,614 | 01IZB2 | 5,763,755 | ZSO1172 | 5,723,724 | ZS01591 | 5,589,606 | ZS1679 | 5,536,900 | CG00653 |
| 5,859,313 | PHKVO | 5,763,757 | PH07D | 5,723,725 | ZS01101 | 5,602,314 | ZS1022 | 5,541,352 | PHRD6 |
| 5,859,316 | PH0HR | 5,763,759 | LH291 | 5,723,726 | ZS01452 | 5,602,315 | ZS1202 | 5,543,575 | PHK46 |
| 5,859,317 | PH22G | 5,767,339 | 85857 | 5,723,727 | ZS01429 | 5,602,316 | ZS1783 | 5,545,809 | PHBG4 |
| 5,859,322 | 3ISI2 | 5,767,340 | PHBR2 | 5,723,728 | ZS01819 | 5,602,318 | PHDG1 | 5,545,811 | LH189 |
| 5,859,353 | AR5252bm3 | 5,767,341 | LH228 | 5,723,729 | ZS01250 | 5,608,138 | PHKV1 | 5,545,812 | PHNJ2 |
| 5,859,354 | PH09B | 5,770,790 | PH41E | 5,723,730 | ZS01595 | 5,608,139 | PHO5F | 5,545,813 | PHRF5 |
| 5,859,355 | 17DHD12 | 5,773,683 | LH283 | 5,728,923 | CG3ND97 | 5,608,140 | PH38B | 5,545,814 | PHFR8 |
| 5,861,541 | PH10A | 5,777,196 | 01CSI6 | 5,728,924 | NP938(934) | 5,618,987 | PH42B | 5,557,034 | PHN18 |
| 5,866,763 | ZS01220 | 5,777,197 | 3INI2 | 5,731,491 | PHNG2 | 5,625,129 | PHDD6 | 5,557,038 | PHTP9 |
| 5,866,767 | PH79A | 5,780,705 | ZS01301 | 5,731,502 | CG5NA58 | 5,625,131 | ZS0541 | 5,563,320 | PH54B |
| 5,866,768 | PHO2T | 5,792,905 | NP982 | 5,731,503 | NP948 | 5,625,132 | PH08B | 5,563,321 | PHGF5 |
| 5,880,337 | 4IBZIA | 5,792,906 | NP2034 | 5,731,504 | LH236 | 5,625,133 | PHOC7 | 5,563,322 | PHAG6 |
| 5,880,338 | 7523 | 5,792,911 | PH24M | 5,731,506 | CG00766 | 5,625,135 | LH233 | 5,563,323 | PHAP9 |
| 5,880,339 | SNBK | 5,792,912 | PH00M | 5,750,829 | PHOAA | 5,723,731 | ASG05 | 5,563,325 | PHBE2 |
| 5,880,341 | 91INH2 | 5,792,915 | PHOAV | 5,750,830 | PH15A | 5,723,739 | LH281 | 5,563,327 | ZS0510 |
| 5,880,342 | 17DIA1 | 5,811,637 | PH40B | 5,750,831 | PH25A | 5,728,919 | PHBF0 | 5,602,317 | PHAAO |
| 5,880,347 | LH264 | 5,811,641 | LH179 | 5,750,832 | PH44G | 5,728,922 | CG5NA01 | 5,880,348 | LH273 |
| 6,127,608 | 7791 | 6,143,961 | RQAA8 | 6,084,160 | PH0CD | 5,977,458 | AR5651bm3 | 5,880,349 | 7571 |
| 6,242,675 | LH293 | 6,143,962 | PH2KN | 6,084,161 | ASG25 | 5,977,459 | LH266 | 5,880,350 | LH237 |
| 6,245,975 | LH245 | 6,147,284 | PH2E4 | 6,084,162 | 86ISI15 | 5,977,460 | LH303 | 5,889,188 | PH0B4 |
| 6,248,941 | 17DHD16 | 6,150,590 | LH267 | 6,084,163 | BE4547 | 5,981,855 | LH301 | 5,902,922 | FEBS |
| 6,252,146 | 90DHQ2 | 6,153,817 | PH0DH | 6,091,007 | PH21T | 5,986,182 | 4SQ601 | 5,905,191 | 8F286 |
| 6,252,147 | LH279 | 6,156,957 | NP991 | 6,096,952 | 01DHD16 | 5,986,184 | PH1TB | 5,910,625 | 3AZA1 |
| 6,252,148 | LH244 | 6,160,210 | 8982-11-4-2 | 6,096,953 | PH224 | 5,986,185 | PH24D | 5,910,635 | 91DFA-5 |
| 6,259,004 | PH2VE | 6,166,303 | PH1CP | 6,103,958 | ASG26 | 5,986,186 | LH229 | 5,910,636 | ASG20 |
| 6,265,645 | 8849 | 6,166,304 | NP2208 | 6,103,959 | ASG28 | 5,986,187 | LH277 | 5,912,420 | ZS03940 |
| 6,281,414 | LH287 | 6,169,230 | 29MIFI2 | 6,107,550 | PH0V0 | 5,990,393 | PH1CN | 5,912,421 | 91ISI6 |
| 6,291,748 | WDHQ11 | 6,169,231 | RQAB7 | 6,111,171 | 90LCL6 | 5,990,394 | LH261 | 5,914,452 | MF1113B |
| 6,303,850 | 09DSS1 | 6,169,233 | NP2031 | 6,111,172 | 22DHD11 | 5,990,395 | W1498A | 5,917,125 | PH03D |
| 6,310,274 | PH36E | 6,172,284 | ZS02461 | 6,114,606 | ASG17 | 5,994,631 | WQDS2 | 5,917,134 | PHDN7 |
| 6,313,381 | PH50P | 6,172,285 | LH198Bt810 | 6,114,609 | AR5253bm3 | 5,998,710 | NL085B | 5,920,003 | 01DIB2 |
| 6,313,382 | PH8V0 | 6,175,063 | 3DHA9 | 6,114,610 | ASG27 | 5,998,711 | PH09E | 5,922,935 | 82DHB1 |
| 6,313,383 | PH4TV | 6,175,064 | LH200BT810 | 6,114,611 | WDHQ2 | 6,015,944 | LH284 | 5,922,936 | 8M222 |
| 6,313,384 | PH2JR | 6,180,858 | LH172Bt810 | 6,114,613 | PH3GR | 6,020,543 | PH1B5 | 5,929,313 | PHMJ2 |
| 6,316,701 | NP2138 | 6,184,444 | NP2115 | 6,118,051 | PH1NF | 6,025,547 | PH1CA | 5,932,787 | SBB1 |
| 6,316,702 | PH4PV | 6,184,445 | PH3P0 | 6,118,053 | PH0JG | 6,031,160 | 7OLDL5 | 5,932,788 | 86ISI3 |
| 6,316,703 | PH3DT | 6,184,446 | GSC3 | 6,118,054 | PH189 | 6,031,161 | GM9215 | 5,936,144 | ZS01231 |
| 6,316,704 | PH5D6 | 6,184,447 | GSC1 | 6,118,055 | PH12J | 6,031,162 | 90LDI1 | 5,936,145 | 87DIA4 |
| 6,320,106 | PH9K0 | 6,187,999 | NP2017 | 6,118,056 | PH1EM | 6,034,304 | 90LDC2 | 5,936,146 | 79310J2 |
| 6,323,403 | GF6150 | 6,188,001 | PH1W0 | 6,121,519 | 90DJD28 | 6,034,305 | 90QDD1 | 5,936,148 | PH1GC |
| 6,329,575 | 2227BT | 6,188,002 | 01IZB2 | 6,121,520 | PH12C | 6,034,306 | R398D | 5,939,606 | 01DHD10 |
| 6,329,578 | ZS02433 | 6,188,003 | R762 | 6,121,522 | PH55C | 6,037,531 | RDBQ2 | 5,939,607 | PH2CB |
| 6,333,451 | PH0B3 | 6,191,344 | ASG10 | 6,121,523 | PH3EV | 6,040,506 | HX621 | 5,939,608 | PH080 |
| 6,333,453 | PH2EJ | 6,191,345 | LH253 | 6,121,525 | ZS4199 | 6,040,507 | HX622 | 5,942,670 | PH14T |
| 6,340,786 | 17INI30 | 6,194,642 | GSC2 | 6,124,529 | PH2V7 | 6,040,508 | 01HG12 | 5,942,671 | PH185 |
| 6,346,660 | MR724 | 6,201,171 | 86ISI27 | 6,124,530 | PH4TF | 6,043,416 | HX740 | 5,948,957 | PH19V |
| 6,353,158 | ZS02234 | 6,204,438 | NP2141 | 6,124,531 | PH3KP | 6,043,417 | 79314N1 | 5,952,551 | ZS09247 |
| 6,353,159 | NP2213 | 6,211,446 | 91ISI5 | 6,124,532 | PH2MW | 6,043,418 | 17INI20 | 5,952,552 | CRAUGSH2W- |
| 6,353,160 | 4SCQ3 | 6,211,447 | 22DHQ3 | 6,124,533 | PH2N0 | 6,046,387 | 17DHD7 | | 89 |
| 6,353,161 | 01HF13 | 6,215,050 | 91INI12 | 6,124,534 | PH1K2 | 6,046,388 | 83INI8 | 5,962,770 | 91DHA1 |
| 6,355,867 | 87ATD2 | 6,222,103 | PH45A | 6,124,535 | PH226 | 6,046,389 | 83InI14 | 5,965,798 | LH300 |
| 6,355,868 | 1874WS | 6,222,104 | NP948 | 6,127,609 | PH2VJ | 6,046,390 | 01INL1 | 5,965,799 | 91ISI4 |
| 6,359,200 | 8M116 | 6,222,105 | 86ISI26 | 6,127,610 | PH1M8 | 6,049,030 | LH286 | 5,969,212 | 79103A1 |
| 6,362,403 | FBLL | 6,222,106 | 01IUL6 | 6,130,369 | WQCD10 | 6,054,640 | ASG29 | 5,969,220 | ASG22 |
| 6,362,404 | 17QFB1 | 6,225,538 | 89ADH11 | 6,130,370 | PH1B8 | 6,060,649 | ASG07 | 5,969,221 | 82IUH1 |
| 6,365,805 | 83DNQ2 | 6,229,075 | R412H | 6,133,512 | 17DHD5 | 6,069,303 | QH111 | 5,969,222 | |
| 6,365,806 | 94INK1A | 6,229,076 | 01HGI4 | 6,133,513 | PH0WD | 6,072,108 | 09DSQ1 | 5,973,238 | LH302 |
| 6,372,969 | NL054B | 6,232,532 | LH185Bt810 | 6,133,514 | PH3GK | 6,072,109 | JCRNR113 | 5,973,239 | LH265 |
| 6,380,467 | 6F545 | 6,232,533 | R372H | 6,137,036 | PH2VK | 6,072,110 | NP2029 | 5,977,451 | PHFW4 |
| 6,384,303 | ZS02338 | 6,232,534 | R660H | 6,137,037 | PH1MD | 6,077,996 | ASG09 | 5,977,452 | 01IBH10 |
| 6,388,177 | F274 | 6,232,535 | 16IUL2 | 6,137,038 | SM4603 | 6,077,997 | PHOWE | 5,977,453 | 91CSI-1 |
| 6,388,178 | MBZA | 6,232,536 | F307W | 6,140,562 | PH04G | 6,077,999 | 86AQV2 | 5,977,455 | WKBC5 |
| 6,388,179 | PH4TW | 6,239,334 | F351 | 6,140,563 | NP2151 | 6,080,919 | PH1GG | 5,977,456 | PH1M7 |
| 6,395,967 | FR3351 | 6,812,388 | I244225 | 6,727,413 | PH5DR | 6,506,965 | RPK7346 | 6,399,860 | R327H |
| 6,914,177 | PH86T | 6,815,592 | PH3AV | 6,730,833 | LH254 | 6,573,438 | NP2044BT | 6,407,320 | FR2108 |
| 6,927,327 | PHAVN | 6,815,593 | G0302 | 6,730,834 | PH5WB | 6,600,095 | PH8W4 | 6,410,830 | FR3383 |
| 6,930,230 | PHB6R | 6,815,594 | G1202 | 6,730,835 | PH7CH | 6,617,500 | M42618 | 6,414,227 | IT302 |
| 6,933,425 | PH91C | 6,818,811 | PH3PG | 6,730,836 | PH54M | 6,624,345 | MV7100 | 6,414,228 | FR3303 |
| 6,933,426 | BE8736 | 6,818,812 | G2202 | 6,730,837 | PH726 | 6,627,800 | 3JP286 | 6,420,634 | 9034 |
| 6,946,590 | MV5125 | 6,822,145 | N16028 | 6,734,348 | PH48V | 6,632,986 | BE4207 | 6,420,635 | G1500 |
| 6,949,699 | PHCWK | 6,828,492 | G4901 | 6,737,566 | PH3PV | 6,632,987 | CI9805 | 6,420,636 | FR3311 |
| 6,956,151 | E24018 | 6,831,216 | LH247 | 6,740,795 | PH77V | 6,635,808 | JCR503 | 6,420,637 | I389972 |
| 6,956,152 | MV8735 | 6,833,499 | KW7648 | 6,740,796 | PH7JB | 6,635,809 | NR401 | 6,423,888 | PH77C |
| 6,956,153 | PHC5H | 6,833,500 | HX894 | 6,740,797 | NP2316 | 6,635,810 | 4VP500 | 6,426,451 | IT201 |
| 6,958,438 | PHACE | 6,835,878 | LH322 | 6,740,798 | PH70R | 6,642,440 | 7SH385 | 6,426,453 | G3000 |
| 6,967,267 | PH77P | 6,835,879 | WICY418C | 6,747,194 | RAA1 | 6,642,441 | KW4773 | 6,429,363 | 94INK1B |
| 6,967,268 | PHB6V | 6,838,601 | LH289 | 6,747,195 | VMM1 | 6,646,187 | NP2073 | 6,433,259 | PH3HH |
| 6,967,269 | PH8JR | 6,844,489 | NP2174 | 6,747,196 | PH3RC | 6,646,188 | PSA104 | 6,433,260 | 6TR512 |
| 6,969,788 | PHBAB | 6,846,976 | PH6WA | 6,753,465 | MNI1 | 6,653,536 | 5XH755 | 6,433,261 | 89AHD12 |
| 6,969,790 | PHB1V | 6,849,790 | G3601 | 6,756,527 | 5750 | 6,653,537 | 1445-008-1 | 6,433,262 | I889291 |
| 6,972,356 | PH3PR | 6,849,791 | PH6CF | 6,756,528 | PH6KW | 6,657,109 | NP2015 | 6,437,223 | 2070BT |
| 6,972,357 | PH8TN | 6,852,914 | HC53 | 6,756,530 | PH951 | 6,660,916 | 7SH383 | 6,437,224 | 3323 |
| 6,974,900 | PH5WA | 6,852,915 | LH283BtMON810 | 6,759,578 | PH6ME | 6,664,451 | LH310 | 6,441,279 | G1900 |
| 6,979,764 | PH58C | 6,855,877 | 85DGD1 | 6,759,579 | NP2171 | 6,670,531 | I880S | 6,441,280 | 16IUL6 |
| 6,979,765 | G6103 | 6,855,878 | PH76T | 6,759,580 | PH87H | 6,677,509 | RR728-18 | 6,444,881 | 7RN401 |
| 6,984,779 | G1103 | 6,858,786 | I390185 | 6,765,132 | PH26N | 6,683,234 | LH320 | 6,444,882 | UBB3 |
| 6,987,218 | KW4U110 | 6,864,409 | WDDQ1 | 6,765,133 | RII1 | 6,686,519 | 11084BM | 6,444,883 | 6077 |
| 6,989,476 | 3633BM | 6,864,410 | N10018 | 6,770,802 | PH9AH | 6,686,520 | W60028 | 6,444,884 | I014738 |
| 6,989,477 | 5020 | 6,864,411 | PH6MN | 6,774,289 | PH51H | 6,693,231 | PH1GD | 6,452,074 | TDC1 |
| 6,989,478 | PH6HR | 6,872,873 | PH7BW | 6,774,290 | PH94T | 6,693,232 | LH295 | 6,452,075 | GF6151 |
| 7,002,063 | BT751-31 | 6,872,874 | PH890 | 6,777,599 | PH7AB | 6,700,041 | PH1BC | 6,452,076 | 7180 |
| 7,002,064 | A60059 | 6,878,863 | PH876 | 6,781,042 | PH5FW | 6,706,954 | PH4V6 | 6,455,764 | WQDS7 |
| 7,009,093 | PH183 | 6,878,864 | PHAPV | 6,781,043 | PH75K | 6,706,955 | NP2276 | 6,459,021 | X532Y |
| 7,012,177 | PH714 | 6,878,865 | PHB5R | 6,784,348 | KW7606 | 6,710,233 | NP2222 | 6,459,022 | I465837 |
| 7,015,386 | PHA9G | 6,881,880 | PH8DB | 6,784,349 | PH8CW | 6,717,036 | Ph0R8 | 6,469,232 | 1784S |
| 7,022,903 | W69079 | 6,881,881 | PH51K | 6,784,350 | PH8PG | 6,717,037 | PH581 | 6,469,233 | LH176Bt810 |
| 7,022,904 | PH8BC | 6,884,930 | 4XA321 | 6,797,869 | RBO1 | 6,717,038 | PH6WR | 6,469,234 | 6RC172 |
| 7,030,302 | PHBBP | 6,888,051 | PH87P | 6,803,509 | 9SM990 | 6,717,039 | PH5HK | 6,469,235 | 3327 |
| 7,034,213 | PHAKC | 6,897,361 | PH8KG | 6,806,408 | PH5TG | 6,717,040 | PH5W4 | 6,476,298 | 7SH382 |
| 7,041,884 | 291B | 6,897,363 | PH4CV | 6,806,409 | I501150 | 6,720,486 | PH0KT | 6,476,299 | I181664 |
| 7,041,885 | KWU7104 | 6,903,254 | PH705 | 6,806,410 | I390186 | 6,720,487 | PH4GP | 6,483,014 | NP2010 |
| 7,049,498 | 413A | 6,906,250 | LH331 | 6,809,240 | PH6JM | 6,723,900 | PHJ8R | 6,483,015 | FR3361 |
| 7,057,100 | W23129 | 6,906,251 | PH5DP | 6,809,243 | KW4636 | 6,723,901 | NP2052 | 6,486,386 | 1778S |
| 7,060,880 | G1704 | 6,909,036 | BX20010 | 6,809,244 | I363128 | 6,723,902 | PH7CP | 6,492,581 | I362697 |
| 7,060,880 | G1704 | 6,909,037 | BX20033 | 6,812,386 | LH246 | 6,723,903 | PH6WG | 6,506,964 | RPK7250 |
| RE38,768 | G1900 | 6,909,039 | PH77N | 6,812,387 | 2JK221 | 6,727,412 | PH54H | 6,911,588 | 6RT321 |
| 7,071,393 | PHB18 | 7,135,629 | 4XP811 | 7,166,776 | LH351 | 7,186,904 | I900429 | 7,208,660 | I292796 |
| 7,071,394 | PHD90 | 7,138,571 | SE8505 | 7,166,780 | I180421 | 7,186,906 | I139593 | 7,211,717 | I285291 |
| 7,071,395 | PHACV | 7,141,724 | I450436 | 7,166,779 | I294213 | 7,186,899 | I918166 | 7,211,716 | I082216 |
| 7,071,396 | PHCEG | 7,145,061 | G0603 | 7,166,778 | I104774 | 7,186,900 | I156024 | 7,214,863 | I130247 |
| 7,074,991 | PHB00 | 7,148,410 | LH306 | 7,166,783 | NP2391 | 7,186,902 | PH0GC | 7,214,864 | PH7JD |
| 7,078,601 | PHCND | 7,151,208 | LH268 | 7,169,981 | 1015036 | 7,189,904 | I285287 | 7,217,872 | I028727 |
| 7,078,602 | PHCMV | 7,154,031 | I119148 | 7,169,983 | HOI001 | 7,189,903 | I533766 | 7,217,873 | I135168 |
| 7,084,334 | CC2 | 7,157,624 | LHE323 | 7,173,171 | I180580 | 7,193,143 | LH332 | 7,217,871 | I216556 |
| 7,084,333 | CB1 | 7,157,630 | I119135 | 7,173,172 | I325369 | 7,193,144 | PHCJP | 7,220,900 | I180581 |
| 7,087,822 | PHADA | 715626 | I390171 | 7,176,361 | BE9514 | 7,199,290 | I090372 | 7,223,908 | LH324 |
| 7,087,820 | PHAJE | 7,157,628 | I322683 | 7,176,365 | I059908 | 7,199,291 | I136799 | 7,227,064 | PH8JV |
| 7,091,406 | PH8WD | 7,157,631 | NP2467 | 7,179,972 | I006605 | 7,202,402 | PH5CT | 7,227,063 | PHDWA |
| 7,091,407 | PH907 | 7,161,070 | I180576 | 7,179,969 | I181334 | 7,202,401 | 366C | 7,232,943 | I133314 |
| 7,091,405 | G4803 | 7,161,069 | HOI002 | 7,179,970 | PH7GD | 7,205,460 | I026458 | 7,232,945 | I226211 |
| 7,094,955 | N61060 | 7,164,068 | I113752 | 7,179,973 | GS05- | 7,205,459 | LIZL5 | 7,232,944 | I119163 |
| 7,094,954 | G0502 | 7,164,065 | I060064 | | 32431 | 7,205,464 | I135160 | 7,235,721 | MN7224 |
| 7,112,730 | W16090 | 7,164,070 | PHCER | 7,183,470 | LH321 | 7,205,463 | G1505 | 7,235,720 | LH370 |
| 7,122,725 | PH6WR | 7,166,785 | WBB53 | 7,183,471 | I000091 | 7,208,662 | I285302 | 7,235,722 | I130248 |
| 7,122,726 | NP2460 | 7,166,776 | LH311 | 7,186,901 | LH249 | 7,208,661 | I062695 | 7,235,726 | I170094 |
| 7,235,725 | I217345 | 7,235,723 | PHCAM | 7,186,903 | I029010 | 7,157,625 | I325350 | 7,109,403 | PHADP |
| 7,235,724 | I900105 | 7,241,944 | I119149 | 7,247,776 | G7403 | 7,109,404 | M10138 | | |
| | | | | 7,253,346 | I116412 | | | | |

TABLE 12
Examples of corn inbred lines suitable
for use in the present invention.

| Application No. | Inbred line |
|---|---|
| US20030093826A1 | CI9805 |
| US20030106086A1 | LH321 |
| US20030154524A1 | HOI002 |
| US20030172416A1 | HOI001 |
| US20030177541A1 | 5750 |
| US20030177543A1 | G0502 |
| US20030177544A1 | G1102 |
| US20040068771A1 | HX879 |
| US20040088767A1 | 6803 |
| US20040088768A1 | 5020 |
| US20040098768A1 | G3001 |
| US20040111770A1 | LH268 |
| US20040111771A1 | LH311 |
| US20040111772A1 | LH306 |
| US20040111773A1 | LH351 |
| US20040111774A1 | LHE323 |
| US20040123352A1 | 402A |
| US20040139491A1 | 366C |
| US20040143866A1 | NP2315 |
| US20040194170A1 | PH0GC |
| US20050015834A1 | SE8505 |
| US20050028236A1 | D201 |
| US20050076402A1 | BE1146BMR |
| US20050114944A1 | PHCAM |
| US20050114945A1 | PHCK5 |
| US20050114951A1 | PHC77 |
| 2005/0177894 | D501 |
| 2005/0177896 | D601 |
| 2005/0177904 | D603 |
| 2005/0076402 | BE11466MR |
| 2005/0125864 | 6XN442 |
| 2006/0070146 | BS112 |
| 2007/0033672 | BE9515 |
| 2006/0265778 | EX6077 |
| 2007/0028317 | I015011 |
| 2005/0028236 | D201 |
| 2007/0022490 | 3B603 |
| 2007/0022491 | 3B703 |
| 2007/0022492 | 3650HO |
| 2007/0028325 | W701BC |
| 2005/0114951 | PHC77 |
| 2007/0180557 | NP2405 |
| US20050114952A1 | PHCND |
| US20050114953A1 | PHCMV |
| US20050114955A1 | PHB00 |
| US20050114956A1 | PHCER |
| US20050120437A1 | PHCJP |
| US20050120439A1 | PHADA |
| US20050120443A1 | PHB8V |
| US20050125864A1 | 6XN442 |
| US20050125865A1 | 4XP811 |
| US20050125866A1 | PHCCW |
| US20050132433A1 | MN7224 |
| US20050132449A1 | BE9514 |
| US20050138697A1 | PHCA5 |
| US20050144687A1 | PHCPR |
| US20050144688A1 | PHAR1 |
| US20050144689A1 | PHACV |
| US20050144690A1 | PHEHG |
| US20050160487A1 | NP2391 |
| US20050172367A1 | PH8WD |
| US20050177894A1 | D501 |
| US20050177896A1 | D601 |
| US20050177904A1 | D603 |
| US20050223443A1 | PHCEG |
| US20050273876A1 | W16090 |
| US20050273877A1 | M10138 |
| US20050273878A1 | N61060 |
| 2005/0120443 | PHB8V |
| 2005/0125866 | PHCCW |
| 2005/0138697 | PHCA5 |
| 2005/0144687 | PHCPR |
| 2005/0144688 | PHAR1 |
| 2005/0144690 | PHEHG |
| 2006/0107398 | PHEWW |
| 2006/0107399 | PHEDR |
| 2006/0107400 | PHE67 |
| 2006/0107408 | PHE72 |
| 2006/01074110 | PHF1J |
| 2006/0107412 | PHE35 |
| 2006/0107415 | PHEHR |
| 2006/0107416 | PHDPP |
| 2006/0107418 | PHEHC |
| US20060048243A1 | NP2460 |
| US20060070146A1 | BS112 |
| US20060107393A1 | PHDWA |
| US20060107394A1 | PH8JV |
| US20060107398A1 | PHEWW |
| US20060107399A1 | PHEDR |
| US20060107400A1 | PHE67 |
| US20060107408A1 | PHE72 |
| US20060107410A1 | PHF1J |
| US20060107412A1 | PHE35 |
| US20060107415A1 | PHEHR |
| US20060107416A1 | PHDPP |
| US20060107418A1 | PHEHC |
| US20060107419A1 | PHANF |
| US20060107420A1 | PHC78 |
| US20060107421A1 | PH8T0 |
| US20060107422A1 | PHDRW |
| US20060107423A1 | PHEGV |
| US20060107426A1 | PHEBA |
| US20060112463A1 | PHENE |
| US20060112464A1 | PHEJW |
| US20060112465A1 | PHAPT |
| US20060130188A1 | PHCND |
| US20060130189A1 | PHCEG |
| US20060130190A1 | PHADA |
| US20060143744A1 | PHEED |
| 2006/0107419 | PHANF |
| 2006/0107420 | PHC78 |
| 2006/0107421 | PH8T0 |
| 2006/0107422 | PHDRW |
| 2006/0107423 | PHEGV |
| 2006/0107426 | PHEBA |
| 2006/0112463 | PHENE |
| 2006/0112464 | PHEJW |
| 2006/0112465 | PHAPT |
| 2006/0143744 | PHEED |
| 2007/0079402 | PHCK5 |
| 2007/0180553 | NP2482 |
| 2007/0180554 | NP2414 |
| 2007/0180555 | NP2546 |
| 2007/0180556 | NP2536 |

### EXAMPLES

The following examples are included to illustrate embodiments of the invention. It should be appreciated by those of skill in the art that the techniques disclosed in the examples that follow represent techniques discovered by the inventor to function well in the practice of the invention. However, those of skill in the art should, in light of the present disclosure, appreciate that many changes can be made in the specific embodiments which are disclosed and still obtain a like or similar result without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### Examples 1-12

Examples 1-12 are outlined in Table 13. In these examples several trait combinations for enhancing profitability of hybrid corn seed production system are provided. In general, traits having high impact on decreasing cost of production are provided on the female parent. These traits include herbicide tolerance, male sterility system, yield, and nutrient use efficiency. Traits having low impact on decreasing cost of production are provided on the male parent and/or the female parent. These traits include cold tolerance, drought tolerance, disease resistance, and insect resistance. For example, in example 5 or 6, by providing an enhanced yield, corn borer resistance, male sterility system trait, and herbicide tolerance on the female parent, producers will be able to decrease their cost of production by an estimated 28.8% (=14.2+10.8+3.7+0.1; based on Table 1). Similarly, as shown in example 7, by providing two herbicide tolerance traits, and 2 or 3 insect resistance traits on the female parent, producers will be able to decrease their cost of production by 28.6% or 28.7%.

Because the cost of breeding uneven number of traits into a hybrid is similar to the cost of breeding even number traits, it is of benefit to balance similar number of traits on each parent. The trait combinations exemplified here have a similar or identical number of traits on each parent, wherein similar is defined as±one or two or three traits to reduce the cost of breeding these traits into a hybrid. If a trait is found to have an adverse effect on a parent then that traits can be provided on another parent to remove the adverse effect.

Also, if a herbicide tolerance trait is used as a part of an MSS on the female parent then the same herbicide tolerance trait is also provided on the male parent as shown in examples 4-6, 9-12.

TABLE 13
Enhancing profitability of a hybrid corn seed production system by combining
traits having an impact on decreasing cost of production.

| | | | Percent | | |
|---|---|---|---|---|---|
| | | | decrease | | |
| Examples | Female Parent | | in cost | Male Parent | |
| 1 | G2 or CRW2-G2 | CB2 | 14.3/14.4 | CRW3 | CB3 |

| | | | | | | |
|---|---|---|---|---|---|---|
| 2 | G2 or CRW2-G2 | CB2 | 14.3/14.4 | D1 | CRW3 | CB3 |

| | | | | | | | |
|---|---|---|---|---|---|---|---|
| 3 | CB2 | MSS | 10.9 | D1 | G2 or CRW2-G2 | CRW3 | CB3 |

| | | | | | | | | |
|---|---|---|---|---|---|---|---|---|
| 4 | IY1 | CB2 | MSS | 14.6 | D1 | CRW4 | G2 | CB3 |

| | | | | | | | | | | | |
|---|---|---|---|---|---|---|---|---|---|---|---|
| 5 | NUE1 | IY1 | CB2 | MSS | 28.8 | D1 | CRW4 | G3 | CB3 | D2 | C1 |
| 6 | NUE1 | IY1 | CB4 | MSS | 28.8 | D1 | CRW4 | G3 | D2 | C1 | |

| 7 | G2 or CRW2-G2 | | | CB2 | CB3-Glu | 28.6/28.7 | CRW3-Glu | | | | | |
|---|---|---|---|---|---|---|---|---|---|---|---|---|
| 8 | G2 or CRW2-G2 | | | CB2 | CB3-Glu | 28.6/28.7 | CRW3-Glu | | | D1 | | |
| 9 | IY1 | CB2 | | CB3-Glu | MSS | 28.9 | D1 | | | G2 or CRW2-G2 | | |
| 10 | NUE1 | IY1 | CB2 | CB-3-Glu | MSS | 32.5 | D1 | G2 or CRW2-G2 | | CRW4 | D2 | |
| 11 | NUE1 | IY1 | CB2 | | MSS | 18.3 | C1 | D1 | CRW2-G2 | CRW4 | D2 | G3 |

| | | | | | | | | | | | |
|---|---|---|---|---|---|---|---|---|---|---|---|
| 12 | NUE1 | IY1 | CB2 or CB4 | MSS | 18.3 | C1 | D1 | CRW2-G2 | CRW4 | D2 | G3 |

Description of various abbreviations used for trait combinations is given below. The sources of trait genes or trait events are shown in parentheses, which are all incorporated herein by reference. G2: glyphosate tolerance mechanism 2 (Event NK603; U.S. Pat. No. 6,825,400); CRW2-G2: corn root worm resistance mechanism 2 (Event MON88017; WO05059103) linked to glyphosate tolerance mechanism 2 (Event NK603; U.S. Pat. No. 6,825,400); CB2: corn borer resistance mechanism 2 (Event MON89034; U.S. Application No. 60/808,834); CB3-Glu: corn borer resistance mechanism 3 and glufosinate tolerance (Event 1507; US20060037095, US 20050039226); CRW3-Glu: corn root worm resistance mechanism 3 and glufosinate tolerance (Event 59122; US20060070139); D1: drought tolerance mechanism 1 (Tables 2A and 2B to 9); MSS: male sterility system (Tables 2A and 2B); IY1: intrinsic yield mechanism 1 (Tables 2A and 2B to 9); CRW4: corn root worm resistance mechanism 4 (US20060021087); NUE1: nitrogen use efficiency mechanism 1 (Tables 2A and 2B to 9); G3: glyphosate tolerance mechanism 3; D2: drought tolerance mechanism 2 (Tables 2A and 2B to 9); C1: cold tolerance mechanism 1 (Tables 2A to 2B to 9); and CB4: corn borer resistance mechanism 4.

### Examples 13-28

Examples 13-28 are outlined in Table 14. In these examples several trait combinations for enhancing profitability of a hybrid corn seed production system are provided. In general, traits having high impact on decreasing cost of production are provided on the female parent. These traits include herbicide tolerance, male sterility system, yield, and nutrient use efficiency, Traits having low impact on decreasing cost of production are provided on the male parent or the female parent. These traits include cold tolerance, drought tolerance, diseases resistance, and insect resistance. In addition, end user traits, which are considered neutral in terms of enhancing profitability of a hybrid corn production system to a producer can be provided on the male and/or the female parent. These traits will be of benefit to the end users, such as farmers and processors. These traits may include, among others, enhanced amino acid, protein, fatty acid, carbohydrate, and oil content.

Because the cost of breeding an uneven number of traits into a hybrid is similar to the cost of breeding an even number traits, it is of benefit to balance similar number of traits on each parent. The traits combinations exemplified here have a similar number of traits on each parent, wherein similar means±one, two, or three traits to reduce the cost of breeding these traits into a hybrid. If a trait is found to have an adverse effect on a parent then that trait can be provided on another parent to remove the adverse effect. Also, a trait may be provided in a heterozygous state on each parent to remove the adverse effect such as the LI. Also, if a herbicide tolerance trait is used as a part of an MSS on the female parent then the same herbicide tolerance trait is also provided on the male parent as shown for instance in examples 17-20 and 24-27.

TABLE 14
Enhancing profitability of hybrid corn seed production system and end users by combining
traits having an impact on decreasing cost of production and end user traits.

| Example | Female Parent | | Male Parent | | |
|---|---|---|---|---|---|
| 13 | G2 | L1 | CB1 | CRW1 | L1 |

| | | | | |
|---|---|---|---|---|
| 14 | G2 or CRW2-G2 | L1 | CB1 | L1 |

| 15 | G2 or CRW2-G2 | | CB2 | | L1 | | L1 | | CRW3 | | CB3 | |
|---|---|---|---|---|---|---|---|---|---|---|---|---|
| 16 | G2 or CRW2-G2 | | CB2 | | L1 | | D1 | L1 | CRW3 | | CB3 | |
| 17 | CB2 | | L2 | | MSS | | D1 | L1 | G2 or CRW2-G2 | | CRW3 | CB3 |
| 18 | IY1 | CB2 | L2 | | MSS | | D1 | CRW4 | | G2 | | CB3 |
| 19 | NEU1 | IY1 | CB2 | L2-O | T-P | MSS | D1 | CRW4 | G3 | CB3 | D2 | C1 |
| 20 | NEU1 | IY1 | CB4 | L2-O | T-P | MSS | D1 | CRW4 | G3 | | D2 | C1 |

| | | |
|---|---|---|
| 21 | CB1 | L1 |

| | | | | | | | |
|---|---|---|---|---|---|---|---|
| 22 | CRW2-G2 | CB2 | | L1 | | | |
| 23 | CRW2-G2 | CB2 | CB3-Glu | D1 | CRW3-Glu | L1 | |
| 24 | CB2 | CB3-Glu | MSS | D1 | CRW2-G2 | CRW3-Glu | L1 |

| | | | | | | | | | |
|---|---|---|---|---|---|---|---|---|---|
| 25 | IY1 | CB2 | CB3-Glu | MSS | O | D1 | CRW2-G2 | CRW4 | L1 |

| | | | | | | | | | | | | |
|---|---|---|---|---|---|---|---|---|---|---|---|---|
| 26 | IY1 | CB2 | CB3-Glu | MSS | O | C1 | D1 | CRW2-G2 | CRW4 | D2 | G3 | L1 |

| | | | | | | | | | | | |
|---|---|---|---|---|---|---|---|---|---|---|---|
| 27 | IY1 | CB2 or CB4 | MSS | O | C1 | D1 | CRW2-G2 | CRW4 | D2 | G3 | L1 |

| | | | |
|---|---|---|---|
| 28 | CRW2-G2 | CB1 | L1 |

Descriptions of various abbreviations used for trait combinations are given below. The sources of trait genes or trait events are shown in parentheses, which are all incorporated herein by reference. G2: glyphosate tolerance mechanism 2 (Event NK603; U.S. Pat. No. 6,825,400); L1: enhanced lysine content mechanism 1 (Event LY038; US20050132437); CB1: corn borer resistance mechanism 1 (Event MON810; U.S. Pat. No. 6,713,259); CRW1—corn root worm resistance mechanism (Event MON863; US20060095986); CRW2-G2: corn root worm resistance mechanism 2 (Event MON88017; WO05059103) linked to glyphosate tolerance mechanism 2 (Event NK603; U.S. Pat. No. 6,825,400); CB3-Glu: corn borer resistance mechanism 3 and glufosinate tolerance (Event 1507; US20060037095, US 20050039226); CRW3-Glu: corn root worm resistance mechanism 3 and glufosinate tolerance (Event 59122; US20060070139); D1: drought tolerance mechanism 1 (Tables 2A and 2B to 9); L2: enhanced lysine content mechanism 2; MSS: male sterility system (Tables 2A and 2B); IY1: intrinsic yield mechanism 1 (Tables 2A and 2B to 9); NUE1: nitrogen use efficiency mechanism 1 (Tables 2A and 2B to 9); CRW4: corn root worm resistance mechanism 4; L2-O: enhanced lysine content mechanism 2 linked to enhanced oil content trait (U.S. Pat. No. 6,822,141); T-P: enhanced tryptophan content (US20030213010) linked to enhanced protein content; G3: glyphosate tolerance mechanism 3; D2: drought tolerance mechanism 2 (Tables 2A and 2B to 9); and C1: cold tolerance mechanism 1 (Tables 2A and 2B to 9).

All of the compositions and/or methods disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods and in the steps or in the sequence of steps of the method described herein without departing from the concept, spirit and scope of the invention. More specifically, it will be apparent that certain agents that are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

1-27. (canceled)

28. A method for producing hybrid corn seed with at least four transgenic traits, comprising:
a) providing a female parent comprising a transgenic event conferring tolerance to glyphosate herbicide and a transgenic event conferring a different herbicide tolerance trait that provides tolerance to a different herbicide selected from the group consisting of glufosinate, dicamba, and 2,4-D and a male parent comprising at least two transgenic events that confer at least two transgenic traits selected from the group consisting of an insect resistance trait, a drought tolerance trait, a nutrient use efficiency trait, a cold tolerance trait, an intrinsic yield trait, and a combination thereof, wherein the number of transgenic events is balanced on the female and male parents;
b) pollinating said female parent with pollen only from the male parent; and
c) allowing the female parent to produce hybrid seeds.

30. The method of claim 28, wherein the female parent further comprises at least one lepidopteran resistance trait.

31. The method of claim 28, wherein the female parent further comprises a transgenic event that confers a male sterility system trait.

32. The method of claim 28, wherein the female parent comprises a glyphosate tolerance trait and a glufosinate tolerance trait.

33. The method of claim 28, wherein one of the transgenic events of the male or female parent further confers an end user trait.

34. The method of claim 33, wherein the end user trait is selected from the group consisting of traits that enhance amino acid content, protein content, fatty acid composition, oil content, carbohydrate content, and a combination thereof, relative to a plant not comprising the end user trait.

35. A hybrid corn seed produced according to the method of claim 28, wherein the hybrid corn seed comprises said transgenic event conferring tolerance to glyphosate herbicide and said transgenic event conferring a different herbicide tolerance trait inherited from the female parent and said at least two transgenic events inherited from the male parent.

36. A plant produced by growing the hybrid seed of claim 35.

37. A commodity product produced from the plant of claim 36, wherein the product comprises a cell of said plant.

40. The method of claim 1, wherein the number of transgenic traits is balanced as ±one or two or three traits on the female or male parent.
